(19) United States
(12) Patent Application Publication
(10) Pub. No.: US 2024/0336622 A1
(43) Pub. Date: Oct. 10, 2024

(54) NIMBOLIDE ANALOGS AND METHODS OF USE THEREOF

(71) Applicant: The Board of Regents of The University of Texas System, Austin, TX (US)

(72) Inventors: Yonghao YU, Dallas, TX (US); Tian QIN, Dallas, TX (US); Yuanli ZHEN, Dallas, TX (US); Peng LI, Dallas, TX (US); Chiho KIM, Dallas, TX (US); Heping DENG, Dallas, TX (US); Hejun DENG, Dallas, TX (US)

(73) Assignee: The Board of Regents of The University of Texas System, Austin, TX (US)

(21) Appl. No.: 18/260,653

(22) PCT Filed: Jan. 8, 2022
(86) PCT No.: PCT/US2022/011733

§ 371 Date: Jul. 7, 2023

Related U.S. Application Data
(60) Provisional application No. 63/252,438, filed on Oct. 5, 2021.
(60) Provisional application No. 63/135,274, filed on Jan. 8, 2021.

(51) Int. Cl. C07D 493/06; A61K 31/365; A61K 31/443; A61K 45/06; A61P 35/00; C07D 307/77

(52) U.S. Cl. CPC C07D 493/06; A61K 31/365; A61K 31/443; A61K 45/06; A61P 35/00; C07D 307/77

(57) ABSTRACT

Disclosed herein are compounds of the formula (I) or (II) wherein the variables are defined herein. Also provided are methods of manufacturing and pharmaceutical compositions thereof. In some aspects, the compounds and compositions provided herein may be used as PARP1 inhibitors. In some aspects, the present disclosure provides methods wherein the compounds and compositions described herein are used for the treatment of diseases and disorders, such as cancer.

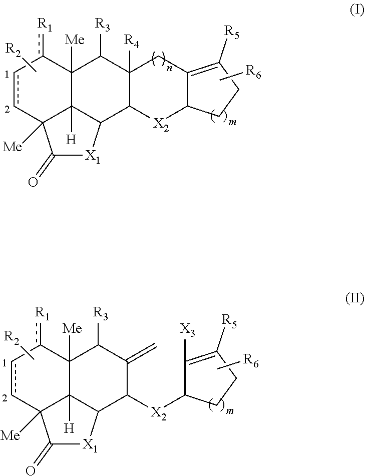

### NIMBOLIDE ANALOGS AND METHODS OF USE THEREOF

This application claims the benefit of priority to U.S. Provisional Applications No. 63/135,274, filed on Jan. 8, 2021 and 63/252,438, filed on Oct. 5, 2021, the entire contents of which are hereby incorporated by reference.

### GOVERNMENT SUPPORT CLAUSE

This invention was made with government support under Grant No. R35GM134883 and R01GM141088 awarded by the National Institute of General Medical Sciences. The government has certain rights in the invention.

### BACKGROUND

### I. Field

The present disclosure relates generally to the fields of biology, chemistry, and medicine. More particularly, it concerns compounds, compositions and methods for the treatment and prevention of diseases and disorders, such as cancer.

### II. Description of Related Art

Poly-ADP-ribosylation (PARylation) is an important protein post-translational modification (PTM), which is involved in an array of biological processes including cell stress response (Kraus, 2015). PARP1, the most studied member of the poly-ADP-ribose polymerases (PARPs), is responsible for the initiation of the DNA damage repair by inducing PARylation on numbers of proteins. Based on the theory of synthetic lethality (Bryant et al., 2005; Farmer et al., 2005), clinically developed PARPi have been approved by the FDA and widely used for the treatment of human BRCA-mutant malignancies (Ledermann et al., 2012; Litton et al., 2018; Mirza et al., 2016; Swisher et al., 2017).

All the clinical PARPi were developed to block PARylation by competitive binding to the NAD⁺-binding pocket of PARP1 and PARP2 (Rouleau et al., 2010). However, PARPi with equivalent potency as the inhibitors of PARP1 catalytic activity have dramatically different cytotoxicities in both BRCA-proficient and BCRA-deficient cells (Murai et al., 2012; Murai et al., 2014), thus, the mechanism of action of PARPi as anticancer agents has been reconsidered beyond the initial hypothesis that PARPi act as DNA repair inhibitors by blocking the catalytic activity of PARP. Now, it is widely accepted that the cytotoxicity of PARPi is driven by their ability and potency to stabilize PARP-DNA complexes at DNA lesions, which induces PARP1 trapping (Murai et al., 2012; Murai et al., 2014). A recent study further confirmed that clinical PARPi with different PARP1 trapping potency correspond to their cytotoxic potency (Murai et al., 2017). Therefore, all these studies suggest that PARPi should be categorized according to PARP1 trapping capability in addition to catalytic PARP1 inhibition, and a higher potency of PARP1 trapping will result in a higher potency of cytotoxicity against malignancies.

However, not all the BRCA-mutant tumors are sensitive to PARPi (Pommier et al., 2016). Particularly, some BRCA-mutant TNBC (triple negative breast cancer) cells are still resistant to PARPi treatment (Lehmann et al., 2011). In addition, long time treatment with PARPi also induced the drug-resistant effect among BRCA-mutant tumors. Therefore, identifying new targets to induce a higher potency of PARP1 trapping will be important for the understanding of the biological consequence of PARP1 trapping, and the impact of trapped PARP1 on DNA repair, DNA damage responses, and the lethality of cancer cells. In this context, determination of the mechanisms that regulate PARP1 dynamics is much needed for the development of new strategies to induce PARP1 trapping and overcome the PARPi-resistance of BRCA-deficient cancers. However, the mechanisms and regulators of PARP1 dynamics during DNA damage response still await further investigations. As such, there exists a significant need for additional agents for the treatment of cancers beyond PARPi.

### SUMMARY

The present disclosure provides synthetic nimbolide derivatives with anti-cancer properties, pharmaceutical compositions, and methods for their manufacture, and methods for their use.

In some aspects, the present disclosure provides compounds of the formula:

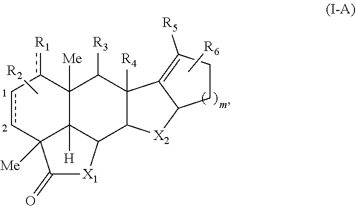

wherein:
the bond between atoms 1 and 2 is a single bond or a double bond;
m is 1 or 2;
X₁ and X₂ are each independently —O— or —NRₐ—, wherein:
   Rₐ is hydrogen, alkyl_{(C≤8)}, or substituted alkyl_{(C≤8)};
R₁ is hydroxy or oxo;
R₂ is hydrogen, amino, halo, hydroxy; or
   alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
   Y₁ is alkanediyl_{(C≤8)} or substituted alkanediyl_{(C≤8)};
   R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₄ is hydrogen, alkyl_{(C≤8)}, or substituted alkyl_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
   -A₁-R₇, wherein:
      A₁ is heteroarenediyl_{(C≤12)} or substituted heteroarenediyl_{(C≤12)};
      R₇ is hydrogen, amino, hydroxy, or an amino protecting group; or
         alkyl_{(C≤8)}, cycloalkyl_{(C≤8)}, heterocycloalkyl_{(C≤12)}, heteroaryl_{(C≤12)}, arylsulfonyloxy_{(C≤18)}, or a substituted version of any of these groups; or
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and Rd are each independently a monovalent amino protecting group, or R_{c} and R_{d} are taken together and are a divalent amino protecting group;
a group of the formula:

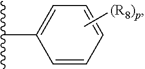

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)},
         alkylamino_{(C≤8)}, or
         dialkylamino_{(C≤8)}, or a substituted version of any of these groups; or
a group of the formula:

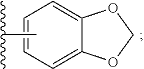

or
A₂-R₉, wherein:
   A₂ is cycloalkanediyl_{(C≤8)}, heterocycloalkanediyl_{(C≤8)}, arenediyl_{(C≤12)} or a substituted version of any of these groups;
   R₉ is hydrogen, amino, halo, hydroxy, or an amino protecting group; or alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤18)}, or a substituted version of any of these groups;
      a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

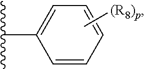

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

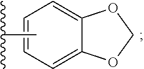

or
R₆ is a group of the formula:

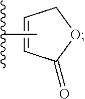

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
provided the compound is not:

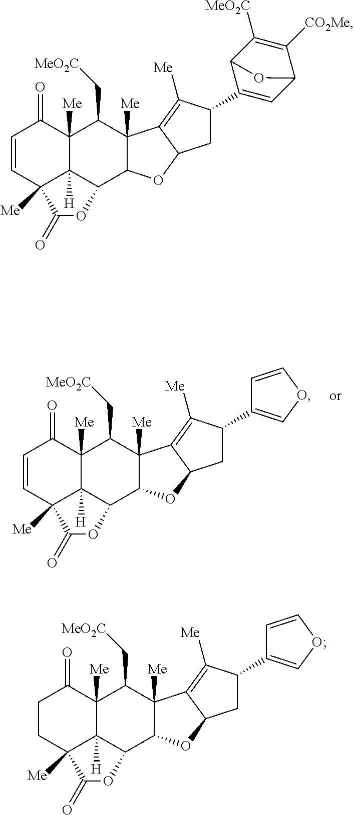

or
a compound of the formula:

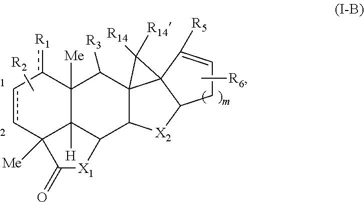

wherein:
the bond between atoms 1 and 2 is a single bond or a double bond;
m is 1 or 2;
X₁ and X₂ are each independently —O— or —NRₐ—, wherein:
   Rₐ is hydrogen, alkyl_{(C≤8)}, or substituted alkyl_{(C≤8)};
R₁ is hydroxy or oxo;
R₂ is hydrogen, amino, halo, hydroxy; or
   alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
   Y₁ is alkanediyl_{(C≤8)} or substituted alkanediyl_{(C≤8)};
   R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₅ is hydrogen, alkyl_{(C≤8)}, substituted alkyl_{(C≤8)}, or R₅ is taken together with R₆ as defined below;
R₆ is hydrogen, amino, halo, or hydroxy; or
   -A₁-R₇, wherein:
      A₁ is heteroarenediyl_{(C≤12)} or substituted heteroarenediyl_{(C≤12)}; and
      R₇ is hydrogen, amino, hydroxy, or an amino protecting group; or
         alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and Rd are taken together and are a divalent amino protecting group;
a group of the formula:

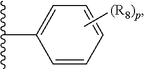

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

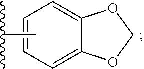

or
-A₂-R₉, wherein:
   A₂ is cycloalkanediyl_{(C≤8)}, heterocycloalkanediyl_{(C≤8)}, arenediyl_{(C≤12)} or a substituted version of any of these groups; and
   R₉ is hydrogen, amino, halo, hydroxy, or an amino protecting group; or alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤18)}, or a substituted version of any of these groups;
      a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

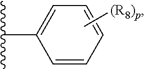

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, acyloxy_{(C≤8)}, alkylsilyloxy_{(C≤8)}, alkylamino_{(C≤8)}, or
      dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

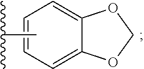

or
R₆ is a group of the formula:

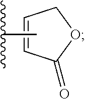

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)}; and
R₁₄ and R_{14′} are each independently hydrogen, alkyl_{(C≤8)}, or substituted alkyl_{(C≤8)}; or
   a compound of the formula:

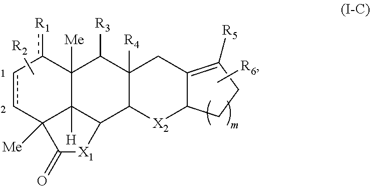

wherein:
the bond between atoms 1 and 2 is a single bond or a double bond;
m is 1 or 2;
X₁ and X₂ are each independently —O— or —NRₐ—, wherein:
   Rₐ is hydrogen, alkyl_{(C≤8)}, or substituted alkyl_{(C≤8)};
R₁ is hydroxy or oxo;
R₂ is hydrogen, amino, halo, hydroxy; or
   alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
   Y₁ is alkanediyl_{(C≤8)} or substituted alkanediyl_{(C≤8)};
   R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₄ is hydrogen, alkyl_{(C≤8)}, or substituted alkyl_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
   -A₁-R₇, wherein:
      A₁ is heteroarenediyl_{(C≤12)} or substituted heteroarenediyl_{(C≤12)};
      R₇ is hydrogen, amino, hydroxy, or an amino protecting group; or alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

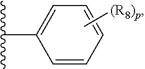

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

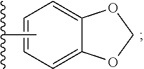

or
-A₂-R₉, wherein:
   A₂ is cycloalkanediyl_{(C≤8)}, heterocycloalkanediyl_{(C≤8)}, arenediyl_{(C≤12)} or a substituted version of any of these groups;
   R₉ is hydrogen, amino, halo, hydroxy, or an amino protecting group; or alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤8)}, or a substituted version of any of these groups;
      a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

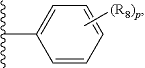

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

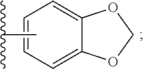

or
R₆ is a group of the formula:

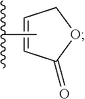

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)}; or
   a compound of the formula:

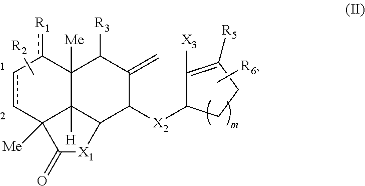

wherein:
the bond between atoms 1 and 2 is a single bond or a double bond;
m is 1 or 2;
n is 0 or 1;
X₁ and X₂ are each independently —O— or —NRₐ—, wherein:
   Rₐ is hydrogen, alkyl_{(C≤8)}, or substituted alkyl_{(C≤8)};
X₃ is hydrogen, amino, halo, or hydroxy;
R₁ is hydroxy or oxo;
R₂ is hydrogen, amino, halo, hydroxy; or
   alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
   Y₁ is alkanediyl_{(C≤8)} or substituted alkanediyl_{(C≤8)};
   R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
   -A₃-R₁₀, wherein:
      A₃ is cycloalkanediyl_{(C≤8)}, heterocycloalkanediyl_{(C≤8)}, arenediyl_{(C≤12)} heteroarenediyl_{(C≤12)}, or a substituted version of any of these groups; and
      R₁₀ is hydrogen, amino, hydroxy, or an amino protecting group; or alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

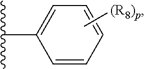

wherein:
   p is 0, 1, 2, 3, 4, or 5; and
   R₈ is, in each instance independently, hydrogen, hydroxy, halo, amino, cyano, or nitro; or alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

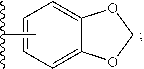

or
R₆ is a group of the formula:

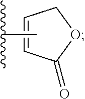

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
   or a pharmaceutically accepted salt of any of these formulae.

In some embodiments, the compounds are further defined as:

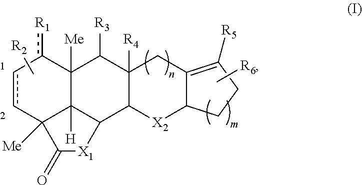

wherein:
the bond between atoms 1 and 2 is a single bond or a double bond;
m is 1 or 2;
n is 0 or 1;
X₁ and X₂ are each independently —O— or —NRₐ—, wherein:
   Rₐ is hydrogen, alkyl_{(C≤8)}, or substituted alkyl_{(C≤8)};
R₁ is hydroxy or oxo;
R₂ is hydrogen, amino, halo, hydroxy; or
   alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
   Y₁ is alkanediyl_{(C≤8)} or substituted alkanediyl_{(C≤8)};
   R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₄ is hydrogen, alkyl_{(C≤8)}, or substituted alkyl_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
   -A₁-R₇, wherein:
      A₁ is heteroarenediyl_{(C≤12)} or substituted heteroarenediyl_{(C≤12)};
      R₇ is hydrogen, amino, hydroxy, or an amino protecting group; or alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

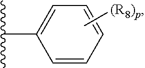

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

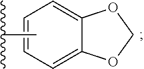

or
-A₂-R₉, wherein:
   A₂ is cycloalkanediyl_{(C≤8)}, heterocycloalkanediyl_{(C≤8)}, arenediyl_{(C≤12)} or a substituted version of any of these groups;
   R₉ is hydrogen, amino, halo, hydroxy, or an amino protecting group; or alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤18)}, or a substituted version of any of these groups;
      a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

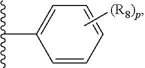

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

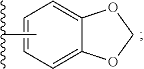

or
R₅ is a group of the formula:

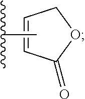

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
   provided the compound is not:

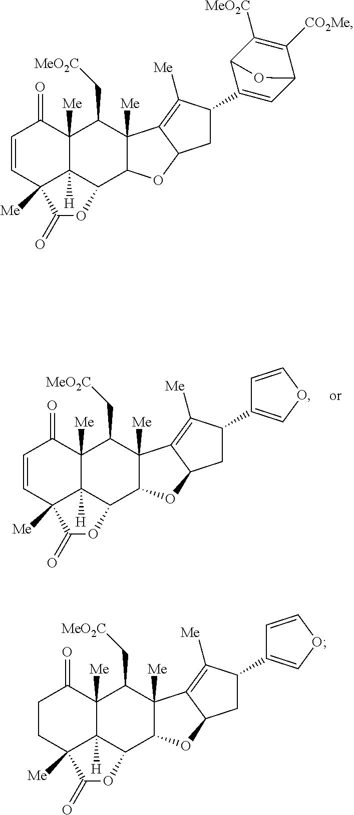

or
or compounds of the formula:

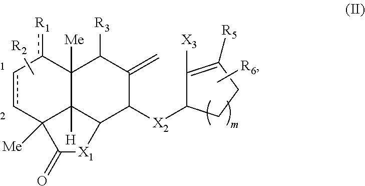

wherein:
the bond between atoms 1 and 2 is a single bond or a double bond;
m is 1 or 2;
X₁ and X₂ are each independently —O— or —NRₐ—, wherein:
   Rₐ is hydrogen, alkyl_{(C≤8)}, or substituted alkyl_{(C≤8)};
X₃ is hydrogen, amino, halo, or hydroxy;
R₁ is hydroxy or oxo;
R₂ is hydrogen, amino, halo, hydroxy; or
   alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
   Y₁ is alkanediyl_{(C≤8)} or substituted alkanediyl_{(C≤8)};
   R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
   -A₃-R₁₀, wherein:
      A₃ is cycloalkanediyl_{(C≤8)}, heterocycloalkanediyl_{(C≤8)}, arenediyl_{(C≤12)} heteroarenediyl_{(C≤12)}, or a substituted version of any of these groups;
      R₁₀ is hydrogen, amino, halo, hydroxy, or an amino protecting group; or alkyl_{(C≤12)}, cycloalkyl_{(C≤12)}, heterocycloalkyl_{(C≤12)}, aryl_{(C≤12)}, heteroaryl_{(C≤12)}, arylsulfonyloxy_{(C≤8)}, or a substituted version of any of these groups;
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

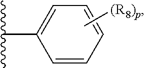

wherein:
   p is 0, 1, 2, 3, 4, or 5; and
   R₈ is, in each instance independently, hydrogen, hydroxy, halo, amino, cyano, or nitro; or alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

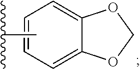

or
R₆ is a group of the formula:

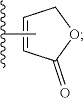

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically accepted salt of either of these formulae.

In some embodiments, the compounds are further defined as:

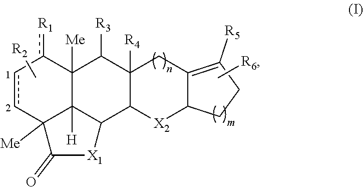

wherein:
the bond between atoms 1 and 2 is a single bond or a double bond;
m is 1 or 2;
n is 0 or 1;
X₁ and X₂ are each independently —O— or —NRₐ—, wherein:
   Rₐ is hydrogen, alkyl_{(C≤8)}, or substituted alkyl_{(C≤8)};
R₁ is hydroxy or oxo;
R₂ is hydrogen, amino, halo, hydroxy; or
   alkyl_{(C≤8)} or substituted alkyl_{(C≤8)};
R₃ hydrogen, alkyl_{(C≤8)}, substituted alkyl_{(C≤8)}, or —Y₁C(O)R_{b}, wherein:
   Y₁ is alkanediyl_{(C≤8)} or substituted alkanediyl_{(C≤8)};
   R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₄ is hydrogen, alkyl_{(C≤8)}, or substituted alkyl_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
   -A₁-R₇, wherein:
      A₁ is heteroarenediyl_{(C≤12)} or substituted heteroarenediyl_{(C≤12)};
      R₇ is hydrogen, amino, hydroxy, or an amino protecting group; or
         alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

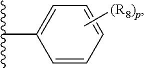

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

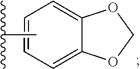

or
-A₂-R₉, wherein:
   A₂ is cycloalkanediyl_{(C≤8)}, heterocycloalkanediyl_{(C≤8)}, arenediyl_{(C≤12)} or a substituted version of any of these groups;
   R₉ is hydrogen, amino, halo, hydroxy, or an amino protecting group; or alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤18)}, or a substituted version of any of these groups;
      a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

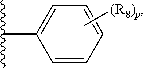

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

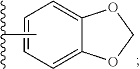

or
R₆ is a group of the formula:

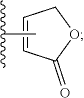

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
provided the compound is not:

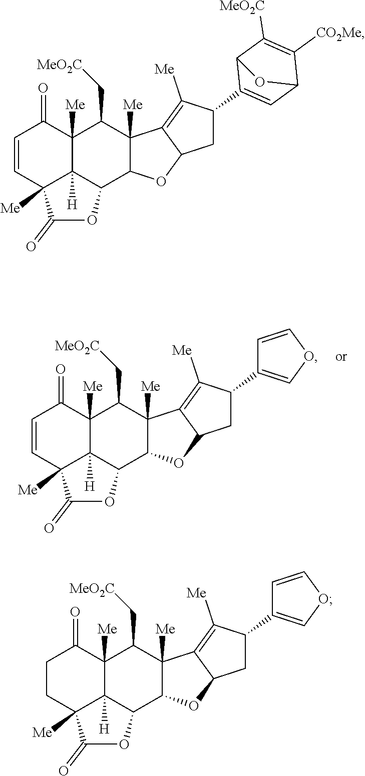

or pharmaceutically acceptable salts thereof.

In some embodiments, the compounds are further defined as:

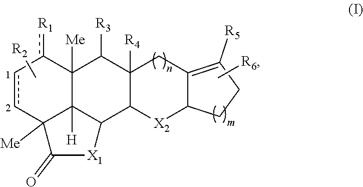

wherein:
the bond between atoms 1 and 2 is a single bond or a double bond;
m is 1 or 2;
n is 0 or 1;
X₁ and X₂ are each independently —O— or —NRₐ—, wherein:
   Rₐ is hydrogen, alkyl_{(C≤8)}, or substituted alkyl_{(C≤8)};
R₁ is hydroxy or oxo;
R₂ is hydrogen, amino, halo, hydroxy; or
   alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
   Y₁ is alkanediyl_{(C≤8)} or substituted alkanediyl_{(C≤8)};
   R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₄ is hydrogen, alkyl_{(C≤8)}, or substituted alkyl_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
   A₁-R₇, wherein:
      A₁ is an oxygen-containing heteroarenediyl_{(C≤12)} or a substituted oxygen-containing heteroarenediyl_{(C≤12)};
      R₇ is hydrogen, amino, hydroxy, or an amino protecting group; or
         alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

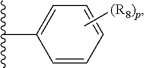

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

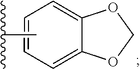

or
-A₂-R₉, wherein:
   A₂ is a nitrogen-containing heteroarenediyl_{(C≤12)}, cycloalkanediyl_{(C≤8)}, heterocycloalkanediyl_{(C≤8)}, arenediyl_{(C≤12)} or a substituted version of any of these groups;
   R₉ is hydrogen, amino, halo, hydroxy, or an amino protecting group; or alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤18)}, or a substituted version of any of these groups;
      a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

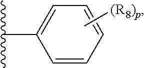

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, acyloxy_{(C≤8)}, alkylsilyloxy_{(C≤8)}, alkylamino_{(C≤8)}, or
      dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

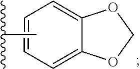

or
R₆ is a group of the formula:

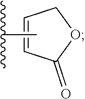

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
   or a pharmaceutically accepted salt thereof.

In some embodiments, the compounds are further defined as:

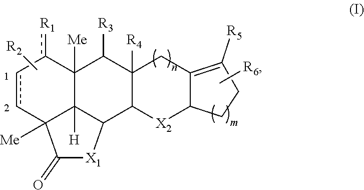

wherein:
the bond between atoms 1 and 2 is a single bond or a double bond;
m is 1 or 2;
n is 0 or 1;
X₁ and X₂ are each independently —O— or —NRₐ—, wherein:
   Rₐ is hydrogen, alkyl_{(C≤8)}, or substituted alkyl_{(C≤8)};
R₁ is hydroxy or oxo;
R₂ is hydrogen, amino, halo, hydroxy; or
   alkyl_{(C≤8)} or substituted alkyl_{(C≤8)};
R₃ hydrogen, alkyl_{(C≤8)}, substituted alkyl_{(C≤8)}, or —Y₁C(O)R_{b}, wherein:
   Y₁ is alkanediyl_{(C≤8)} or substituted alkanediyl_{(C≤8)};
   R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₄ is hydrogen, alkyl_{(C≤8)}, or substituted alkyl_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
   -A₁-R₇, wherein:
      A₁ is heteroarenediyl_{(C≤12)} or substituted heteroarenediyl_{(C≤12)};
      R₇ is hydrogen, amino, hydroxy, or an amino protecting group; or
         alkyl_{(C≤8)}, cycloalkyl_{(C≤8)}, heterocycloalkyl_{(C≤8)}, heteroaryl_{(C≤12)}, arylsulfonyloxy_{(C≤8)}, or a substituted version of any of these groups; or
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

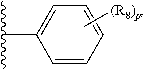

wherein:
   p is 0, 1, 2, 3, 4, or 5; and
   R₈ is, in each instance independently, hydrogen, hydroxy, halo, amino, cyano, or nitro; or alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

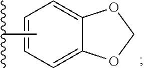

or
-A₂-R₉, wherein:
   A₂ is cycloalkanediyl_{(C≤8)}, heterocycloalkanediyl_{(C≤8)}, arenediyl_{(C≤12)} or a substituted version of any of these groups;
   R₉ is hydrogen, amino, halo, hydroxy, or an amino protecting group; or alkyl_{(C≤12)}, cycloalkyl_{(C≤12)}, heterocycloalkyl_{(C≤12)}, aryl_{(C≤12)}, heteroaryl_{(C≤12)}, arylsulfonyloxy_{(C≤18)}, or a substituted version of any of these groups;
      a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

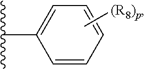

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

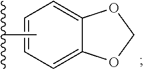

or
R₆ is a group of the formula:

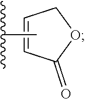

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
   or pharmaceutically acceptable salts thereof.

In some embodiments, the compounds are further defined as:

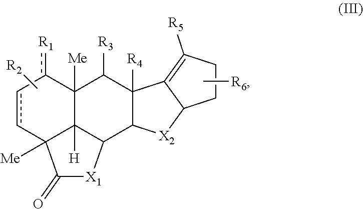

wherein:
the bond between atoms 1 and 2 is a single bond or a double bond;
X₁ and X₂ are each independently —O— or —NRₐ—, wherein:
   Rₐ is hydrogen, alkyl_{(C≤8)}, or substituted alkyl_{(C≤8)};
R₁ is hydroxy or oxo;
R₂ is hydrogen, amino, halo, hydroxy; or
   alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
   Y₁ is alkanediyl_{(C≤8)} or substituted alkanediyl_{(C≤8)};
   R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₄ is hydrogen, alkyl_{(C≤8)}, or substituted alkyl_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
   -A₁-R₇, wherein:
      A₁ is heteroarenediyl_{(C≤12)} or substituted heteroarenediyl_{(C≤12)};
      R₇ is hydrogen, amino, hydroxy, or an amino protecting group; or alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

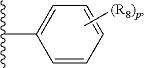

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

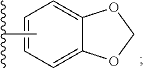

or
-A₂-R₉, wherein:
   A₂ is cycloalkanediyl_{(C≤8)}, heterocycloalkanediyl_{(C≤8)}, arenediyl_{(C≤12)} or a substituted version of any of these groups;
   R₉ is hydrogen, amino, halo, hydroxy, or an amino protecting group; or alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤18)}, or a substituted version of any of these groups;
      a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

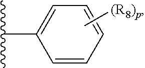

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

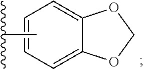

or
R₆ is a group of the formula:

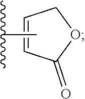

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
   or pharmaceutically acceptable salts thereof.

In some embodiments, the compounds are further defined as:

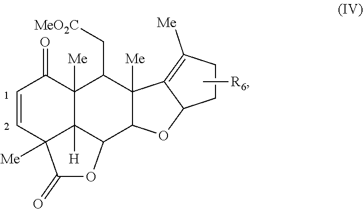

wherein:
R₆ is hydrogen, amino, halo, or hydroxy; or
   -A₁-R₇, wherein:
      A₁ is heteroarenediyl_{(C≤12)} or substituted heteroarenediyl_{(C≤12)};
      R₇ is hydrogen, amino, hydroxy, or an amino protecting group; or
         alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

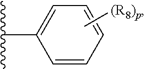

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

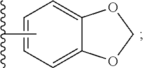

or
-A₂-R₉, wherein:
   A₂ is cycloalkanediyl_{(C≤8)}, heterocycloalkanediyl_{(C≤8)}, arenediyl_{(C≤12)} or a substituted version of any of these groups;
   R₉ is hydrogen, amino, halo, hydroxy, or an amino protecting group; or alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤18)}, or a substituted version of any of these groups;
      a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

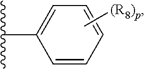

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

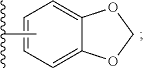

or
R₆ is a group of the formula:

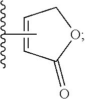

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
   or pharmaceutically acceptable salts thereof.

In some embodiments, the compounds are further defined as:

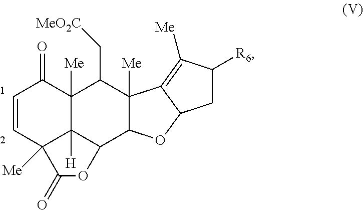

wherein:
R₆ is hydrogen, amino, halo, or hydroxy; or
   -A₁-R₇, wherein:
      A₁ is heteroarenediyl_{(C≤12)} or substituted heteroarenediyl_{(C≤12)};
      R₇ is hydrogen, amino, hydroxy, or an amino protecting group; or alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

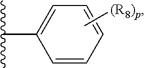

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

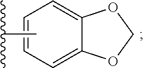

or
-A₂-R₉, wherein:
   A₂ is cycloalkanediyl_{(C≤8)}, heterocycloalkanediyl_{(C≤8)}, arenediyl_{(C≤12)} or a substituted version of any of these groups;
   R₉ is hydrogen, amino, halo, hydroxy, or an amino protecting group; or alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤18)}, or a substituted version of any of these groups;
      a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group; R_{c} and R_{d} are each independently a monovalent amino protecting group, or R_{c} and R_{d} are taken together and are a divalent amino protecting group;
a group of the formula:

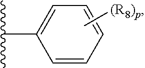

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, acyloxy_{(C≤8)}, alkylsilyloxy_{(C≤8)}, alkylamino_{(C≤8)}, or
      dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

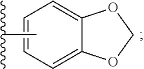

or
R₆ is a group of the formula:

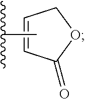

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
   or pharmaceutically acceptable salts thereof.

In some embodiments, the compounds are further defined as:

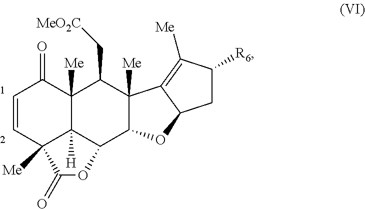

wherein:
R₆ is hydrogen, amino, halo, or hydroxy; or
   -A₁-R₇, wherein:
      A₁ is heteroarenediyl_{(C≤12)} or substituted heteroarenediyl_{(C≤12)};
      R₇ is hydrogen, amino, hydroxy, or an amino protecting group; or alkyl_{(C≤8)}, cycloalkyl_{(C≤8)}, heterocycloalkyl_{(C≤8)}, heteroaryl_{(C≤12)}, or a substituted version of any of these groups; or
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

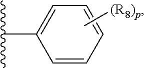

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

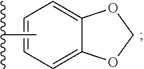

or
-A₂-R₉, wherein:
   A₂ is cycloalkanediyl_{(C≤8)}, heterocycloalkanediyl_{(C≤8)}, arenediyl_{(C≤12)} or a substituted version of any of these groups;
   R₉ is hydrogen, amino, halo, hydroxy, or an amino protecting group; or alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤18)}, or a substituted version of any of these groups;
      a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

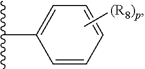

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

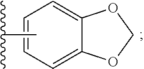

or
R₆ is a group of the formula:

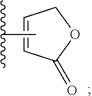

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or pharmaceutically acceptable salts thereof.

In some embodiments, the compounds are of formula (I-A). In other embodiments, the compounds are of formula (I-B). In still other embodiments, the compounds are of formula (I-C).

In some embodiments, the compounds are further defined as:

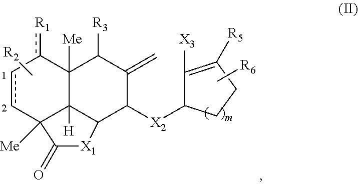

wherein:
   the bond between atoms 1 and 2 is a single bond or a double bond;
   m is 1 or 2;
   X₁ and X₂ are each independently —O— or —NRₐ—, wherein:
      Rₐ is hydrogen, alkyl_{(C≤8)}, or substituted alkyl_{(C≤8)};
   X₃ is hydrogen, amino, halo, or hydroxy;
   R₁ is hydroxy or oxo;
   R₂ is hydrogen, amino, halo, hydroxy; or
      alkyl_{(C≤8)} or substituted alkyl_{(C≤8)};
   R₃ is hydrogen, alkyl_{(C≤8)}, substituted alkyl_{(C≤8)}, or —Y₁C(O)R_{b}, wherein:
      Y₁ is alkanediyl_{(C≤8)} or substituted alkanediyl_{(C≤8)};
      R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
   R₅ is hydrogen, alkyl_{(C≤8)}, substituted alkyl_{(C≤8)}, or R₅ is taken together with R₆ as defined below; and
   R₆ is hydrogen, amino, halo, or hydroxy; or
      A₃-R₁₀, wherein:
         A₃ is cycloalkanediyl_{(C≤8)}, heterocycloalkanediyl_{(C≤8)}, arenediyl_{(C≤12)} heteroarenediyl_{(C≤12)}, or a substituted version of any of these groups;
         R₁₀ is hydrogen, amino, hydroxy, or an amino protecting group; or
         alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or
   substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

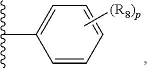

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups;
      or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

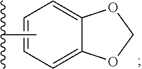

or
R₆ is a group of the formula:

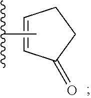

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
   or a pharmaceutically accepted salt thereof.

In some embodiments, the compounds are further defined as:

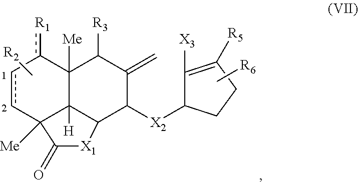

wherein:
   the bond between atoms 1 and 2 is a single bond or a double bond;
   X₁ and X₂ are each independently —O— or —NRₐ—, wherein:
      Rₐ is hydrogen, alkyl_{(C≤8)}, or substituted alkyl_{(C≤8)};
   X₃ is hydrogen, amino, halo, or hydroxy;
   R₁ is hydroxy or oxo;
   R₂ is hydrogen, amino, halo, hydroxy; or
      alkyl_{(C≤8)} or substituted alkyl_{(C≤8)};
   R₃ is hydrogen, alkyl_{(C≤8)}, substituted alkyl_{(C≤8)}, or —Y₁C(O)R_{b}, wherein:
      Y₁ is alkanediyl_{(C≤8)} or substituted alkanediyl_{(C≤8)};
      R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
   R₅ is hydrogen, alkyl_{(C≤8)}, substituted alkyl_{(C≤8)}, or R₅ is taken together with R₆ as defined below; and
   R₆ is hydrogen, amino, halo, or hydroxy; or
      A₃-R₁₀, wherein:
         A₃ is cycloalkanediyl_{(C≤8)}, heterocycloalkanediyl_{(C≤8)}, arenediyl_{(C≤12)} heteroarenediyl_{(C≤12)}, or a substituted version of any of these groups;
         R₁₀ is hydrogen, amino, hydroxy, or an amino protecting group; or
         alkyl_{(C≤8)}, cycloalkyl_{(C≤8)}, heterocycloalkyl_{(C≤8)}, heteroaryl_{(C≤12)}, arylsulfonyloxy_{(C≤8)}, or a substituted version of any of these groups; or
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

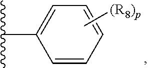

wherein:
   p is 0, 1, 2, 3, 4, or 5; and
   R₈ is, in each instance independently, hydrogen, hydroxy, halo, amino, cyano, or nitro; or alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups;
      or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

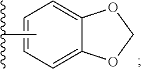

or
R₆ is a group of the formula:

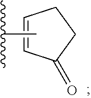

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically accepted salt thereof.

In some embodiments, the compounds are further defined as:

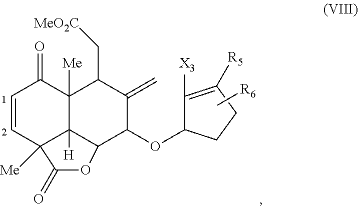

wherein:
   X₃ is hydrogen, amino, halo, or hydroxy;
   R₅ is hydrogen, alkyl_{(C≤8)}, substituted alkyl_{(C≤8)}, or R₅ is taken together with R₆ as defined below; and
   R₆ is hydrogen, amino, halo, or hydroxy; or
      A₃-R₁₀, wherein:
         A₃ is cycloalkanediyl_{(C≤8)}, heterocycloalkanediyl_{(C≤8)}, arenediyl_{(C≤12)} heteroarenediyl_{(C≤12)}, or a substituted version of any of these groups;
         R₁₀ is hydrogen, amino, hydroxy, or an amino protecting group; or
         alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

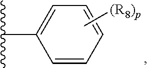

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups;
      or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

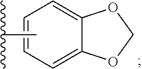

or
R₆ is a group of the formula:

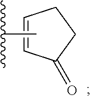

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically accepted salt thereof.

In some embodiments, the compounds are further defined as:

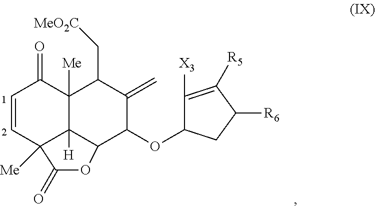

wherein:
   X₃ is hydrogen, amino, halo, or hydroxy;
   R₅ is hydrogen, alkyl_{(C≤8)}, substituted alkyl_{(C≤8)}, or R₅ is taken together with R₆ as defined below; and
   R₆ is hydrogen, amino, halo, or hydroxy; or
      -A₃-R₁₀, wherein:
         A₃ is cycloalkanediyl_{(C≤8)}, heterocycloalkanediyl_{(C≤8)}, arenediyl_{(C≤12)} heteroarenediyl_{(C≤12)}, or a substituted version of any of these groups;
         R₁₀ is hydrogen, amino, hydroxy, or an amino protecting group; or
         alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

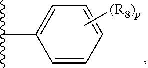

wherein:
   p is 0, 1, 2, 3, 4, or 5; and
   R₈ is, in each instance independently, hydrogen, hydroxy, halo, amino, cyano, or nitro; or alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or dialkylamino_{(C≤8)}, or a substituted version of any of these groups; or
a group of the formula:

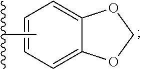

or
R₆ is a group of the formula:

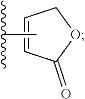

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically accepted salt thereof.

In some embodiments, the compounds are further defined as:

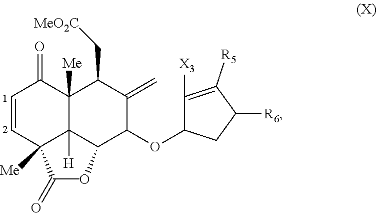

wherein:
   X₃ is hydrogen, amino, halo, or hydroxy;
   R₅ is hydrogen, alkyl_{(C≤8)}, substituted alkyl_{(C≤8)}, or R₅ is taken together with R₆ as defined below; and
   R₆ is hydrogen, amino, halo, or hydroxy; or
      -A₃-R₁₀, wherein:
         A₃ is cycloalkanediyl_{(C≤8)}, heterocycloalkanediyl_{(C≤8)}, arenediyl_{(C≤12)} heteroarenediyl_{(C≤12)}, or a substituted version of any of these groups;
         R₁₀ is hydrogen, amino, hydroxy, or an amino protecting group; or
         alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
         a group of the formula:

—(CH₂)ₓC(O)Rₐ
wherein:
   x is 0, 1, or 2;
   Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:

—(CH₂)_{y}OR_{b}

or

—(CH₂)_{y}NR_{c}R_{d}
wherein:
   y is 0, 1, or 2
   R_{b} is a hydroxy protecting group;
   R_{c} and R_{d} are each independently a monovalent amino protecting group, or R_{c} and R_{d} are taken together and are a divalent amino protecting group;
a group of the formula:

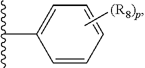

wherein:
   p is 0, 1, 2, 3, 4, or 5; and
   R₈ is, in each instance independently, hydrogen, hydroxy, halo, amino, cyano, or nitro; or alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups; or
a group of the formula:

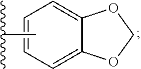

or
R₆ is a group of the formula:

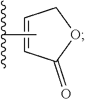

or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically accepted salt thereof.

In some embodiments, m is 1. In some embodiments, n is 0. In other embodiments, n is 1. In some embodiments, the bond between atoms 1 and 2 is a single bond. In other embodiments, the bond between atoms 1 and 2 is a double bond. In some embodiments, R₁ is hydroxy. In other embodiments, R₁ is oxo. In some embodiments, R₂ is hydrogen. In some embodiments, R₃ is alkyl_{(C≤8)} or substituted alkyl_{(C≤8)}. In further embodiments, R₃ is substituted alkyl_{(C≤8)}, such as (methoxycarbonyl)methyl. In other embodiments, R₃ is —Y₁C(O)R_{b}. In some embodiments, Y₁ is alkanediyl_{(C≤8)} such as —CH₂—. In some embodiments, R_{b} is alkoxy_{(C≤8)} such as methoxy or t-butoxy.

In some embodiments, R₄ is alkyl_{(C≤8)} or substituted alkyl_{(C≤8)}. In further embodiments, R₄ is alkyl_{(C≤8)}, such as methyl. In some embodiments, R₅ is alkyl_{(C≤8)} or substituted alkyl_{(C≤8)}. In further embodiments, R₅ is alkyl_{(C≤8)}, such as methyl or isopropyl.

In some embodiments, R₆ is hydrogen. In some embodiments, A₂ is heterocycloalkanediyl_{(C≤8)} or substituted heterocycloalkanediyl_{(C≤8)}. In further embodiments, A₂ is substituted heterocycloalkanediyl_{(C≤8)}, such as 2-acetoxy-5-oxo-2,5-dihydrofuran-2,3-diyl. In some embodiments, A₂ is cycloalkanediyl_{(C≤8)} or substituted cycloalkanediyl_{(C≤8)}. In further embodiments, A₂ is cycloalkanediyl_{(C≤8)}, such as cyclopentanediyl or cyclohexanediyl. In some embodiments, A₂ is arenediyl_{(C≤12)} or substituted arenediyl_{(C≤12)}. In further embodiments, A₂ is arenediyl_{(C≤12)}, such as benzenediyl. In other embodiments, A₂ is substituted arenediyl_{(C≤12)}, such as 4-methoxybenzen-1,3-diyl. In some embodiments, R₉ is hydrogen.

In some embodiments, A₁ is heteroarenediyl_{(C≤12)} or substituted heteroarenediyl_{(C≤12)}. In further embodiments, A₁ is heteroarenediyl_{(C≤12)}, such as furan-2,3-diyl. In some embodiments, R₇ is hydrogen. In other embodiments, R₇ is halo, such as bromo. In some embodiments, R₇ is aryl_{(C≤12)} or substituted aryl(c12). In further embodiments, R₇ is substituted aryl(c12), such as 4-nitrophenyl, 4-(methoxycarbonyl)phenyl, or 4-methoxy-3-methylphenyl. In other embodiments, R₇ is heteroaryl_{(C≤12)} or substituted heteroaryl_{(C≤12)}. In further embodiments, R₇ is heteroaryl_{(C≤12)}, such as furan-3-yl or 1-methyl-1H-indol-4-yl. In other embodiments, R₇ is substituted heteroaryl_{(C≤12)}, such as 2-methoxypyridin-5-yl. In some embodiments, R₇ is a group of the formula:

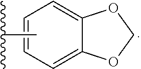

In further embodiments, R₇ is a group of the formula:

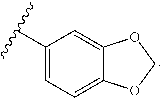 [SMILES: *c1ccc2c(c1)OCO2]

In other embodiments, R₇ is a group of the formula:

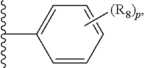

wherein:
   p is 0, 1, 2, 3, 4, or 5; and
   R₈ is, in each instance independently, hydrogen, hydroxy, halo, amino, cyano, or nitro; or
      alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
      —C(O)R₁₃, wherein:
         R₁₃ is amino; or
         alkyl_{(C≤8)}, alkoxy_{(C≤8)}, alkylamino_{(C≤8)}, or
         dialkylamino_{(C≤8)}, or a substituted version of any of these groups.

In some embodiments, R₇ or R₉ are —(CH₂)ₓC(O)Rₐ; wherein: x is 0, 1, or 2; Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)}. In some embodiments, x is 0. In other embodiments, x is 1. In some embodiments, Rₐ is hydrogen. In other embodiments, Rₐ is hydroxy. In other embodiments, Rₐ is alkoxy_{(C≤8)} or substituted alkoxy_{(C≤8)}. In other embodiments, Rₐ is alkylamino_{(C≤8)} or substituted alkylamino_{(C≤8)} such as t-butylamino. In other embodiments, R₇ or R₉ is —(CH₂)_{y}OR_{b}; wherein: y is 0, 1, or 2; and R_{b} is a hydroxy protecting group. In some embodiments, y is 0. In other embodiments, y is 1. In some embodiments, the hydroxy protecting group is an acyl_{(C≤8)} or a alkylsilyl_{(C≤12)} group such as a pivaloyl group or a tbutyldimethylsilyl group.

In some embodiments, p is 0, 1, or 2. In further embodiments, p is 0. In other embodiments, p is 1. In still other embodiments, p is 2. In some embodiments, R₈ is alkyl_{(C≤8)} or substituted alkyl_{(C≤8)}. In further embodiments, R₈ is alkyl_{(C≤8)}, such as methyl. In some embodiments, R₈ is alkoxy_{(C≤8)} or substituted alkoxy_{(C≤8)}. In further embodiments, R₈ is alkoxy_{(C≤8)}, such as methoxy. In some embodiments, R₈ is alkylsilyloxy_{(C≤8)} or substituted alkylsilyloxy_{(C≤8)}. In further embodiments, alkylsilyloxy_{(C≤8)}, such as t-butylsilyloxy. In other embodiments, R₈ is nitro. In some embodiments, R₁₃ is alkoxy_{(C≤8)} or substituted alkoxy_{(C≤8)}. In further embodiments, R₁₃ is alkoxy_{(C≤8)}, such as methoxy. In some embodiments, R₁₃ is alkylamino_{(C≤8)} or substituted alkylamino_{(C≤8)}. In further embodiments, alkylamino_{(C≤8)}, such as s-butylamino or t-butylamino. In some embodiments, R₁₄ is hydrogen. In some embodiments, R_{14′} is hydrogen.

In some embodiments, A₃ is heteroarenediyl_{(C≤12)} or substituted heteroarenediyl_{(C≤12)}. In further embodiments, A₃ is heteroarenediyl_{(C≤12)}, such as furan-2,3-diyl. In some embodiments, R₁₀ is hydrogen. In some embodiments, X₃ is hydrogen. In other embodiments, X₃ is halo, such as iodo.

In some embodiments, the compound is further defined as:

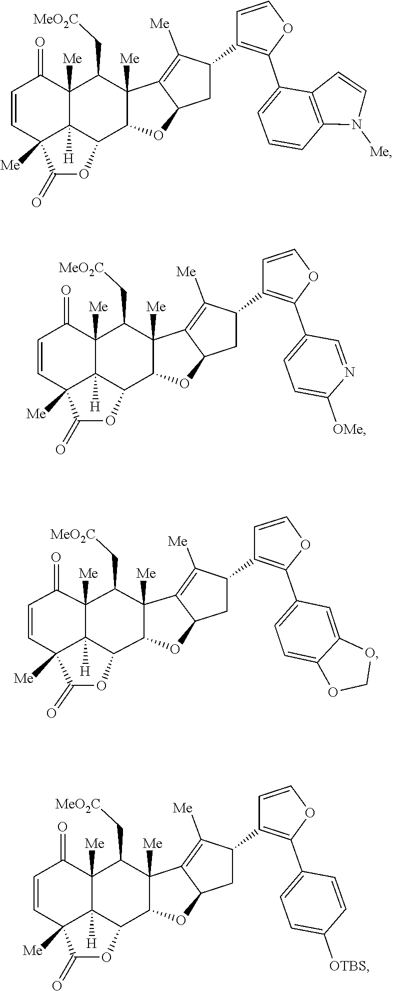

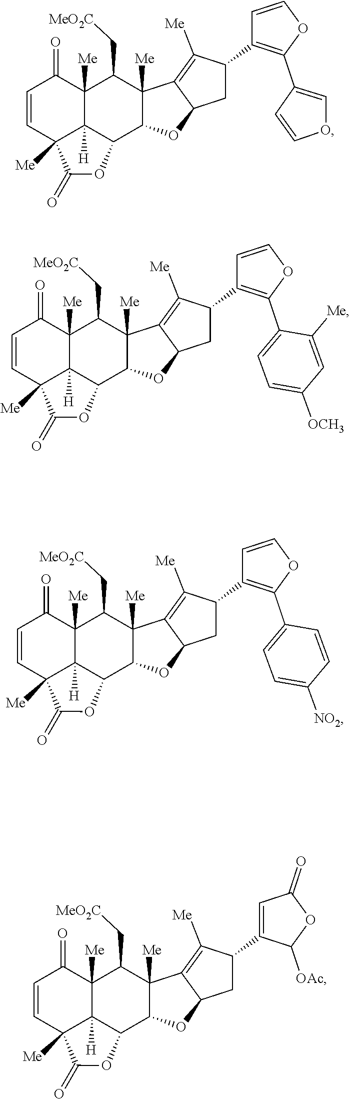

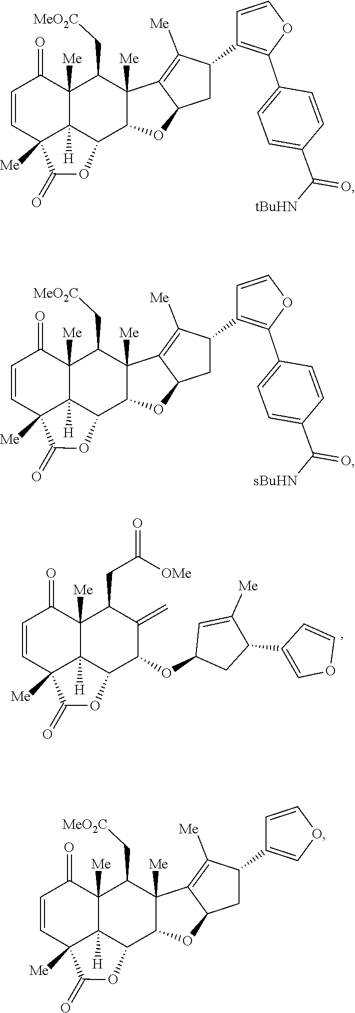

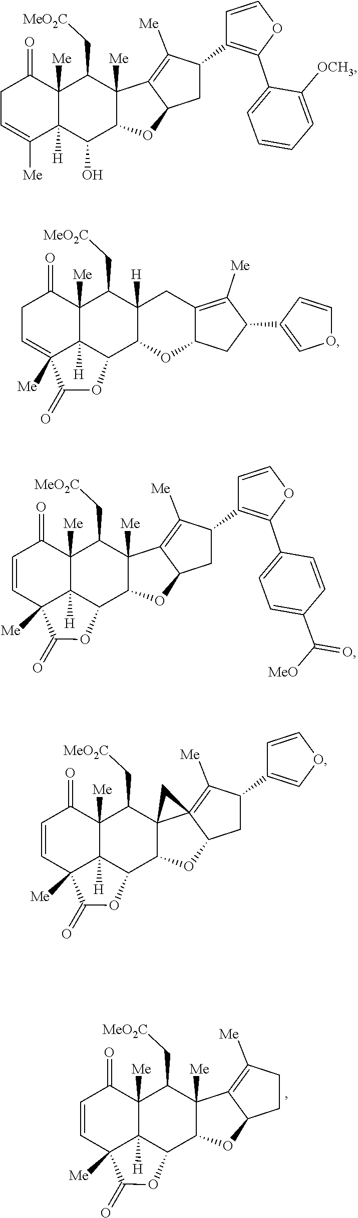

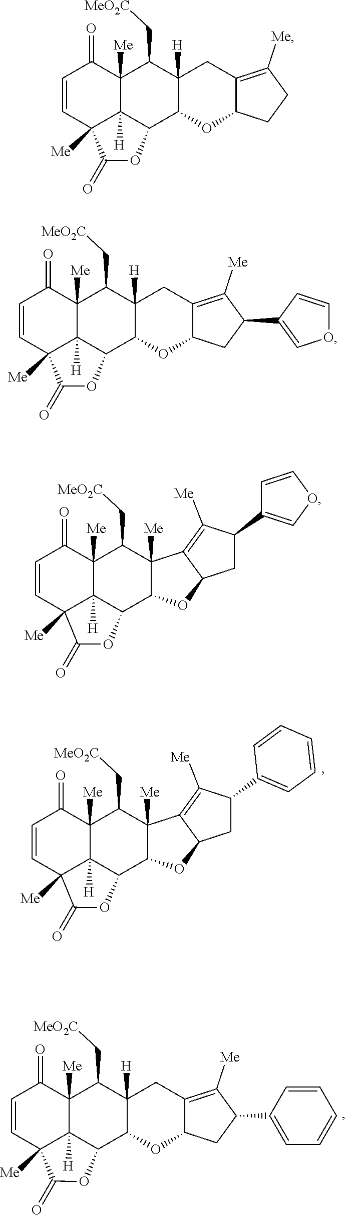

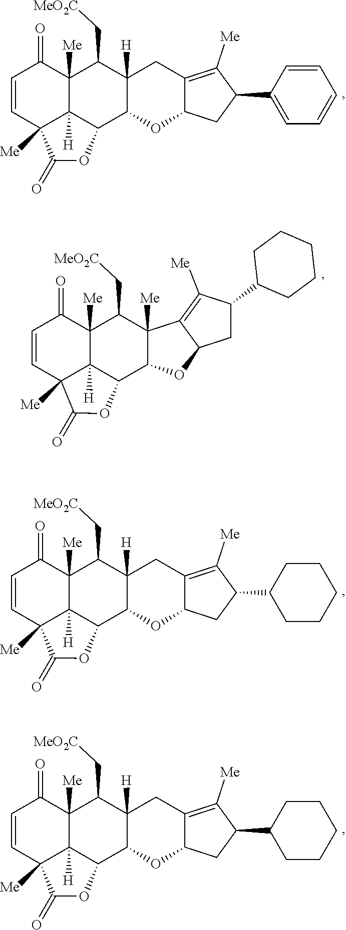

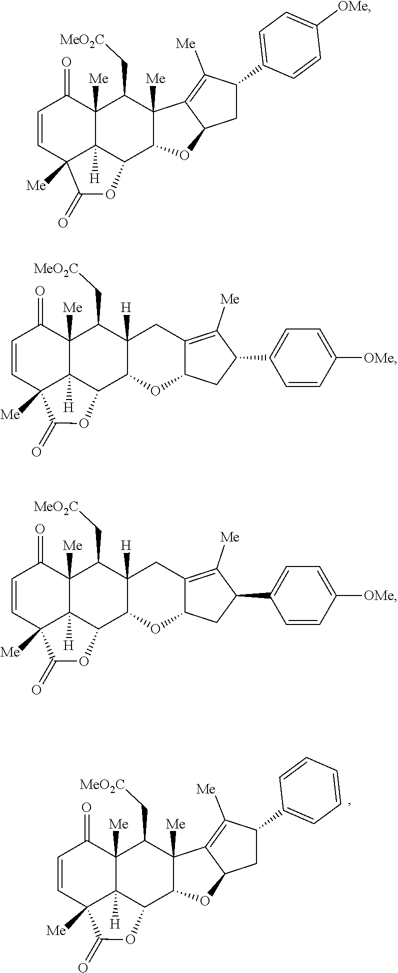

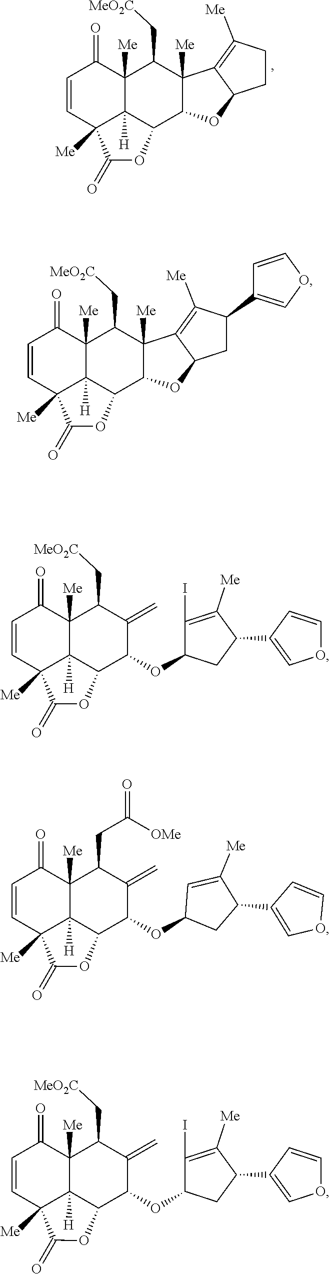

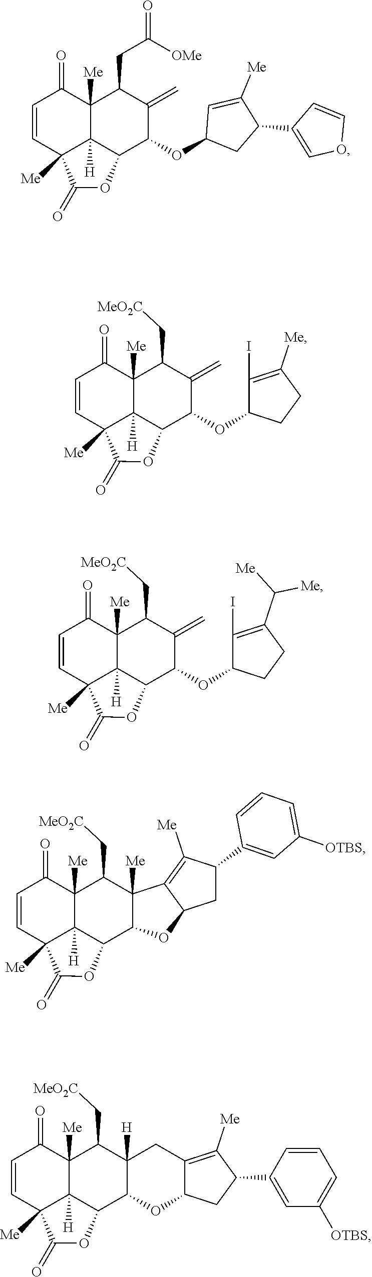

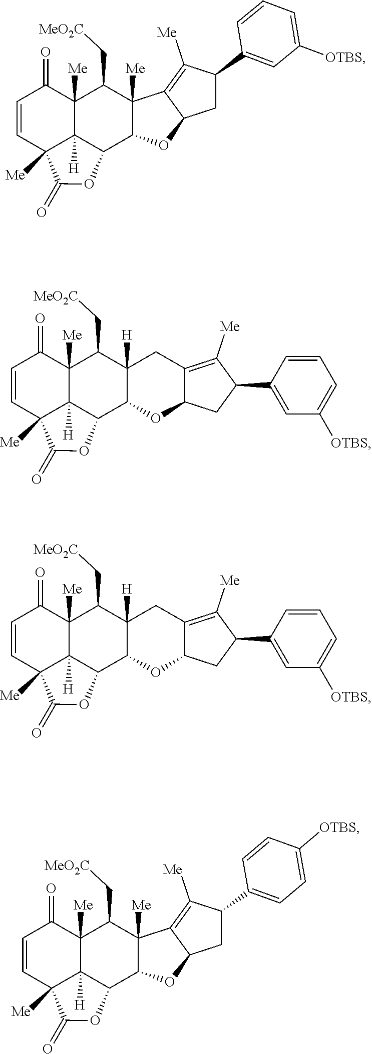

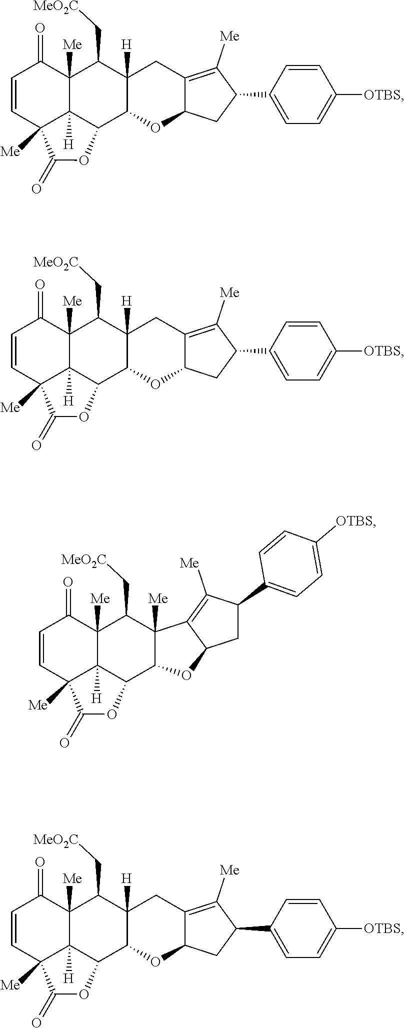

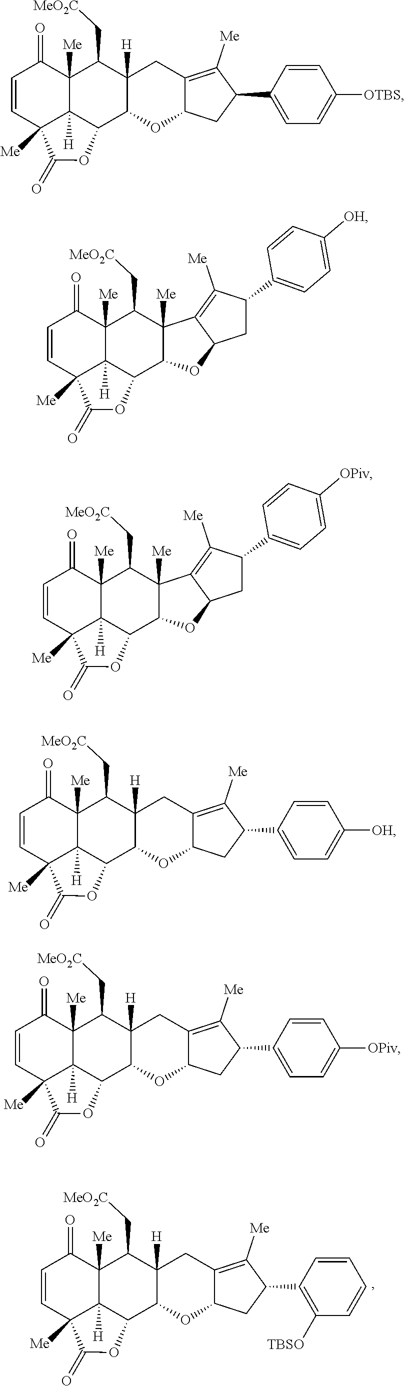

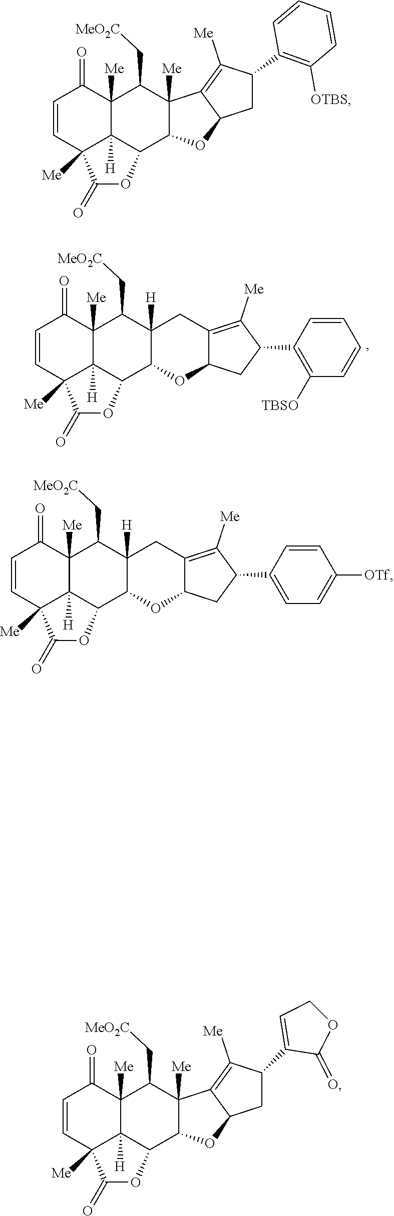

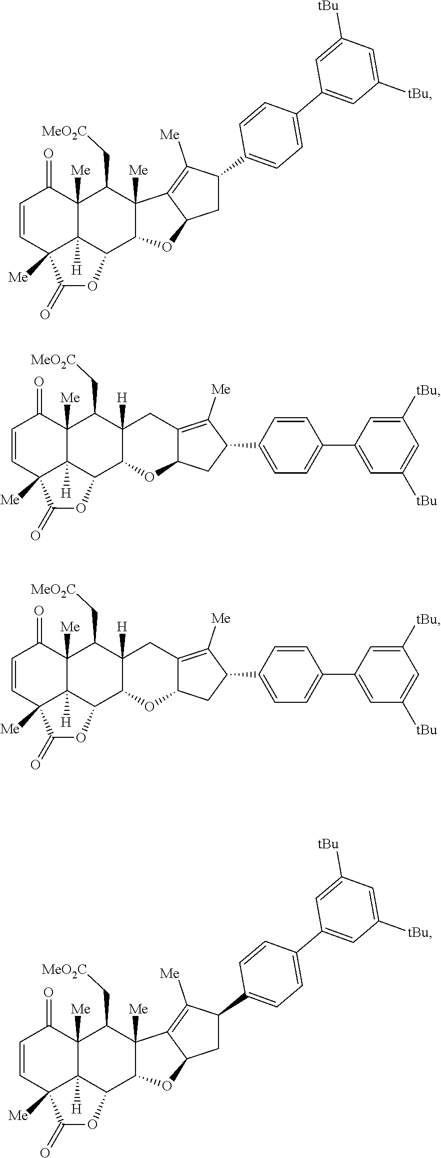

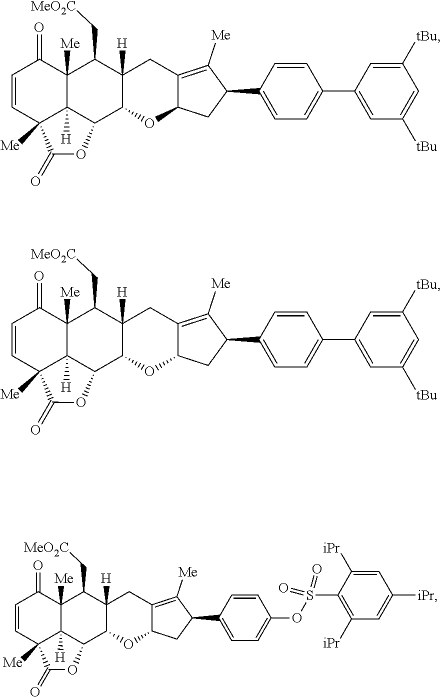

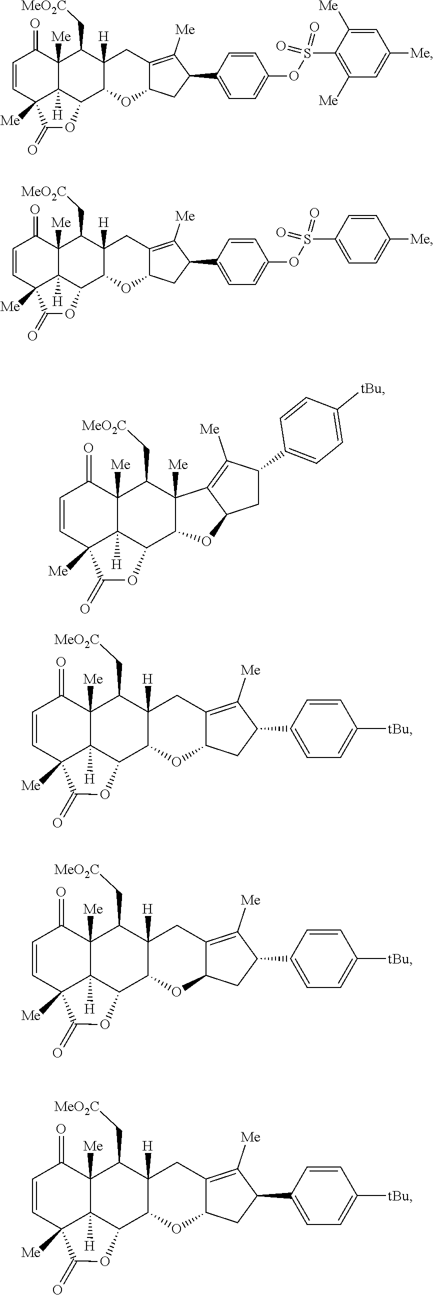

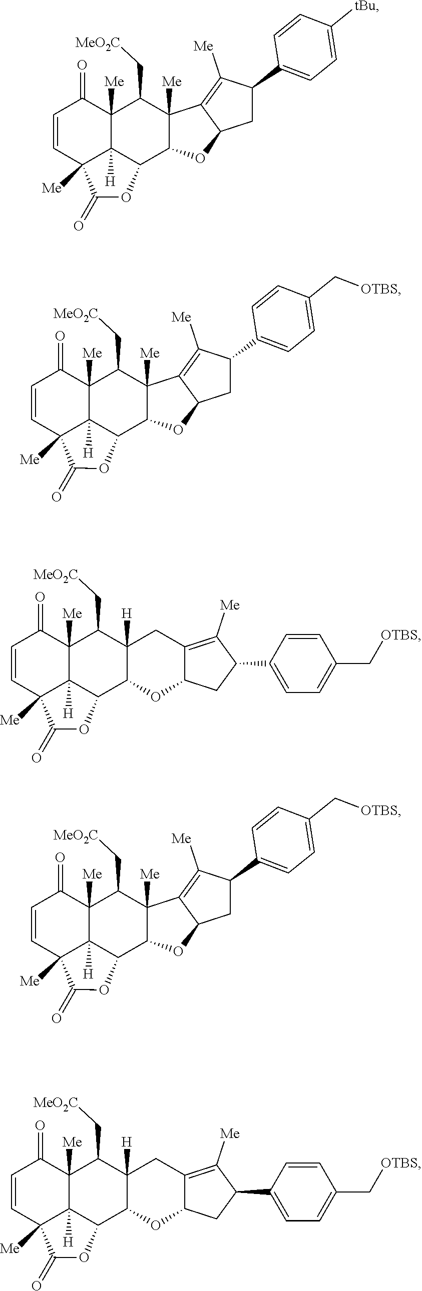

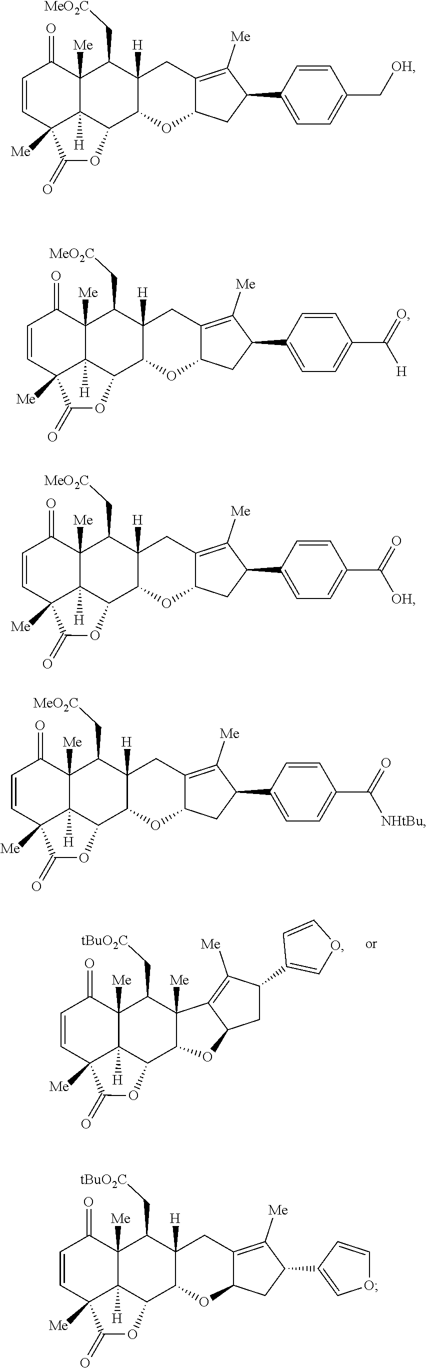

or a pharmaceutically acceptable salt thereof.
In some embodiments, the compound is further defined as:

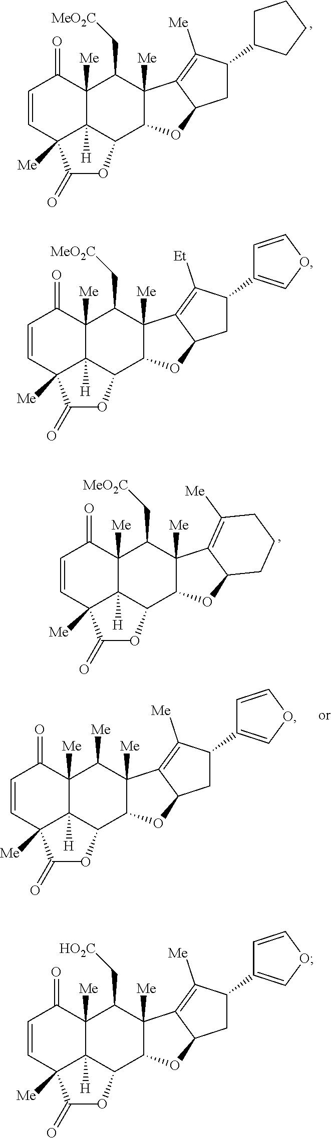

or a pharmaceutical salt thereof.

In other aspects, the present disclosure provides a compound of the formula:

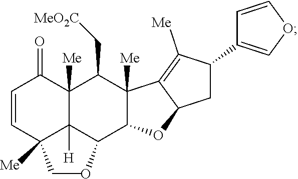 [SMILES: CC[C@]1(C)C=CC(=O)[C@@]2(C)C1[C@@H](O)[C@H]1O[C@@H]3C[C@@H](c4ccoc4)C(C)=C3[C@@]1(C)[C@@H]2CC(=O)OC]

or a pharmaceutical salt thereof.

In still other aspects, the present disclosure provides pharmaceutical compositions comprising:
(A) a compound of the present disclosure; and
(B) an excipient.

In some embodiments, the pharmaceutical composition is formulated for administration orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crémes, in lipid compositions, via a catheter, via a lavage, via continuous infusion, via infusion, via inhalation, via injection, via local delivery, or via localized perfusion. In some embodiments, the pharmaceutical composition is formulated as a unit dose.

In other aspects, the present disclosure provides methods of treating or preventing a disease or disorder in a patient in need thereof comprising administering to the patient a pharmaceutically effective amount of a compound or composition of the present disclosure. In some embodiments, the disease or disorder is a cancer or proliferative disease.

In still other aspects, the present disclosure provides methods of treating a cancer in a patient in need thereof, the method comprising administering a therapeutically effective amount of a compound that induces the trapping of both PARylated-PARP1 and PAR-binding proteins at DNA lesions.

In other aspects, the present disclosure provides methods of treating a cancer in a patient in need thereof, the method comprising administering a therapeutically effective amount of a compound that inhibits RNF114.

In some embodiments, said compound is not nimbolide. In other embodiments, said compound is nimbolide or an analogue or derivative thereof. In further embodiments, said compound is a compound of the present disclosure. In some embodiments, said cancer is a PARP inhibitor resistant cancer. In some embodiments, said cancer is a lung cancer, a breast cancer, a liver cancer, a kidney cancer, a brain cancer, a head and neck cancer, a testicular cancer, a prostate cancer, an ovarian cancer, a breast cancer, a uterine cancer, a bladder cancer, a skin cancer, an esophageal cancer, a stomach cancer, a pancreatic cancer, a colon cancer, a bone cancer, a Ewing's sarcoma, a thyroid cancer, an endometrial, or a leukemia. In some embodiments, said cancer is deficient in a homologous recombination (HR) dependent deoxyribonucleic acid (DNA) double strand break (DSB) repair pathway. In some embodiments, said cancer is deficient in breast cancer 1 (BRCA1) and/or breast cancer 2 (BRCA2). In some embodiments, said cancer is deficient in ATM, ATR, CHK1, CHK2, Rad51, RPA, XRCC3, Fanconi anemia complementation group A (FANCA), Fanconi anemia complementation group (FANCC), Fanconi anemia complementation group D2 (FANCD2), Fanconi anemia complementation group F (FANCF), Fanconi anemia complementation group G (FANCG) or Fanconi anemia complementation group M (FANCM).

In some embodiments, said cancer is a PARP inhibitor resistant cancer. In some embodiments, said PARP inhibitor resistant cancer is intrinsically resistant to PARP inhibitor therapy. In other embodiments, said PARP inhibitor resistant cancer has acquired resistance to PARP inhibitor therapy. In some embodiments, said compound or composition traps PARP1 at sites of DNA damage. In some embodiments, said compound or composition traps a PAR-binding protein at sites of DNA damage. In some embodiments, said compound or composition traps a PAR-binding DNA repair factor at sites of DNA damage. In some embodiments, said compound or composition traps XRCC1 at sites of DNA damage. In some embodiments, said compound or composition traps PARylated-PARP1 at sites of DNA damage. In some embodiments, said compound or composition prevents the degradation of PARylated PARP1. In some embodiments, said compound or composition inhibits the function of a ubiquitin E3 ligase. In some embodiments, said compound or composition inhibits the E3 ligase activity of RNF114.

In some embodiments, the methods further comprise administering the patient multiple doses of said compound or composition. In some embodiments, said compound or composition is administered daily, every other day, twice weekly, weekly, every two weeks, monthly or every other month. In some embodiments, wherein administering comprises oral, intravenous, intra-arterial, or subcutaneous administration. In some embodiments, said patient is a human patient. In other embodiments, said patient is a non-human animal patient.

In some embodiments, the methods further comprise administering to said patient at least one additional therapeutic. In some embodiments, said at least one additional therapeutic is an anti-cancer therapy. In further embodiments, said at least one additional therapeutic is a chemotherapy, a radiation therapy, a hormonal therapy, a toxin therapy, a surgical therapy, a cytokine therapy, or an immunotherapy. In some embodiments, said immunotherapy is an immune checkpoint inhibitor therapy. In some embodiments, said immune checkpoint inhibitor therapy targets PD1, PD-L1, CTLA4, STING, cGAS, BTLA, VISTA, TIM-3, LAG3, CD47, CD137, CD40L, ICOS, CD27, KIR, 4-1BB, CD28, TCR, TIGIT, OX40, or GITR. In some embodiments, said chemotherapy is a DNA damaging agent or an inhibitor of homologous recombination (HR) dependent DNA DSB repair. In some embodiments, said chemotherapy is an ATM/ATR inhibitor. In some embodiments, said chemotherapy is a CHK inhibitor. In some embodiments, said radiation therapy is an ionizing radiation therapy.

In some embodiments, the cancer comprises cancer cells defective in homologous recombination. In some embodiments, said cancer has previously been identified as a cancer that is deficient in a homologous recombination (HR) dependent deoxyribonucleic acid (DNA) double strand break (DSB) repair pathway. In some embodiments, the methods comprise (a) determining or having determined whether the cancer is defective in homologous recombination; (b) selecting or having selected the patient for treatment with the compound or composition when the cancer is defective in homologous recombination; and (c) administering or having administered to the selected patient the compound or composition. In further embodiments, step (a) comprises (i) obtaining or having obtained a biological sample from the patient; and (ii) performing or having performed an assay on the biological sample to determine whether the cancer is defective in homologous recombination. In some embodiments, said cancer comprises one or more cancer cells having a reduced or abrogated ability to repair DNA DSB by HR. In further embodiments, said one or more cancer cells have a reduced or abrogated ability to repair DNA DSB by HR relative to normal cells. In some embodiments, the methods further comprise identifying a cancer cell obtained from the patient as deficient in homologous recombination (HR) dependent deoxyribonucleic acid (DNA) double strand break (DSB) repair relative to normal cells. In some embodiments, said cancer cells are deficient in breast cancer 1 (BRCA1), breast cancer 2 (BRCA2), ATM, ATR, CHK1, CHK2, Rad51, RPA, XRCC3, Fanconi anemia complementation group A (FANCA), Fanconi anemia complementation group (FANCC), Fanconi anemia complementation group D2 (FANCD2), Fanconi anemia complementation group F (FANCF), Fanconi anemia complementation group G (FANCG) and Fanconi anemia complementation group M (FANCM).

In some embodiments, said cancer cells are homozygous for a mutation in BRCA1, BRCA2, ATM, ATR, CHK1, CHK2, Rad51, RPA, XRCC3, FANCA, FANCC, FANCD2, FANCF, FANCG, and FANCM. In some embodiments, said cancer is identified as a HR dependent DNA DSB repair deficient cancer by determining the HR dependent DNA DSB repair activity of cancer cells from the individual relative to normal cells. In some embodiments, said cancer is identified as an HR dependent DSB repair deficient cancer by determining the presence in cancer cells from the individual of one or more mutations or polymorphisms in a nucleic acid sequence encoding a component of the HR dependent DNA DSB repair pathway. In some embodiments, the patient is heterozygous for a mutation in a gene encoding a component of the HR dependent DNA DSB repair pathway. In some embodiments, the individual is heterozygous for a mutation in ATM, ATR, CHK1, CHK2, Rad51, RPA, XRCC3, BRCA1, and/or BRCA2.

In some aspects, the present disclosure provides intermediates of the formula:

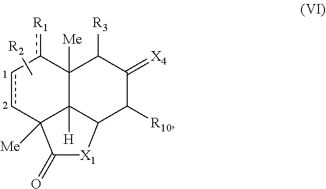

wherein:
   the bond between atoms 1 and 2 is a single bond or a double bond;
   X₁ is —O— or —NRₐ—, wherein:
      Rₐ is hydrogen, alkyl_{(C≤8)}, or substituted alkyl_{(C≤8)};
   X₄ is ═O or ═CH₂;
   R₁ is hydroxy or oxo;
   R₂ is hydrogen, amino, halo, hydroxy; or
      alkyl_{(C≤8)} or substituted alkyl_{(C≤8)};
   R₃ is hydrogen, alkyl_{(C≤8)}, substituted alkyl_{(C≤8)}, or —Y₁C(O)R_{b}, wherein:
      Y₁ is alkanediyl_{(C≤8)} or substituted alkanediyl_{(C≤8)};
      R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)}; and
   R₁₀ is amino or hydroxy; or
      alkoxy_{(C≤8)}, acyloxy_{(C≤8)}, alkylsilyloxy_{(C≤8)}, alkylamino_{(C≤8)}, dialkylamino_{(C≤8)}, amido_{(C≤8)}, or a substituted version of any of these groups; or
or intermediates of the formula:

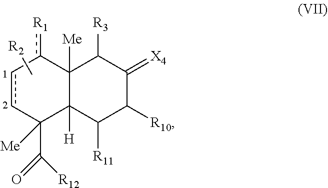

wherein:
   the bond between atoms 1 and 2 is a single bond or a double bond;
   X₄ is ═O or ═CH₂;
   R₁ is hydroxy or oxo;
   R₂ is hydrogen, amino, halo, hydroxy; or
      alkyl_{(C≤8)} or substituted alkyl_{(C≤8)};
   R₃ is hydrogen, alkyl_{(C≤8)}, substituted alkyl_{(C≤8)}, or —Y₁C(O)R_{b}, wherein:
      Y₁ is alkanediyl_{(C≤8)} or substituted alkanediyl_{(C≤8)};
      R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)}; and
   R₁₀ and Rn are each independently amino or hydroxy; or
      alkoxy_{(C≤8)}, acyloxy_{(C≤8)}, alkylsilyloxy_{(C≤8)}, alkylamino_{(C≤8)}, dialkylamino_{(C≤8)}, amido(c8), or a substituted version of any of these groups; or
   R₁₂ is hydroxy or amino; or
      alkoxy_{(C≤8)}, alkylamino_{(C≤8)}, dialkylamino_{(C≤8)}, or a substituted version of any of these groups;
      or
or intermediates of the formula:

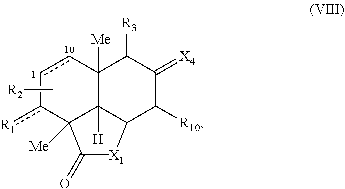

wherein:
   the bond between atoms 1 and 10 is a single bond or a double bond;
   X₁ is —O— or —NRₐ—, wherein:
      Rₐ is hydrogen, alkyl_{(C≤8)}, or substituted alkyl_{(C≤8)};
   X₄ is ═O or ═CH₂;
   R₁ is hydroxy or oxo;
   R₂ is hydrogen, amino, halo, hydroxy; or
      alkyl_{(C≤8)} or substituted alkyl_{(C≤8)};
   R₃ is hydrogen, alkyl_{(C≤8)}, substituted alkyl_{(C≤8)}, or —Y₁C(O)R_{b}, wherein:
      Y₁ is alkanediyl_{(C≤8)} or substituted alkanediyl_{(C≤8)};
      R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)}; and
   R₁₀ is amino or hydroxy; or
      alkoxy_{(C≤8)}, acyloxy_{(C≤8)}, alkylsilyloxy_{(C≤8)}, alkylamino_{(C≤8)}, dialkylamino_{(C≥8)}, amido_{(C≤8)}, or a substituted version of any of these groups; or
or a salt thereof.

In some embodiments, the intermediates are further defined as:

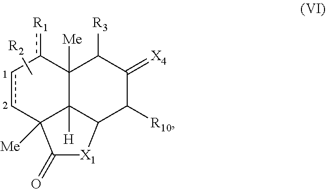

wherein:
   the bond between atoms 1 and 2 is a single bond or a double bond;
   X₁ is —O— or —NRₐ—, wherein:
      Rₐ is hydrogen, alkyl_{(C≤8)}, or substituted alkyl_{(C≤8)};
   X₄ is ═O or ═CH₂;
   R₁ is hydroxy or oxo;
   R₂ is hydrogen, amino, halo, hydroxy; or
      alkyl_{(C≤8)} or substituted alkyl_{(C≤8)};
   R₃ is hydrogen, alkyl_{(C≤8)}, substituted alkyl_{(C≤8)}, or —Y₁C(O)R_{b}, wherein:
      Y₁ is alkanediyl_{(C≤8)} or substituted alkanediyl_{(C≤8)};
      R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)}; and
   R₁₀ is amino or hydroxy; or
      alkoxy_{(C≤8)}, acyloxy_{(C≤8)}, alkylsilyloxy_{(C≤8)}, alkylamino_{(C≤8)}, dialkylamino_{(C≤8)}, amido_{(C≤8)}, or a substituted version of any of these groups;
or a salt thereof.

In some embodiments, the intermediates are further defined as:

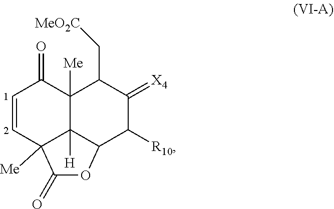

wherein:
   X₄ is ═O or ═CH₂; and
   R₁₀ is amino or hydroxy; or
      alkoxy_{(C≤8)}, acyloxy_{(C≤8)}, alkylsilyloxy_{(C≤8)}, alkylamino_{(C≤8)}, dialkylamino_{(C≤8)}, amido_{(C≤8)}, or a substituted version of any of these groups;
or a salt thereof.

In some embodiments, the intermediates are further defined as:

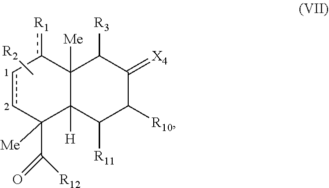

wherein:
   the bond between atoms 1 and 2 is a single bond or a double bond;
   X₄ is ═O or ═CH₂;
   R₁ is hydroxy or oxo;
   R₂ is hydrogen, amino, halo, hydroxy; or
      alkyl_{(C≤8)} or substituted alkyl_{(C≤8)};
   R₃ is hydrogen, alkyl_{(C≤8)}, substituted alkyl_{(C≤8)}, or —Y₁C(O)R_{b}, wherein:
      Y₁ is alkanediyl_{(C≤8)} or substituted alkanediyl_{(C≤8)};
      R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)}; and
   R₁₀ and R₁₁ are each independently amino or hydroxy; or
      alkoxy_{(C≤8)}, acyloxy_{(C≤8)}, alkylsilyloxy_{(C≤8)}, alkylamino_{(C≤8)}, dialkylamino_{(C≤8)}, amido_{(C≤8)}, or a substituted version of any of these groups; or
   R₁₂ is hydroxy or amino; or
      alkoxy_{(C≤8)}, alkylamino_{(C≤8)}, dialkylamino_{(C≤8)}, or a substituted version of any of these groups;
or a salt thereof.

In some embodiments, the intermediates are further defined as:

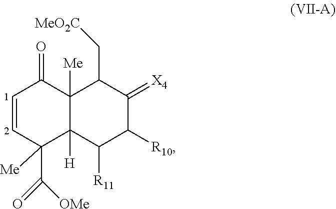

wherein:
   the bond between atoms 1 and 2 is a single bond or a double bond;
   X₄ is ═O or ═CH₂;
   R₁₀ and Rn are each independently amino or hydroxy; or
      alkoxy_{(C≤8)}, acyloxy_{(C≤8)}, alkylsilyloxy_{(C≤8)}, alkylamino_{(C≤8)}, dialkylamino_{(C≤8)}, amido_{(C≤8)}, or a substituted version of any of these groups;
or a salt thereof.

In some embodiments, the intermediates are further defined as:

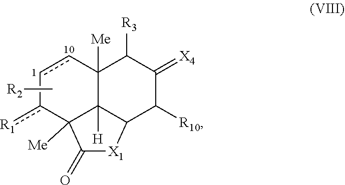

wherein:
   the bond between atoms 1 and 10 is a single bond or a double bond;
   X₁ is —O— or —NRₐ—, wherein:
      Rₐ is hydrogen, alkyl_{(C≤8)}, or substituted alkyl_{(C≤8)};
   X₄ is ═O or ═CH₂;
   R₁ is hydroxy or oxo;
   R₂ is hydrogen, amino, halo, hydroxy; or
      alkyl_{(C≤8)} or substituted alkyl_{(C≤8)};
   R₃ is hydrogen, alkyl_{(C≤8)}, substituted alkyl_{(C≤8)}, or —Y₁C(O)R_{b}, wherein:
      Y₁ is alkanediyl_{(C≤8)} or substituted alkanediyl_{(C≤8)};
      R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)}; and
   R₁₀ is amino or hydroxy; or
      alkoxy_{(C≤8)}, acyloxy_{(C≤8)}, alkylsilyloxy_{(C≤8)}, alkylamino_{(C≤8)}, dialkylamino_{(C≤8)}, amido_{(C≤8)}, or a substituted version of any of these groups; or
or a salt thereof.

In some embodiments, the intermediates are further defined as:

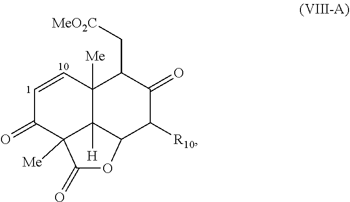

wherein:
   R₁₀ is amino or hydroxy; or
      alkoxy_{(C≤8)}, acyloxy_{(C≤8)}, alkylsilyloxy_{(C≤8)}, alkylamino_{(C≤8)}, dialkylamino_{(C≤8)}, amido_{(C≤8)}, or a substituted version of any of these groups;
or a salt thereof.

In some embodiments, the bond between atoms 1 and 2 is a double bond. In some embodiments, the bond between atoms 1 and 10 is a double bond. In some embodiments, R₁ is oxo. In some embodiments, R₂ is hydrogen. In some embodiments, R₃ is alkyl_{(C≤8)} or substituted alkyl_{(C≤8)}. In further embodiments, R₃ is substituted alkyl_{(C≤8)}, such as (methoxycarbonyl)methyl. In some embodiments, X₁ is —O—. In some embodiments, X₄ is ═O. In other embodiments, X₄ is ═CH₂. In some embodiments, R₁₀ is hydroxy. In other embodiments, R₁₀ is alkylsilyloxy_{(C≤8)} or substituted alkylsilyloxy_{(C≤8)}. In further embodiments, R₁₀ is alkylsilyloxy_{(C≤8)}, such as trimethylsilyloxy or triethylsilyloxy. In some embodiments, R₁₁ is hydroxy. In other embodiments, R₁₁ is alkylsilyloxy_{(C≤8)} or substituted alkylsilyloxy_{(C≤8)}. In further embodiments, Rn is alkylsilyloxy_{(C≤8)}, such as trimethylsilyloxy. In some embodiments, R₁₂ is alkoxy_{(C≤8)} or substituted alkoxy_{(C≤8)}. In further embodiments, R₁₂ is alkoxy_{(C≤8)}, such as methoxy.

In some embodiments, the intermediates are further defined as:

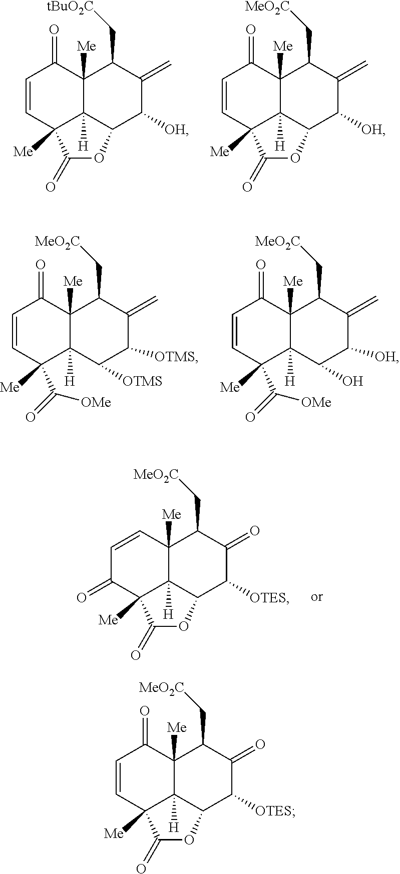

or a salt thereof.

In other aspects, the present disclosure provides methods of manufacturing a compound of the present disclosure comprising contacting an intermediate of the present disclosure with a base. In some embodiments, the base is an inorganic base. In some embodiments, the base is a salt, such as K₂CO₃.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

For a more complete understanding of the features and advantages of the present disclosure, reference is now made to the detailed description of the disclosure along with the accompanying figures and in which:

FIGS. 1A-1D show identification of the candidates involved in the PARylation dependent DNA damage response. (FIG. 1A) Overall scheme of the experimental procedures for quantitative proteomic experiment to analysis the protein dynamics in response to DNA damage (FIG. 2). (FIG. 1B) Immunoblot analysis of the whole cell lysates for (FIG. 1A). (FIG. 1 C) Hierarchical clustering of the Chromatin-On and Off proteins based on the Protein Dynamics map. (FIG. 1D) Protein Dynamics of the Chromatin-On and PARylation-dependent candidates.

FIGS. 2A-F show RNF114 is recruited to DNA lesions via its PAR-binding domains. (FIG. 2A) The abundance of RNF114 from the chromatin samples under the indicated treatment conditions. (FIG. 2B) The domain structure of RNF114. (FIG. 2C) Immuno-dot-blot analysis of the binding between RNF114 (wt or different mutants) and PAR polymers. (FIG. 2D) RNF114 binds to PAR in vivo. HCT116 cells were transfected with Flag or Flag-pRNF114 for 48 h. Cells were pre-treated with Talazoparib (1 μM for 1 h), and followed by the treatment with H₂O₂ (2 mM for 5 min). Cells were lysed, and were subject to immunoprecipitation (against Flag). (FIG. 2E) The PBZ domain mediates the PAR-dependent recruitment of RNF114 to chromatin. The chromatin-bound fraction was isolated from HCT116-RNF114-KO cells stably expressing the wild-type or the PBZ mutated pRNF114. Cells were pre-treated with Talazoparib (1 μM for 1 h), and followed by the treatment with H₂O₂ (2 mM for 5 min). The chromatin bound proteins were extracted from the cells using a subcellular fractionation kit. (FIG. 2F) The recruitment of RNF114-WT or RNF114-*PBZ to DNA damage sites. Hela cells were transfected with RNF114-WT-GFP or RNF114-*PBZ-GFP. When indicated, the cells were pre-treated with DMSO or Talazoparib (1 μM for 1 h). Cells were subject to laser microirradiation assays. Scale Bars, 10 μm.

FIGS. 3A & 3B show GO analysis of the Chromatin-On and Off proteins. (Related to FIG. 1). (FIG. 3A) Gene ontology (GO) analysis (Biological process) of the Chromatin-On proteins. (FIG. 3B) Gene ontology (GO) analysis (Biological process) of the Chromatin-Off proteins.

FIGS. 4A & 4B show GO analysis of the Chromatin-On/Off and PARylation-dependent/independent proteins. (Related to FIG. 1). (FIG. 4A) Gene ontology (GO) analysis (Biological process) of the Chromatin-On-PARylation-dependent proteins. (FIG. 4B) Gene ontology (GO) analysis (Biological process) of the Chromatin-On-PARylation-independent proteins.

FIG. 5 shows validation of the protein dynamics during DNA damage response. (Related to FIG. 1). The protein dynamics and immunoblot analysis of candidate regulators' expression on chromatin according to the MS analysis in (FIG. 1A).

FIG. 6 shows examples of protein dynamics during PARylation-dependent DNA damage response. (Related to FIG. 1). The protein dynamics of XRCC1, POLβ, and LIG3 according to the MS analysis in (FIG. 1A).

FIGS. 7A-7F show RNF114 targets PARylated-PARP1 for ubiquitin proteasomal degradation. (FIG. 7A) PAR chains stimulate the auto-ubiquitination of RNF114. (FIG. 7B) PARylated PARP1 binds to RNF114, as shown in co-IP/MS (upper panel) and immunoblotting experiments (bottom). HCT116 cells were transfected with Flag or Flag-RNF114 plasmids for 48 h. The cells were then pre-treated with Talazoparib (1 μM for 1 h), followed by H₂O₂ treatment (2 mM for 5 min). The samples were subjected to co-immunoprecipitation and MS analysis. (FIG. 7C) PARP1 is ubiquitinated by RNF114. PARP1 was immunoprecipitated from HCT116-RNF114-KO or HCT116-RNF114-WT cells, and the immunoprecipitates were analyzed by immunoblot experiments using the indicated antibodies. (FIG. 7D) PARylated PARP1 was degraded by RNF114. PARP1 was immunoprecipitated from HCT116-RNF114-KO cells stably expressing GFP, RNF114-WT, RNF114-*PBZ, or RNF114-*RING. Cells were pre-treated with H₂O₂ (2 mM for 5 min). (FIG. 7E) The recruitment of PARP1 to DNA damage lesions. HeLa-RNF114-WT, HeLa-RNF114-*PBZ, or HeLa-RNF114-*RING cells were transfected with PARP1-GFP and were subject to laser microirradiation assays. Scale Bars, 10 μm. (FIG. 7F) HCT116-RNF114-KO cells stably expressing GFP, RNF114-WT, RNF114-*PBZ, or RNF114-*RING, were treated with MMS (1 mM for 9 h). Data shown are mean±SEM.

FIGS. 8A-8D show RNF114 targets PARylated-PARP1 for ubiquitin proteasomal degradation. (Related to FIG. 7). (FIG. 8A) PARylated PARP1 is ubiquitinated by RNF114. (FIG. 8B) PARylated PARP1 is degraded by RNF114. PARPI was immunoprecipitated from HCT116-RNF114-KO and HCT116-RNF114-WT cells that were treated with H₂O₂ (2 mM for 5 min). (FIG. 8C) PARylated PARP1 interacts with RNF114. PARylated-PARP1 was incubated with the recombinant GST, GST-RNF114, GST-RNF114-*PBZ, or GST-RNF114-*RING. Samples were subject to immunoprecipitation against GST, and were analyzed using the indicated antibodies. (FIG. 8D) RNF114 targets PARylated PARP1 for proteasomal degradation. HCT116-RNF114-KO cells stably expressing GFP, RNF114-WT, RNF114-*PBZ, or RNF114-*RING were pre-treated with MG132 (10 μM for 8 h), and were then treated with H₂O₂ (2 mM for 5 min).

FIGS. 9A-9G show cancer-associated mutations impair the function of RNF114. (FIG. 9A) RNF114 colocalizes with PCNA during DDR. Representative images of GFP-RNF114 (green) and DSRED-PCNA (red) after the DNA damage induced in the laser microirradiation assay. (FIG. 9B) RNF114 protects cells against genotoxic stress. HCT116-RNF114-WT and HCT116-RNF114-KO cells were treated with or without H₂O₂ (2 mM for 5 min), followed by 14 days culture using the colony formation assay. Cells were stained with crystal violet. (FIG. 9C) The domain structure of RNF114 with the two cancer associated mutations indicated. (FIG. 9D) The E37Q mutation impairs the ubiquitin E3 activity of RNF114. Immunoblot analysis of auto-ubiquitination activity of RNF114-WT and RNF114-E37Q with indicated antibodies. (FIG. 9E) The P174S mutation impairs the PAR-binding activity of RNF114. PAR polymers were incubated with the recombinant GST, GST-RNF114, GST-RNF114-P174S, or GST-RNF114-*PBZ mutant. The samples were subject to immunoprecipitation, and were analyzed using the indicated antibodies. (FIG. 9F) The E37Q and P174 mutations impair the RNF114-mediated ubiquitination of PARylated PARP1. PARP1 was immunoprecipitated from HCT116-RNF114-KO cells transfected with GFP, RNF114-WT, RNF114-P174S, or RNF114-E37Q plasmids. Cells were treated with H₂O₂ (2 mM for 5 min), and the samples were analyzed using the indicated antibodies. (FIG. 9G) The recruitment of PARP1 to DNA damage lesions. HeLa-RNF114-WT or HeLa-RNF114-KO cells were transfected with PARP1-GFP. The recruitment kinetics of PARP1 was monitored in a time course following laser microirradiation. Scale Bars, 10 μm.

FIGS. 10A-10I show Nimbolide traps both PARP1 and DNA repair factors. (FIG. 10A) Structure of Nimbolide. (FIG. 10B) The recruitment of PARP1 to DNA damage lesions. Hela-RNF114-WT or Hela-RNF114-KO cells were transfected with PARP1-GFP. Hela-RNF114-WT cells were pre-treated with DMSO, Olaparib (10 μM), or Nimbolide (1 μM) for 1 h before DNA damage. The kinetics of PARP1 was monitored in a time course following laser microirradiation. Scale Bars, 10 μm. (FIG. 10C) Nimbolide induced PARP1 cleavage and DNA damage. HCT116-RNF114-WT or HCT116-RNF114-KO cells were treated with DMSO or Nimbolide (1 μM for 48 h). (FIG. 10D) Cell viability of Hela-RNF114-WT and Hela-RNF114-KO cells with the treatment of Nimbolide. Hela-RNF114-WT or Hela-RNF114-KO cells were treated with DMSO or Nimbolide (1 μM for 96 h), and then subjected to cell titer glo analysis. Data represent mean±SEM. (FIG. 10E) Cell viability of Hela-PARP1-WT and Hela-PARP1-KO cells with the treatment of Nimbolide. Hela-PARP1-WT or Hela-PARP1-KO cells were treated with DMSO, Nimbolide (1 μM for 96 h), and then subjected to cell titer glo analysis. Data represent mean±SEM. (FIG. 10F) Cell viability of UWB1 and UWB1+BRCA1 cells with the treatment of Nimbolide. UWB1 or UWB1+BRCA1 cells were treated with DMSO or Nimbolide (0.1 μM, 0.25 μM, 0.5 μM, and 1 μM) for 96 hours, and then subjected to cell titer glo analysis. Data represent mean±SEM. (FIG. 10G) The recruitment of XRCC1 to DNA damage lesions. Hela cells transfected with XRCC1-GFP were pre-treated with DMSO, Olaparib (1 μM), or Nimbolide (1 μM) for 1 hour, and then subjected to laser microirradiation. The kinetics of XRCC1 was monitored in a time course following the laser microirradiation assay. Scale Bars, 10 μm. (FIG. 10H) Cell viability of HCC1937 cells with the treatment of Nimbolide. HCC1937 cells were treated with DMSO, Nimbolide (1 μM), or PARPi (Olaparib, Rucaparib) (1 μM) for 96 hours, and then subjected to cell titer glo analysis. Data present mean±SEM. (FIG. 10I) Cell viability of Olaparib-resistant UWB1 (SYr12) cells with the treatment of Nimbolide or Olaparib. UWB1 (SYr12) cells were treated with Olaparib or Nimbolide (0.001 μM, 0.01 μM, 0.1 μM, 0.25 μM, 0.5 μM, 1 μM, 2 μM and 5 μM) for 96 hours, and then subjected to cell titer glo analysis. Data present mean±SEM.

FIG. 11 shows cytotoxicity of Nimbolide for cell lines with different genetic background and mutation spectra (Related to FIG. 10). UWB1, A673, Fadu, H2058, H1048, or MDA-MB-468 cells were treated with DMSO, Nimbolide (1 μM), or PARPis (Olaparib, Rucaparib) (1 μM) for 96 hours. Cells were then subjected to cell titer glo analysis. Data represent mean±SEM.

FIG. 12 shows BRCA1-null UWB1 cells are sensitive to nimbolide. (Related to FIG. 10). Nimbolide demonstrates superior cytotoxicity than PARPi for BRCA-null cells. UWB1 cells were treated with Olaparib or Nimbolide (0.001 μM, 0.01 μM, 0.1 μM, 0.25 μM, 0.5 μM, 1 μM, 2 μM and 5 μM) for 96 hours. The cells were then subjected to cell titer glo analysis. Data represent mean±SEM.

FIGS. 13A & 13B show two Talazoparib-resistant cell lines (i.e., H889 and H82) were highly sensitive to nimbolide.

FIGS. 14A & 14B show synergistic effects between Nimbolide and various DNA-damaging agents (Related to FIG. 10). (FIG. 14A) UWB1 cells were treated with Nimbolide and DNA-damaging agents (MMS, Doxorubicin, Temozolomide). Nimbolide (0.25 μM) was used alone or together with MMS (10 μM), Doxorubicin (0.01 μM), or Temozolomide (10 μM) in UWB1 for 96 hours. The cells were subjected to cell titer glo analysis. Data represent mean±SEM. (FIG. 14B) UWB1 cells were treated with Nimbolide and agents that target various component of DNA repair machinery (ATM/ATRi or CHK1i). Nimbolide (0.25 μM) was used alone or together with AZD6738 (0.1 μM), CGK733 (1 μM), LY2603618 (0.1 μM), or SCH900776 (0.1 μM) in UWB1 for 96 hours. The cells were then subjected to cell titer glo analysis. Data represent mean±SEM.

FIGS. 15A-15H show Nimbolide triggers innate immune response. (FIG. 15A) Representative immunofluorescence images of PicoGreen staining in Hela cells treated with DMSO or Nimbolide (1 μM) for 48 hours. DAPI (blue) was used to visualize the nuclei. Scale Bars, 10 μm. (FIG. 15B) Immunoblot analysis of the Hela cells treated with DMSO or Nimbolide (1 μM for 48 h). Scale Bars, 10 μm. (FIGS. 15C & 15D) Immunofluorescence images of phosphorylated TBK1 (p-TBK1) (FIG. 15C) and phosphorylated IRF3 (p-IRF3) (FIG. 15D) in Hela cells treated with DMSO or Nimbolide (1 μM for 48 h). Scale Bars, 10 μm. (FIG. 15E) qPCR of IFN-β, CXCL10, or CCL5 in Hela cells treated with DMSO or Nimbolide (1 μM for 48 h). Data represent mean±SEM. (FIG. 15F) Nimbolide induced PD-L1 expression in UWB1 cells. UWB1 cells were treated with DMSO or Nimbolide (1 μM) for 48 hours. (FIGS. 15G & 15H) Representative tumor images (FIG. 15G) and tumor volume measurements (FIG. 15H) of B16 tumors in C57BL/6 mice (n=5) with continuous treatment.

FIGS. 16A & 16B show Nimbolide activates innate immune signaling. (Related to FIG. 15). (FIG. 16A) Nimbolide induces phosphorylated TBK1 (p-TBK1) signaling in an RNF114-dependent fashion. Hela-RNF114-WT and Hela-RNF114-KO cells were treated with DMSO or Nimbolide (1 μM for 48 h). (FIG. 16B) Nimbolide induces phosphorylated TBK1 (p-TBK1) signaling in a PARP1-dependent fashion. Hela-PARP1-WT and Hela-PARPI-KO cells were treated with DMSO or Nimbolide (1 μM for 48 h).

FIGS. 17A & 17B show Nimbolide triggers more potent innate immune signaling compared to PARPi (Related to FIG. 15). (FIG. 17A) Nimbolide induces stronger phosphorylated TBK1 (p-TBK1) compared to PARPi. Hela cells were treated with DMSO, Nimbolide (1 μM) or Olaparib (1 μM, 10 μM) for 48 hours. (FIG. 17B) Nimbolide induces stronger PD-L1 compared to PARPi. UWB1 cells were treated with DMSO, Nimbolide (1 μM, 2 μM) or Olaparib (5 μM, 10 μM) for 48 hours.

FIGS. 18A-18E show synergistic effects between Nimbolide and anti-PD-L1 antibodies. (Related to FIG. 15). (FIG. 18A) Cell viability of B16 cells with the treatment of Nimbolide. UWB1 cells were treated with Nimbolide (0.1 μM, 0.25 μM, 0.5 μM, 1 μM, 2 μM and 5 μM) for 96 hours, and then subjected to cell titer glo analysis. Data represent mean±SEM. (FIG. 18B) Nimbolide induces PD-L1 expression in B16 cells. B16 cells were treated with DMSO or Nimbolide (1 μM, 2 μM) for 48 hours. Relative PD-L1 expression with the treatment of Nimbolide was calculated with ImageJ according to the immunoblot. (FIG. 18C) Schematic of the combination treatment in C57BL/6 mice bearing B16 tumors. Intraperitoneal injections of isotype control IgG or anti-PD-L1 antibody (aPD-L1, 100 mg/mouse) started at day 7 after B16 cell inoculation. DMSO or Nimbolide (20 mg/kg) was orally administered daily. (FIGS. 18D & 18E) Toxicity assay of Nimbolide in mice: Male C57BL/6 adult mice were dosed intraperitoneal injections of isotype control IgG or anti-PD-L1 antibody (aPD-L1, 100 mg/mouse) started at day 7 after B16 cell inoculation. DMSO or Nimbolide (20 mg/kg) was orally administered daily. They were weighed (FIG. 18D) and observed for signs of pain and distress (FIG. 18E).

FIG. 19 shows Nimbolide analogs (S-46 and S-39) induce PARP1 trapping.

FIG. 20 shows Nimbolide analogs (S-84 and S-26) induce profound PARP1 trapping.

FIG. 21 shows a model for the mechanism of action of Nimbolide.

FIGS. 22A-22C show RNF114 mediates Nimbolide-induced PARP1 trapping. (FIG. 22A) Generation of RNF114 deletion HCT116 cells. RNF114-WT and -KO HCT116 cells were lysed and subjected to immunoblotting experiments using the indicated antibodies. (FIG. 22B) Depletion of RNF114 abrogated Nimbolide-induced PARP1 trapping. RNF114-WT and —KO HCT116 cells were treated with or without Nimbolide (1 μM for 48 h). The chromatin-bound fraction was isolated from these cells and was subjected to immunoblotting experiments using the indicated antibodies. (FIG. 22C) Nimbolide induces PARP1 trapping in ID8-sgBRCA1 cells. ID8-sgBRCA1 cells were treated with or without Nimbolide (1 μM for 48 h). The chromatin-bound fraction was isolated from these cells and was subjected to immunoblotting experiments using the indicated antibodies.

FIGS. 23A-23D show the biochemical characterization of additional RNF114 inhibitors. (FIG. 23A) Structure of Nimbolide and EN62. (FIG. 23B) PARP1 trapping induced by the various RNF114 inhibitors. UWB1 cells were treated with either Nimbolide (1 μM) or EN62 (1 μM) for 48 hrs. Cells were subject to subcellular fractionation, and the chromatin-bound fraction was isolated. Toxicity of the various RNF114 inhibitors (FIG. 23C) and (FIG. 23D). UWB1 cells were treated with either Nimbolide (1 μM) or EN62 (1 μM) for 4 days. Cell viability was measured using the CellTiter-Glo assay (FIG. 23C). The IC₅₀ of Nimbolide and EN62 against UWB1 cells was measured as shown in (FIG. 23D).

### DESCRIPTION OF ILLUSTRATIVE EMBODIMENTS

Disclosed herein are new compounds and compositions with anti-cancer properties, methods for their manufacture, and methods for their use, including for the treatment and/or prevention of disease.

### I. Compounds of the Present Disclosure

The compounds of the present disclosure are shown, for example, above, in the summary section, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

TABLE 1
Structure of Nimbolide and Examples of Nimbolide
Derivatives Provided Herein

| Compound | |
|---|---|
| ID | Structural Formula |
| Nimbolide | 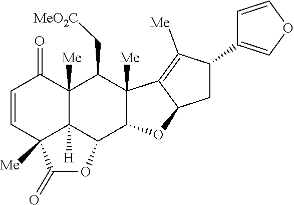 |
| S-16 | 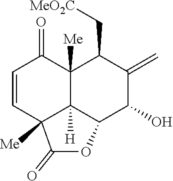 |
| S-16-a | 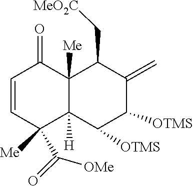 |
| S-16-b | 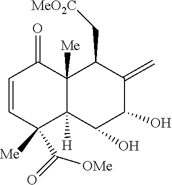 [SMILES: C=C1[C@H](O)[C@H](O)[C@@H]2[C@](C)(C(=O)C=C[C@@]2(C)C(=O)OC)[C@H]1CC(=O)OC] |
| S-26 | 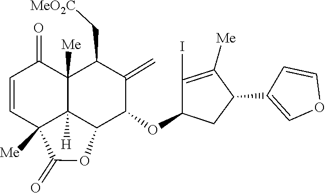 |
| S-26-1 | 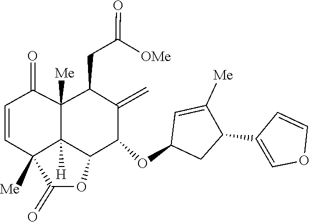 |
| S-27 | 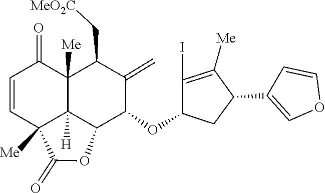 |
| S-38 | 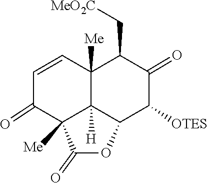 |
| S-39 | 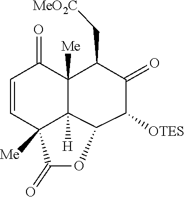 |
| S-41 | 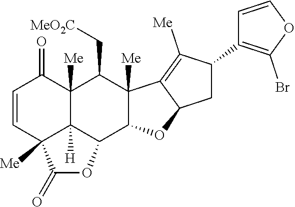 |
| S-42 | 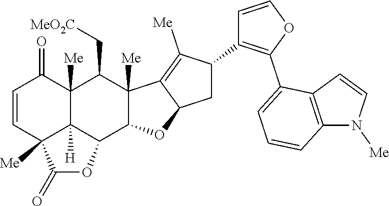 |
| S-43 | 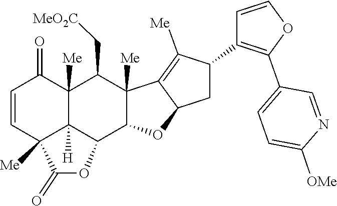 |
| S-44 | 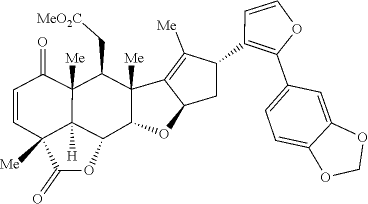 |
| S-45 | 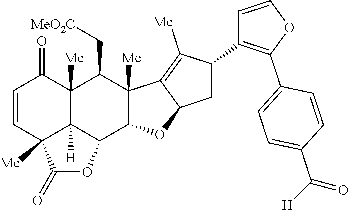 |
| S-46 | 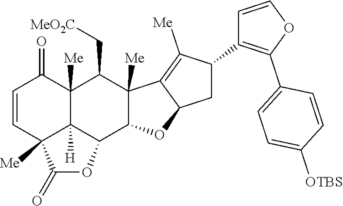 |
| S-47 | 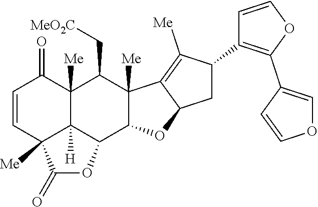 |
| S-48 | 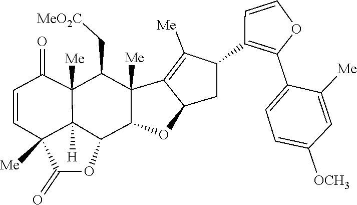 |
| S-49 | 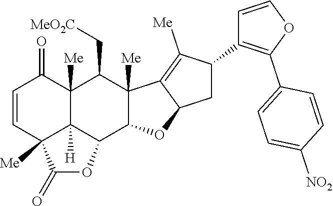 |
| S-50/51 | 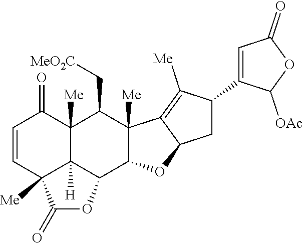 |
| S-52 | 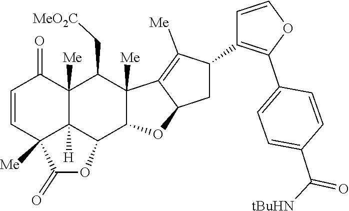 |
| S-53 | 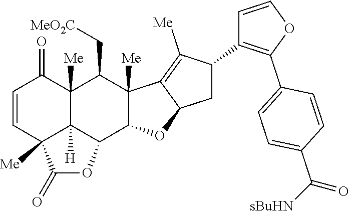 |
| S-54 | 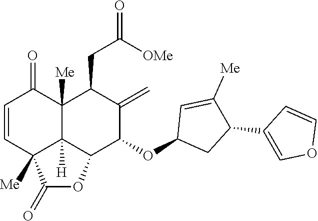 |
| S-55 | 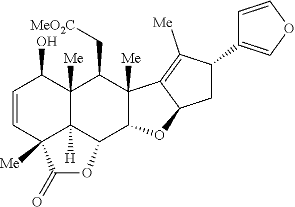 |
| S-56 | 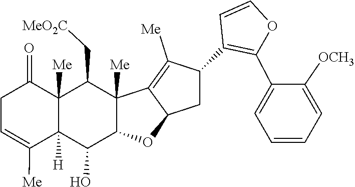 [SMILES: COC(=O)C[C@H]1[C@]2(C)C3=C(C)[C@H](c4ccoc4-c4ccccc4OC)C[C@H]3O[C@@H]2[C@H](O)[C@H]2C(C)=CCC(=O)[C@]12C] |
| S-57 | 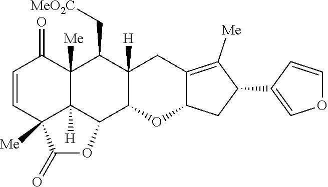 |
| S-58 | 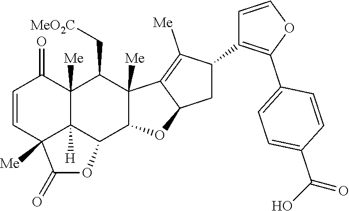 |
| S-59 | 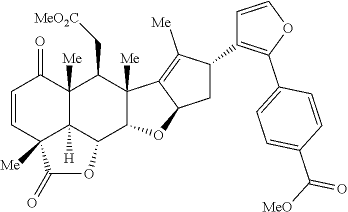 |
| S-60 | 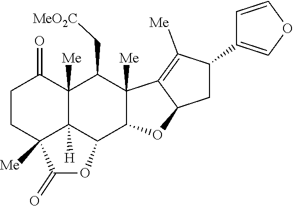 |
| S-61 | 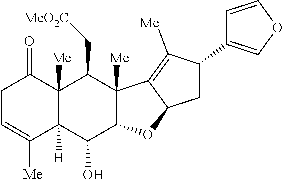 [SMILES: COC(=O)C[C@H]1[C@]2(C)C3=C(C)[C@H](c4ccoc4)C[C@H]3O[C@@H]2[C@H](O)[C@H]2C(C)=CCC(=O)[C@]12C] |
| S-62 | 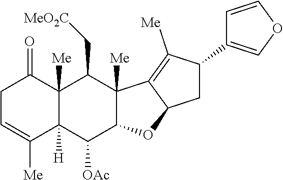 [SMILES: COC(=O)C[C@H]1[C@]2(C)C3=C(C)[C@H](c4ccoc4)C[C@H]3O[C@@H]2[C@H](OC(C)=O)[C@H]2C(C)=CCC(=O)[C@]12C] |
| S-63 | 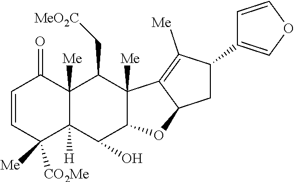 [SMILES: COC(=O)C[C@H]1[C@]2(C)C3=C(C)[C@H](c4ccoc4)C[C@H]3O[C@@H]2[C@H](O)[C@@H]2[C@]1(C)C(=O)C=C[C@@]2(C)C(=O)OC] |
| S-64 | 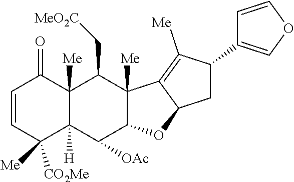 [SMILES: COC(=O)C[C@H]1[C@]2(C)C3=C(C)[C@H](c4ccoc4)C[C@H]3O[C@@H]2[C@H](OC(C)=O)[C@@H]2[C@]1(C)C(=O)C=C[C@@]2(C)C(=O)OC] |
| S-65 | 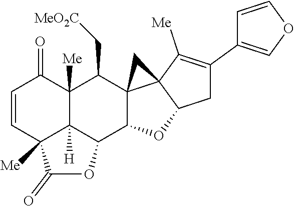 |
| S-84 | 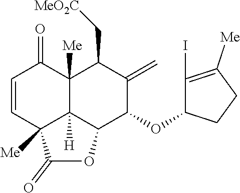 |
| S-97 | 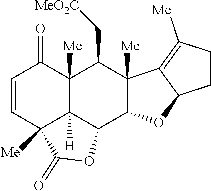 |
| S-98 | 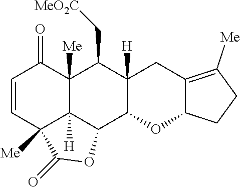 |
| S-99 | 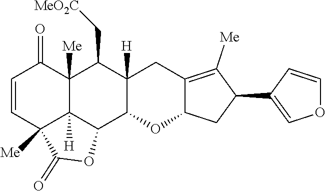 |
| S-100 | 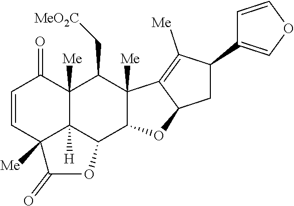 |
| S-101 | 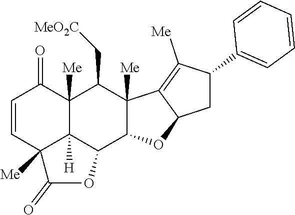 |
| S-102 | 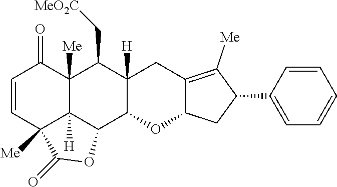 |
| S-103 | 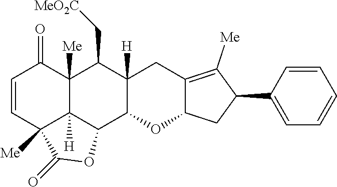 |
| S-104 | 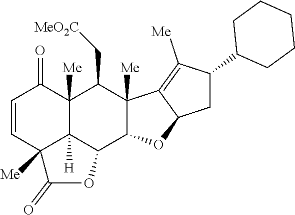 |
| S-105 | 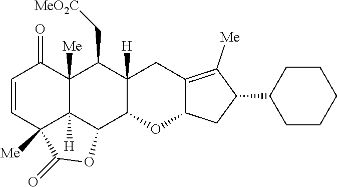 |
| S-106 | 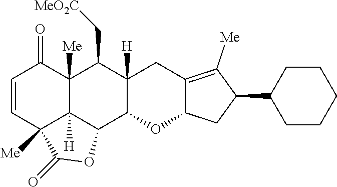 |
| S-107 | 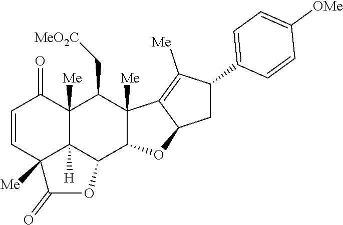 |
| S-108 | 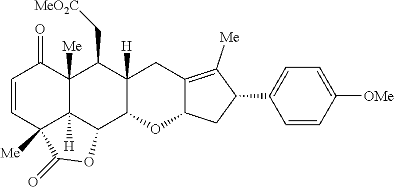 |
| S-109 | 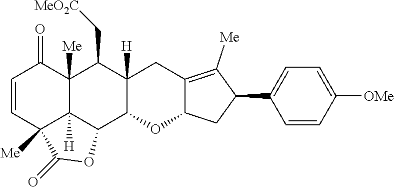 |
| S-111 | 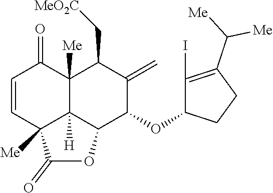 |
| S-112a | 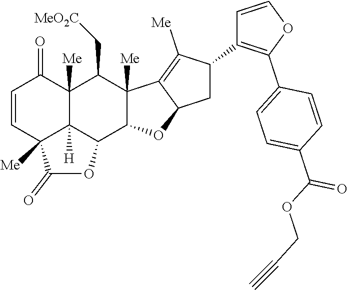 |
| S-101 | 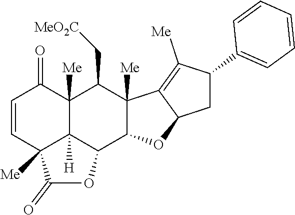 |
| P2 | 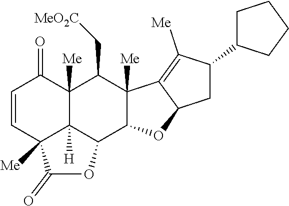 |
| S-97 | 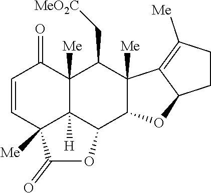 |
| P4 | 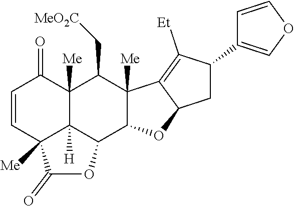 |
| S-100 | 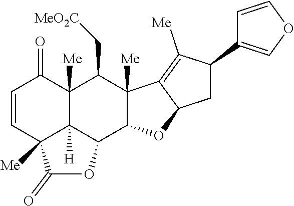 |
| P6 | 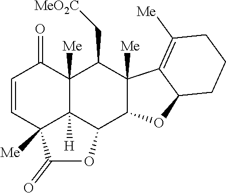 |
| P7 | 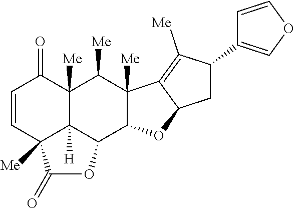 |
| P8 | 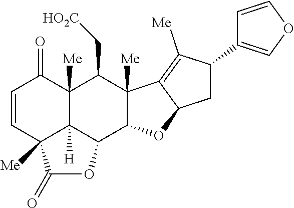 |
| P9 | 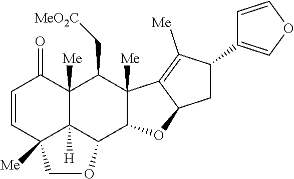 |
| S-112 | 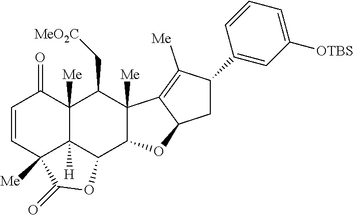 |
| S-113 | 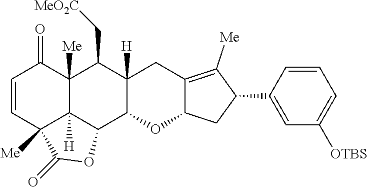 |
| S-114 | 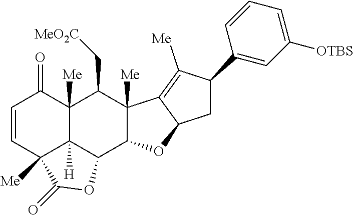 |
| S-115 | 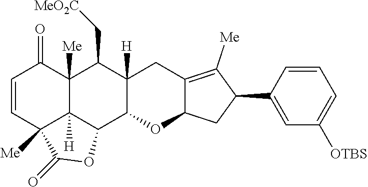 |
| S-116 | 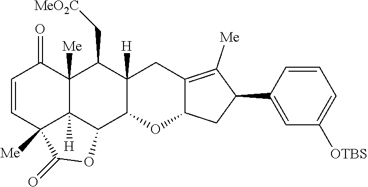 |
| S-117 | 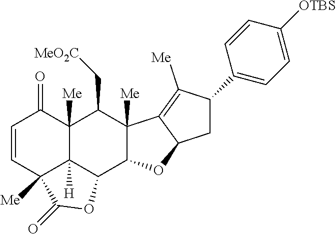 |
| S-118 | 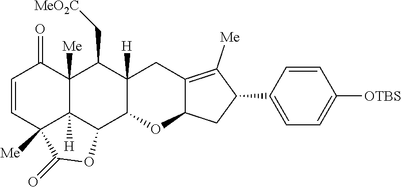 |
| S-119 | 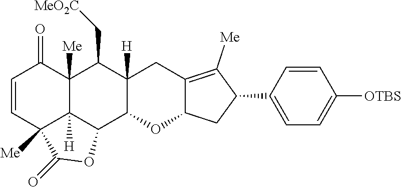 |
| S-120 | 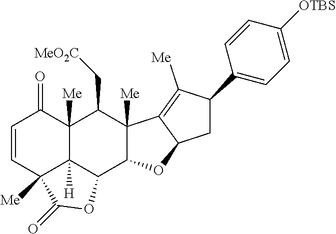 |
| S-121 | 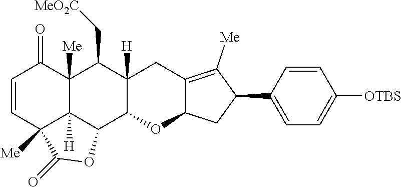 |
| S-122 | 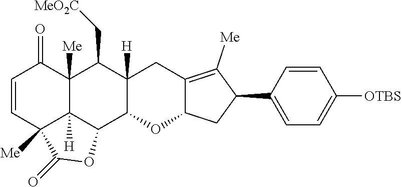 |
| S-123 | 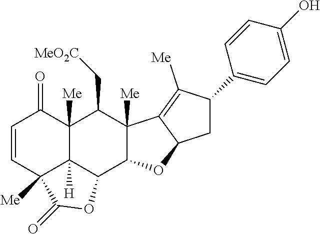 |
| S-124 | 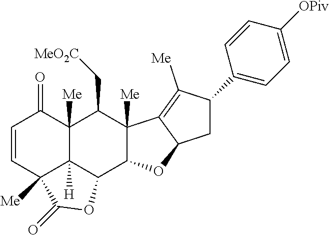 |
| S-125 | 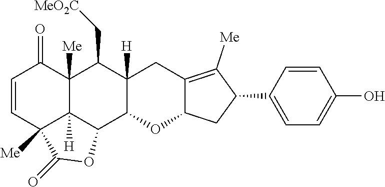 |
| S-126 | 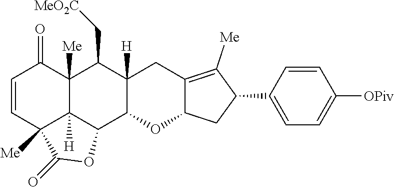 |
| S-127 | 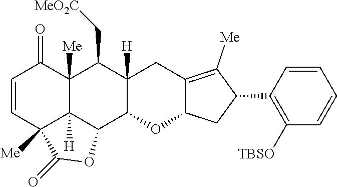 |
| S-128 | 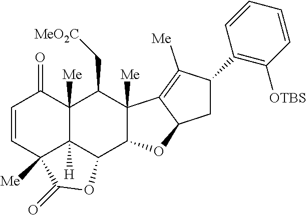 |
| S-129 | 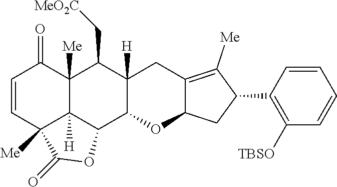 |
| S-130 | 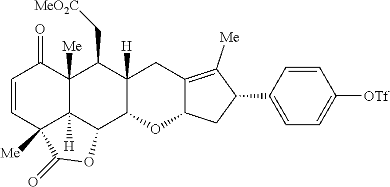 |
| S-131 | 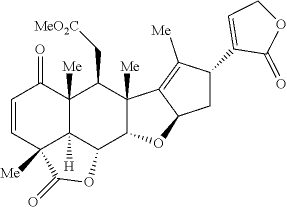 |
| S-132 | 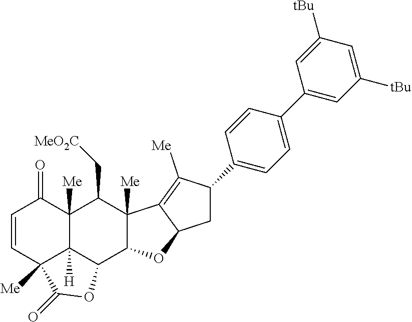 |
| S-133 | 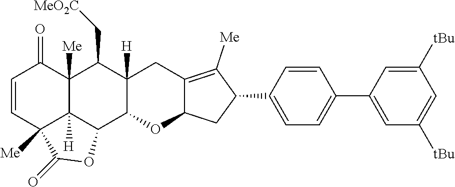 |
| S-134 | 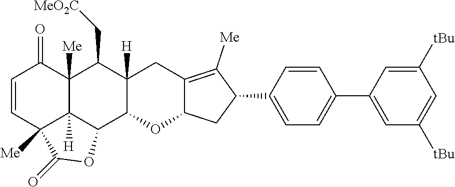 |
| S-135 | 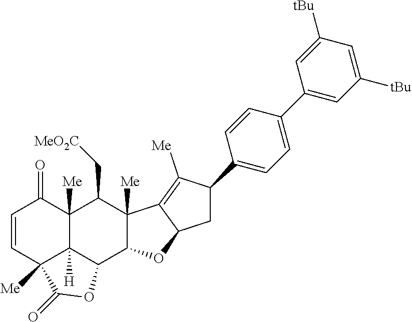 |
| S-136 | 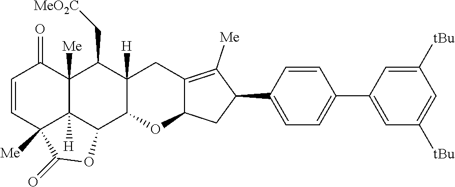 |
| S-137 | 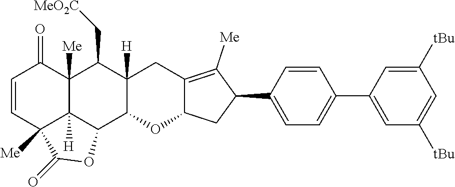 |
| S-138 | 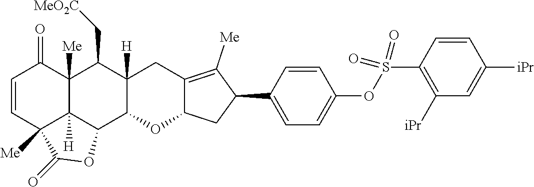 |
| S-139 | 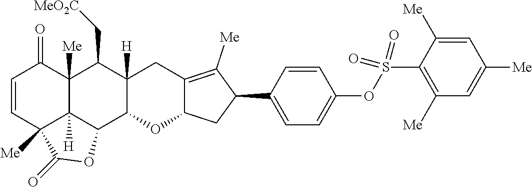 |
| S-140 | 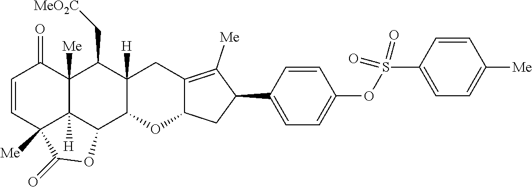 |
| S-141 | 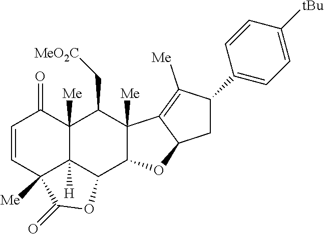 |
| S-142 | 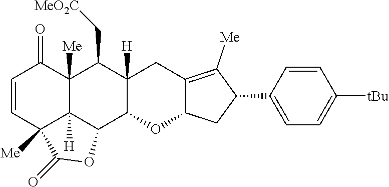 |
| S-143 | 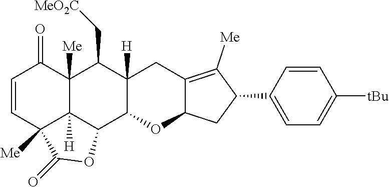 |
| S-144 | 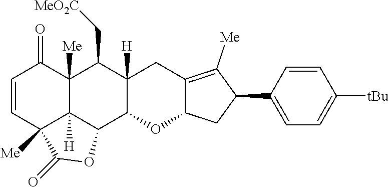 |
| S-145 | 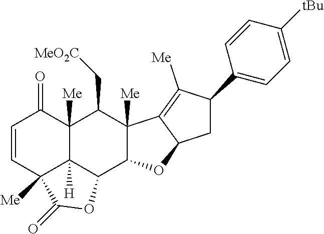 |
| S-146 | 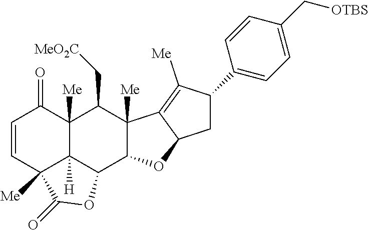 |
| S-147 | 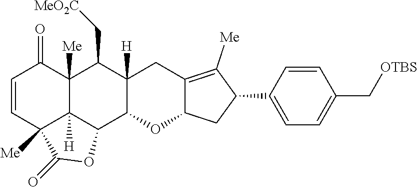 |
| S-148 | 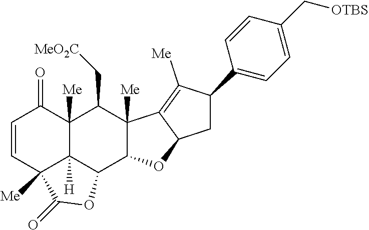 |
| S-149 | 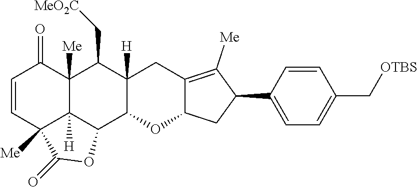 |
| S-150 | 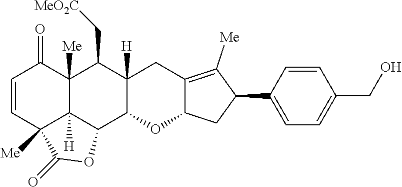 |
| S-151 | 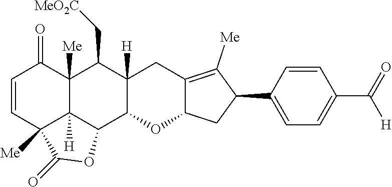 |
| S-152 | 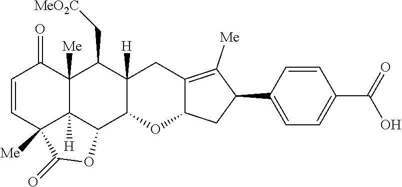 |
| S-153 | 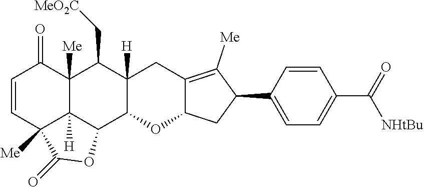 |
| S-154 | 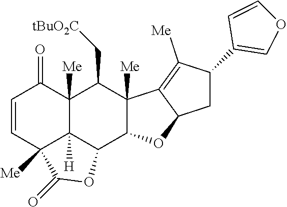 |
| S-155 | 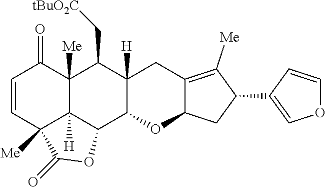 |
| S-156 | 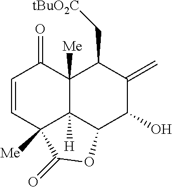 |

All the compounds of the present disclosure may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present disclosure are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present disclosure have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present disclosure may contain one or more asymmetrically-substituted carbon, sulfur, or phosphorus atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration. In some embodiments, the present compounds may contain two or more atoms which have a defined stereochemical orientation.

Chemical formulas used to represent compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended. In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include 13C and ¹⁴C.

In some embodiments, compounds of the present disclosure function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present disclosure exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present disclosure.

### II. PARP1 Trapping by Nimbolide

The studies described herein identified that a novel regulator RNF114 is involved in the PARylation dependent DNA damage response, and PARylated-PARP1 as the specific RNF114 substrate during DNA damage response. RNF114 specifically targeted PARylated-PARP1 for ubiquitin proteasomal degradation, providing a novel ubiquitination dependent mechanism that removes PARP1 from DNA lesions.

Nimbolide is a natural product from the Neem tree. Although it is able to kill various cancer cells, the underlying mechanism is unknown. The studies described herein found that nimbolide covalently modified RNF114 and impaired the E3 ligase activity of RNF114 (Spradlin et al., 2019). Upon nimbolide treatment, RNF114 remains bound to PARylated-PARP1 by its PAR binding domain (PBZ). However, nimbolide treatment compromised the ability of RNF114 to ubiquitinate PARylated-PARP1 for proteasomal degradation. This prevented the release of PARylated-PARP1 from DNA lesions, leading to the formation of trapped PARP1. Thus, nimbolide treatment provides a novel ubiquitination inhibition mechanism for PARP1 trapping.

Compared to a regular PARPi, the mechanism of action for nimbolide is unique, which leads to it being a “super trapper”. First, the data provided herein point to a dominant-negative effect of nimbolide-mediated inhibition of RNF114. Upon the treatment of nimbolide, the binding between RNF114 and PARylated-PARP1 is maintained through the PBZ motifs on RNF114. However, the E3 ligase activity of RNF114 to degrade the PARylated-PARP1 is inhibited. This stabilizes and traps the PARylated-PARP1 without degrading it, leading to a more dramatic PARP1 trapping.

Second, upon sensing DNA strand breaks, PARP1 modifies itself and those proteins in proximity through PARylation. These protein-linked PAR polymers recruit many proteins that contain PAR-binding domains. As an example of these proteins, XRCC1 binds to PAR chains on PARP1, and thus triggers the formation of a large protein complex involved in the repair of DNA single strand breaks (SSBs) (Masson et al., 1998; Zhen and Yu, 2018). Hence, PARylated PARP1 has to be removed so that DNA damage machinery can directly access the DNA lesions and repair them (Fisher et al., 2007). Otherwise, the repair machinery, including XRCC1, will be trapped by PARylation close to DNA lesions. Upon nimbolide exposure, PARylated-PARP1 was trapped on the DNA lesions, and then, XRCC1 and other PAR-binding proteins were recruited by PAR chains of PARylated-PARP1 and trapped close to DNA lesions, suggesting that nimbolide traps DNA repair factors by suppression of the removal of PARylated-PARP1. Thus, nimbolide is also a trapper for the DNA repair machinery, in addition to being a PARP1 trapper. In contrast, regular PARPi block the formation of PAR chains, and therefore are unable to induce the trapping of the PAR-binding DNA repair complex. Collectively, these results identify nimbolide as a PARP1 super trapper that traps hoth PARP1 and PAR-dependent DNA repair factors. Though all the clinical PARPi were developed to block the catalytic activity of PARPI (Rouleau et al., 2010), PARPi with equivalent potency as PARylation inhibitors have dramatically different cytotoxicities in cancer cells (Murai et al., 2012; Murai et al., 2014), which lead to the reconsideration of the mechanism of action of PARPi beyond blocking the catalytic activity of PARP1. Now, it is widely accepted that the cytotoxicity of PARPi is driven by their ability and potency to induce PARP trapping (Murai et al., 2012; Murai et al., 2014).

Though both PARPi and nimbolide induce PARP1 trapping, we found that nimbolide traps both PARylated-PARP1 and PAR-binding proteins, while PARPi only traps PARP1. The difference is that nimbolide traps PARylated-PARP1 which also recruits and traps DNA damage repair factors on the DNA lesions, while PARPi inhibits PARP1 catalytic activity and only traps PARP1 itself. In this context, the inventors have designated nimbolide as a super trapper. Thus, in this study, a novel PARP1 trapping mechanism was identified: utilizing the PARP1 catalytic activity, rather than inhibiting the PARP1 catalytic activity (like PARPi), to induce the trapping of both PARP1 and DNA damage repair factors upon nimbolide treatment.

Besides, it was also confirmed that nimbolide as a super trapper showed advantages over PARPi in suppressing PARPi-resistant malignancies, providing evidence that the cytotoxicity to malignancies is driven by the potency of PARP1 trapping, a higher potency of PARP1 trapping will result in a higher potency of cytotoxicity against malignancies.

In summary, these studies revealed that RNF114 regulates PARP1 dynamics by specifically targeting PARylated-PARP1 for ubiquitin proteasomal degradation in the PARylation dependent DNA damage response. Nimbolide, as the specific RNF114 inhibitor, inhibits the degradation and dissociation of PARylated-PARP1 from DNA lesions, which induces trapping of both PARP1 and DNA repair factors on DNA lesions, leading to fatal DNA damage and lethality in malignancies. We showed that nimbolide and its analogs selectively kill cancer cells with mutations in the double strand repair pathways (e.g., BRCA1/2 mutations). Nimbolide, as a PARP1 super trapper, kills cancer cells with intrinsic and acquired resistance to regular PARPi. Given that nimbolide activates innate immune signaling and up-regulates PD-L1 expression, we identified a cross-talk between nimbolide and tumor-associated immune response and provided evidence to support the combination of nimbolide and immune checkpoint inhibitors (e.g., PD-L1/PD-1 antibodies) as a potential therapeutic approach to treat cancer. Finally, we also showed that nimbolide synergizes with agents that target other DDR enzymes, including ATM, ATRi, and CHK, providing support for the combination of nimbolide with chemo-/radio-therapeutic agents. In all, the results discussed in the Examples below provide a novel mechanism of the regulation of PARP1 trapping by RNF114, suggesting that nimbolide as a super trapper offers promising approaches for the malignancies suppression and cancer-immune therapeutics.

### III. Treatment of Cancer and Other Hyperproliferative Diseases

While hyperproliferative diseases can be associated with any disease which causes a cell to begin to reproduce uncontrollably, the prototypical example is cancer. Psoriasis is another example. One of the key elements of cancer is that the cell's normal apoptotic cycle is interrupted and thus agents that interrupt the growth of the cells are important as therapeutic agents for treating these diseases. In some embodiments, the caffeic acid derivatives described herein may be used to decreased cell counts and as such may be used to treat a variety of cancers or other malignancies.

In some embodiments, cancer, cancer tissue, or cancer cells may be treated by the compounds, methods, and compositions disclosed herein. In some embodiments, cancer cells or tissue that may be treated include but are not limited to cells or tissue from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In some embodiments, the cancer that may be treated may be of the following histological types: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lvmnhoarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia, including hairy cell leukemia; chronic lymphocytic leukemia (CLL); acute lymphoblastic leukemia (ALL); acute myeloid leukemia (AML); and chronic myeloblastic leukemia.

In another aspect, the compounds, compositions, and methods disclosed herein may be used to treat cancer or other hyperproliferative diseases. While hyperproliferative diseases can be associated with any disease which causes a cell to begin to reproduce uncontrollably, the prototypical example is cancer. One of the elements of cancer is that the cell's normal apoptotic cycle is interrupted. As such, agents that interrupt the growth of the cells are important as therapeutic agents for treating these diseases. In this disclosure, the compounds of the present disclosure thereof may be used to lead to decreased cell counts and may be used to treat a variety of types of cancer.

In some embodiments, cancer cells that may be treated with the compounds or compositions of the present disclosure include, but are not limited to, bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, and uterus cells.

In some embodiments, tumors for which the present treatment methods are useful include any malignant cell type, such as those found in a solid tumor or a hematological tumor.

Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma. In certain embodiments regarding methods of treating cancer in a patient, comprising administering to the patient a pharmaceutically effective amount of a compound of the present disclosure, the pharmaceutically effective amount is 0.1-1000 mg/kg. In certain embodiments, the pharmaceutically effective amount is administered in a single dose per day.

In certain embodiments, the pharmaceutically effective amount is administered in two or more doses per day. The compound may be administered by contacting a tumor cell during ex vivo purging, for example. The method of treatment may comprise any one or more of the following: a) inducing cytotoxicity in a tumor cell; b) killing a tumor cell; c) inducing apoptosis in a tumor cell; d) inducing differentiation in a tumor cell; or e) inhibiting growth in a tumor cell. The tumor cell may be any type of tumor cell, such as a brain cell. Other types of cells include, for example, a bladder cancer cell, a breast cancer cell, a lung cancer cell, a colon cancer cell, a prostate cancer cell, a liver cancer cell, a pancreatic cancer cell, a stomach cancer cell, a testicular cancer cell, a brain cancer cell, an ovarian cancer cell, a lymphatic cancer cell, a skin cancer cell, a brain cancer cell, a bone cancer cell, or a soft tissue cancer cell.

In some embodiments, treatment methods further comprise monitoring treatment progress. In some of these embodiments, the method includes the step of determining a level of changes in hematological parameters and/or cancer stem cell (CSC) analysis with cell surface proteins as diagnostic markers or diagnostic measurement (e.g., screen, assay) in a patient suffering from or susceptible to a disorder or symptoms thereof associated with cancer in which the patient has been administered a therapeutic amount of a compound or composition as described herein. The level of the marker determined in the method can be compared to known levels of marker in either healthy normal controls or in other afflicted patients to establish the patient's disease status. In some embodiments, a second level of the marker in the patient is determined at a time point later than the determination of the first level, and the two levels are compared to monitor the course of disease or the efficacy of the therapy. In some embodiments, a pre-treatment level of marker in the patient is determined prior to beginning treatment according to the methods described herein; this pre-treatment level of marker can then be compared to the level of marker in the patient after the treatment commences, to determine the efficacy of the treatment.

In some embodiments, the patient is a mammal, e.g., a primate, preferably a higher primate, e.g., a human (e.g., a patient having, or at risk of having, a disorder described herein).

In some embodiments, the patient is in need of enhancing the patient's immune response. In certain embodiments, the patient is, or is at risk of being, immunocompromised. For example, in mrme embodiments, the patient is undergoing or has undergone a chemotherapeutic treatment and/or radiation therapy. Alternatively, or in combination, the patient is, or is at risk of being, immunocompromised as a result of an infection.

### IV. Pharmaceutical Formulations and Routes of Administration

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-wnter CGF emulsions as well as conventional liposomes. The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the nermenhility of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier.

In some embodiments, the specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in HED values based on body surface area (BSA) rather than only on body mass. Kₘ values for humans and various animals are well known.

For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Km of 5 (given a weight of 0.08 kg and BSA of 0.02); rat Km of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine cchedile refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between.

Alternatively, the predetermined routine schedule may involve administration on a twice daily basis for the first week, followed by a daily basis for several months, etc. In other embodiments, the disclosure provides that the agent(s) may be taken orally and that the timing of which is or is not dependent upon food intake. Thus, for example, the agent can be taken every morning and/or every evening, regardless of when the patient has eaten or will eat.

### IV. Immune Checkpoint Inhibitors

In certain embodiments, the present disclosure provides methods of combining the blockade of immune checkpoints with nimbolide and its derivatives. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO 2015/016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

It is contemplated that any of the immune checkpoint inhibitors that are known in the art to stimulate immune responses may be used. This includes inhibitors that directly or indirectly stimulate or enhance antigen-specific T-lymphocytes. These immune checkpoint inhibitors include, without limitation, agents targeting immune checkpoint proteins and pathways involving PD-L2, LAG3, BTLA, B7H4 and TIM3. For example, LAG3 inhibitors known in the art include soluble LAG3 (IMP321, or LAG3-Ig disclosed in WO 2009/044273) as well as mouse or humanized antibodies blocking human LAG3 (e.g., IMP701 disclosed in WO 2008/132601), or fully human antibodies blocking human LAG3 (such as disclosed in EP 2320940). Another example is provided by the use of blocking agents towards BTLA, including without limitation antibodies blocking human BTLA interaction with its ligand (such as 4C7 disclosed in WO 2011/014438). Yet another example is provided by the use of agents neutralizing B7H4 including without limitation antibodies to human B7H4 (disclosed in WO 2013/025779, and in WO 2013/067492) or soluble recombinant forms of B7H4 (such as disclosed in US 2012/0177645). Yet another example is provided by agents neutralizing B7-H3, including without limitation antibodies neutralizing human B7-H3 (e.g. MGA271 disclosed as BRCA84D and derivatives in US 20120294796). Yet another example is provided by agents targeting TIM3, including without limitation antibodies targeting human TIM3 (e.g. as disclosed in WO 2013/006490 or the anti-human TIM3, blocking antibody F38-2E2 disclosed by Jones et al., J Exp Med. 2008; 205(12):2763-79).

In addition, more than one immune checkpoint inhibitor (e.g., anti-PD-1 antibody and anti-CTLA-4 antibody) may be used in combination with the nimbolide derivatives. For example, nimbolide derivatives and immune checkpoint inhibitors (e.g., anti-KIR antibody and/or anti-PD-1 antibody) can be administered to enhance innate anti-tumor immunity followed by IL24 gene therapy and immune checkpoint inhibitors (e.g., anti-PD-1 antibody) to induce adaptive anti-tumor immune responses.

### A. PD-1 Axis Antagonists

cell dysfunction or anergy occurs concurrently with an induced and sustained expression of the inhibitory receptor, programmed death 1 polypeptide (PD-1). Thus, therapeutic targeting of PD-1 and other molecules which signal through interactions with PD-1, such as programmed death ligand 1 (PD-L1) and programmed death ligand 2 (PD-L2) is provided herein. PD-L1 is overexpressed in many cancers and is often associated with poor prognosis (Okazaki T et al., Intern. Immun. 2007 19(7):813). Thus, inhibition of the PD-L1/PD-1 interaction in combination with nimbolide derivatives is provided herein such as to enhance CD8+ T cell-mediated killing of tumors.

Provided herein is a method for treating or delaying progression of cancer in an indlividnl comprising administering to the individual an effective amount of a PD-1 axis binding antagonist in combination with nimbolide derivatives. Also provided herein is a method of enhancing immune function in an individual in need thereof comprising administering to the individual an effective amount of a PD-1 axis binding antagonist and a nimbolide derivative.

For example, a PD-1 axis binding antagonist includes a PD-1 binding antagonist, a PDL1 binding antagonist and a PDL2 binding antagonist. Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesion, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO@, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 binding antagonists include Pidilizumab, also known as CT-011, MEDIO680, also known as AMP-514, and REGN2810.

In some aspects, the immune checkpoint inhibitor is a PD-L1 antagonist such as Durvalumab, also known as MEDI4736, atezolizumab, also known as MPDL3280A, or avelumab, also known as MSB00010118C. In certain aspects, the immune checkpoint inhibitor is a PD-L2 antagonist such as rHIgM12B7. In some aspects, the immune checkpoint inhibitor is a LAG-3 antagonist such as, but not limited to, IMP321, and BMS-986016. The immune checkpoint inhibitor may be an adenosine A2a receptor (A2aR) antagonist such as PBF-509.

In some aspects, the antibody described herein (such as an anti-PD-1 antibody, an anti-PDL1 antibody, or an anti-PDL2 antibody) further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further specific aspect, the antibody has reduced or minimal effector function. In a still further specific aspect, the minimal effector function results from production in prokaryotic cells. In a still further specific aspect, the minimal effector function results from an “effector-less Fc mutation” or aglycosylation.

Accordingly, an antibody used herein can be aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxy amino acid, most commonly serine or threonine, although 5-hydroxyproline or 5-hydroxy lysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

The antibody or antigen binding fragment thereof, may be made using methods known in the art, for example, by a process comprising culturing a host cell containing nucleic acid encndinu any of the previously described anti-PDL1, anti-PD-1, or anti-PDL2 antibodies or antigen-binding fragment in a form suitable for expression, under conditions suitable to produce such antibody or fragment, and recovering the antibody or fragment.

### B. Immunomodulatory Agents

Immunomodulatory agents include immune checkpoint inhibitors, agonists of co-stimulatory molecules, and antagonists of immune inhibitory molecules. The immunomodulatory agents may be drugs, such as small molecules, recombinant forms of ligand or receptors, or antibodies, such as human antibodies (e.g., International Patent Publication WO2015/016718; Pardoll, Nat Rev Cancer, 12(4): 252-264, 2012; both incorporated herein by reference). Known inhibitors of immune checkpoint proteins or analogs thereof may be used, in particular chimerized, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

Co-stimulatory molecules are ligands that interact with receptors on the surface of the immune cells, e.g., CD28, 4-1BB, OX40 (also known as CD134), ICOS, and GITR. As an example, the complete protein sequence of human OX40 has Genbank accession number NP_003318. In some embodiments, the immunomodulatory agent is an anti-OX40 antibody (e.g., a human antibody, a humanized antibody, or a chimeric antibody), an antigen binding fragment thereof, an immunoadhesin, a fusion protein, or oligopeptide. Anti-human-OX40 antibodies (or VH and/or VL domains derived therefrom) suitable for use in the present methods can be generated using methods well known in the art. Alternatively, art recognized anti-OX40 antibodies can be used. An exemplary anti-OX40 antibody is PF-04518600 (see, e.g., WO 2017/130076). ATOR-1015 is a bispecific antibody targeting CTLA4 and OX40 (see, e.g., WO 2017/182672, WO 2018/091740, WO 2018/202649, WO 2018/002339).

Another co-stimulatory molecule that can be targeted in the methods provided herein is ICOS, also known as CD278. The complete protein sequence of human ICOS has Genbank accession number NP_036224. In some embodiments, the immune checkpoint inhibitor is an anti-ICOS antibody (e.g., a human antibody, a humanized antibody, or a chimeric antibody), an antigen binding fragment thereof, an immunoadhesin, a fusion protein, or oligopeptide.

Anti-human-ICOS antibodies (or VH and/or VL domains derived therefrom) suitable for use in the present methods can be generated using methods well known in the art. Alternatively, art reCnnized anti-ICOS antibodies can be used. Exemplary anti-ICOS antibodies include JTX-2011 (see, e.g., WO 2016/154177, WO 2018/187191) and GSK3359609 (see, e.g., WO 2016/059602).

Yet another co-stimulatory molecule that can be targeted in the methods provided herein is glucocorticoid-induced tumour necrosis factor receptor-related protein (GITR), also known as TNFRSF18 and AITR. The complete protein sequence of human GITR has Genbank accession number NP_004186. In some embodiments, the immunomodulatory agent is an anti-GITR antibody (e.g., a human antibody, a humanized antibody, or a chimeric antibody), an antigen binding fragment thereof, an immunoadhesin, a fusion protein, or oligopeptide. Anti-human-GITR antibodies (or VH and/or VL domains derived therefrom) suitable for use in the present methods can be generated using methods well known in the art. Alternatively, art recognized anti-GITR antibodies can be used. An exemplary anti-GITR antibody is TRX518 (see, e.g., WO 2006/105021).

Immune checkpoint proteins that may be targeted by immune checkpoint blockade include adenosine A2A receptor (A2AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCR5, HLA-DRB1, HLA-DQA1, HLA-E, killer-cell immunoglobulin (KIR), lymphocyte activation gene-3 (LAG-3, also known as CD223), Mer tyrosine kinase (MerTK), NKG7, programmed death 1 (PD-1), programmed death-ligand 1 (PD-L1, also known as CD274), PDCD1LG2, PSMB10, STAT1, T cell immunoreceptor with Ig and ITIM domains (TIGIT), T-cell immunoglobulin domain and mucin domain 3 (TIM-3), and V-domain Ig suppressor of T cell activation (VISTA, also known as C10orf54). In particular, immune checkpoint inhibitors targeting the PD-1 axis and/or CTLA-4 have received FDA approval broadly across diverse cancer types.

In some embodiments, a PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, a PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesin, a fusion protein, or an oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art, such as described in U.S. Patent Application Publication Nos. 2014/0294898, 2014/022021, and 2011/0008369, all of which are incorporated herein by reference.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

Another immune checkpoint protein that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in U.S. Pat. No. 8,119,129; PCT Publn. Nos. WO 01/14424, WO 98/42752, WO 00/37504 (CP675,206, also known as tremelimumab; formerly ticilimumab); U.S. Pat. No. 6,207,156; Hnrwt7 t al. (1998) Proc Nat Acad Sci USA, 95(17): 10067-10071; Camacho et al. (2004) J Clin Oncology, 22 (145): Abstract No. 2505 (antibody CP-675206); and Mokyr et al. (1998) Cancer Res, 58:5301-5304 can be used in the methods disclosed herein. The teachings of each of the aforementioned publications are hereby incorporated by reference. Antibodies that compete with any of these art-recognized antibodies for binding to CTLA-4 also can be used.

For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies.

In another embodiment, the antibody has an at least about 90% variable region amino acid sequence identity with the above-mentioned antibodies (e.g., at least about 90%, 95%, or 99% variable region identity with ipilimumab). Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Another immune checkpoint protein that can be targeted in the methods provided herein is lymphocyte-activation gene 3 (LAG-3), also known as CD223. The complete protein sequence of human LAG-3 has the Genbank accession number NP-002277. LAG-3 is found on the surface of activated T cells, natural killer cells, B cells, and plasmacytoid dendritic cells. LAG-3 acts as an “off” switch when bound to MHC class II on the surface of antigen-presenting cells. Inhibition of LAG-3 both activates effector T cells and inhibitor regulatory T cells. In some embodiments, the immune checkpoint inhibitor is an anti-LAG-3 antibody (e.g., a human antibody, a humanized antibody, or a chimeric antibody), an antigen binding fragment thereof, an immunoadhesin, a fusion protein, or oligopeptide. Anti-human-LAG-3 antibodies (or VH and/or VL domains derived therefrom) suitable for use in the present methods can be generated using methods well known in the art. Alternatively, art recognized anti-LAG-3 antibodies can be used. An exemplary anti-LAG-3 antibody is relatlimab (also known as BMS-986016) or nntiuen hinding fragments and variants thereof (see, e.g., WO 2015/116539). Other exemplary anti-LAG-3 antibodies include TSR-033 (see, e.g., WO 2018/201096), MK-4280, and REGN3767. MGD013 is an anti-LAG-3/PD-1 bispecific antibody described in WO 2017/019846. FS118 is an anti-LAG-3/PD-L1 bispecific antibody described in WO 2017/220569.

Another immune checkpoint protein that can be targeted in the methods provided herein is V-domain Ig suppressor of T cell activation (VISTA), also known as C10orf54. The complete protein sequence of human VISTA has the Genbank accession number NP_071436. VISTA is found on white blood cells and inhibits T cell effector function. In some embodiments, the immune checkpoint inhibitor is an anti-VISTA3 antibody (e.g., a human antibody, a humanized antibody, or a chimeric antibody), an antigen binding fragment thereof, an immunoadhesin, a fusion protein, or oligopeptide. Anti-human-VISTA antibodies (or VH and/or VL domains derived therefrom) suitable for use in the present methods can be generated using methods well known in the art. Alternatively, art recognized anti-VISTA antibodies can be used. An exemplary anti-VISTA antibody is JNJ-61610588 (also known as onvatilimab) (see, e.g., WO 2015/097536, WO 2016/207717, WO 2017/137830, WO 2017/175058). VISTA can also be inhibited with the small molecule CA-170, which selectively targets both PD-L1 and VISTA (see, e.g., WO 2015/033299, WO 2015/033301).

Another immune checkpoint protein that can be targeted in the methods provided herein is CD38. The complete protein sequence of human CD38 has Genbank accession number NP_001766. In some embodiments, the immune checkpoint inhibitor is an anti-CD38 antibody (e.g., a human antibody, a humanized antibody, or a chimeric antibody), an antigen binding fragment thereof, an immunoadhesin, a fusion protein, or oligopeptide. Anti-human-CD38 antibodies (or VH and/or VL domains derived therefrom) suitable for use in the present methods can be generated using methods well known in the art. Alternatively, art recognized anti-CD38 antibodies can be used. An exemplary anti-CD38 antibody is daratumumab (see, e.g., U.S. Pat. No. 7,829,673).

Another immune checkpoint protein that can be targeted in the methods provided herein is T cell immunoreceptor with Ig and ITIM domains (TIGIT). The complete protein sequence of human TIGIT has Genbank accession number NP_776160. In some embodiments, the immune checkpoint inhibitor is an anti-TIGIT antibody (e.g., a human antibody, a humanized antibody, or a chimeric antibody), an antigen binding fragment thereof, an immunoadhesin, a fusion protein, or oligopeptide. Anti-human-TIGIT antibodies (or VH and/or VL domains derived therefrom) suitable for use in the present methods can be generated using methods well known in the art. Alternatively, art recognized anti-TIGIT antibodies can be used. An exemplary anti-TIGIT antibody is MK-7684 (see, e.g., WO 2017/030823, WO 2016/028656).

Other immune inhibitory molecules that can be targeted for immunomodulation include STAT3 and indoleamine 2,3-dioxygenase (IDO). By way of example, the complete protein sequence of human IDO has Genbank accession number NP_002155. In some embodiments, the immunomodulatory agent is a small molecule IDO inhibitor. Exemplary small molecules include BMS-986205, epacadostat (INCB24360), and navoximod (GDC-0919).

### V. Methods of Treatment

Provided herein are methods for treating or delaying progression of cancer in an individual comprising administering to the individual an effective amount of at least one nimbolide derivative. The therapy may further comprise at least one immune checkpoint inhibitor (e.g., PD-1 axis binding antagonist and/or CTLA-4 antibody).

In some embodiments, the treatment results in a sustained response in the individual after cessation of the treatment. The methods described herein may find use in treating conditions where enhanced immunogenicity is desired such as increasing tumor immunogenicity for the treatment of cancer. In some embodiments, the individual is a human.

In some aspects, the subject is further administered a tumor suppressor immune gene therapy (see, PCT/US2016/060833, which is incorporated herein by reference in its entirety).

In some aspects, the subject is further administered additional viral and non-viral gene therapies (PCT/US2017/065861; incorporated herein by reference in its entirety). In some aspects, the replication competent and/or replication incompetent viral and/or non-viral gene therapy may deliver one or more therapeutic genes which could be tumor suppressor genes or immune stimulatory genes.

Examples of cancers contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer.

In some embodiments, the individual has cancer that is resistant (has been demonstrated to be resistant) to one or more anti-cancer therapies. In some embodiments, resistance to anti-cancer therapy includes recurrence of cancer or refractory cancer. Recurrence may refer to the reappearance of cancer, in the original site or a new site, after treatment. In some embodiments, resistance to anti-cancer therapy includes progression of the cancer during treatment with the anti-cancer therapy. In some embodiments, the cancer is at early stage or at late stage. In some embodiments, the subject is also treated with an immune checkpoint inhibitor such as a PD-1 axis binding antagonist and/or an anti-CTLA-4 antibody. The individual may have a cancer that expresses (has been shown to express e.g., in a diagnostic test) PD-L1 biomarker or have a high tumor mutational burden. In some embodiments, the patient's cancer expresses low PD-L1 biomarker. In some embodiments, the patient's cancer expresses high PD-L1 biomarker. The PD-L1 biomarker can be detected in the sample using a method selected from the group consisting of FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometery, HPLC, qPCR, RT-qPCR, multiplex qPCR or RT-qPCR, RNA-seq, microarray analysis, SAGE, MassARRAY technique, and FISH, and combinations thereof. Measurement of a high mutational tumor burden may be determined by genomic sequencing (e.g., Foundation One CDx assay).

The efficacy of any of the methods described herein (e.g., combination treatments including administering an effective amount of a combination of at least one nimbolide derivative and at least one immune checkpoint inhibitor may be tested in various models known in the art, such as clinical or pre-clinical models.

The present disclosure is useful for any human cell that participates in an immune reaction either as a target for the immune system or as part of the immune system's response to the foreign target. The methods include ex vivo methods, in vivo methods, and various other methods that involve injection of polynucleotides or vectors into the host cell. The methods also include injection directly into the tumor or tumor bed as well as local or regional to the tumor.

### A. Administration

The combination therapy provided herein comprises administration of one or more immune checkpoint inhibitors and a nimbolide derivative. The combination therapy may be administered in any suitable manner known in the art. For example, the immune checkpoint inhibitor and nimbolide derivative may be administered sequentially (at different times) or concurrently (at the same time). In some embodiments, the one or more immune checkpoint inhibitors are in a separate composition as the nimbolide derivative. In some embodiments, the one or more immune checkpoint inhibitors are in the same composition as the nimbolide derivative. In certain aspects, the subject is administered the nimbolide derivative before, simultaneously, or after the at least one immune checkpoint inhibitor. The one or more immune checkpoint inhibitors and the nimbolide derivative may be administered by the same route of administration or by different routes of administration. In some embodiments, the one or more immune checkpoint inhibitors is administered intravenously, intramuscularly, subcutaneously, topically, orally, transdermally, intraperitoneally, intraorbitally, by implantation, by inhalation, intrathecally, intraventricularly, or intranasally. In some embodiments, the nimbolide derivative is administered intravenously, intramuscularly, subcutaneously, topically, orally, transdermally, intraperitoneally, intraorbitally, by implantation, by inhalation, intrathecally, intraventricularly, or intranasally. An effective amount of the one or more immune checkpoint inhibitors and the nimbolide derivative may be administered for prevention or treatment of disease. The appropriate dosage of one or more immune checkpoint inhibitors and/or the nimbolide derivative may be determined based on the type of disease to be treated, severity and course of the disease, the clinical condition of the individual, the individual's clinical history and response to the treatment, and the discretion of the attending physician. In some embodiments, combination treatment with the at least one or more immune checkpoint inhibitors and a nimbolide derivative are synergistic, whereby there is more than an additive effect of separate doses of a nimbolide derivative in the combination with at the least one or more immune checkpoint inhibitors compared to the treatment as a single agent.

For example, the therapeutically effective amount of the one or more immune checkpoint inhibitors is administered in doses ranging between 5-100 pg/kg given either SQ or IV at intervals ranging from weekly to every 2-4 weeks.

For example, when the therapeutically effective amount of the nimbolide derivative is administered in further combination with an immune checkpoint inhibitor, such as an antibody, will be in the range of about 0.01 to about 50 mg/kg of patient body weight whether by one or more administrations. In some embodiments, the antibody used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In some embodiments, the antibody is administered at 15 mg/kg. However, other dosage regimens may be useful. In one embodiment, an anti-PD-L1 antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on dav 1 of 21-day cycles. The dose may be administered as a single dose or as multiple doses (e.g., 2 or 3 doses), such as infusions. The progress of this therapy is easily monitored by conventional techniques.

Intratumoral injection, or injection into the tumor vasculature is specifically contemplated for nimbolide derivative component of the combined therapy. Local, regional or systemic administration also may be appropriate. For tumors of >4 cm, the volume to be administered will be about 4-10 mL (in particular 10 mL), while for tumors of <4 cm, a volume of about 1-3 mL will be used (in particular 3 mL). Multiple injections delivered as single dose comprise about 0.1 to about 0.5 mL volumes. For example, adenoviral particles may advantageously be contacted by administering multiple injections to the tumor.

Treatment regimens may vary as well, and often depend on tumor type, tumor location, disease progression, and health and age of the patient. Obviously, certain types of tumors will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor being treated may not, at least initially, be resectable.

The combined treatments may increase the resectability of the tumor due to shrinkage at the margins or by elimination of certain particularly invasive portions. Following the combined treatments, resection is performed. Additional treatments subsequent to resection will serve to eliminate residual disease.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time.

### B. Additional Anti-Cancer Therapies

In order to increase the effectiveness of the nimbolide derivative and the at least one immune checkpoint inhibitor, they can be combined with at least one additional agent effective in the treatment of cancer. More generally, these other compositions would be provided in a combined amount effective to kill or inhibit proliferation of the cell. This process may involve contacting the cells with the agent(s) or multiple factor(s) at the same time. This may be achieved by contacting the cell with a single composition or pharmacological formulation that includes both agents, or by contacting the cell with two distinct compositions or formulations, at the same time, wherein one composition includes the nimbolide derivative and the other inclndes the second agent(s). Alternatively, the nimbolide derivative may contact the proliferating cell and the additional therapy may affect other cells of the immune system or the tumor microenvironment to enhance anti-tumor immune responses and therapeutic efficacy.

The at least one additional anticancer therapy may be, without limitation, a surgical therapy, chemotherapy (e.g., administration of a protein kinase inhibitor or a EGFR-targeted therapy), radiation therapy, cryotherapy, hyperthermia treatment, phototherapy, radioablation therapy, hormonal therapy, immunotherapy including but not limited to immune checkpoint inhibitors, small molecule therapy, receptor kinase inhibitor therapy, anti-angiogenic therapy, cytokine therapy or a biological therapies such as monoclonal antibodies, siRNA, miRNA, antisense oligonucleotides, ribozymes or gene therapy. Without limitation the biological therapy may be a gene therapy, such as a cell death protein gene therapy, a cell cycle regulator gene therapy, a cytokine gene therapy, a toxin gene therapy, an immunogene therapy, a suicide gene therapy, a prodrug gene therapy, an anti-cellular proliferation gene therapy, an enzyme gene therapy, or an anti-angiogenic factor gene therapy.

The gene therapy may precede or follow the other agent treatment by intervals ranging from minutes to weeks. In embodiments where the other agent and nimbolide derivative are applied separately to the cell, one would generally ensure that a significant period of time did not expire between the time of each delivery, such that the agent and nimbolide derivative would still be able to exert an advantageously combined effect on the cell. In such instances, it is contemplated that one may contact the cell with both modalities within about 12-24 hours of each other and, more preferably, within about 6-12 hours of each other. In some situations, it may be desirable to extend the time period for treatment significantly, however, where several days (e.g., 2, 3, 4, 5, 6 or 7) to several weeks (e.g., 1, 2, 3, 4, 5, 6, 7 or 8) lapse between the respective administrations. In certain embodiments, one or more of the therapies may be continued either with or without the others as maintenance therapy.

Various combinations may be employed, nimbolide derivative is “A” and the secondary agent, i.e. an immune checkpoint inhibitor, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

### 1. Chemotherapy

Cancer therapies in general also include a variety of combination therapies with both chemical and radiation-based treatments. Combination chemotherapies include, for example, cisplatin (CDDP), carboplatin, procarbazine, mechlorethamine, cyclophosphamide, cnmntothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The chemotherapy may also be administered at low, continuous doses which is known as metronomic chemotherapy.

Yet further combination chemotherapies include, for example, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaIl; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercnntnourine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; carboplatin, procarbazine, plicomycin, gemcitabien, navelbine, farnesyl-protein transferase inhibitors, transplatinum; and pharmaceutically acceptable salts, acids or derivatives of any of the above.

In certain embodiments, the compositions provided herein may be used in combination with histone deacetylase inhibitors. In certain embodiments, the compositions provided herein may be used in combination with gefitinib. In other embodiments, the present embodiments may be practiced in combination with Gleevec (e.g., from about 400 to about 800 mg/day of Gleevec may be administered to a patient). In certain embodiments, one or more chemotherapeutic may be used in combination with the compositions provided herein.

DNA-dependent protein kinase (DNA-PK) is a serine/threonine protein kinase which is activated in conjunction with DNA. Biochemical and genetic data show that DNA-PK consists (a) of a catalytic sub-unit, which is called DNA-PKcs, and (b) two regulatory components (Ku70 and Ku80). In functional terms, DNA-PK is a crucial constituent on the one hand of the repair of DNA double-strand breaks (DSBs) and on the other hand of somatic or V(D)J recombination. In addition, DNA-PK and its components are connected with a multiplicity of further physiological processes, including modulation of the chromatin structure and telomeric maintenance. Exemplary DNAPK inhibitors include those disclosed in WO2016/210046, WO2018/178040, AZD7648, MSC-2490484, and M-3814.

DNA polymerase theta (also referred to as PolQ; Gene ID No. 10721) is a DNA polymerase that also functions as an DNA-dependent ATPase. PolQ is implicated in a pathway required for the repair of double-stranded DNA breaks, referred to as the error-prone microhomology-mediated end-joining (MMEJ) pathway. As used herein, a “PolQ inhibitor” is any agent that reduces, slows, halts, and/or prevents PolQ activity in a cell relative to vehicle, or an agent that reduces or prevents expression of PolQ protein. Typically, PolQ comprises two distinct enzymatic (catalytic) domains, an N-terminal ATPase and a C-terminal polymerase domain. Thus, a PolQ inhibitor can be an agent (e.g., a small molecule, peptide or antisense molecule) that inhibits polymerase function, ATPase function, or polymerase function and ATPase function of PolQ. In some embodiments, the inhibitor reduces, slows, halts, and/or prevents the ATPase activity of PolQ. A PolQ inhibitor can be any molecule or compound that inhibits PolQ as described above, including a small molecule, antibody or antibody fragments, peptide or antisense compound, siRNA and shRNA, and DNA and RNA aptamers. In some embodiments, a PolQ inhibitor is a molecule that reduces or prevents expression of PolQ, such as one or more antisense molecules (e.g., siRNA, shRNA, dsRNA, miRNA, amiRNA, antisense oligonucleotides (ASO)) that target DNA or mRNA encoding PolQ. In some embodiments, the antisense molecule is an interfering RNA (e.g., dsRNA, siRNA, shRNA, miRNA, amiRNA, ASO). In some embodiments, a PolQ inhibitor is as disclosed WO2020160213, as disclosed in WO2020160134, or novobiocin.

Homology-dependent repair at a DNA double-strand break starts with the localization of the MRE11-RAD50-NBS1 (MRN) complex to the double stranded break. The break is resected, long-range chromatin modifications take place and the resected DNA invades the homologous sequence in a Rad51/52 dependent reaction. Additional factors are then required, including Rad paralogs and BRCA1/2 in vertebrates. Exemplary MRE11 inhibitors include Mirin; PFMO1; and PFM39. Also contemplated are any of the DNA repair pathway inhibitors recited in Hengel et al. (2017), which is incorporated by reference herein in its entirety. “CHK inhibitor” or “CHKi” refers to an inhibitor of a checkpoint kinase, CHK1 and/or CHK2. Preferably, the CHK inhibitor is a molecule that inhibits the enzymatic activity of a checkpoint kinase (CHK). Examples of CHK inhibitors that are useful in the treatment method, medicaments and uses of the present disclosure include, but are not limited to, SCH900776, LY2603618, MK-8776, CCT245737, GDC-0575, BLM-277, V158411, XL-844, PF-477736, UCN-01 AZD7762, and EXEL-9844. “ATR/ATM inhibitor” or “ATRi” or “ATMi” or “ATR/ATMi” refers to an inhibitor of the ATR/ATM kinase pathway, which mediates the DNA damage response. Preferably, the ATR/ATM inhibitor is a molecule that inhibits the enzymatic activity of the ATR/ATM kinase.

Examples of ATR/ATM inhibitors that are useful in the treatment method, medicaments and uses of the present disclosure include, but are not limited to, AZD6738, CGK733, and any of the compounds described in WO 2013/049726, WO 2013/152298, WO 2013/049859, US 2013-0089625, US 2013-0115312, US 2014-0107093, US 2013-0096139, WO 2011/143426, US 2013-0095193, WO 2014/055756, WO 2011/143419, WO 2011/143422, WO 2011/143425, US 2013-0115311, US 2013-0115312, US 2013-0115313, US 2013-0115314, WO 2011/163527, WO 2012/178123, WO 2012/178124, WO 2012/178125, US 2014-0113005, WO 2013/049726, WO 2013/071085, WO 2010/071837, WO 2014/089379, WO 2014/143242, WO 2014/143241, WO 2015/084384, WO 2014/143240, WO 2015/187451, WO 2015/085132, WO 2014/062604, WO 2014/143240, WO 2013/071094, WO 2013/071093, WO 2013/071090, WO 2013/071088, WO 2013/049859, WO 2013/049719, WO 2013/049720, WO 2013/049722, WO 2012/138938, WO 2011/163527, WO 2011/143423, WO 2011/143426, WO 2011/143399, and WO 2010/054398.

### 2. Radiotherapy

Other factors that cause DNA damage and have been used extensively include what are commonly known as y-rays, X-rays, and/or the directed delivery of radioisotopes to tumor cells. Other forms of DNA damaging factors are also known such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens.

Dosage ranges for radioisotopes vary widely, and depend on the half-life of the isotope, the strength and type of radiation emitted, and the uptake by the neoplastic cells.

### 3. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells as well as genetically engineered variants of these cell types modified to express chimeric antigen receptors. Mda-7 gene transfer to tumor cells causes tumor cell death and apoptosis. The apoptotic tumor cells are scavenged by reticuloendothelial cells including dendritic cells and macrophages and presented to the immune system to generate anti-tumor immunity (Rovere et al., 1999; Steinman et al., 1999).

It will be appreciated by those skilled in the art of cancer immunotherapy that other complementary immune therapies may be added to the regimens described above to further enhance their efficacy including but not limited to GM-CSF to increase the number of myeloid derived innate immune system cells, low dose cyclophosphamide or PI3K inhibitors (e.g., PI3K delta inhibitors) to eliminate T regulatory cells that inhibit innate and adaptive immunity and 5FU (e.g., capecitabine), PI3K inhibitors or histone deacetylase inhibitors to remove inhibitory myeloid derived suppressor cells. For example, PI3K inhibitors include, but are not limited to, LY294002, Perifosine, BKM120, Duvelisib, PX-866, BAY 80-6946, BEZ235, SF1126, GDC-0941, XL147, XL765, Palomid 529, GSK1059615, PWT33597, IC87114, TG100-15, CAL263, PI-103, GNE-477, CUDC-907, and AEZS-136. In some aspects, the PI3K inhibitor is a PI3K delta inhibitor such as, but not limited to, Idelalisib, RP6530, TGR1202, and RP6503. Additional PI3K inhibitors are disclosed in U.S. Patent Application Nos. US20150291595, US20110190319, and International Patent Application Nos. WO2012146667, WO2014164942, WO2012062748, and WO2015082376. The immunotherapy may also comprise the administration of an interleukin such as IL-2, or an interferon such as INFu.

Examples of immunotherapies that can be combined with the nimbolide derivatives are immune adjuvants (e.g., Mycobacterium bovis, Plasmodium falciparum, dinitrochlorobenzene and aromatic compounds) (U.S. Pat. Nos. 5,801,005; 5,739,169; Hui and Hashimoto, 1998; Christodoulides et al., 1998), cytokine therapy (e.g., interferons u, R and γ; interleukins (IL-1, IL-2), GM-CSF and TNF) (Bukowski et al., 1998; Davidson et al., 1998; Hellstrand et al., 1998) gene therapy (e.g., TNF, IL-1, IL-2, p53) (Qin et al., 1998; Austin-Ward and Villaseca, 1998; U.S. Pat. Nos. 5,830,880 and 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Combination therapy of cancer with herceptin and chemotherapy has been shown to be more effective than the individual therapies. Thus, it is contemplated that one or more anti-cancer therapies may be employed with the nimbolide derivatives described herein.

Additional immunotherapies that may be combined with the nimbolide derivatives include immune checkpoint inhibitors, a co-stimulatory receptor agonist, a stimulator of innate immune cells, or an activator of innate immunity. In certain aspects the immune checkpoint inhibitor is an inhibitor of CTLA-4, PD-1, PD-L1, PD-L2, LAG-3, BTLA, B7H3, B7H4, TIM3, KIR, or A2aR. In some aspects, the at least one immune checkpoint inhibitor is an anti-CTLA-4 antibody. In some aspects, the anti-CTLA-4 antibody is tremelimumab or ipilimumab.

In certain aspects, the at least one immune checkpoint inhibitor is an anti-killer-cell immunoglobulin-like receptor (KIR) antibody. In some embodiments, the anti-KIR antibody is lirilumab. In some aspects, the inhibitor of PD-L1 is durvalumab, atezolizumab, or avelumab. In some aspects, the inhibitor of PD-L2 is rHIgM12B7. In some aspects, the LAG3 inhibitor is IMP321, or BMS-986016. In some aspects, the inhibitor of A2aR is PBF-509.

In some aspects, the at least one immune checkpoint inhibitor is a human programmed cell death 1 (PD-1) axis binding antagonist. In certain aspects, the PD-1 axis binding antagonist is selected from the group consisting of a PD-1 binding antagonist, a PDL1 binding antagonist and a PDL2 binding antagonist. In some aspects, the PD-1 axis binding antagonist is a PD-1 binding antagonist. In certain aspects, the PD-1 binding antagonist inhibits the binding of PD-1 to PDL1 and/or PDL2. In particular, the PD-1 binding antagonist is a monoclonal antibody or antigen binding fragment thereof. In some embodiments, the PD-1 binding antagonist is nivolumab, pembrolizumab, pidilizumab, AMP-514, REGN2810, CT-011, BMS 936559, MPDL3280A or AMP-224.

In certain aspects, the at least one checkpoint inhibitor is selected from an inhibitor of CTLA-4, PD-1, PD-L1, PD-L2, LAG-3, BTLA, B7H3, B7H4, TIM3, KIR, or A2aR. In some aspects, the at least one immune checkpoint inhibitor is an anti-CTLA-4 antibody. In some aspects, the anti-CTLA-4 antibody is tremelimumab or ipilimumab. In certain aspects, the at least one immune checkpoint inhibitor is an anti-killer-cell immunoglobulin-like receptor (KIR) antibody. In some embodiments, the anti-KIR antibody is lirilumab. In some aspects, the inhibitor of PD-L1 is durvalumab, atezolizumab, or avelumab. In some aspects, the inhibitor of PD-L2 is rHIgM12B7. In some aspects, the LAG3 inhibitor is IMP321, or BMS-986016. In some aspects, the inhibitor of A2aR is PBF-509.

The co-stimulatory receptor agonist may be an anti-OX40 antibody (e.g., MEDI6469, MEDI6383, MEDI0562, and MOXR0916), anti-GITR antibody (e.g., TRX518, and MK-4166anti-CD137 antibody (e.g., Urelumab, and PF-05082566), anti-CD40 antibody (e.g., CP-870,893, and Chi Lob 7/4), or an anti-CD27 antibody (e.g., Varlilumab, also known as CDX-1127). The stimulators of innate immune cells include, but are not limited to, a KIR monoclonal antibody (e.g., lirilumab), an inhibitor of a cytotoxicity-inhibiting receptor (e.g., NKG2A, also known as KLRC and as CD94, such as the monoclonal antibody monalizumab, and anti-CD96, also known as TACTILE), and a toll like receptor (TLR) agonist. The TLR agonist may be BCG, a TLR7 agonist (e.g., poly0ICLC, and imiquimod), a TLR8 agonist (e.g., resiquimod), or a TLR9 agonist (e.g., CPG 7909). The activators of innate immune cells, such as natural killer (NK) cells, macrophages, and dendritic cells, include IDO inhibitors, TGFβ inhibitor, IL-10 inhibitor. An exemplary activator of innate immunity is Indoximod. In some aspects, the immunotherapy is a stimulator of interferon genes (STING) agonist (Corrales et al., 2015).

The immunotherapy may be a cancer vaccine comprising one or more cancer antigens, in particular a protein or an immunogenic fragment thereof, DNA or RNA encoding said cancer antigen, in particular a protein or an immunogenic fragment thereof, cancer cell lysates, and/or protein preparations from tumor cells. As used herein, a cancer antigen is an antigenic substance present in cancer cells. In principle, any protein produced in a cancer cell that is upregulated in cancer cells compared to normal cells or has an abnormal structure due to mutation can act as a cancer antigen. In principle, cancer antigens can be products of mutated or overexpressed oncogenes and tumor suppressor genes, products of other mutated genes, overexpressed or aberrantly expressed cellular proteins, cancer antigens produced by oncogenic viruses, oncofetal antigens, altered cell surface glycolipids and glycoproteins, or cell type-specific differentiation antigens. Examples of cancer antigens include the abnormal or overexpressed products of ras and p53 genes. Other examples include tissue differentiation antigens, mutant protein antigens, oncogenic viral antigens, cancer-testis antigens and vascular or stromal specific antigens. Tissue differentiation antigens are those that are specific to a certain type of tissue. Mutant protein antigens are likely to be much more specific to cancer cells because normal cells shouldn't contain these proteins. Normal cells will display the normal protein antigen on their MHC molecules, whereas cancer cells will display the mutant version.

Some viral proteins are implicated in forming cancer, and some viral antigens are also cancer antigens. Cancer-testis antigens are antigens expressed primarily in the germ cells of the testes, but also in fetal ovaries and the trophoblast. Some cancer cells aberrantly express these proteins and therefore present these antigens, allowing attack by T-cells specific to these antigens.

Exemplary antigens of this type are CTAG1 B and MAGEA1 as well as Rindopepimut, a 14-mer intrndermal injectable peptide vaccine targeted against epidermal growth factor receptor (EGFR) vlll variant. Rindopepimut is particularly suitable for treating glioblastoma when used in combination with an inhibitor of the CD95/CD95L signaling system as described herein.

Also, proteins that are normally produced in very low quantities, but whose production is dramatically increased in cancer cells, may trigger an immune response. An example of such a protein is the enzyme tyrosinase, which is required for melanin production. Normally tyrosinase is produced in minute quantities but its levels are very much elevated in melanoma cells. Oncofetal antigens are another important class of cancer antigens. Examples are alphafetoprotein (AFP) and carcinoembryonic antigen (CEA). These proteins are normally produced in the early stages of embryonic development and disappear by the time the immune system is fully developed. Thus, self-tolerance does not develop against these antigens. Abnormal proteins are also produced by cells infected with oncoviruses, e.g. EBV and HPV. Cells infected by these viruses contain latent viral DNA which is transcribed and the resulting protein produces an immune response. A cancer vaccine may include a peptide cancer vaccine, which in some embodiments is a personalized peptide vaccine. In some embodiments. the peptide cancer vaccine is a multivalent long peptide vaccine, a multi-peptide vaccine, a peptide cocktail vaccine, a hybrid peptide vaccine, or a peptide-pulsed dendritic cell vaccine

The immunotherapy may be an antibody, such as part of a polyclonal antibody preparation, or may be a monoclonal antibody. The antibody may be a humanized antibody, a chimeric antibody, an antibody fragment, a bispecific antibody or a single chain antibody. An antibody as disclosed herein includes an antibody fragment, such as, but not limited to, Fab, Fab′ and F(ab′)₂, Fd, single-chain Fvs (scFv), single-chain antibodies, disulfide-linked Fvs (sdfv) and fragments including either a VL or VH domain. In some aspects, the antibody or fragment thereof specifically binds epidermal growth factor receptor (EGFR1, Erb-B1), HER2/neu (Erb-B2), CD20, Vascular endothelial growth factor (VEGF), insulin-like growth factor receptor (IGF-1R), TRAIL-receptor, epithelial cell adhesion molecule, carcino-embryonic antigen, Prostate-specific membrane antigen, Mucin-1, CD30, CD33, or CD40.

Examples of monoclonal antibodies that may be used in combination with the compositions provided herein include, without limitation, trastuzumab (anti-HER2/neu antibody); Pertuzumab (anti-HER2 mAb); cetuximab (chimeric monoclonal antibody to epidermal growth factor receptor EGFR); panitumumab (anti-EGFR antibody); nimotuzumab (anti-EGFR antibody); Zalutumumab (anti-EGFR mAb); Necitumumab (anti-EGFR mAb); MDX-210 (humanized anti-HER-2 bispecific antibody); MDX-210 (humanized anti-HER-2 bispecific antibody); MDX-447 (humanized anti-EGF receptor bispecific antibody); Rituximab (chimeric murine/human anti-CD20 mAb); Obinutuzumab (anti-CD20 mAb); Ofatumumab (anti-CD20 mAb); Tositumumab-I131 (anti-CD20 mAb); Ibritumomab tiuxetan (anti-CD20 mAb); Bevacizumab (anti-VEGF mAb); Ramucirumab (anti-VEGFR2 mAb); Ranibizumab (anti-VEGF mAb); Aflibercept (extracellular domains of VEGFR1 and VEGFR2 fused to IgG1 Fc); AMG386 (angiopoietin-1 and -2 binding peptide fused to IgG1 Fc); Dalotuzumab (anti-IGF-1R mAb); Gemtuzumab ozogamicin (anti-CD33 mAb); Alemtuzumab (anti-Campath-1/CD52 mAb); Brentuximab vedotin (anti-CD30 mAb); Catumaxomab (bispecific mAb that targets epithelial cell adhesion molecule and CD3); Naptumomab (anti-5T4 mAb); Girentuximab (anti-Carbonic anhydrase ix); or Farletuzumab (anti-folate receptor). Other examples include antibodies such as Panorex™ (17-1A) (murine monoclonal antibody); Panorex (@ (17-1A) (chimeric murine monoclonal antibody); BEC2 (ami-idiotypic mAb, mimics the GD epitope) (with BCG); Oncolym (Lym-1 monoclonal antibody); SMART M195 Ab, humanized 13′ 1 LYM-1 (Oncolym), Ovarex (B43.13, anti-idiotypic mouse mAb); 3622W94 mAb that binds to EGP40 (17-1A) pancarcinoma antigen on adenocarcinomas; Zenapax (SMART Anti-Tac (IL-2 receptor); SMART M195 Ab, humanized Ab, humanized); NovoMAb-G2 (pancarcinoma specific Ab); TNT (chimeric mAb to histone antigens); TNT (chimeric mAb to histone antigens); Gliomab-H (Monoclonals-Humanized Abs); GNI-250 Mab; EMD-72000 (chimeric-EGF antagonist); LymphoCide (humanized IL.L.2 antibody); and MDX-260 bispecific, targets GD-2, ANA Ab, SMART IDIO Ab, SMART ABL 364 Ab or ImmuRAIT-CEA. Examples of antibodies include those disclosed in U.S. Pat. Nos. 5,736,167, 7,060,808, and 5,821,337.

Further examples of antibodies include Zanulimumab (anti-CD4 mAb), Keliximab (anti-CD4 mAb); Ipilimumab (MDX-101; anti-CTLA-4 mAb); Tremilimumab (anti-CTLA-4 mAb); (Daclizumab (anti-CD25/IL-2R mAb); Basiliximab (anti-CD25/IL-2R mAb); MDX-1106 (anti-PD1 mAb); antibody to GITR; GC1008 (anti-TGF-0 antibody); metelimumab/CAT-192 (anti-TGF-0 antibody); lerdelimumab/CAT-152 (anti-TGF-0 antibody); ID11 (anti-TGF-0 antibody); Denosumab (anti-RANKL mAb); BMS-663513 (humanized anti-4-1BB mAb); SGN-40 (humanized anti-CD40 mAb); CP870,893 (human anti-CD40 mAb); Infliximab (chimeric anti-TNF mAb; Adalimumab (human anti-TNF mAb); Certolizumab (humanized Fab anti-TNF); Golimumab (anti-TNF); Etanercept (Extracellular domain of TNFR fused to IgG1 Fc); Belatacept (Extracellular domain of CTLA-4 fused to Fc); Abatacept (Extracellular domain of CTLA-4 fused to Fc); Belimumab (anti-B Lymphocyte stimulator); Muromonab-CD3 (anti-CD3 mAb); Otelixizumab (anti-CD3 mAb); Teplizumab (anti-CD3 mAb); Tocilizumab (anti-IL6R mAb); REGN88 (anti-IL6R mAb); Ustekinumab (nnti-IT-12/23 mAb); Briakinumab (anti-IL-12/23 mAb); Natalizumab (anti-u4 integrin); Vedolizumab (anti-α4 β7 integrin mAb); T1 h (anti-CD6 mAb); Epratuzumab (anti-CD22 mAb); Efalizumab (anti-CD11a mAb); and Atacicept (extracellular domain of transmembrane activator and calcium-modulating ligand interactor fused with Fc).

a. Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient. Human monoclonal antibodies to ganglioside antigens have been administered intralesionally to patients suffering from cutaneous recurrent melanoma (Irie & Morton, 1986). Regression was observed in six out of ten patients, following, daily or weekly, intralesional injections. In another study, moderate success was achieved from intralesional injections of two human monoclonal antibodies (Irie et al., 1989).

It may be favorable to administer more than one monoclonal antibody directed against two different antigens or even antibodies with multiple antigen specificity. Treatment protocols also may include administration of lymphokines or other immune enhancers as described by Bajorin et al. (1988). The development of human monoclonal antibodies is described in further detail elsewhere in the specification.

b. Active Immunotherapy

In active immunotherapy, an antigenic peptide, polypeptide or protein, or an autologous or allogenic tumor cell composition or “vaccine” is administered, generally with a distinct bacterial adjuvant (Ravindranath & Morton, 1991; Morton & Ravindranath, 1996; Morton et al., 1992; Mitchell et al., 1990; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

c. Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated antigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond. More recently, higher response rates have been observed when such adoptive immune cellular therapies have incorporated genetically engineered T cells that express chimeric antigen receptors (CAR) termed CAR T cell therapy. Similarly, natural killer cells both autologous and allogenic have been isolated, expanded and genetically modified to express receptors or ligands to facilitate their binding and killing of tumor cells.

### 4. Other Agents

It is contemplated that other agents may be used in combination with the compositions provided herein to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; or MIP-1, MIP-Ibeta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the compositions provided herein by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the compositions provided herein to improve the anti-hyperproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the compositions provided herein to improve the treatment efficacy.

In certain embodiments, hormonal therapy may also be used in conjunction with the present embodiments or in combination with any other cancer therapy previously described. The use of hormones may be employed in the treatment of certain cancers such as breast, prostate ovarian, or cervical cancer to lower the level or block the effects of certain hormones such as testosterone or estrogen. This treatment is often used in combination with at least one other cancer therapy as a treatment option or to reduce the risk of metastases

In some aspects, the additional anti-cancer agent is a protein kinase inhibitor or a monoclonal antibody that inhibits receptors involved in protein kinase or growth factor signaling pathways such as an EGFR, VEGFR, AKT, Erb1, Erb2, ErbB, Syk, Bcr-Abl, JAK, Src, GSK-3, PI3K, Ras, Raf, MAPK, MAPKK, mTOR, c-Kit, eph receptor or BRAF inhibitors. Nonlimiting examples of protein kinase or growth factor signaling pathways inhibitors include afatinib, axitinib, bevacizumab, bosutinib, cetuximab, crizotinib, dasatinib, erlotinib, fostamatinib, gefitinib, imatinib, lapatinib, lenvatinib, mubritinib, nilotinib, panitumumab, pazopanib, pegaptanib, ranibizumab, ruxolitinib, saracatinib, sorafenib, sunitinib, trastuzumab, vandetanib, AP23451, vemurafenib, MK-2206, GSK690693, A-443654, VQD-002, miltefosine, perifosine, CAL101, PX-866, LY294002, rapamycin, temsirolimus, everolimus, ridaforolimus, alvocidib, genistein, selumetinib, AZD-6244, vatalanib, P1446A-05, AG-024322, ZD1839, P276-00, GW572016 or a mixture thereof.

In some aspects, the PI3K inhibitor is selected from the group of PI3K inhibitors consisting of buparlisib, idelalisib, BYL-719, dactolisib, PF-05212384, pictilisib, copanlisib, copanlisib dihydrochloride, ZSTK-474, GSK-2636771, duvelisib, GS-9820, PF-04691502, SAR-245408, SAR-245409, sonolisib, Archexin, GDC-0032, GDC-0980, apitolisib, pilaralisib, DLBS 1425, PX-866, voxtalisib, AZD-8186, BGT-226, DS-7423, GDC-0084, GSK-21 26458, INK-1117, SAR-260301, SF-1126, AMG-319, BAY-1082439, CH-51 32799, GSK-2269557, P-71 70, PWT-33597, CAL-263, RG-7603, LY-3023414, RP-5264, RV-1729, taselisib, TGR-1 202, GSK-418, INCB-040093, Panulisib, GSK-105961 5, CNX-1351, AMG-51 1, PQR-309, 17beta-Hydroxywortmannin, AEZS-129, AEZS-136, HM-5016699, IPI-443, ONC-201, PF-4989216, RP-6503, SF-2626, X-339, XL-499, PQR-401, AEZS-132, CZC-24832, KAR-4141, PQR-31 1, PQR-316, RP-5090, VS-5584, X-480, AEZS-126, AS-604850, BAG-956, CAL-130, CZC-24758, ETP-46321, ETP-471 87, GNE-317, GS-548202, HM-032, KAR-1 139, LY-294002, PF-04979064, PI-620, PKI-402, PWT-143, RP-6530, 3-HOI-BA-01, AEZS-134, AS-041 164, AS-252424, AS-605240, AS-605858, AS-606839, BCCA-621 C, CAY-10505, CH-5033855, CH-51 08134, CUDC-908, CZC-1 9945, D-106669, D-87503, DPT-NX7, ETP-46444, ETP-46992, GE-21, GNE-123, GNE-151, GNE-293, GNE-380, GNE-390, GNE-477, GNE-490, GNE-493, GNE-614, HMPL-51 8, HS-104, HS-1 06, HS-1 16, HS-173, HS-196, IC-486068, INK-055, KAR 1 141, KY-1 2420, Wortmannin, Lin-05, NPT-520-34, PF-04691503, PF-06465603, PGNX-01, PGNX-02, PI 620 PT-103, PI-509, PI-516, PI-540, PIK-75, PWT-458, RO-2492, RP-5152, RP-5237, SB-201 5, SB-2312, SB-2343, SHBM-1009, SN 32976, SR-13179, SRX-2523, SRX-2558, SRX-2626, SRX-3636, SRX-5000, TGR-5237, TGX-221, UCB-5857, WAY-266175, WAY-266176, EI-201, AEZS-131, AQX-MN100, KCC-TGX, OXY-111A, PI-708, PX-2000, and WJD-008.

It is contemplated that the additional cancer therapy can comprise an antibody, peptide, polypeptide, small molecule inhibitor, siRNA, miRNA or gene therapy which targets, for example, epidermal growth factor receptor (EGFR, EGFR1, ErbB-1, HER1), ErbB-2 (HER2/neu), ErbB-3/HER3, ErbB-4/HER4, EGFR ligand family; insulin-like growth factor receptor (IGFR) family, IGF-binding proteins (IGFBPs), IGFR ligand family (IGF-1R); platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family: TRK receptor family; ephrin (EPH) receptor family; AXL receptor family; leukocyte tyrosine kinase (LTK) receptor family; TIE receptor family, angiopoietin 1, 2; receptor tyrosine kinase-like orphan receptor (ROR) receptor family; discoidin domain receptor (DDR) family; RET receptor family; KLG receptor family; RYK receptor family; MuSK receptor family; Transforming growth factor alpha (TGF-α), TGF-α receptor; Transforming growth factor-beta (TGF-β), TGF-β receptor; Interleukin 13 receptor alpha2 chain (1L13Ralpha2), Interleukin-6 (IL-6), 1L-6 receptor, Interleukin-4, IL-4 receptor, Cytokine receptors, Class I (hematopoietin family) and Class II (interferon/1L-10 family) receptors, tumor necrosis factor (TNF) family, TNF-α, tumor necrosis factor (TNF) receptor superfamily (TNTRSF), death receptor family, TRAIL-receptor; cancer-testis (CT) antigens, lineage-specific antigens, differentiation antigens, alpha-actinin-4, ARTC1, breakpoint cluster region-Abelson (Bcr-abl) fusion products, B-RAF, caspase-5 (CASP-5), caspase-8 (CASP-8), beta-catenin (CTNNB1), cell division cycle 27 (CDC27), cyclin-dependent kinase 4 (CDK4), CDKN2A, COA-1, dek-can fusion protein, EFTUD-2, Elongation factor 2 (ELF2), Ets variant gene 6/acute myeloid leukemia 1 gene ETS (ETC6-AML1) fusion protein, fibronectin (FN), GPNMB, low density lipid receptor/GDP-L fucose: beta-Dgalactose 2-alpha-Lfucosyltraosferase (LDLR/FUT) fusion protein, HLA-A2, arginine to isoleucine exchange at residue 170 of the alpha-helix of the alpha2-domain in the HLA-A2 gene (HLA-A*201-R170I), MLA-A11, heat shock protein 70-2 mutated (HSP70-2M), KIAA0205, MART2, melanoma ubiquitous mutated 1, 2, 3 (MUM-1, 2, 3), prostatic acid phosphatase (PAP), neo-PAP, Myosin class 1, NFYC, OGT, OS-9, pml-RARalpha fusion protein, PRDX5, PTPRK, K-ras (KRAS2), N-ras (NRAS), HRAS, RBAF600, SIRT2, SNRPD1, SYT-SSX1 or -SSX2 fusion protein, Triosephosphate Isomerase, BAGE, BAGE-1, BAGE-2,3,4,5, GAGE-1,2,3,4,5,6,7,8, GnT-V (aberrant N-acetyl giucosaminyl transferase V, MGAT5), HERV-K-MEL, KK-LC, KM-HN-1, LAGE, LAGE-1, CTL-recognixed antigen on melanoma (CAMEL), MAGE-A1 (MAGE-1), MAGE-A2, MAGE-A3, MAGE-A4, MAGE-AS, MAGE-A6, MAGE-A8, MAGE-A9, MAGE-A10, MAGE-A11, MAGE-A12, MAGE-3, MAGE-B1, MAGE-B2, MAGE-B5, MAGE-B6, MAGE-C1, MAGE-C2, mucin 1 (MUC1), MART-1/Melan-A (MLANA), gp100, gp100/Pme117 (S1LV), tyrosinase (TYR), TRP-1, HAGE, NA-88, NY-ESO-1, NY-ESO-1/LAGE-2, SAGE, Sp17, SSX-1,2,3,4, TRP2-1NT2, carcino-embryonic antigen (CEA), Kallikfein 4, mammaglobm-A, OA1, prostate specific antigen (PSA), prostate specific membrane antigen, TRP-1/gp75, TRP-2, adipophilin, interferon inducible protein absent in nielanorna 2 (AIM-2), BING-4, CPSF, cyclin D1, epithelial cell adhesion molecule (Ep-CAM), EpbA3, fibroblast growth factor-5 (FGF-5), glycoprotein 250 (gp250intestinal carboxyl esterase (iCE), alpha-feto protein (AFP), M-CSF, mdm-2 (e.g., small molecule inhibitor of HDM2, also known as MDM2, and/or HDM4, such as to reverse its inhibition of p53 activity, such as HDM201, cis-imidazolines (e.g., Nutlins), benzodiazepines (BDPs), spiro-oxindoles), MUCI, p53 (TP53), PBF, FRAME, PSMA, RAGE-1, RNF43, RU2AS, SOX10, STEAPI, survivin (BIRCS), human telomerase reverse transcriptase (hTERT), telomerase, Wilms' tumor gene (WT1), SYCP1, BRDT, SPANX, XAGE, ADAM2, PAGE-5, LIP1, CTAGE-1, CSAGE, MMA1, CAGE, BORIS, HOM-TES-85, AF15q14, HCA66I, LDHC, MORC, SGY-1, SPO11, TPX1, NY-SAR-35, FTHLI7, NXF2 TDRD1, TEX 15, FATE, TPTE, immunoglobulin idiotypes, Bence-Jones protein, estrogen receptors (ER), androgen receptors (AR), CD40, CD30, CD20, CD19, CD33, CD4, CD25, CD3, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), beta-human chorionic gonadotropin, 1-2 microglobulin, squamous cell carcinoma antigen, neuron-specific enoJase, heat shock protein gp96, GM2, sargramostim, CTLA-4, 707 alanine proline (707-AP), adenocarcinoma antigen recognized by T cells 4 (ART-4), carcinoembryogenic antigen peptide-1 (CAP-1), calcium-activated chloride channel-2 (CLCA2), cyclophilin B (Cyp-B), human signet ring tumor-2 (HST-2), Human papilloma virus (HPV) proteins (HPV-E6, HPV-E7, major or minor capsid antigens, others), Epstein-Barr vims (EBV) proteins (EBV latent membrane proteins-LMP1, LMP2; others), Hepatitis B or C virus proteins, and HIV proteins.

### V. Definitions

The definitions below supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

When used in the context of a chemical group: “hydrogen” means —H; “hydroxy” means —OH; “oxo” means═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof, “mercapto” means —SH; and “thio” means ═S; “thiocarbonyl” means —C(═S)—; “sulfonyl” means —S(O)₂—; and “sulfinyl” means —S(O)-.

In the context of chemical formulas, the symbol “ [symbol] ” means a single bond, “ [symbol] ” means a double bond, and “≡” means triple bond. The symbol “ [symbol] ” represents an optional bond, which if present is either single or double. The symbol “ [symbol] ” represents a single bond or a double bond. Thus, the formula

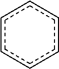

covers, for example,

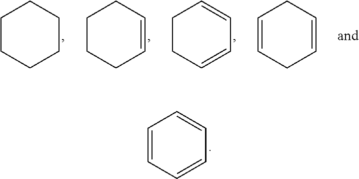

And it is understood that no one such ring atom forms part of more than one double bond. Furthermore, it is noted that the covalent bond symbol “ [symbol] ”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

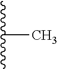

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

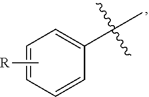

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

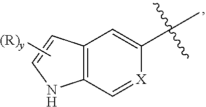

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C═n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C1-4-alkyl”, “alkyl_{(C1-4)}”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C12)} group; however, it is not an example of a dialkylamino_{(C6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

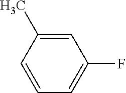

is also taken to refer to

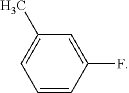

Aromatic compounds may also be depicted using a circle to represent the delocalized nature of the electrons in the fully conjugated cyclic 7L system, two non-limiting examples of which are shown below;

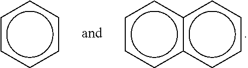

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

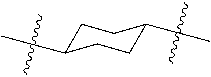 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

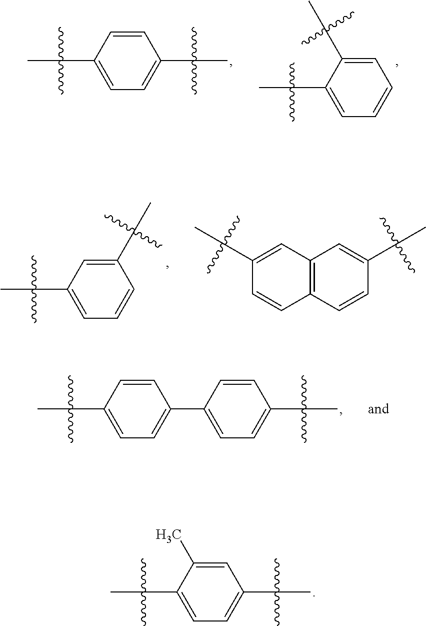

An “arene” refers to the class of compounds having the formula H—R, wherein R is aryl as that term is defined above. Benzene and toluene are non-limiting examples of arenes.

The term “aralkyl” refers to the monovalent group-alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes. The term “heteroarenediyl” refers to a divalent aromatic group, with two aromatic carbon atoms, two aromatic nitrogen atoms, or one aromatic carbon atom and one aromatic nitrogen atom as the two points of attachment, said atoms forming part of one or more aromatic ring structures, each with three to eight ring atoms, wherein at least one of the ring atoms of the aromatic ring structure(s) is nitrogen, oxygen or sulfur, and wherein the divalent group consists of no atoms other than carbon, hydrogen, aromatic nitrogen, aromatic oxygen and aromatic sulfur. If more than one ring is present, the rings are fused; however, the term heteroarenediyl does not preclude the presence of one or more alkyl or aryl groups (carbon number limitation permitting) attached to one or more ring atoms. Non-limiting examples of heteroarenediyl groups include:

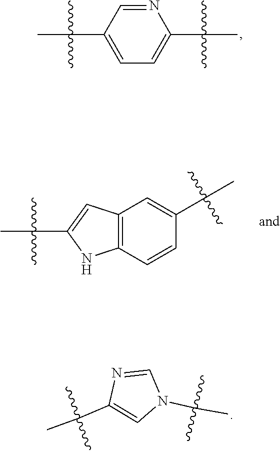 [SMILES: *c1ccc(*)nc1 ; *c1ccc2[nH]c(*)cc2c1 ; *c1cn(*)cn1]

The term “oxygen-containing heteroaryl” refers to a monovalent aromatic group with an aromatic carbon atom as the point of attachment, said carbon atom forming part of one or more aromatic ring structures, each with three to eight ring atoms, wherein at least one of the ring atoms of the aromatic ring structure(s) is oxygen, and wherein the heteroaryl group consists of no atoms other than carbon, hydrogen, and aromatic oxygen. If more than one ring is present, the rings are fused; however, the term oxygen-containing heteroaryl does not preclude the presence of one or more alkyl or aryl groups (carbon number limitation permitting) attached to one or more ring atoms. Non-limiting examples of heteroaryl groups include furanyl and benzofuranyl. The term “oxygen-containing heteroarenediyl” refers to a divalent aromatic group, with two aromatic carbon atoms as the two points of attachment, said atoms forming part of one or more aromatic ring structures, each with three to eight ring atoms, wherein at least one of the ring atoms of the aromatic ring structure(s) is oxygen, and wherein the divalent group consists of no atoms other than carbon, hydrogen, and aromatic oxygen. If more than one ring is present, the rings are fused; however, the term oxygen-containing heteroarenediyl does not preclude the presence of one or more alkyl or aryl groups (carbon number limitation permitting) attached to one or more ring atoms. Non-limiting examples of oxygen-containing heteroarenediyl groups include:

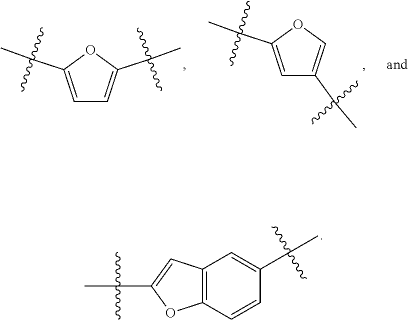 [SMILES: *c1ccc(*)o1 ; *c1coc(*)c1 ; *c1ccc2oc(*)cc2c1]

The term “nitrogen-containing heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, and wherein the heteroaryl group consists of no atoms other than carbon, hydrogen, aromatic nitrogen, aromatic oxygen, and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “nitrogen-containing heteroarenediyl” refers to a divalent aromatic group, with two aromatic carbon atoms, two aromatic nitrogen atoms, or one aromatic carbon atom and one aromatic nitrogen atom as the two points of attachment, said atoms forming part of one or more aromatic ring structures, each with three to eight ring atoms, wherein at least one of the ring atoms of the aromatic ring structure(s) is nitrogen, and wherein the divalent group consists of no atoms other than carbon, hydrogen, aromatic nitrogen, aromatic oxygen, and aromatic sulfur. If more than one ring is present, the rings are fused; however, the term nitrogen-containing heteroarenediyl does not preclude the presence of one or more alkyl or aryl groups (carbon number limitation permitting) attached to one or more ring atoms. Non-limiting examples of nitrogen-containing heteroarenediyl groups include:

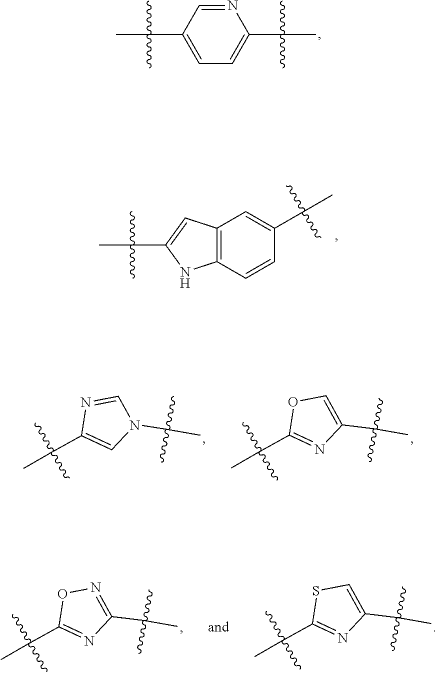 [SMILES: *c1ccc(*)nc1 ; *c1ccc2[nH]c(*)cc2c1 ; *c1cn(*)cn1 ; *c1coc(*)n1 ; *c1noc(*)n1 ; *c1csc(*)n1]

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused or spirocyclic. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. Non-limiting examples of N-heterocycloalkyl groups include N-pyrrolidinyl and

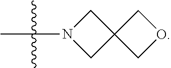 [SMILES: *N1CC2(COC2)C1]

When the term “heterocycloalkyl” is used with the “substituted” modifier, one or more hydrogen atom has been replaced, independently at each instance, by oxo, —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted heterocycloalkyl groups (more specifically, substituted N-heterocycloalkyl groups):

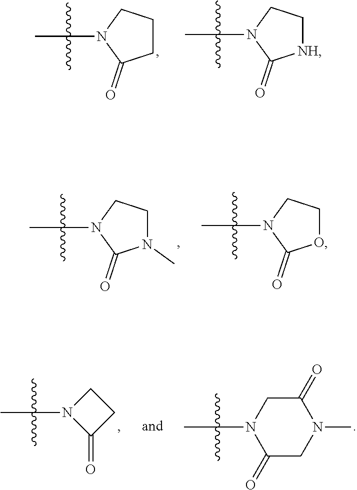

The term “heterocycloalkanediyl” refers to a divalent cyclic group, with two carbon atoms, two nitrogen atoms, or one carbon atom and one nitrogen atom as the two points of attachment, said atoms forming part of one or more ring structure(s) wherein at least one of the ring atoms of the non-aromatic ring structure(s) is nitrogen, oxygen or sulfur, and wherein the divalent group consists of no atoms other than carbon, hydrogen, nitrogen, oxygen and sulfur. If more than one ring is present, the rings are fused. As used herein, the term heterocycloalkanediyl does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkanediyl groups include:

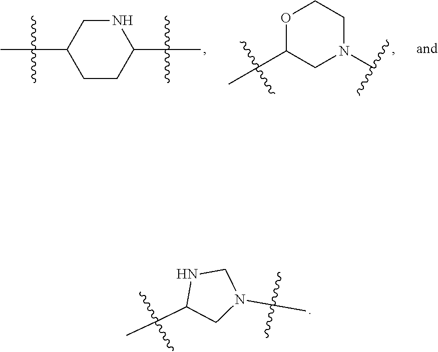 [SMILES: *C1CCC(*)NC1 ; *C1CN(*)CCO1 ; *C1CN(*)CN1]

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H5, and —C(O)C₆H4CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, “acyloxy”, and “arylsulfonyloxy” when used without the “substituted” modifier, refers to groups, defined as —OR, in which R is cycloalkyl, alkenyl, alkynyl, aryl, aralkyl, heteroaryl, heterocycloalkyl, acyl, and arylsulfonyl, respectively. The term “alkylthio” and “acylthio” refers to the group —SR, in which R is an alkyl and acyl, respectively. The term “alkylsilyl” refers to the group —SiR₃, in which each R is, independently, an alkyl. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

The terms “alkylsulfonyl” and “alkylsulfinyl” refers to the groups —S(O)₂R and —S(O)R, respectively, in which R is an alkyl, as that term is defined above. The terms “cycloalkylsulfonyl”, “alkenylsulfonyl”, “alkynylsulfonyl”, “arylsulfonyl”, “aralkylsulfonyl”, “heteroarylsulfonyl”, and “heterocycloalkylsulfonyl” are defined in an analogous manner.

An “amine protecting group” or “amino protecting group” is well understood in the art. An amine protecting group is a group which modulates the reactivity of the amine group during a reaction which modifies some other portion of the molecule. Amine protecting groups can be found at least in Greene and Wuts, 1999, which is incorporated herein by reference. Some non-limiting examples of amino protecting groups include formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxycarbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxvbenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; alkylaminocarbonyl groups (which form ureas with the protect amine) such as ethylaminocarbonyl and the like; aralkyl groups such as benzyl, triphenylmethyl, benzyloxymethyl and the like; and silyl groups such as trimethylsilyl and the like. Additionally, the “amine protecting group” can be a divalent protecting group such that both hydrogen atoms on a primary amine are replaced with a single protecting group. In such a situation the amine protecting group can be phthalimide (phth) or a substituted derivative thereof wherein the term “substituted” is as defined above. In some embodiments, the halogenated phthalimide derivative may be tetrachlorophthalimide (TCphth). When used herein, a “protected amino group”, is a group of the formula PG_{MA}NH— or PG_{DA}N— wherein PG_{MA} is a monovalent amine protecting group, which may also be described as a “monovalently protected amino group” and PG_{DA} is a divalent amine protecting group as described above, which may also be described as a “divalently protected amino group”.

A “hydroxy protecting group” or “hydroxyl protecting group” is well understood in the art. A hydroxy protecting group is a group which prevents the reactivity of the hydroxyl group during a reaction which modifies some other portion of the molecule and can be easily removed to generate the desired hydroxyl. Hydroxy protecting groups can be found at least in Greene and Wuts, 1999, which is incorporated herein by reference. Some non-limiting examples of hydroxy protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, u-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methnxvhenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl, tbutyldimehtylsilyl, and the like. When used herein, a protected hydroxy group is a group of the formula PG_{H}O— wherein PG_{H} is a hydroxy protecting group as described above.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂C1, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂C1. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂C1 is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value or the variation that exists among the study subjects or patients.

An “active ingredient” (A1) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be, formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present disclosure. The prodrug itself may or may not have activity in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### VI. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: PARP1 Trapping by Nimbolide

### A. Materials and Methods

Materials. The antibodies as following: PAR (Trevigen), GAPDH (Thermo Fisher Scientific), Flag-tag (Thermo Fisher Scientific), GST (CST), RNF114 (Santa Cruze), histone (CST), ubiquitin (Santa Cruze), PARP1 (CST), γH2AX (CST), p-TBK1 (CST), TBK1 (CST), p-IRF3 (CST), IRF3 (CST), cGAS (CST), STING (CST), PD-L1 (CST) were obtained from commercial sources as indicated. Nimbolide was purchased from Sigma-Aldrich. PARP inhibitors: veliparib (Selleck), rucaparib (Selleck), olaparib (LC laboratory), niraparib (Selleck), talazoparib (Selleck) were purchased from the indicated sources. All other reagents including MG132 (Selleck), H₂O₂(Thermo Fisher Scientific), MMS (Sigma-Aldrich), doxorubicin (Sigma-Aldrich), temozolomide (Sigma-Aldrich), AZD6738 (Cayman), CGK733 (Cayman), LY2603618 (Apexbio), SCH900776 (Cayman) were obtained from commercial sources.

Cell culture. All the cells were purchased from ATCC and cultured according to the directions from ATCC. HCT116 cells, A673 cells, Fadu cells, Hela cells, MDA-MB-468 cells, and B16 cells were maintained in the high-glucose DMEM medium supplemented with 10% FBS. H2058 cells were maintained in the RPMI-1640 medium supplemented with 5% FBS. H1048 cells were maintained in the DMEM/F12 medium supplemented with 5% FBS, 0.005 mg/mL Insulin, 0.01 mg/mL Transferrin, 30 nM Sodium selenite, 10 nM Hydrocortisone, 10 nM beta-estradiol, and extra 2 mM L-glutamine. UWB1 and UWB1+BRCA1 cells were maintained in RPMI1640 (ATCC) and MEGM bullet kit (1:1; Lonza) with 3% FBS. UWB1 (SYr12) cells were maintained in RPMI1640 (ATCC) and MEGM bullet kit (1:1; Lonza) with 3% FBS and 1 μM PARPi (olaparib; SelleckChem) (Yazinski et al., 2017).

Plasmid and construction. The RNF114 clone was obtained from the Center for Human Growth and Development of UTSW. TAP-RNF114 plasmid was constructed by insertion of RNF114 cDNA into the pCDNA5-ZZvTEV-Flag vector (addgene). The RNF114 cDNA was subcloned into the pcDNA3 (addgene) or pGEX-4T-3 (addgene) vectors for transient transfections. Besides, RNF114 cDNA was transferred into plenti-6.3-V5-Dest vector (Thermo Fisher) to construct stable cell lines. Various site mutations in RNF114 were introduced using standard site-directed mutagenesis techniques. All the mutant constructs were confirmed by DNA sequencing analysis. PARP1-GFP and XRCC1-GFP plasmids were gifts from Dr. Xiaochun Yu (City of Hope).

Construction of stable cell lines. The plenti or pLKO.1 construct (8 μg), VSVG (6 g), and delta8.9 (6 μg) were co-transfected into HEK293TD cells in 10 cm dishes with Lipo-2000 (Sigma). The medium was changed 6 h after transfection. Viruses were collected twice at 24 h and 48 h after transfection respectively, and then combined together. Subsequently, 3 mL of virus was added to each well of HCT116 or Hela cells in 6-well plates with Polybrene (8 μg/mL). After splitting the cells once, HCT116 or Hela cells were infected with previously collected virus again using the same procedure. The culture medium was replaced after 48 h with a fresh growth medium containing 2 μg/mL blasticidin or puromycin.

Immunoblot analysis. Cells were collected and washed once with cold 1×PBS. Then, cells were lysed with the 1% SDS lysis buffer (1% SDS, 10 mM HEPES, pH 7.0, 2 mM MgCl₂ and 500 U universal nuclease). Protein concentrations were measured by the BCA assay kit (Thermo Fisher). The same amount of protein was loaded onto an SDS-PAGE gel. After electrophoretic separation, proteins were transferred to a NC (nitrocellulose) membrane (GE Healthcare). As for the Dot-immunoblot analysis, samples were loaded directly to the NC membrane. The membrane was blocked, and then blotted with the primary antibodies overnight at 4° C. followed by incubation with the secondary antibody for 1 h at room temperature. The blots were developed using enhanced chemiluminescence and were exposed on autoradiograph films.

Co-immunoprecipitation analysis. Cells were collected and lysed in the IP lysis buffer (50 mM Tris, pH 7.4, 1 mM EDTA, 150 mM NaCl, 0.5% NP-40 and 1×protease inhibitor cocktail). After incubation for 1 h at 4° C., cell lysates were centrifuged at 14,000 g for 10 min at 4° C. The supernatants were transferred and incubated with the corresponding agarose beads overnight at 4° C. The beads were washed three times with IP wash buffer, and the immunocomplexes were eluted from the beads by boiling at 95° C. for 30 min and subjected to immunoblot analysis. The TAP-IP-MS experiments were performed according to a previously described protocol (Tsai and Carstens, 2006).

Immunofluorescence microscopy. After the treatment with DMSO or nimbolide, Hela cells were washed once with 1×PBS, and then, fixed with 4% paraformaldehyde for 20 min at room temperature followed by three times wash with 1×PBS. The cells were permeabilized with 0.25% Triton X-100 in 1×PBS for 10 min and blocked with 1×PBS containing 2% BSA for 1 h. Fixed cells were incubated with primary antibodies at 4° C. overnight, followed by the incubation with fluorescent secondary antibody for 1 h at room temperature (RT). Cells were washed three times with 1×PBS for 5 min and stained with DAPI (Thermo Fisher Scientific) for 2 min. Cells were washed with 1×PBS and mounted with the FluorSave reagent (Millipore). The fluorescence images were then collected with a Zeiss LSM 880 Airyscan inverted confocal microscope.

qRT-PCR. The cells with the treatment of DMSO or nimbolide were lysed with TRIzol (Thermo Fisher). Then, the total RNA was extracted according to the manufacturer's protocol and subjected to reverse transcription with SuperScript® III One-Step RT-PCR System (Thermo Fisher). The qRT-PCR experiments were performed with a Power SYBR® Green PCR Master Mix (Thermo Fisher) and specific primers listed as following: GAPDH, sense: ACAACTTTGGCATTGTGGAA, anti-sense: GATGCAGGGATGATGTTCTG; IFN-β, sense: AGCTGAAGCAGTTCCAGAAG, anti-sense: AGTCTCATTCCAGCCAGTGC; CXCL10, sense: GGCCATCAAGAATTTACTGAAAGCA; anti-sense: TCTGTGTGGTCCATCCTTGGAA; CCL5, sense: ATCCTCATTGCTACTGCCCTC; anti-sense: GCCACTGGTGTAGAAATACTCC.

In vitro PARylation assays. PARP1 (500 ng, Tulip Biolabs), sheared Salmon Sperm DNA (100 ng, Thermo Fisher) and NAD⁺ (500 μM) were incubated in the reaction buffer (50 mM Tris, pH 7.5, 4 mM MgCl₂, 20 mM NaCl and 250 μM DTT) with at RT for 1 h. Reactions were terminated by SDS loading buffer and the samples were subjected to immunoblot analysis by an anti-PAR antibody.

In vitro ubiquitination assays. To measure the auto-ubiquitination activation of RNF114 by PAR polymers, UBE1 (50 nM, Boston Biochem), UBE2D1 (50 nM, Boston Biochem) and Ubiquitin (200 mM, Boston Biochem) were incubated with recombinant RNF114 at 37° C. in the ubiquitination reaction buffer (50 mM Tris-Cl, pH 7.5, 2.5 mM MgCl₂, 2 mM DTT, 2 mM ATP). Reactions were terminated by SDS loading buffer and boiled. The supernatant samples were subjected to immunoblot analysis by an anti-ubiquitin antibody. As for the in vitro PARylated-PARP1 ubiquitination assays, PARP1 or PARylated-PARP1 was incubated together with UBE1 (50 nM, Boston Biochem), UBE2D1 (50 nM, Boston Biochem), Ubiquitin (200 mM, Boston Biochem) and recombinant RNF114 at 37° C. in the ubiquitination reaction buffer as above. Reactions were terminated by SDS loading buffer and boiled. The supernatant samples were subjected to immunoblot analysis by anti-ubiquitin and anti-PAR antibodies.

Colony formation assays. HCT116 RNF114-WT or RNF114-KO cells were seeded into 6-cm dishes (about 1000 cells per dish). The cells were treated with or without H₂O₂ (2 mM for 5 min) and followed by 14 days culture. The viable cells were fixed by methanol and stained with crystal violet.

Laser microirradiation assays. Cells grown on 35-mm glass-bottomed culture dishes (Mattek) were transfected with a GFP-tagged (RNF114, PARP1, or XRCC1) plasmid for 24 h. After the compound treatment as indicated in each experiment, laser microirradiation was performed using a Zeiss LSM 780 inverted confocal microscope coupled with the MicroPoint laser illumination and ablation system (Photonic Instruments). The GFP fluorescence at the laser line was recorded at the indicated time points and then analyzed with ImageJ software.

Syngeneic tumor model. All animal experiments were conducted under a protocol approved by the Institutional Animal Care and Use Committee (IACUC) of UT Southwestern Medical Center. C57BL/6 mice (8 weeks old, male) were inoculated with B16 (1×10⁶) cells subcutaneously in 100 mL of serum-free medium containing 50% Matrigel (BD Biosciences). Six days after inoculation, mice were assigned randomly into four groups (n=5 per group). Tumor bearing mice were injected intraperitoneally with isotype control IgG or anti-mouse-PD-L1 antibody (aPD-L1, 100 mg/mouse, Bio X Cell) every 3 days. Vehicle control (10% DMSO, 90% Olive Oil) or nimbolide (20 mg/kg in 10% DMSO, 90% Olive Oil) was administered by daily oral gavage. Mice were weighed and observed for signs of pain and distress every 3 days. Tumor size was measured by a caliper every 3 days and calculated using a standard formula: 0.5×length×width².

Sample preparation and mass spectrometry analysis. To analysis the protein dynamics in response to DNA damage, HCT116 cells were pre-treated with talazoparib (1 μM for 1 h) to block PARylation and followed by the treatment with H₂O₂ (2 mM for 5 min) or MMS (0.01% for 1 h) as indicated. Cells were washed with cold 1×PBS and were subjected to chromatin-bound proteins extraction using the subcellular fractionation kit (Thermo Fisher). Protein concentrations were measured by the BCA assay kit (Thermo Fisher). For the TMT experiments, proteins were reduced with dithiothreitol (2 mM for 10 min) and alkylated with iodoacetamide (50 mM for 30 min) in the dark. Proteins were then extracted by methanol/chloroform precipitation and were washed by ice-cold methanol. Protein pellets were re-dissolved in 400 μl of 8 M freshly prepared urea buffer (50 mM Tris-HCl and 10 mM EDTA, pH 7.5). The proteins were digested by Lys-C at a 1:100 enzyme/protein ratio for 2 h at room temperature, followed by trypsin digestion at a 1:100 enzyme/protein ratio overnight at room temperature. Peptides were desalted with Oasis HLB cartridges and resuspended in 200 mM HEPES, pH 8.5. For each sample, 100 μg of peptides were reacted with the corresponding amine-based TMT six-plex reagents (Thermo Fisher) for 1 h at RT. The labeling scheme was as following: 126: control, 127: H₂O₂, 128: MMS, 129: talazoparib, 130: talazoparib and H₂O₂ and 131: talazoparib and MMS. The reactions were quenched with hydroxylamine solution and the peptide samples were combined.

The TMT samples were desalted and fractionated by basic pH reversed phase HPLC on a ZORBAX 300 Extend-C18 column (narrow bore RR 2.1 mm×100 mm, 3.5-μm particle size, 300-A pore size). Buffer A was 10 mM ammonium formate in water, pH 10.0. A gradient was developed at a flow rate of 0.2 mL/min from 0% to 70% buffer B (1 mM ammonium formate, pH 10.0, 90% acetonitrile). Seventeen fractions were collected, which were lyophilized, desalted and analyzed by LC-MS/MS as described previously (36-38).

Statistics. All of the statistical analyses (t tests) were performed using GraphPad Prism software (v. 8). Data were derived from the average of three biological replicate experiments and presented as the mean±SEM. *P<0.05, **P<0.01 and ***P<0.001.

### B. Results

### RNF114 is Involved in the PARylation Dependent DNA Damage Response.

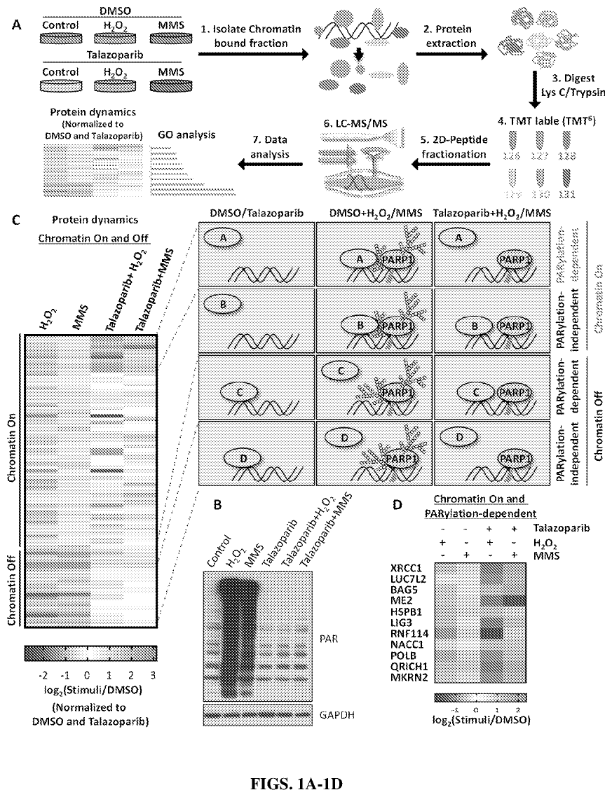

To identify the regulatory factors involved in the PARylation dependent DNA damage response, we performed the quantitative proteomic profiling experiments (FIG. 1A). We pre-treated the HCT116 cells with DMSO or Talazoparib, a potent PARPi (FIG. 1B). The cells were then treated with H₂O₂ or MMS to induce DNA damage, and the subsequent PARP1 activation (Zhen et al., 2017). We harvested the treated cells and isolated the chromatin fraction. After protein extraction and proteolytic digestion, we used isobaric labeling-based quantitative mass spectrometry for global protein expression profiling experiments. The digested peptides were labeled with the corresponding TMT⁶ reagent, combined, and subjected to multidimensional HPLC separation to ensure deep coverage of the proteome (Masson et al., 1998). From the combined dataset for the quantitative proteomic MS experiments, we were able to identify and quantify a total of 2346 proteins on chromatin. Using a threshold for Chromatin-On proteins (log 2>0.5 in H₂O₂/DMSO and MMS/DMSO samples) and Chromatin-Off proteins (log₂<−0.5 in H₂O₂/DMSO and MMS/DMSO samples), we identified 57 Chromatin-On proteins and 22 Chromatin-Off proteins.

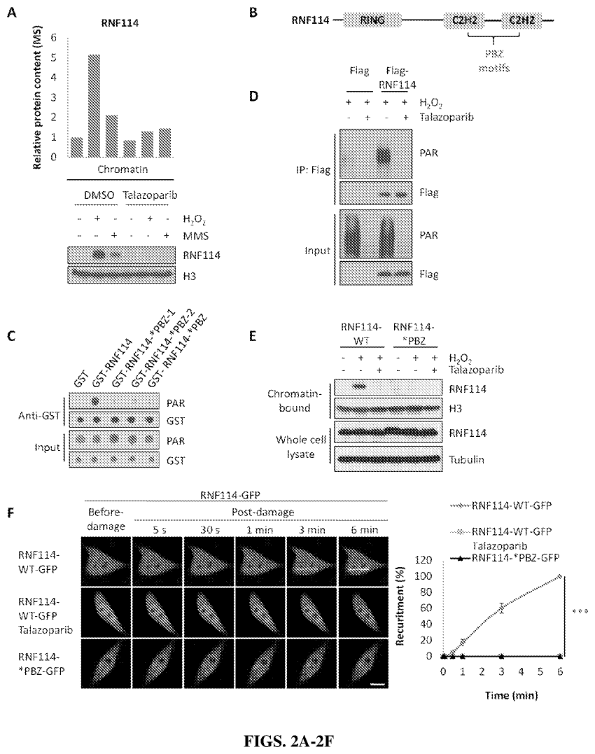

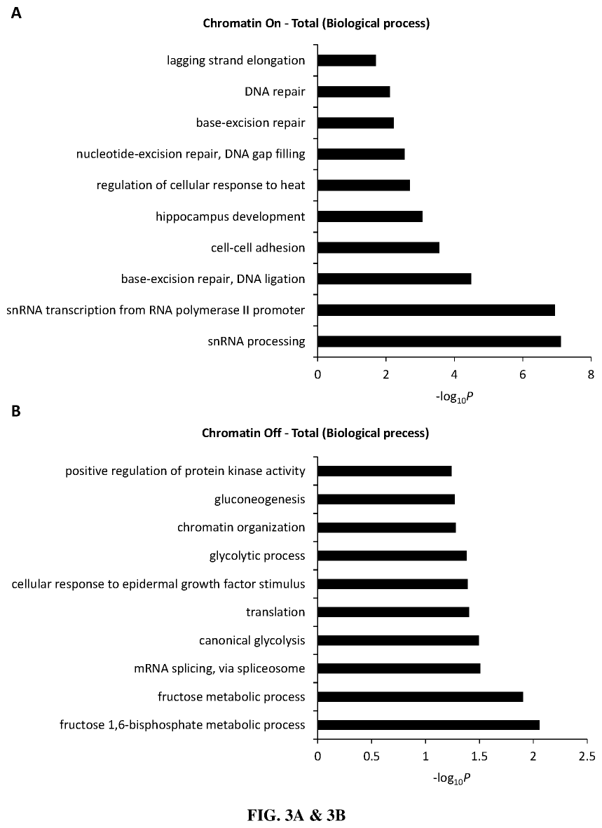

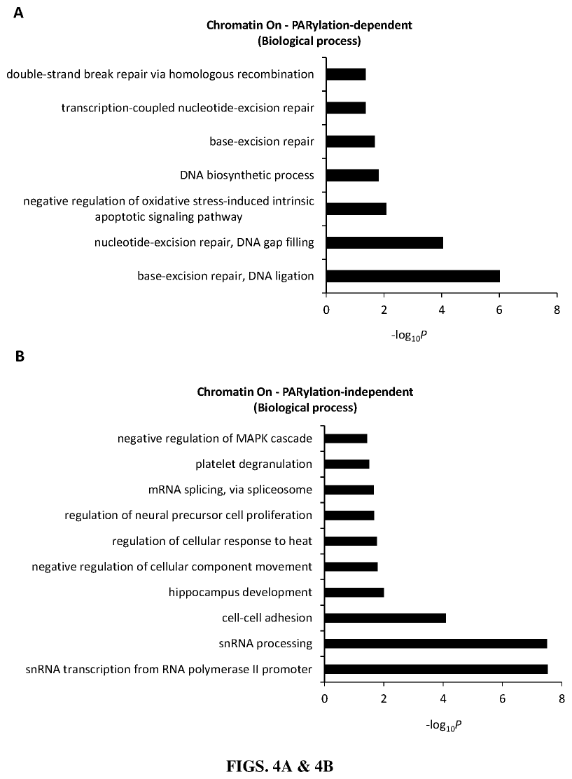

To characterize, in more detail, the proteomic changes that dependent on PARylation, these Chromatin-On and Chromatin-Off proteins were subjected to unsupervised hierarchical clustering analysis, and the results revealed that PARPi pre-treatment induced changes in protein dynamics patterns on chromatin and tended to converge into four closely clusters, i.e., Group I: Chromatin-On-PARylation-dependent proteins (11 proteins); Group II: Chromatin-On-PARylation-independent proteins (46 proteins); Group III: Chromatin-Off-PARylation-dependent proteins (6 proteins); Group IV: Chromatin-Off-PARylation-independent proteins (16 proteins) (FIG. 2C). Additionally, we performed GO analysis of the Chromatin-On and Off proteins and each of the four groups from the hierarchical clustering analysis (FIG. 3 and FIG. 4). We validated the dynamics of some factors in the four groups, including RNF114, INTS5, INTS3, SOX11, ZC3H3 and TAF10, and found excellent agreement (FIG. 2A and FIG. 5). Collectively, all these datasets will serve as an invaluable resource to provide the foundation for future research that helps delineate the molecular mechanisms of DNA damage response.

As our aim is to identify the regulatory factors that regulate the PARylation-dependent DDR, thus, we focused on the core set of 11 Chromatin-On-PARylation-dependent proteins (FIG. 1D). GO analysis of the Chromatin-On-PARylation-dependent group showed that most of these proteins were nucleic proteins involved in DNA damage repair, including base-excision repair, DNA ligation, nucleotide-excision repair, and DNA gap filling (FIG. 4A). Additionally, in consistent with previous studies (Zhen et al., 2018; Bijlmakers et al, 2011), we revealed that several well-known DNA damage repair factors, such as XRCC1, POLO, LIG3, were recruited to the chromatin in response to DNA damage in a PARylation dependent manner (FIG. 6).

Among the 11 chromatin-On-PARylation-dependent proteins, we were particularly intrigued by the proteins that could potentially regulate the protein dynamics, including the ubiquitination pathway that is connected to protein degradation through the ubiquitin proteasomal degradation pathway. Intriguingly, these analyses pointed to a ubiquitin E3 ligase that showed dramatic changes on chromatin (FIG. 1D). This protein was recruited to the chromatin during DDR. In contrast, its recruitment was blocked in cells that were pre-treated with PARPi (FIG. 2A). Thus, these data suggest that RNF114 could be a potential regulator involved in the PARylation-dependent DNA damage response.

### RNF114 is Recruited to DNA Lesions Via its PAR Binding Domains.

RNF114 has several distinct protein domains, including an amino-terminal RING domain (an E3 ligase domain), which is followed by two C2H2 [Cys(2)-His(2)]-type zinc finger domains (as potential PAR-Binding Zone motifs) (FIG. 2B). To test whether RNF114 is a PAR-binding protein, we performed in vitro and in vivo binding experiments. Using dot-blot experiments, we found that only WT RNF114, but not the RNF114 PBZ mutants (*PBZ-1, C143A/C146A; *PBZ-2, C173A/C176A; *PBZ, C143A/C146A/C173A/C176A), interacted with PAR polymers (FIG. 2C). Besides, with the treatment of H₂O₂ to induce DNA damage and initiate PARylation, it was found that RNF114 interacted with PAR chains and the interaction was dependent on PARylation, which was confirmed by the pre-treatment of PARPi to block PARylation (FIG. 2D). Furthermore, only RNF114 WT, but not the RNF114 PBZ mutant (*PBZ), was recruited to the chromatin, indicating that RNF114 was recruited to the DNA lesions through its PBZ motifs (FIG. 2E).

We further used laser microirradiation assays to study the recruitment kinetics of RNF114 during DDR. Notably, only RNF114-WT, but not the RNF114-PBZ mutant (compromised in PAR binding), was recruited to DNA lesions after DNA damage (FIG. 2F). The recruitment of RNF114 was also abolished in cells that were pre-treated with PARPi (FIG. 2F). Together, these findings suggest that the recruitment of RNF114 to the DNA lesions is dependent on its PAR binding domains.

### RNF114 Targets PARylated-PARP1 for its Ubiquitin-Proteasomal Degradation

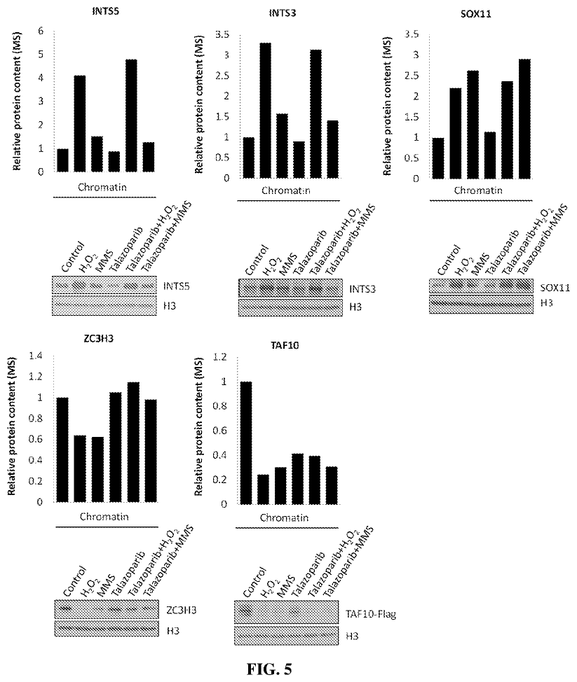

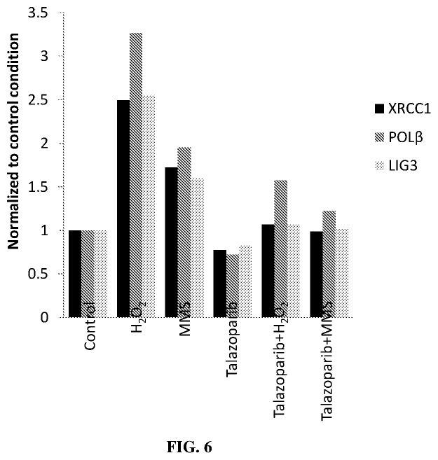

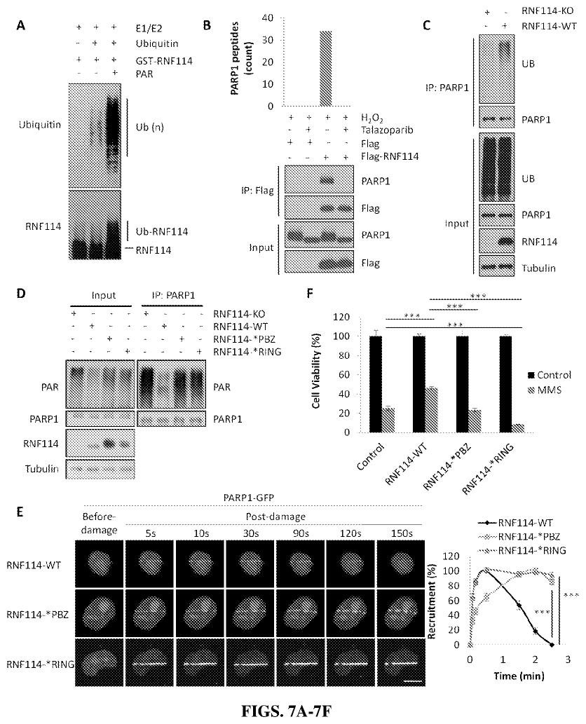

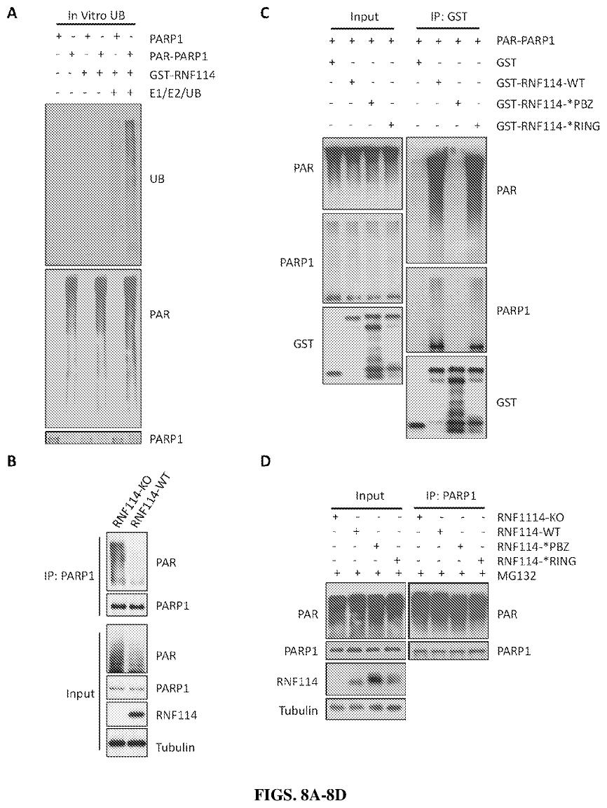

Because RNF114 interacts with PAR chains, we further used an in vitro ubiquitination assay to test whether PAR is a regulator of the E3 ligase activity of RNF114. We found that PAR chains dramatically up-regulated the ubiquitination E3 activity of RNF114 (FIG. 7A), suggesting that RNF114 is a PAR-dependent E3 ligase. To identify the substrates of RNF114, an IP-MS analysis was performed. PARP1 peptides were identified as the main target of RNF114 (FIG. 7B). However, pre-treatment of the cells using a PARPi (Talazoparib) completely blocked the binding between PARP1 and RNF114 (FIG. 7B), suggesting that the specific target of RNF114 is PARylated-PARP1, but not PARP1. These results are also consistent with RNF114 being a PAR-binding protein (FIG. 2C). In vivo and in vitro ubiquitination assays then revealed that RNF114 ubiquitinated PARylated-PARP1 (FIG. 7C and FIG. 8A). We also found that upon the treatment of H₂O₂, PARylated PARP1 was only degraded in control cells, but not in cells with RNF114 KO (FIG. 7D and FIG. 8B). Furthermore, cells expressing either the RNF114-*RING mutant (*RING, C29A/C32A, compromised in E3 ligase activity; Bijlmakers et al., 2011) or the RNF114-PBZ mutant (*PBZ, compromised in PARylated-PARP1 binding ability) (FIG. 8C) also failed to degrade PARylated PARP1 (FIG. 7D). RNF114-mediated degradation of PARylated PARP1 is via the ubiquitin proteasomal pathway, because the degradation of PARylated PARP1 was completely blocked in cells that were pre-treated with MG132 (FIG. 8D). Taken together, these results demonstrate that RNF114 is a PAR-dependent E3 ligase that specifically targets PARylated-PARP1 for ubiquitin proteasomal degradation.

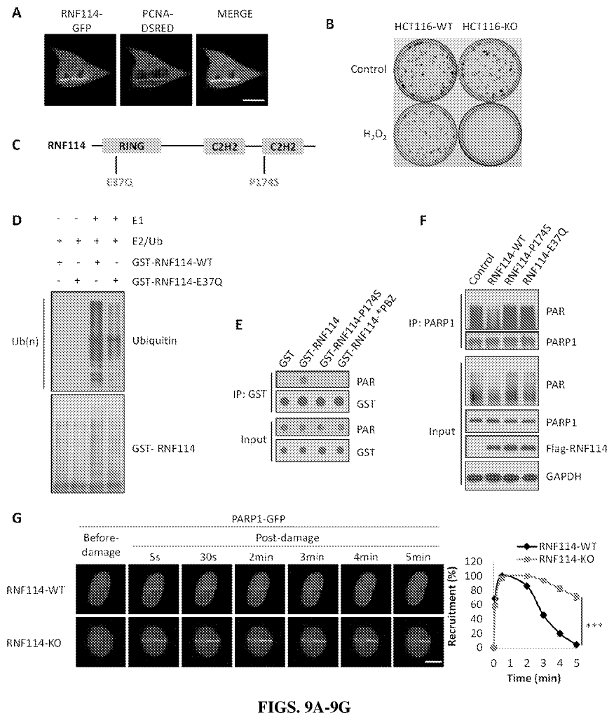

Using a laser microirradiation assay, we found that RNF114 co-localized with PCNA, further suggesting that RNF114 is involved in the DNA damage repair process (FIG. 9A). Furthermore, compared to cells expressing wild-type RNF114, RNF114-KO cells were more susceptible to various genotoxic agents (e.g., H₂O₂) (FIG. 9B). Next, we performed laser microirradiation assays to examine how RNF114 regulates the recruitment of PARP1 during DDR. In RNF114-WT cells, PARP1 was recruited to DNA lesions within a few seconds and then was released from DNA lesions within 2 min (FIG. 7E and FIG. 9G). However, PARP1 remained at the DNA lesion for a prolonged period in RNF114-KO cells (FIG. 9G). Additionally, we also tested the PARP1 relocation kinetics in the cells expressing RNF114 with either PBZ or RING mutations. Compared to RNF114-WT cells, PARP1 in RNF114-*PBZ mutant cells was retained on DNA lesions for a prolonged time (i.e., PARP1 trapping), because of the compromised PAR binding ability and PAR-mediated recruitment of this RNF114 mutant (FIG. 7E). PARP1 was also retained at the DNA lesions for a prolonged time in RNF1114-*RING mutant cells, because of its compromised E3 ligase activity (FIG. 7E). Intriguingly, the RNF1114-*RING mutant induced a much more pronounced level of PARP1 trapping, compared to the RNF114-*PBZ mutant or RNF114 KO (FIG. 7E). These data suggest a dominant-negative effect of the RNF114-*RING mutant: this mutant does not degrade PARylated-PARP1, although it maintains its binding to PARylated-PARP1 through the intact PBZ motifs. This binding could prevent PARylated PARP1 from being removed from DNA lesions by other PAR-dependent degradation mechanisms. Because PARP1 maintains its PARylation under these conditions, these data also suggest that RNF114-mediated degradation, rather than PARylation-induced steric hinderance/charge repulsion, functions as the main driver for the removal of PARylated PARP1 from the DNA damage site.

Trapped PARP1 is known to be cytotoxic, which could interfere with the recruitment of DNA repair factors. Furthermore, trapped PARP1 could lead to the stalling of replication forks. This, if left unresolved, may ultimately result in replication fork collapse, and cell death. Indeed, we found that MMS treatment induced more cell death in cells expressing the RNF114-*RING mutant, compared to those expressing RNF114-WT or the RNF114-*PBZ mutant (FIG. 7F).

It has been postulated that upon auto-PARylation, PARP1 is dissociated from DNA, owing to steric hindrance and charge repulsion. However, our results indicate that RNF114 is recruited to the DNA lesions by its binding to PAR chains. RNF114 then degrades PARylated-PARP1, which provides a novel ubiquitination-dependent mechanism to remove PARP1 from DNA lesions. Furthermore, the dominant-negative effect of PARP1 trapping caused by RNF114-*RING mutant suggests that the degradation pathway, but not the repulsion model, is the main mechanism to explain the dissociation of PARP1 from the DNA damage site.

### Cancer-Associated Mutations Impair the Function of RNF114

Several genes (e.g., BRCA) involved in DDR are known to be mutated in cancer. We queried the COSMIC database, and identified a number of mutations on RNF114. For example, two of these mutations, RNF114-E37Q and RNF114-P174S were localized in the RING domain, and the PBZ motif, respectively (FIG. 9C). Using an in vitro ubiquitination assay, we found that the RNF114-E37Q mutation compromised the E3 ligase activity of RNF114 (FIG. 9D). The RNF114-P174S mutation, on the other side, completely abolished the ability of RNF114 to bind to PAR polymers (FIG. 9E). Furthermore, both the RNF-E37Q and —P174S mutant were unable to degrade PARylated-PARP1 (FIG. 9F). These data suggest that both E3 ligase activity and PAR chain binding ability are necessary for RNF114 to regulate the degradation of PARylated-PARP1. Furthermore, these results raised the possibility that cancer-associated mutations of RNF114 could impair the proper removal of PARP1, contributing to tumorigenesis.

### Nimbolide is a Super Trapper of PARP1 and the PAR-Dependent DNA Repair Factors

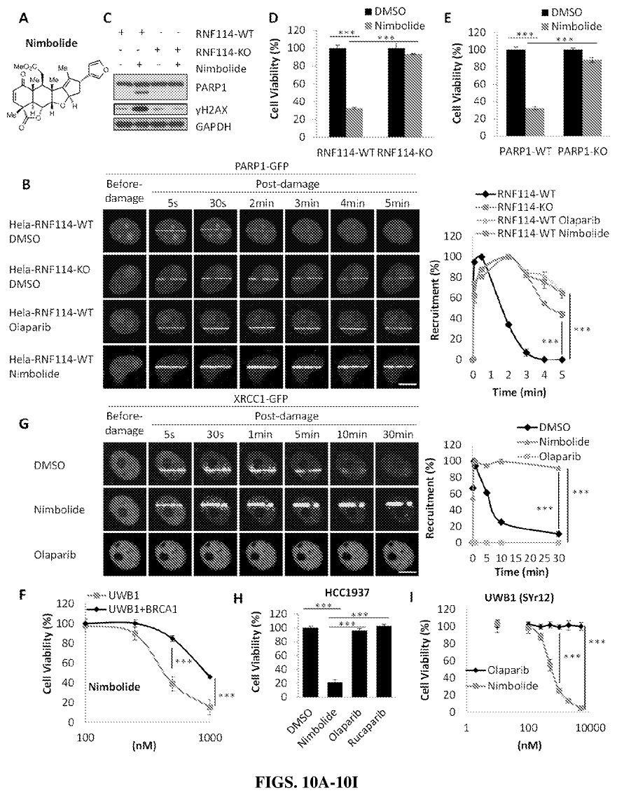

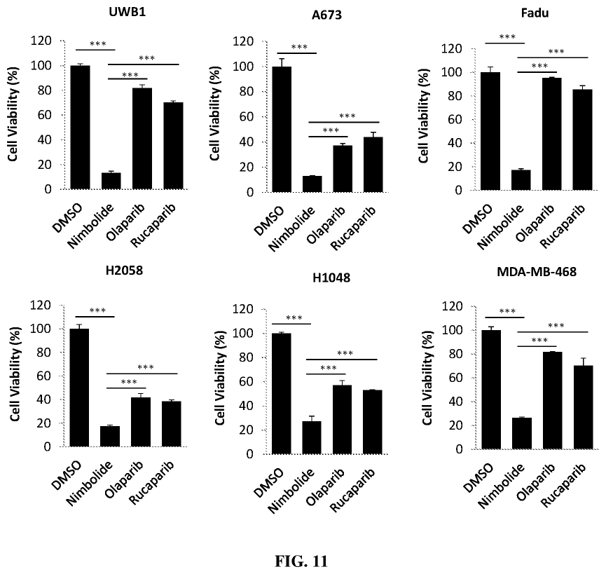

Nimbolide (FIG. 10A) is a natural product that is derived from the Neem tree. Previous studies suggest that Nimbolide inhibits tumorigenesis and metastasis through modulating different biological processes, including cell growth, invasion, survival, angiogenesis, inflammation and oxidative stress (Wang et al., 2016; Babykutty et al., 2012; Mahapatra et al., 2012; Raja Singh et al., 2014). A recent study showed that Nimbolide covalently modifies an N-terminal Cys (Cys8) of RNF114 and, in doing so, inhibits the E3 ligase activity of RNF114 (Spradlin et al., 2019). However, the functional consequence of this modification event was not demonstrated. Based on our data, we hypothesized that the treatment of Nimbolide could interfere with the RNF114-mediated degradation of PARP1, which could lead to PARP1 trapping, DDR and cell death. In this case, similar to the RNF114-*RING mutant (FIG. 7E), nimbolide-modified RNF114 maintains its binding to PARylated-PARP1. However, it is unable to degrade PARylated-PARP1, owing to nimbolide inhibition. Indeed, using laser microirradiation assays, we found that Nimbolide treatment resulted in profound PARP1 trapping. PARP1 trapping resulting from nimbolide treatment was very similar to that in the RNF114-*RING mutant cells (FIG. 7E and FIG. 10B). Intriguingly, the kinetics of PARP1 trapping induced by Nimbolide was similar to that of Olaparib, which is a PARPi and a potent trapper of PARP1 (FIG. 10B). However, nimbolide induced a greater level of PARP1 trapping compared to Olaparib (FIG. 10B).

Although both PARPi and nimbolide trap PARP1, a unique distinction between these two classes of compounds is that PARPi inhibit and trap PARP1, whereas nimbolide inhibits RNF114 and traps PARylated PARP1. PAR polymers on PARylated-PARP1 are known to recruit many DNA repair factors (e.g., XRCC1), triggering the formation of a large protein complex involved in the repair of DNA single strand breaks (SSBs) (Zhen et al., 2018; Bijlmakers et al., 2011). We therefore hypothesize that nimbolide treatment could induce the trapping of not only PARylated PARP1, but also other PAR-binding DNA repair proteins. To test this hypothesis, we performed laser microirradiation assays and found that, upon sensing DNA strand breaks, XRCC1 was rapidly recruited to the DNA lesions, through its PAR binding domains (FIG. 10G). Remarkably, because PARylated PARP1 is no longer removed (as a result of nimbolide treatment), XRCC1 accumulated and persisted at the DNA lesions in nimbolide-treated cells (FIGS. 9A & 9B). In contrast, PARPi treatment blocked the formation of PAR chains, which completely abolished the recruitment of XRCC1 to the DNA lesions (FIG. 10G).

Taken together, these data suggest that unlike regular PARPi, Nimbolide is a super trapper of not only PARP1 (PARylated), but also the PAR-dependent DNA repair factors.

Nimbolide is Synthetic Lethal with BRCA Mutations

Consistent with the notion that PARP1 trapping induces DNA damage and is cytotoxic, we found that Nimbolide treatment greatly upregulated γH2AX (a marker for DNA double strand breaks), and also induced PARP1 cleavage (FIG. 10C). However, nimbolide treatment did not increase γH2AX levels or PARP1 cleavage in RNF114-KO cells, indicating the “on-target” effect of nimbolide (FIGS. 10C & 10D). Additionally, nimbolide also failed to induced cell death in PARP1-KO cells, supporting the notion that the cytotoxic effects of nimbolide are mediated through PARP1 trapping induced by this compound (FIG. 10E).

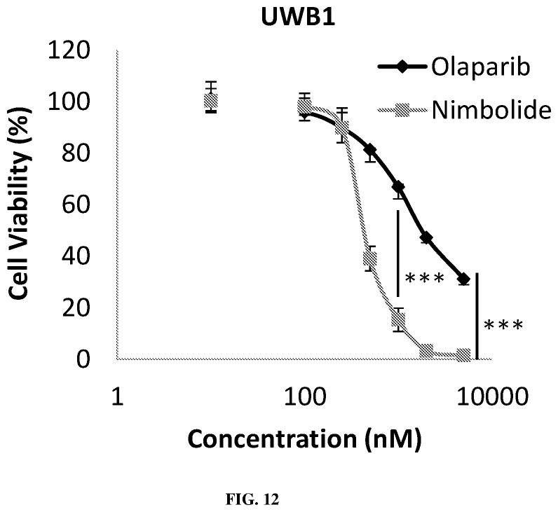

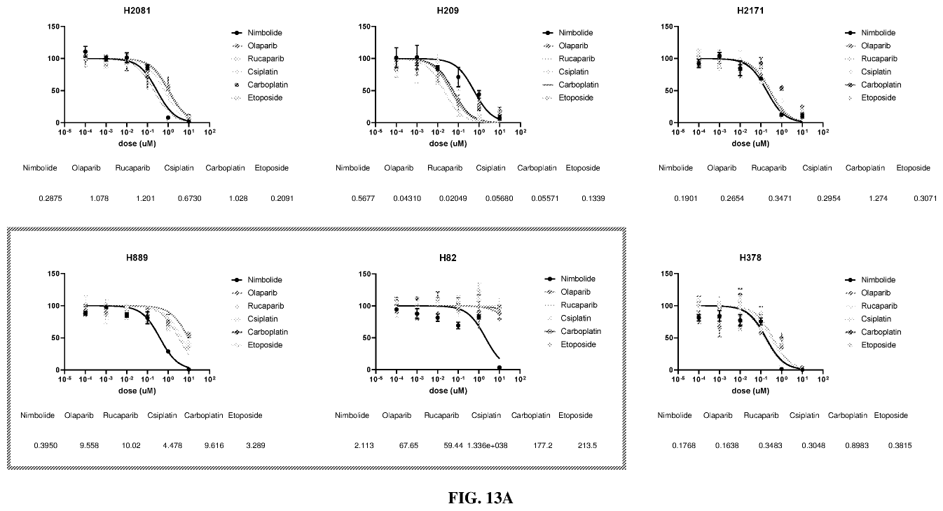

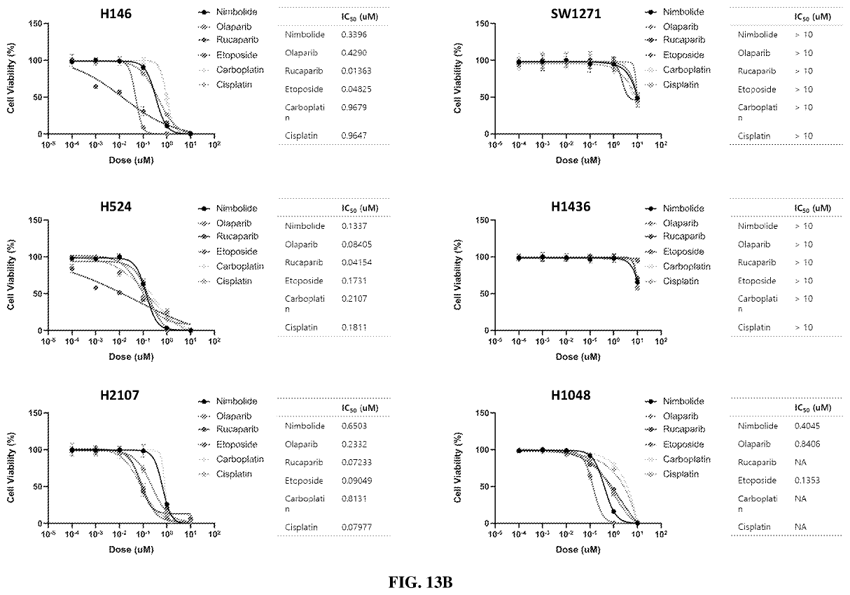

We surveyed the cytotoxicity of nimbolide in a series of cancer cell lines with different genetic background and mutation spectra, including UWB1 (ovarian carcinoma), A673 (Ewing's sarcoma), Fadu (Squamous cell carcinoma), H2058 (Non-small cell lung carcinoma), H1048 (Small cell lung carcinoma), MDA-MB-468 (breast adenocarcinoma) (FIG. 11). Although these cell lines displayed different degrees of sensitivity to PARPi (e.g., Olaparib and Rucaparib), they all showed profound sensitivity to nimbolide treatment (FIG. 11). It has been established that BRCA1/2-mutated cells are particularly sensitive to PARPi-induced trapping, and these cells are selectively killed by PARPi based on the “synthetic lethality” mechanism. Indeed, nimbolide demonstrated superior cytotoxicity against the BRCA1-null UWB1 cells, compared to Olaparib (FIG. 12). Furthermore, compared to control cells, UWB1 cells reconstituted with wt-BRCA1 showed greatly reduced sensitivity to nimbolide (FIG. 10F), again showing that the synthetic lethality between nimbolide and BRCA1/2 mutations. These results also suggest that mutations of genes in the homologous repair (HR) pathway will serve as important predictive biomarkers for nimbolide sensitivity. Besides ovarian cancer, we also tested a panel of small cell lung cancer cells that display intrinsic resistance to PARPi (talazoparib). Indeed, we found that two Talazoparib-resistant cell lines (i.e., H889 and H82) were highly sensitive to nimbolide (FIGS. 13A & 13B).

We also tested whether Nimbolide acts synergistically with other DNA-damaging agents, including methyl methanesulfonate (MMS), Doxorubicin, and Temozolomide (TMZ). Compared to nimbolide alone, the combination of nimbolide with these agents showed significantly increased toxicity in UWB1 cells (FIG. 14A). Furthermore, recent studies showed that “RR ACness” can also be induced by pharmacologically inhibiting key enzymes in the HR and DDR pathways (Chabanon et al., 2019). Towards this, we treated UWB1 cells with Nimbolide together with AZD6738 (an ATM/ATR inhibitor), CGK733 (an ATM/ATR inhibitor), LY2603618 (a CHK1 inhibitor), or SCH900776 (a CHK1 inhibitor) (FIG. 14B). Indeed, we found that Nimbolide was also able to synergize with these agents to further enhance its cytotoxicity (FIG. 14B).

### Nimbolide Overcomes Intrinsic and Acquired Resistance to PARP1 Inhibitors

BRCA1/2 mutations have been found in tumors originated in many different tissues, including breast, ovarian, prostate and pancreas. These mutations remain the best predictors for PARPi sensitivity. Although several PARPi have been approved for the treatment of breast and/or ovarian cancers with BRCA mutations, a significant fraction of the patients with BRCA^{mut} tumors showed de novo resistance, who failed to respond to these agents (intrinsic resistance) (Lehmann et al., 2011). Furthermore, similar to other targeted therapies, those patients who showed initial response to PARPi often develop resistance (acquired resistance) and relapsed disease is commonly observed. Thus, a strategy to overcome PARPi resistance is much needed to improve PARPi in order to achieve a more complete and durable response.

Because of the superior trapping activity of nimbolide (for both PARP1 and PAR-dependent DNA repair factors), we asked whether it is able to overcome intrinsic and acquired resistance to regular PARPi. HCC1937 is a BRCA1^{mut}, triple negative breast cancer cell line that is resistant to PARPi (FIG. 10H). This cell line, however, is exquisitely sensitive to nimbolide (FIG. 10H). A previous study reported the UWB1 (SYr12) cells, which is a PARPi-resistant UWB1 clone derived from long-term culturing of the parental UWB1 cells in the presence of a PARPi (Olaparib). The resistance mechanism of these cells and the related clones have been ascribed to the transcriptionally rewired DNA damage response network (Roy et al., 2007). Intriguingly, we found that UWB1 (SYr12) cells showed sensitivity to Nimbolide, but not Olaparib (FIG. 10I). Therefore, these data indicate that Nimbolide is able to kill tumors with intrinsic and acquired PARPi-resistance.

Nimbolide Triggers Innate Immune Response and Synergizes with Immune Checkpoint Inhibitors

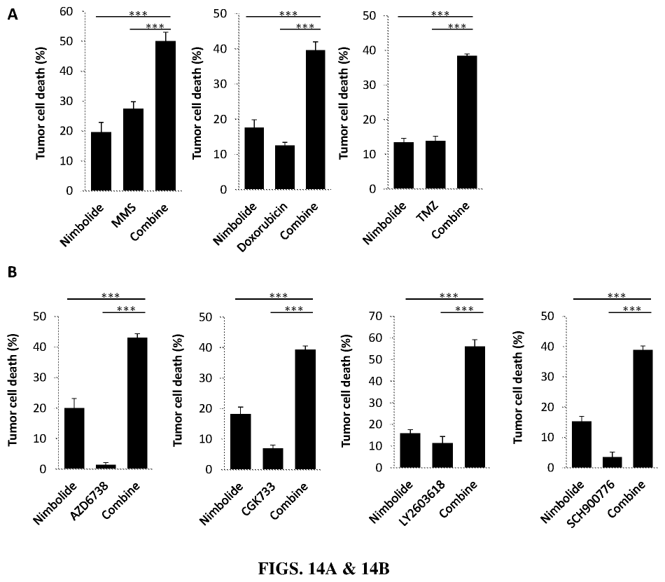

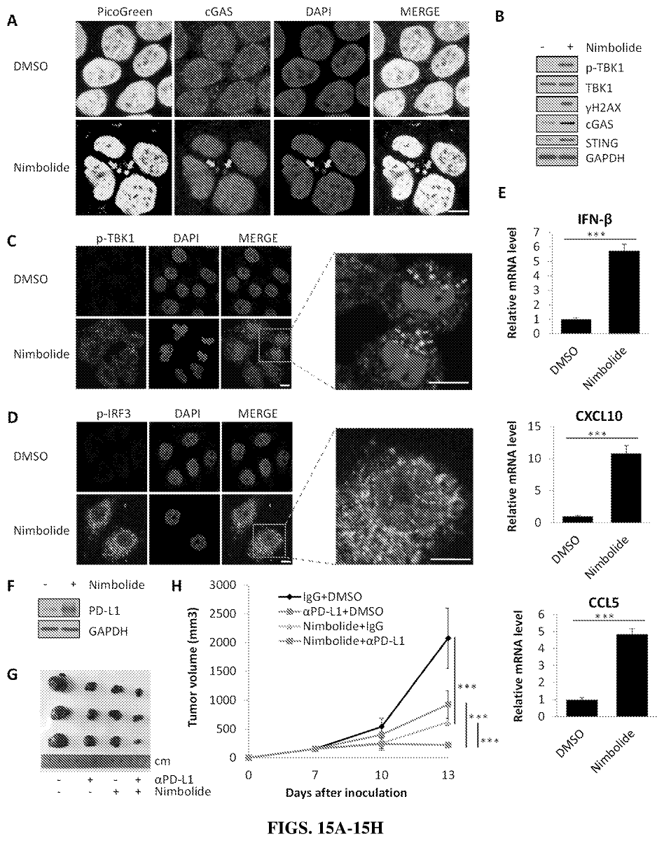

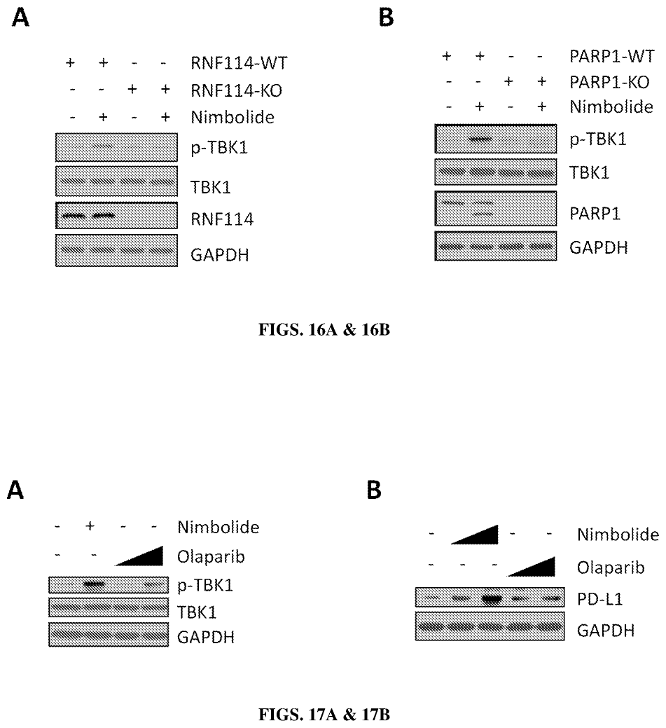

We recently showed that PARPi trigger innate immune signaling by PARP1 trapping-induced DNA damage response (Kim et al., 2020). Because nimbolide induces potent PARP1 trapping and the subsequent DDR (FIGS. 10B & 10C), we tested whether Nimbolide could have any immunomodulatory roles. We found that nimbolide treatment caused a significant accumulation of cytosolic dsDNA and micronuclei in HeLa cells (FIG. 15A). Immunofluorescence staining experiments then revealed the colocalization of cGAS and cytosolic dsDNA in nimbolide-treated Hela cells (FIG. 15A). Cyclic GMP-AMP synthase (cGAS) is a critical sensor of cytosolic dsDNA. After the recognition of cytosolic DNA, cGAS generates the second messenger cGAMP (cyclic GMP-AMP), which then binds to and activate STING. This binding event results in the recruitment and activation of Tank-binding kinase I (TBK1). TBK1 phosphorylates a transcription factor IRF3, which leads to its nuclear translocation, and the activation of type I interferon (IFN) signaling (Berger et al., 2018). We found that p-TBK1, a key downstream effecter of cGAS/STING signaling, was dramatically upregulated in nimbolide-treated cells (FIGS. 15B & 15C). Furthermore, nimbolide treatment induced the nuclear translocation of p-TBK1 and p-IRF3, suggesting the activation of STING signaling in these cells (FIGS. 15C & 15D). To further assess the activation of the cGAS/STING pathway, we examined the mRNA level of a number of target genes downstream of STING. Indeed, the mRNA levels of IFN-β, CXCL10 and CCL5 were dramatically increased in nimbolide-treated cells (FIG. 15E). Remarkably, nimbolide failed to induce the activation of cGAS/STING signaling in RNF114-KO or PARP1-KO cells, indicating the specificity of the immunomodulatory role of nimbolide (FIGS. 16A & 16B). Finally, consistent with the superior trapping activity of nimbolide, it was able to induce stronger activation of cGAS/STING signaling (as shown by higher levels of p-TBK1), compared to other PARPi (i.e., Olaparib) (FIG. 17A). Collectively, these results demonstrate that nimbolide induces the accumulation of cytosolic dsDNA, which then activates the cGAS-STING-TBK1-IRF3 innate immune signaling.

A critical downstream target of cGAS/STING signaling is programmed death-ligand 1 (PD-L1), a major ligand of PD-1. The binding of PD-L1 to the immune checkpoint molecule PD-1 transmits an inhibitory signal to reduce the proliferation of antigen-specific T cells. Recent studies suggested that PD-L1 expression is regulated by PARPi (Dawson and Dawson, 2017). Since Nimbolide treatment induces PARP1 trapping, DNA damage, and innate immune response, we tested whether PD-L1 is also regulated by nimbolide treatment. Indeed, we found that the expression of PD-L1 was greatly elevated in nimbolide-treated UWB1 cells (FIG. 15F). Finally, consistent with the superior trapping activity of nimbolide, it was able to induce stronger expression of PD-L1 compared to Olaparib (FIG. 17B).

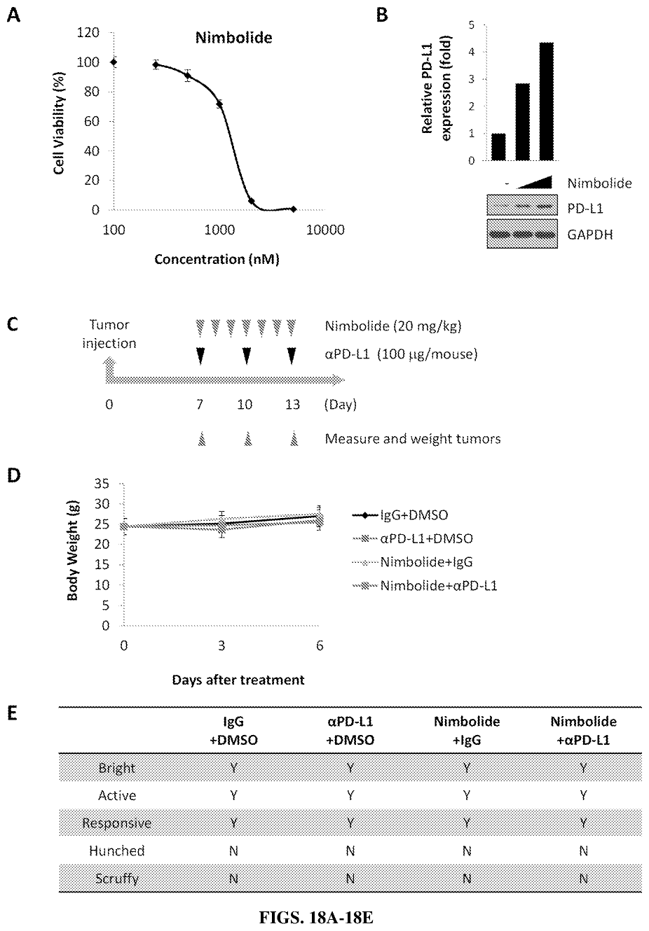

These results point to the critical immunomodulatory role of nimbolide and raise the intriguing hypothesis that nimbolide could synergize with immune checkpoint inhibitors. Consistent with a previous study (Hu et al., 2016), it was found that a murine melanoma cell line, B16 was relatively sensitive to nimbolide (FIG. 18A). Nimbolide treatment was also able to induce PD-L1 expression in these cells (FIG. 18B). Based on these results, the potential synergistic effect between nimbolide and an anti-PD-L1 antibody was evaluated using the C57BL/6 immuno-competent synergistic mouse model (FIG. 18C). Consistent with in vitro observations, Nimbolide significantly suppressed tumor growth in vivo. However, the combination of nimbolide and an anti-PD-L1 antibody demonstrated a dramatic synergistic effect, compared to each treatment alone (FIGS. 15G & 15H). Mice that received each or the combination treatment did not show any significant changes in their body weight or signs of pain and distress (FIGS. 18D & 18E). These results suggest that immune checkpoint inhibitors profoundly synergize with nimbolide to further enhance its anti-cancer effect in vivo.

RNF114-WT and —KO HCT116 cells were used to further examine the role of RNF114 in mediating Nimbolide-induced PARP1 trapping (FIG. 22A). Chromatin fractionation assay was performed, and it was found that Nimbolide treatment induced robust PARP1 trapping in RNF114-WT cells, but not in RNF114-KO cells (FIG. 22B). Furthermore, this PARP1 trapping was also induced in ID8-sgBRCA1 cells treated with Nimbolide (FIG. 22C).

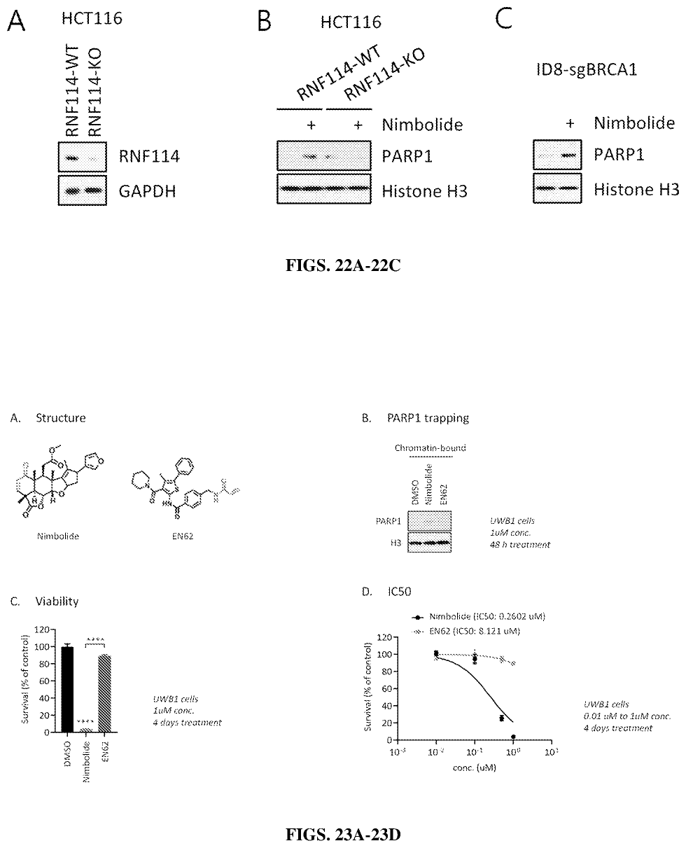

Finally, in a recent publication (Spradlin et al., Nature Chemical Biology, 15, 747, 2019), it was suggested that EN62 was another potential inhibitor of RNF114 (FIG. 23A). It was hypothesized that, similar to Nimbolide, EN62 also covalently modifies RNF114 at Cys8, and in doing so, blocks the ubiquitin E3 ligase activity of RNF114. However, barely detected any PARP1 trapping was detected in EN62-treated cells, compared to that in Nimbolide-treated cells (FIG. 23B). Furthermore, EN62 induced very low levels of toxicity in UWB1 cells, with an IC₅₀ of 8.1 μM. The IC₅₀ of Nimbolide in the same cell line was 0.26 μM (FIGS. 23C and 23D). The mechanistic details of EN62 warrant further studies in the future.

### Example 2: Nimbolide Analogs, Experimental Procedures, and Characterization Data

### A. General Information

Unless otherwise stated, commercially reagents were used as received, and all reactions were run under nitrogen atmosphere. All solvents were of HPLC or ACS grade. Nuclear magnetic resonance (NMR) spectra were recorded on a Varian Inova-400, Bruker-400, or Bruker-600 spectrometer at operating frequencies of 400 MHz (¹H NMR). Chemical shifts (6) are given in ppm relative to residual solvent (usually chloroform δ7.26 ppm for ¹H NMR) and coupling constants (J) in Hz. Multiplicity is tabulated as s for singlet, d for doublet, t for triplet, q for quadruplet, and m for multiplet. Mass spectra were recorded on Agilent 6120 mass spectrometer. The compounds of the present disclosure may be prepared according to the methods outlined in Example 1 as well as methods known to a skilled artisan.

### B. Synthetic Routes, Methodology, and Characterization of Nimbolide Derivatives Fragment a Preparation

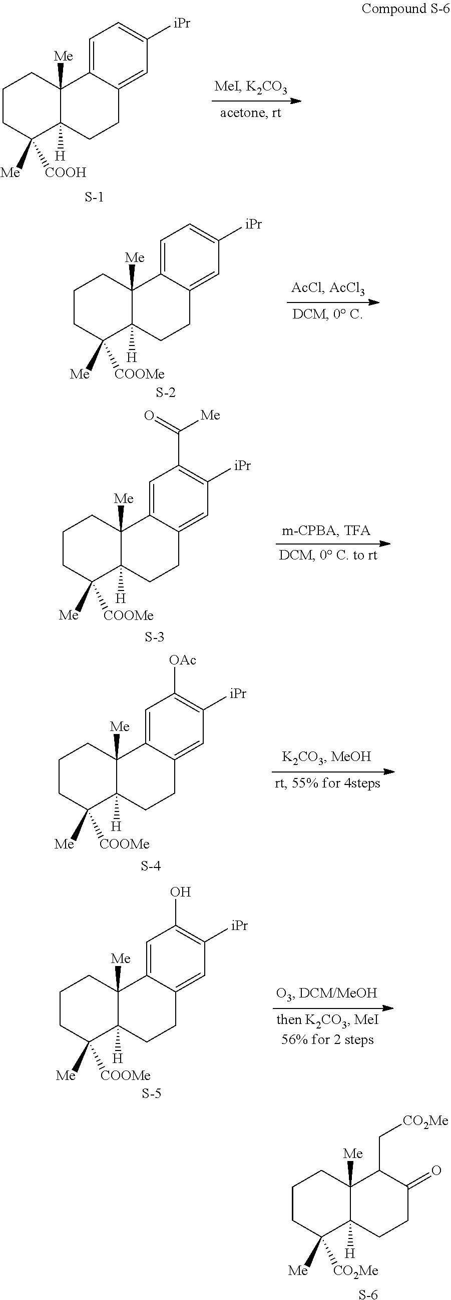

Step 1: To a solution of dehydroabietic acid S-1 (75 g, 250 mmol) in acetone (800 mL) was added K₂CO₃ (103.5, 750 mmol, 3.0 eq.) and MeI (78 mL, 1.25 mol, 5.0 eq.), then the mixture was stirred at 35° C. for 12 hours. After completion, the mixture was filtered by silica and concentrated under reduced pressure to give the crude S-2 which was used for the next step directly.

Step 2: To a solution of the crude product S-2 in CH₂Cl₂ (800 mL) was added acetyl chloride (62.2 ml, 875 mmol) at 0° C. Then AlCl₃ (100 g, 750 mmol) was added to the reaction mixture slowly at 0-5° C. Then the mixture was stirred at 0° C. for 30 minutes. On completion, the mixture was poured into 6N HCl (500 mL) at 0° C. carefully and stirred for 30 minutes. The mixture was diluted with water (200 mL) and extracted with CH₂Cl₂ (150 ml×3). The combined organic layers were washed with brine (100 ml×2) and saturated sodium bicarbonate aqueous solution (500 mL), dried over Na₂SO₄, filtered by silica column and concentrated under reduced pressure to give the crude S-3 which was used for the next step directly.

Step 3: To a solution of the crude product S-3 above in CH₂Cl₂ (700 mL) was added TFA (19.1 ml, 250 mmol) and m-CPBA (143.8 g (75%), 625 mmol) at 0° C. Then the mixture was stirred at 35° C. for 22 hours. After completion, the suspension mixture was filtered to get the filtrate and the filter cake was washed by CH₂Cl₂ (300 mL). The combined organic layers were cooled to 0° C. and then saturated NaHCO₃ aqueous solution (500 mL) was added slowly followed by saturated Na₂S₂O₃ aqueous solution (500 mL). After stirring at room temperature for 30 minutes, the organic phase was collected, and the water phase was extraction with CH₂Cl₂ (250 mL×3). The organic layers were dried over Na₂SO₄ and filtered by silica column, then concentrated via rotary evaporation to give the crude S-4 which was used for the next step directly.

Step 4: To a solution of the above product S-4 in MeOH (700 mL) was added K₂CO₃ (103.5 g, 750 mmol), then the mixture was stirred at 25° C. for 1 hour. After completion, the reaction solution was filtered by 500 g silica gel then concentrated via rotary evaporation to give the crude, which was purified on the silica gel chromatography (SiO₂, hexanes:EA=20:1) affording the product S-5 as white solid (45.7 g) in 55% yield for 4 steps.

Step 5: To a solution of the above product S-5 (6.0 g, 18.2 mmol) in CH₂Cl₂ (150 mL) and MeOH (50 mL) was induced 03 into the solution with stirring at −78° C. for 5 hours. The suspension of Zn (24.0 g, 360 mmol) in HOAc (50 mL) was added to the reaction mixture slowly to quench the reaction at −78° C. After addition, the mixture was allowed to warm up to 25° C. and stirred for 12 hours. After completion, the reaction mixture was filtered and concentrated under reduced pressure to give a residue which was re-dissolved in 100 mL Et₂O. 100 mL KOH (10%) aqueous was added, the water phase was collected, the pH was adjusted to 1-2 by 3 N HCl. The mixture was extracted with EA (200 ml×4), then dried over Na₂SO₄, concentrated via rotary evaporation to give the crude used for next step directly.

Step 6: To a solution of the above product in acetone (100 mL) was added K₂CO₃ (7.5 g, 54.6 mmol) and MeI (5.7 ml, 91 mol). The mixture was stirred at 35° C. for 12 hours. After completion, the reaction mixture was filtered and concentrated under reduced pressure to give a residue. The residue was purified by column chromatography (SiO₂, hexanes:EA=6:1) affording the product S-6 as colorless oil (3.16 g, 56% for 2 steps). [α]_{D}²⁶=−47.82 (c 1.2, CHCl₃). ¹H NMR (400 MHz, CDCl₃): δ 3.64 (s, 3H), 3.59 (s, 3H), 2.85-2.78 (dd, J=2.8, 9.6 Hz, 1H), 2.70-2.60 (dd, J=9.6, 16.4 Hz, 1H), 2.41-2.36 (m, 3H), 2.14 (dd, J=2.8, 16.4 Hz, 1H), 1.76-1.65 (m, 2H), 1.63-1.49 (m, 5H), 1.33-1.23 (m, 1H), 1.17-1.08 (s, 3H), 0.74-0.64 (s, 3H) ppm. ¹³C NMR (100 MHz, CDCl₃): δ 209.83, 178.51, 173.59, 77.00, 59.79, 52.14, 51.73, 47.88, 47.10, 41.17, 40.51, 37.91, 36.82, 27.23, 25.30, 17.59, 16.60, 15.12 ppm. HRMS (ESI-TOF): calc'd for C₁₇H₂₆O₅ [M+H]⁺: 311.1853, found: 311.1875. TLC:R_{f}=0.41 (3:1 hexanes:ethyl acetate).

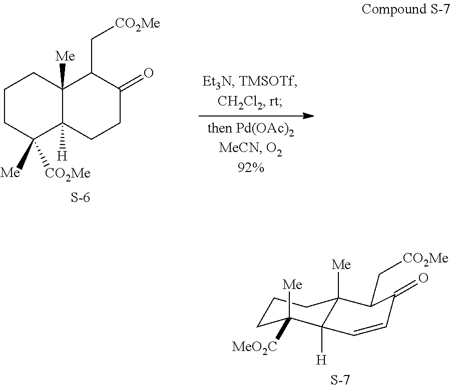

To a 500 mL dry round-bottom flask with stir bars was added S-6 (7.5 g, 24.2 mmol, 1.0 eq). Dry CH₂Cl₂ (250 mL), Et₃N (10.1 mL, 72.5 mmol, 3.0 eq) and TMSOTf (8.7 mL, 48.36 mmol, 2.0 eq) were added sequentially to flask at 0° C. under argon. The reaction was completed after another 3 h. The combined reaction mixture was quenched with saturated aq. NaHCO₃ (250 mL), extracted with CH₂Cl₂ (3×300 mL), washed with brine, dried over Na₂SO₄, and concentrated in vacuo to afford the crude silyl enol ether.

To a solution of the crude intermediate in 250 mL DMSO was added Pd(OAc)₂ (2.17 g, 25.2 mmol, 0.4 eq). The reaction mixture was heated at 60° C. while oxygen was being purged through the solution. The reaction mixture was stirred overnight and then quenched with brine (500 mL), extracted with EtOAc (3×200 mL). The combined organic layers were washed with brine (200 mL), and the aqueous phase was extracted with additional EtOAc (3×200 mL). The combined organic layers were dried over Na₂SO₄ and concentrated in vacuo. The crude residue was purified on the silica gel chromatography (SiO₂, hexane:EA=6:1) afford the product S-7 as yellow oil (6.91 g, 92%).

[α]_{D}²⁶=−117.11 (c 0.140, CHCl₃). ¹H NMR (600 MHz, CDCl₃): δ 6.61 (dd, J=10.1, 1.9 Hz, 1H), 6.07 (dd, J=10.1, 3.2 Hz, 1H), 3.72 (s, 3H), 3.71 (s, 3H), 3.23-3.17 (m, 1H), 2.99 (dd, J=8.0, 4.3 Hz, 1H), 2.75 (dd, J=16.4, 8.1 Hz, 1H), 2.22 (dd, J=16.5, 4.3 Hz, 1H), 1.84-1.68 (m, 2H), 1.68-1.56 (m, 3H), 1.45-1.38 (m, 1H), 1.26 (s, 3H), 0.84 (s, 3H) ppm. ¹³C NMR (150 MHz, CDCl₃): δ 199.05, 177.96, 173.89, 150.16, 129.88, 59.43, 52.57, 52.08, 51.21, 45.89, 42.76, 36.52, 35.87, 27.75, 17.87, 17.35, 14.33 ppm. HRMS (ESI-TOF): calc'd for C₁₇H₂₄05 [M+H]⁺: 309.1697, found: 309.1704. TLC:R_{f}=0.41 (3:1 hexanes:ethyl acetate).

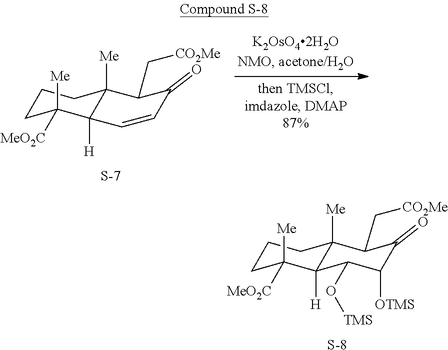

Compound S-7 (14.15 g, 45.89 mmol) was dissolved in 100 mL acetone and 50 mL H₂O stir at rt. NMO (16.13 g, 137.7 mmol, 3.0 eq) was added followed by the K₂OsO₄·2H₂O (676.6 mg, 1.84 mmol, 0.04 eq), and the reaction was completed after stirring at rt for 24 hours. Then removing the solvent under vacuo (water bath 56° C.) gave the crude diol. The diol was re-dissolved in 450 ml CH₂Cl₂ stir at rt. Imidazole (37.5 g, 551 mmol, 12.0 eq) was added followed by TMSCl (37 ml, 291 mmol, 6.3 eq) and DMAP (8.1 g, 66.3 mmol, 1.4 eq). The mixture was stirred at rt for 30 minutes and quenched with saturated aq. NaHCO₃ (500 mL) and saturated aq. Na₂S₂O₃ (500 mL). The organic phase was collected and the water phase was extracted with CH₂Cl₂ (3×300 mL), dried over Na₂SO₄, and concentrated in vacuo to afford the crude. Purification by the silica gel chromatography (SiO₂, hexanes:Et₂O=5:1) afforded the product S-8 as colorless oil (19.43 g, 87%).

[α]_{D}²⁷=−21.41 (c 0.943, CHCl₃). ¹H NMR (600 MHz, CDCl₃): δ 4.02 (d, J=2.6 Hz, 1H), 3.69 (dd, J=11.3, 2.6 Hz, 1H), 3.64 (s, 3H), 3.61 (s, 3H), 3.41 (dd, J=9.4, 3.5 Hz, 1H), 3.31 (d, J=11.2 Hz, 1H), 2.66 (dd, J=16.7, 9.4 Hz, 1H), 2.21 (dd, J=16.7, 3.7 Hz, 1H), 1.78-1.70 (m, 1H), 1.62-1.48 (m, 4H), 1.40-1.33 (m, 1H), 1.22 (s, 3H), 0.68 (s, 3H), 0.13 (s, 9H), 0.11 (s, 9H) ppm. ¹³C NMR (151 MHz, CDCl₃): δ 208.94, 178.82, 173.18, 79.85, 72.07, 53.85, 51.89, 51.36, 45.70, 43.96, 38.72, 37.50, 37.10, 27.02, 18.17, 16.77, 15.45, 0.76, 0.43 ppm. HRMS (ESI-TOF): calc'd for C₂₃H₄₂O₇Si₂ [M+H]⁺: 487.2542, found: 487.2544. TLC:R _{f}=0.40 (3:1 hexanes:ethyl acetate).

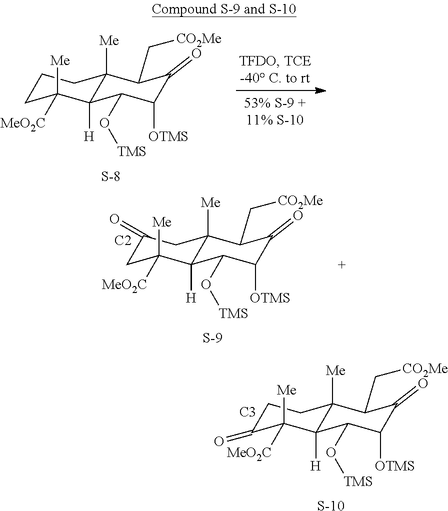

The compound S-8 (1.03 g, 2.12 mmol) was dissolved in 22 ml TCE, and cooled to −40° C. Then the fresh prepared TFDO solution 10 ml (prepared from 25 ml trifluoroacetone) was added to the above reaction mixture then warm to rt. The reaction completed after 4 hours. The solvent was removed via rotary evaporation to give the crude which was re-dissolved in 40 ml CH₂Cl₂ at rt. Imidazole (1.36 g, 20.0 mmol, 10.0 eq) was added followed by TMSCl (1.5 ml, 11.8 mmol, 5.5 eq) and DMAP (262.1 mg, 2.12 mmol, 1.0 eq). 30 minutes later, the reaction was quenched with saturated aq. NaHCO₃ (100 ml). The organic phase was collected and the water phase was extracted by CH₂Cl₂ (60 ml×4), dried over Na₂SO₄, then concentrated via rotary evaporation to give the crude. Purification on the silica gel chromatography (SiO₂, hexanes:EA=6:1→3:1) afforded the C2 ketone product as colorless oil S-9 (557.5 mg) in 53% yield and C3 ketone product S-10 as colorless oil (120.6 mg) in 11% yield.

C2-ketone product S-9 characterization data: [α]_{D}²⁷=−25.58 (c 0.383, CHCl₃). ¹H NMR (600 MHz, CDCl₃): δ 4.09 (d, J=2.5 Hz, 1H), 3.87 (d, J=11.0 Hz, 1H), 3.76 (dd, J=11.1, 2.6 Hz, 1H), 3.71 (dd, J=9.1, 3.8 Hz, 1H), 3.69 (s, 3H), 3.66 (s, 3H), 2.87 (d, J=12.9 Hz, 1H), 2.72 (dd, J=16.9, 9.1 Hz, 1H), 2.58 (d, J=12.2 Hz, 1H), 2.22 (ddd, J=17.0, 12.5, 2.2 Hz, 2H), 2.11 (dd, J=16.9, 3.7 Hz, 1H), 1.23 (s, 3H), 0.70 (s, 3H), 0.17 (s, 9H), 0.14 (s, 9H) ppm. ¹³C NMR (150 MHz, CDCl₃): δ 207.53, 207.48, 175.90, 172.58, 79.47, 71.63, 52.98, 52.97, 52.11, 52.01, 52.01, 47.66, 45.83, 42.12, 26.87, 18.01, 16.45, 0.71, 0.43 ppm. HRMS (ESI-TOF): calc'd for C₂₃H₄₀O₈Si₂ [M+H]⁺:501.2335, found: 501.2338. TLC:R_{f}=0.40 (3:1 hexanes:ethyl acetate).

C3-ketone product S-10 characterization data: [α]_{D}²⁷=−25.58 (c 0.383, CHCl₃). ¹H NMR (600 MHz, CDCl₃): δ 3.95 (d, J=2.5 Hz, 1H), 3.69-3.66 (m, 1H), 3.63 (d, J=11.1 Hz, 1H), 3.55 (s, 3H), 3.52 (s, 3H), 3.37 (dd, J=9.4, 3.6 Hz, 1H), 2.63 (dd, J=16.8, 9.4 Hz, 1H), 2.50 (ddd, J=15.9, 11.8, 8.5 Hz, 1H), 2.28 (dt, J=16.0, 3.7 Hz, 1H), 2.11 (dd, J=16.8, 3.6 Hz, 1H), 1.78-1.71 (m, 2H), 1.29 (s, 3H), 0.75 (s, 3H), 0.00 (s, 9H), 0.00 (s, 9H) ppm. ¹³C NMR (150 MHz, CDCl₃): δ 208.94, 208.43, 172.66, 172.63, 79.21, 70.90, 58.49, 52.42, 52.05, 52.02, 47.07, 36.89, 36.60, 34.25, 27.23, 17.15, 14.92, 0.73, 0.40 ppm. HRMS (ESI-TOF): calc'd for C₂₃H₄₀O₈Si₂ [M+H]⁺: 501.2335, found: 501.2337. TLC:R_{f}=0.21 (3:1 hexanes ethyl acetate).

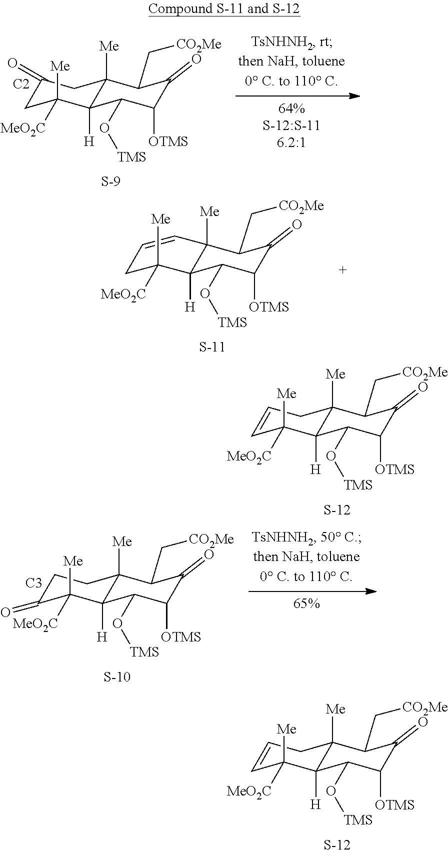

C2 ketone S-9 to S-11 and S-12: The compound S-9 (538.3 mg, 1.075 mmol) was dissolved in 11 ml MeOH, the TsNHNH₂ (240.2 mg, 1.29 mmol, 1.2 eq) was added and stirred for 12 hours. The solvent was removed via rotary evaporation to give the crude. To a 250 ml flask evacuated and backfilled with N₂, NaH 60% (107.5 mg, 2.69 mmol, 2.5 eq) was added. The reaction vessel was cooled to 0° C., 30 mL toluene was added and then the above crude re-dissolved in 55 mL toluene was added dropwise. After addition completed, the mixture warm to rt and stirred for 1 hour. Another 55 ml toluene was added then the reaction was warm to 110° C. for 2.5 hours. After cooling to rt. the reaction mixture was quenched with brine (50 mL) and extracted with EtOAc (4×50 mL). The combined organic layers were dried over Na₂SO₄, and concentrated in vacuo. The crude residue was purified on the silica gel chromatography (SiO₂, hexanes:EA=20:1→15:1) affording the product S-12 as colorless oil (287.4 mg, 55%) and product S-11 as colorless oil (46.3 mg, 9%).

C3 ketone S-10 to S-12: Compound S-10 (120 mg, 0.24 mmol) was dissolved in 0.3 ml toluene, TsNHNH₂ (53.6 mg, 0.288 mmol, 1.2 eq) was added and the reaction was stirred at 50° C. for 20 hours later. The solvent was removed via rotary evaporation to give the crude. To a 50 mL flask evacuated and backfilled with N₂, NaH 60% (24.6 mg, 0.6 mmol, 2.5 eq) was added. To the reaction vessel cooled to 0° C., 4.8 ml toluene was added, after which the above crude re-dissolved in 12 ml toluene was added dropwise. After addition completed, the mixture was stirred at rt for 1 hour. Then another 12 ml toluene was added and the reaction was warmed to 110° C. for 12 hours. After cooling to rt, the reaction mixture was quenched with brine (25 mL), extracted with EtOAc (4×30 mL). The combined organic layers was dried over Na₂SO₄, and concentrated in vacuo. The crude residue was purified on the silica gel chromatography (SiO₂, hexanes:EA=12:1) affording the product S-12 as colorless oil (75.1 mg, 65%)

Compound S-12 characterization data: [α]_{D}²⁷=−90.71 (c 0.507, CHCl₃). ¹H NMR (600 MHz, CDCl₃): δ 5.65 (ddd, J=9.7, 6.2, 1.8 Hz, 1H), 5.42 (dd, J=9.9, 2.6 Hz, 1H), 4.10 (d, J=2.8 Hz, 1H), 3.76 (dd, J=11.3, 2.8 Hz, 1H), 3.65 (s, 3H), 3.64 (s, 3H), 3.60-3.55 (m, 2H), 2.79 (dd, J=16.7, 9.4 Hz, 1H), 2.21 (dd, J=16.7, 3.4 Hz, 2H), 1.93 (dd, J=16.9, 6.2 Hz, 1H), 1.31 (s, 3H), 0.68 (s, 3H), 0.14 (s, 9H), 0.13 (s, 9H) ppm. ¹³C NMR (150 MHz, CDCl₃): δ 209.60, 176.84, 172.93, 131.74, 123.68, 79.97, 72.89, 52.08, 51.95, 51.49, 46.62, 44.08, 39.12, 36.72, 27.34, 17.98, 15.60, 0.96, 0.51 ppm. HRMS (ESI-TOF): not detected. TLC:R_{f}=0.42 (7:1 hexanes:ethyl acetate).

Compound S-11 characterization data: ¹H NMR (600 MHz, CDCl₃): δ 5.64 (ddd, J=10.3, 5.4, 2.4 Hz, 1H), 5.51 (ddd, J=10.3, 3.1, 1.3 Hz, 1H), 4.03 (d, J=2.6 Hz, 1H), 3.87 (dd, J=11.3, 2.6 Hz, 1H), 3.65 (s, 3H), 3.63 (s, 3H), 3.56 (d, J=11.2 Hz, 1H), 3.41 (dd, J=9.8, 3.5 Hz, 1H), 2.70 (dd, J=16.6, 9.8 Hz, 1H), 2.60 (dt, J=17.9, 2.7 Hz, 1H), 2.35 (dd, J=16.6, 3.5 Hz, 1H), 1.94 (ddd, J=17.9, 5.4, 1.4 Hz, 1H), 1.21 (s, 3H), 0.76 (s, 3H), 0.15-0.11 (m, 18H) ppm. ¹³C NMR (150 MHz, CDCl₃): δ 208.05, 178.50, 172.91, 131.05, 124.19, 79.79, 70.68, 52.89, 51.93, 51.31, 44.31, 42.11, 38.05, 37.34, 27.41, 18.39, 17.36, 0.51, 0.38. TLC:R _{f}=0.44 (7:1 hexanes:ethyl acetate).

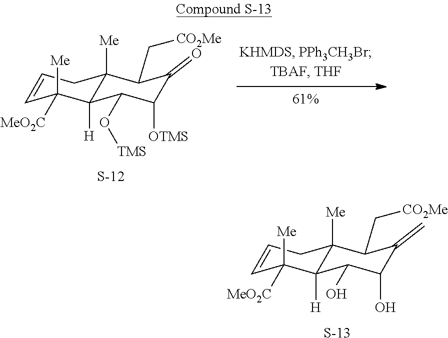

Under Ar (g), PPh₃CH₃Br (1.31 g, 3.67 mmol, 5.2 eq) was added to 70 mL THF and 0.7 mL DMSO. KHMDS solution (1 M in toluene, 7.1 mL, 3.53 mmol, 5.0 eq) was added dropwise. After 1-hour stirring, compound S-12 (342.2 mg, 0.706 mmol) in 36 mL THF was added dropwise and the reaction was stirred for 16 hours at rt. Then TBAF (1 M in THF, 7.1 ml, 7.1 mmol, 10.0 eq) was added. The mixture stirred at rt for 30 minutes then quenched with saturated aq. NH₄Cl (50 mL) and H₂O (100 mL), extracted with EtOAc (4×50 mL). The combined organic layers were dried over Na₂SO₄, and concentrated in vacuo. The crude residue was purified on the silica gel chromatography (SiO₂, hexanes:EA=1:1.5) affording the product S-13 as white solid (147.3 mg, 61%).

[α]_{D}²⁷=−126.95 (c 0.063, CHCl₃). ¹H NMR (600 MHz, CDCl₃): δ 5.71 (ddd, J=9.8, 6.4, 1.9 Hz, 1H), 5.48 (dd, J=9.9, 2.6 Hz, 1H), 5.16-5.11 (m, 1H), 4.78 (d, J=1.6 Hz, 1H), 4.34 (d, J=3.6 Hz, 1H), 3.69 (s, 3H), 3.67 (s, 3H), 3.10 (d, J=11.7 Hz, 1H), 3.08-3.01 (m, 1H), 2.51-2.38 (m, 3H), 2.09 (d, J=16.7 Hz, 1H), 1.89 (dd, J=16.6, 6.4 Hz, 1H), 1.39 (s, 3H), 0.70 (s, 3H) ppm. ¹³C NMR (150 MHz, CDCl₃): δ 178.46, 173.65, 147.52, 130.92, 125.30, 111.72, 76.16, 70.91, 52.66, 51.99, 46.91, 44.79, 44.37, 38.35, 36.58, 30.31, 18.10, 14.39 ppm. HRMS (ESI-TOF): calc'd for C₁₈H₂₆O₆[M+H]⁺: 339.1802, found: 339.1809. TLC:R_{f}=0.27 (1:1 hexanes:ethyl acetate).

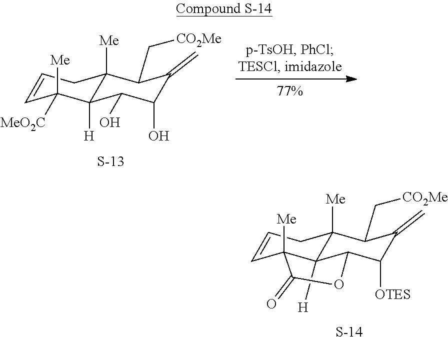

Compound S-13 (89.4 mg, 0.264 mmol) and p-TsOH (30.1 mg, 0.159 mmol, 0.6 eq) were dissolved in 26 ml PhCl and warmed to 90° C. after 1 hour, and 125° C. for additional 1.5 hours under vacuo with P₂O₅ to absorb water. After cooling to rt, 50 ml CH₂Cl₂ was added to the above reaction mixture followed by imidazole (179.8 mg, 2.64 mmol, 10 eq), TESCl (0.3 ml, 1.79 mmol, 6.8 eq) and DMAP (64.5 mg, 0.528 mmol, 2.0 eq). The reaction mixture was stirred at rt for 30 minutes then quenched with saturated aq. NaHCO₃ (100 mL), extracted with CH₂Cl₂ (4×80 mL). The combined organic layers were dried over Na₂SO₄, and concentrated in vacuo. The crude residue was purified on the silica gel chromatography (SiO₂, hexanes:Et₂O=5:1) afford the product S-14 as colorless oil (85.1 mg, 77%).

[α]_{D}²⁷=−20.98 (c 0.067, CHCl₃). ¹H NMR (600 MHz, CDCl₃): δ 6.17 (dt, J=10.0, 2.3 Hz, 1H), 5.65-5.59 (m, 1H), 5.20-5.15 (m, 1H), 4.91 (d, J=1.6 Hz, 1H), 4.62 (d, J=3.4 Hz, 1H), 4.20 (dd, J=12.3, 3.4 Hz, 1H), 3.63 (s, 3H), 3.03-2.94 (m, 1H), 2.82 (d, J=12.2 Hz, 1H), 2.50 (dd, J=15.0, 11.4 Hz, 1H), 2.36 (dd, J=15.0, 3.8 Hz, 1H), 2.23 (dt, J=18.3, 2.8 Hz, 1H), 2.08 (ddd, J=18.3, 4.1, 2.0 Hz, 1H), 1.27 (s, 3H), 1.00-0.89 (m, 9H), 0.84 (s, 3H), 0.66-0.53 (m, 6H) ppm. ¹³C NMR (150 MHz, CDCl₃): δ 178.28, 172.81, 147.37, 129.83, 128.24, 113.94, 77.30, 73.47, 51.90, 47.67, 46.45, 43.25, 40.08, 36.25, 31.05, 19.15, 15.57, 6.82, 4.81 ppm. HRMS (ESI-TOF): not detected. TLC:R_{f}=0.40 (5:1 hexanes:ethyl acetate).

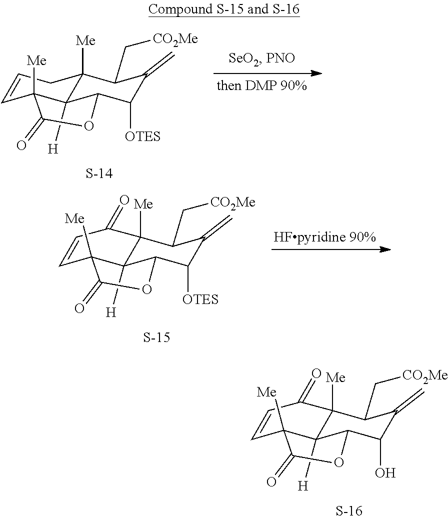

Under argon atmosphere, compound S-14 (279.1 mg, 0.664 mmol), SeO₂ (368.2 mg, 3.32 mmol, 5.0 eq) and PNO (dry with P₂O₅ under vacuo for 24 hours) (631.5 mg, 6.64 mmol, 10.0 eq) were dissolved in 70 mL dioxane then warmed to 100° C. After 4.5 hours, the reaction was completed, and cooled to rt. Saturated Na₂S₂O₃ aq. (200 mL) was added to quench the reaction, which was then extracted with EA (80 ml×5), dried over Na₂SO₄, and concentrated via rotary evaporation to give the crude. The crude was re-dissolved in 15 ml CH₂Cl₂. NaHCO₃ (279.1 mg, 3.32 mmol, 5.0 eq) was added followed by DMP (848.3 mg, 2.0 mmol, 3.0 eq). After stirring for 30 minutes, the reaction was quenched by saturated aq. NaHCO₃ (100 mL) and saturated aq. Na₂S₂O₃ (100 mL). Extraction with CH₂Cl₂ (80 mL×4), drying over Na₂SO₄, remove of the solvent give the crude, which was purified on the silica gel chromatography (SiO₂, hexanes:ethyl acetate=5:1) affording the product enone S-15 as white solid (200.1 mg, 69%).

Enone S-15 characterization data: [α]_{D}²⁷=+3.16 (c 0.063, CHCl₃). ¹H NMR (600 MHz, CDCl₃): δ 7.27 (d, J=9.8 Hz, 1H), 5.96 (d, J=9.7 Hz, 1H), 5.27 (s, 1H), 5.03 (s, 1H), 4.64 (d, J=3.4 Hz, 1H), 4.28 (dd, J=12.2, 3.4 Hz, 1H), 3.62 (s, 3H), 3.59 (dd, J=15.8, 3.2 Hz, 1H), 3.39 (d, J=10.1 Hz, 1H), 3.33 (d, J=12.2 Hz, 1H), 2.55 (dd, J=15.8, 11.7 Hz, 1H), 1.41 (s, 3H), 1.00 (s, 3H), 0.92 (t, J=7.9 Hz, 9H), 0.63-0.54 (m, 6H) ppm. ¹³C NMR (150 MHz, CDCl₃): δ 202.09, 175.82, 172.46, 149.93, 146.20, 131.75, 116.28, 76.31, 73.17, 51.79, 46.99, 46.14, 44.12, 40.64, 31.74, 18.63, 12.87, 6.77, 4.75 ppm. HRMS (ESI-TOF): not detected. TLC:R_{f}=0.40 (3:1 hexanes:ethyl acetate).

The enone S-15 (220.1 mg, 0.46 mmol) was dissolved in 10 ml MeCN at rt. 1 ml HF·Pyridine was added. The reaction completed after 2.5 hours and was quenched by saturated aq. NaHCO₃ (200 ml). Then it was extracted with EA (80 ml×5), dried over Na₂SO₄ and concentrated via rotary evaporation to give the crude. Purification by silica gel chromatography (SiO₂, hexane:EA=1:1) afforded the product S-16 as white solid (132.2 mg, 90%).

Enone S-16 characterization data: [α]_{D}²⁷=+68.31 (c 0.243, CHCl₃). ¹H NMR (600 MHz, CDCl₃): δ 7.28 (d, J=9.7 Hz, 1H), 5.97 (d, J=9.7 Hz, 1H), 5.39 (d, J=1.0 Hz, 1H), 5.07 (d, J=1.6 Hz, 1H), 4.76 (d, J=3.5 Hz, 1H), 4.37 (dd, J=12.3, 3.5 Hz, 1H), 3.67 (s, 3H), 3.61 (dd, J=16.6, 3.2 Hz, 1H), 3.45-3.37 (m, 2H), 2.58 (dd, J=16.5, 11.7 Hz, 1H), 2.43 (s, 1H), 1.43 (s, 3H), 1.02 (s, 3H) ppm. ¹³C NMR (150 MHz, CDCl₃): δ 201.63, 175.54, 172.72, 149.65, 144.61, 131.87, 118.06, 76.29, 72.43, 52.01, 47.18, 46.10, 44.07, 40.70, 31.28, 18.45, 18.43, 12.98 ppm. Note: one of the methyl group observed two peaks in the C NMR (18.45 and 18.43 ppm). HRMS (ESI-TOF): calc'd for C₁₇H₂₀O₆[M+H]⁺: 321.1333, found: 321.1339. TLC:R_{f}=0.30 (1:1 hexanes:ethyl acetate).

### Fragment B Preparation:

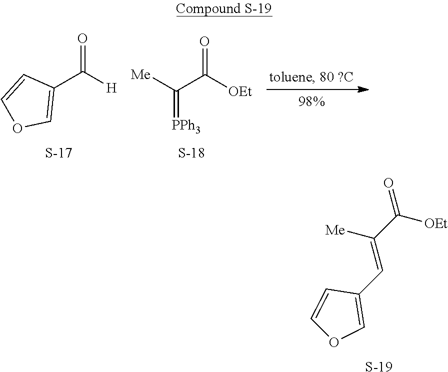

Furan-3-carbaldehyde S-17 (5.00 g, 52.1 mmol) was added to a stirred mixture of ethyl 2-(triphenyl-15-phosphanylidene)propanoate S-18 (28.25 g, 78.2 mmol, 1.5 eq) in dry toluene (250 mL). The mixture was heated to 80° C. and stirred overnight. After cooling to rt, the solvent was removed under reduced pressure. Purification by column chromatography (SiO₂, hexane:EA=20:1) afforded S19 as pale yellow oil (9.2 g, 98%).

¹H NMR (600 MHz, CDCl₃): δ 7.65 (s, 1H), 7.49-7.44 (m, 2H), 6.61 (d, J=1.9 Hz, 1H), 4.24 (q, J=7.1 Hz, 2H), 1.33 (t, J=7.1 Hz, 3H) ppm. ¹³C NMR (151 MHz, CDCl₃): δ 168.61, 144.07, 143.57, 129.17, 126.92, 122.22, 110.99, 60.94, 14.48, 14.37 ppm. HRMS (ESI-TOF): calc'd for C₁₀H₁₂O₃[M+H]⁺: 181.0859, found: 181.0857. TLC:R_{f}=0.54 (15:1 hexanes:ethyl acetate).

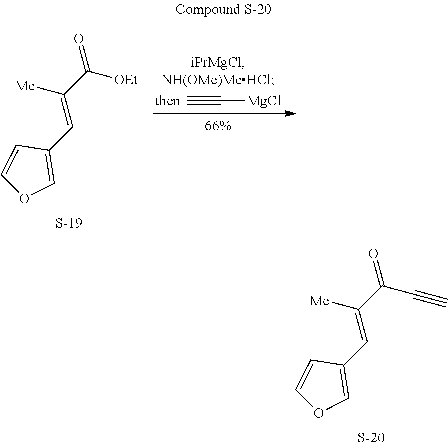

i-PrMgCl (1.3 mol/L in THF, 92 mL, 120.0 mmol, 4.0 eq) was added to the mixture of compound S19 (5.4 g, 30.0 mmol) and N,O-Dimethylhydroxylamine hydrochloride (5.85 g, 60.0 mmol, 2.0 eq) in dry THF (300 mL) at 0° C. The mixture was stirred at 0° C. for 30 min. Saturated aq. NH₄Cl (150 ml) was added to quench the reaction. The layers were separated and the aqueous layer was extracted several times with EtOAc. The combined organic extracts were washed with brine, dried over MgSO₄, and concentrated to yield crude Weinreb amide as an orange oil, which was used without further purification. This Weinreb amide was dissolved in dry THF (120 ml) in a flame dried flask. Ethynylmagnesium chloride (0.5 mol/L in THF, 240 mL, 120 mmol, 4.0 eq) was added at 0° C. The mixture was stirred for 3 hours before quenching with saturated aq. NH₄Cl (150 ml). The layers were separated and the aqueous layer was extracted several times with EtOAc. The combined organic extracts were washed with brine, dried over MgSO₄, and concentrated. Purification by column chromatography (SiO₂, hexane:EA=20:1) afforded the product S-20 as a yellow solid (3.17 g, 66%).

¹H NMR (600 MHz, CDCl₃): δ 7.85 (s, 1H), 7.78 (s, 1H), 7.51 (s, 1H), 6.69 (d, J=1.5 Hz, 1H), 3.30 (s, 1H), 2.07 (s, 3H) ppm. ¹³C NMR (150 MHz, CDCl₃): δ 179.31, 145.59, 144.22, 137.19, 136.25, 122.39, 110.82, 79.86, 79.76, 12.07 ppm. HRMS (ESI-TOF): not detected. TLC:R_{f}=0.45 (4:1 hexanes:ethyl acetate).

Furan-3-carbaldehyde (5 g, 52.1 mmol) was added to a stirred mixture of ethyl 2-(triphenyl-15-phosphanylidene)propanoate (28.25 g, 78.2 mmol, 1.5 equiv.) in dry toluene (250 mL). The mixture was heated to 80° C. and stirred overnight. After cooling to rt, the solvent was removed under reduced pressure. Purification by column chromatography (silica, 20:1 hexanes:EtOAc) afforded 9.2 g (98%) of the compound S-8 as a pale yellow oil.

Physical State: yellow oil. ¹H NMR (600 MHz, CDCl₃): δ 7.65 (s, 1H), 7.48 (s, 1H), 7.46 (t, J=1.5 Hz, 1H), 6.61 (d, J=1.5 Hz, 1H), 4.24 (q, J=7.0 Hz, 2H), 2.10 (d, J=1.2 Hz, 4H), 1.33 (t, J=7.2 Hz, 3H) ppm. ¹³C NMR (151 MHz, CDCl₃): δ 168.61, 144.07, 143.57, 129.17, 126.92, 122.22, 110.99, 60.94, 14.48, 14.37 ppm. TLC:R_{f}=0.66 (4:1 hexanes:ethyl acetate).

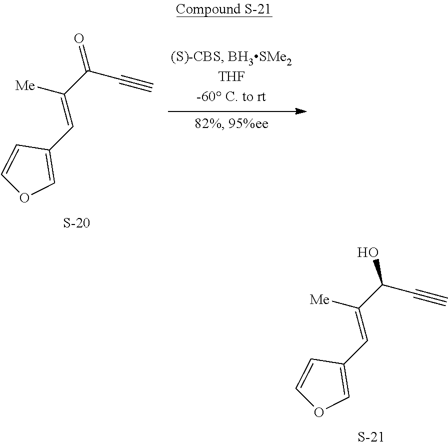

(S)-CBS (2.217 g, 8 mmol, 0.5 eq) was added to the solution of ketone S-20 (2.56 g, 16 mmol) in THF (320 mL) at −60° C. under an argon atmosphere. After the mixture was stirred for 10 min, BH₃·Me₂S (3 ml, 31 mmol, 2.0 eq) was added dropwise and stirred for 5 hours during which the temperature slowly raised to 0° C. MeOH (5.0 mL) was added followed by aq. NaHCO₃ (100 ml). The mixture was extracted by EtOAc (100 ml×5), and the combined extracts were dried (Na₂SO₄) and concentrated via rotary evaporation to give the crude. Purification by column chromatography (SiO₂, hexane:EA=6:1) afforded compound S21 as a pale yellow oil (2.125 g, 82%, ee 95%).

[α]_{D}²⁷=+17.99 (c 0.1, CHCl₃). ¹H NMR (600 MHz, CDCl₃): δ 7.48 (s, 1H), 7.41 (s, 1H), 6.49 (s, 1H), 6.46 (s, 1H), 4.90-4.89 (m, 1H), 2.59 (d, J=2.2 Hz, 1H), 2.00 (s, 3H) ppm. ¹³C NMR (151 MHz, CDCl₃): δ 142.94, 141.52, 135.15, 122.16, 117.95, 111.01, 82.87, 74.58, 68.11, 14.72 ppm. HRMS (ESI-TOF): calc'd for C₁₀H₈O₂ [M+H]⁺: 161.0597, found: 161.0598. TLC:R_{f}=0.57 (2:1 hexanes:ethyl acetate).

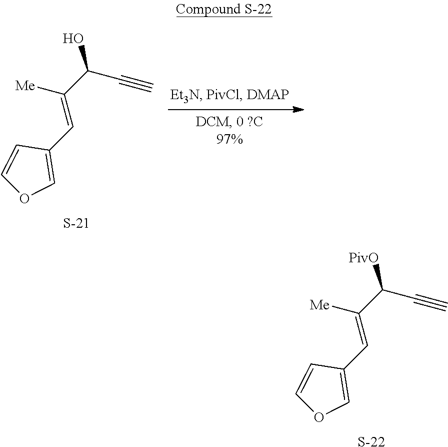

A flame dried flask equipped with a magnetic stir bar was charged with the compound S-21 (2.01 g, 12.34 mmol) in anhydrous dichloromethane (250 mL). Then pivaloyl chloride (2.23 g, 2.28 mL, 18.52 mmol, 1.5 eq) and trimethylamine (6.24 g, 8.6 mL, 61.7 mmol, 5.0 eq) was added at 0° C. followed by catalytic amounts of DMAP (754.3 mg, 0.5 eq). The reaction was stirred at 0° C. for 30 min. Quenched with an aqueous solution of NaHCO₃ and extracted with dichloromethane (100 ml×5). Combined organic layers were dried over anhydrous MgSO₄, filtered and concentrated via rotary evaporation to give the crude. Purification by column chromatography (SiO₂, hexane:EA=20:1) afforded compound S-22 as yellow oil (2.95 g, 97%). Note: if the extra pivaloyl chloride observed in NMR, filtration through basic alumina could remove the pivaloyl chloride.

[α]_{D}²⁷=+15.61 (c 0.1, CHCl₃). ¹H NMR (600 MHz, CDCl₃): δ 7.50 (s, 1H), 6.50 (s, 2H), 5.88 (d, J=1.8 Hz, 1H), 2.55 (d, J=2.2 Hz, 1H), 1.96 (d, J=1.0 Hz, 4H), 1.23 (s, 9H) ppm. ¹³C NMR (151 MHz, CDCl₃): δ 177.25, 143.02, 141.83, 131.65, 122.01, 120.15, 110.97, 79.86, 74.76, 68.93, 39.03, 27.15, 14.83 ppm. HRMS (ESI-TOF): not detected. TLC:R_{f}=0.48 (hex/CH₂Cl₂=1:1).

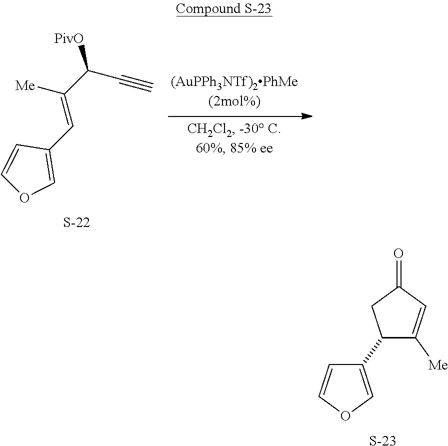

(PPh₃AuNTf)₂-PhMe (317.1 mg, 2 mol %), dichloromethane (100 mL) and water (0.18 mL, 1.0 eq) were added to a 250 mL flask. The mixture was cooled to −30° C. The starting material S-22 (2.51 g, 10.1 mmol) in dichloromethane (10 mL) was added dropwise. After stirring for 10 minutes, solvent was removed via rotary evaporation to give the crude. Purification by column chromatography (SiO₂, hexane:EA=4:1) afforded compound S-23 as yellow oil (0.98 g, 60%, ee 85%).

[α]_{D}²⁷=+50.46 (c 0.21, CHCl₃). ¹H NMR (600 MHz, CDCl₃): δ 7.39 (s, 1H), 7.34 (s, 1H), 6.15 (s, 1H), 6.03 (s, 1H), 3.87 (d, J=6.7 Hz, 1H), 2.85 (dd, J=18.8, 7.0 Hz, 1H), 2.36 (d, J=18.8 Hz, 1H), 1.98 (s, 3H) ppm. ¹³C NMR (151 MHz, CDCl₃): δ 208.57, 179.60, 144.12, 139.60, 131.07, 125.01, 108.64, 44.53, 40.82, 17.68 ppm. HRMS (ESI-TOF): calc'd for C₁₀H₁₀O₂[M+H]⁺: 160.0754, found: 160.0751. TLC:R_{f}=0.45 (2:1 hexanes:ethyl acetate).

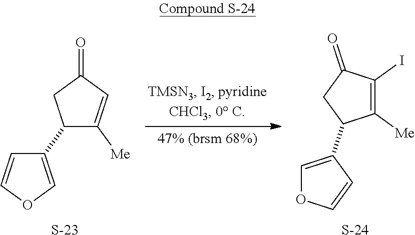

Under Ar (g), the enone S-23 (231.2 mg, 1.43 mmol) was dissolved in 14 ml CHCl₃, and cooled to 0° C. TMSN₃ (0.56 ml, 4.28 mmol, 3.0 eq) was added dropwise. After stirring for 2 hours, I₂ (725.9 mg, 2.86 mmol, 2.0 eq) in 3 ml pyridine and 3 ml CHCl₃ was added dropwise. Then the mixture was allowed to rt gradually and stirred for 24 hours. Then the reaction mixture was cooled to 0° C. again, the another TMSN₃ (0.56 ml, 4.28 mmol, 3.0 eq) was added dropwise. 2 hours later, 12 (725.9 mg, 2.86 mmol, 2.0 eq) in 3 ml pyridine and 3 ml CHCl₃ was added dropwise, then the mixture was warmed to rt gradually. After stirring for another 24 hours, the reaction was quenched with saturated aq. Na₂S₂O₃ (200 mL) and extracted with ethyl acetate (50 mL×4). The combined organic phase was dried over Na₂SO₄, then concentrated via rotary evaporation to give the crude. Purification on the silica gel chromatography (SiO₂, hexane:EA=6:1→3:1) afforded the product S-24 as yellow oil (390.0 mg, 47%) and recovered the starting material S-23 as yellow oil (144.2 mg, 21%).

[α]_{D}²⁷=+91.55 (c 0.487, CHCl₃). ¹H NMR (600 MHz, CDCl₃): δ 7.41 (s, 1H), 7.37 (s, 1H), 6.13 (d, J=1.8 Hz, 1H), 4.02 (dd, J=7.2, 2.3 Hz, 1H), 3.02 (dd, J=18.8, 7.1 Hz, 1H), 2.54 (dd, J=18.8, 2.4 Hz, 1H), 2.09 (s, 3H) ppm. ¹³C NMR (150 MHz, CDCl₃): δ 202.35, 180.77, 144.38, 139.72, 124.48, 108.52, 103.58, 42.23, 41.91, 20.69 ppm. HRMS (ESI-TOF): calc'd for C₁₀H₉O₂I [M+H]⁺: 288.9720, found: 288.9721. TLC:R_{f}=0.56 (3:1 hexanes:ethyl acetate).

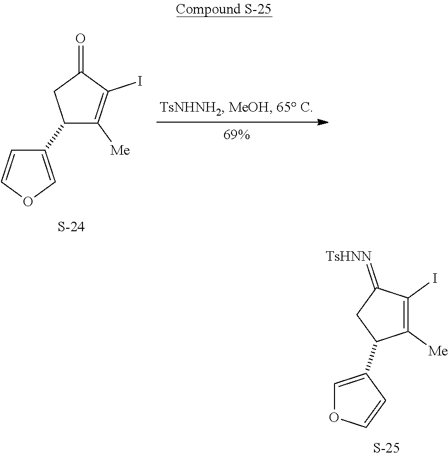

Compound S-24 (336.4 mg, 1.17 mmol) and TsNHNH₂ (1.31 g, 7.01 mmol, 6.0 eq) were dissolved in 12 ml MeOH then warmed to 65° C. After 12 hours the reaction was completed, remove the solvent via rotary evaporation to give the crude, which was purified on the silica gel chromatography (SiO₂, hexane:EA=5:1) to afford the product S-25 as yellow solid (364.7 mg, 69%). Note: this compound was unstable in CDCl₃.

[α]_{D}²⁷=+91.55 (c 0.487, CHCl₃). ¹H NMR (600 MHz, CD₃CN): 6 8.13 (s, 1H), 7.90-7.81 (m, 2H), 7.42 (t, J=1.8 Hz, 1H), 7.39 (dd, J=7.4, 1.5 Hz, 3H), 6.15 (dd, J=1.9, 0.9 Hz, 1H), 3.99 (d, J=7.9 Hz, 1H), 3.01 (dd, J=18.1, 7.9 Hz, 1H), 2.44 (m, 4H), 1.84 (d, J=1.3 Hz, 3H) ppm. ¹³C NMR (150 MHz, CD₃CN): 6 166.10, 164.25, 145.20, 144.95, 140.75, 136.57, 130.31, 129.06, 126.50, 109.70, 95.39, 43.91, 35.18, 21.51, 19.14 ppm. HRMS (ESI-TOF): not detected. TLC:R_{f}=0.34 (3:1 hexanes:ethyl acetate).

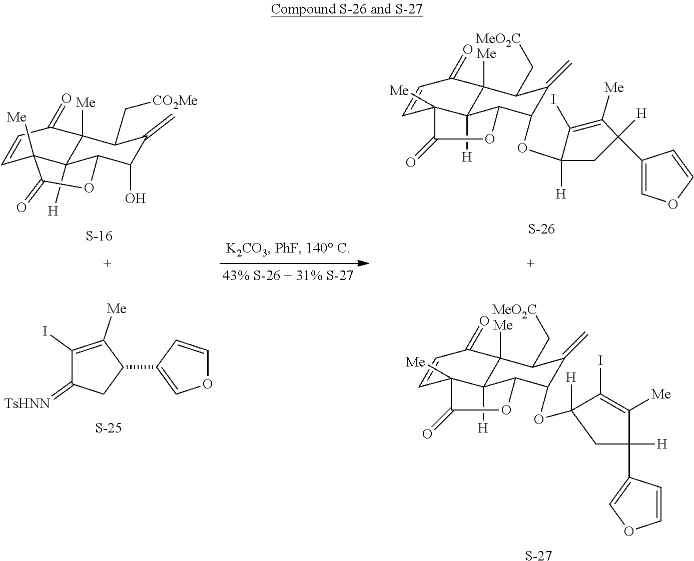

Compound S-16 (41.4 mg, 0.124 mmol), tosylhydrazone S-25 (169.8 mg, 0.372 mmol, 3.0 eq) and K₂CO₃ (68.5 mg, 0.496 mmol, 4.0 eq) were mixed in 3.7 ml fluorobenzene, then the mixture was warmed to 140° C. for 30 minutes. After cooling to rt, removal of the solvent via rotary evaporation gave the crude. Purification on the silica gel chromatography (SiO₂, hexane:EA=5:1) afforded the product S-26 as colorless oil (33.3 mg, 43%) and its diastereomer S-27 as yellow foam (23.5 mg, 31%).

Compound S-26 characterization data: [α]_{D}²⁷=+43.04 (c 0.153, CHCl₃). ¹H NMR (600 MHz, CDCl₃): δ 7.27 (d, J=1.9 Hz, 1H), 7.23 (dd, J=9.7, 1.6 Hz, 1H), 7.18 (s, 1H), 6.05 (s, 1H), 5.89 (dd, J=9.7, 1.6 Hz, 1H), 5.34 (s, 1H), 5.04 (d, J=2.2 Hz, 1H), 4.67 (d, J=3.7 Hz, 1H), 4.64-4.58 (m, 1H), 4.30 (dt, J=12.5, 2.5 Hz, 1H), 3.75 (t, J=6.7 Hz, 1H), 3.58-3.51 (m, 4H), 3.33 (dd, J=12.3, 1.6 Hz, 1H), 3.22 (d, J=11.7 Hz, 1H), 2.56-2.47 (m, 1H), 2.27 (dt, J=13.0, 4.3 Hz, 1H), 2.11-2.05 (m, 1H), 1.60 (s, 3H), 1.36 (s, 3H), 0.96 (s, 3H) ppm. ¹³C NMR (150 MHz, CDCl₃): δ 201.82, 175.45, 172.36, 151.47, 149.86, 144.52, 143.44, 139.13, 131.59, 126.67, 117.97, 109.26, 95.40, 87.56, 79.38, 75.79, 51.68, 47.55, 46.02, 43.98, 43.16, 40.98, 40.04, 31.27, 18.55, 17.80, 12.92 ppm. HRMS (ESI-TOF): calc'd for C₂₇H₂₉O₇₁ [M+H]⁺: 593.1030, found: 593.1021. TLC:R_{f}=0.33 (2:1 hexanes:ethyl acetate).

Compound S-27 characterization data: [α]_{D}²⁵=+23.99 (c 0.30, CHCl₃). ¹H NMR (600 MHz, CDCl₃): δ 7.36 (t, J=1.8 Hz, 1H), 7.31-7.29 (m, 2H), 6.38 (d, J=1.1 Hz, 1H), 5.98 (d, J=9.7 Hz, 1H), 5.40 (d, J=1.3 Hz, 1H), 5.19 (d, J=1.7 Hz, 1H), 4.55-4.52 (m, 1H), 4.51 (d, J=3.7 Hz, 1H), 4.39 (dd, J=12.3, 3.7 Hz, 1H), 3.69-3.65 (m, 4H), 3.64-3.60 (m, 1H), 3.57 (d, J=12.3 Hz, 1H), 3.55-3.51 (m, 1H), 2.62 (dd, J=16.4, 11.7 Hz, 1H), 2.55 (ddd, J=13.5, 8.6, 7.0 Hz, 1H), 1.88 (ddd, J=13.5, 4.5, 3.4 Hz, 1H), 1.63 (s, 3H), 1.43 (s, 3H), 1.04 (s, 3H) ppm. ¹³C NMR (150 MHz, CDCl₃): δ 201.91, 175.65, 172.51, 150.72, 149.97, 143.60, 143.56, 139.17, 131.64, 126.90, 119.24, 110.13, 95.90, 84.27, 77.07, 75.64, 51.85, 47.59, 46.27, 44.15, 43.53, 40.90, 37.13, 31.65, 18.61, 17.76, 13.05 ppm. HRMS (ESI-TOF): calc'd for C₂₇H₂₉O₇I [M+Na]⁺: 615.0850, found: 615.0856. TLC:R_{f}=0.33 (2:1 hexanes:ethyl acetate).

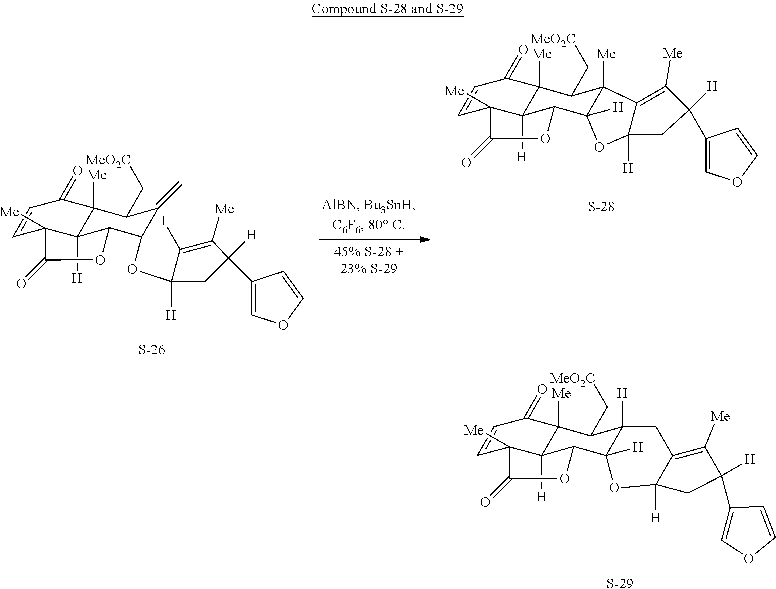

Under Ar (g), compound S-26 (26.1 mg, 0.044 mmol), AIBN (14.7 mg, 0.088 mmol, 2.0 eq) and Bu₃SnH (44.1 mg, 0.177 mmol, 4.0 eq) were dissolved in 4 ml hexafluorobenzene, then the mixture was warmed to 80° C. After 4.5 minutes the reaction was completed then cool to 0° C. to stop the reaction. After that, removal of the solvent via rotary evaporation afforded the crude mixture. Purification on PTLC (hexanes:ethyl acetate=2:1) afforded the nimbolide S-28 as white solid (9.2 mg, 45%) and the 6-endo product S-29 colorless oil (4.7 mg, 23%).

Nimbolide S-26 characterization data: [α]_{D}²¹=+173.22 (c 0.137, CHCl₃) (Synthesized). [α]_{D}²⁵=+206 (c 0.30, CHCl₃) (Natural). ¹H NMR (600 MHz, CDCl₃): δ 7.31 (t, J=1.7 Hz, 1H), 7.28 (d, J=9.7 Hz, 1H), 7.21 (s, 1H), 6.26-6.24 (m, 1H), 5.92 (d, J=9.7 Hz, 1H), 5.56-5.50 (m, 1H), 4.62 (dd, J=12.5, 3.7 Hz, 1H), 4.27 (d, J=3.7 Hz, 1H), 3.66 (d, J=8.8 Hz, 1H), 3.53 (s, 3H), 3.25 (dd, J=16.2, 5.3 Hz, 1H), 3.19 (d, J=12.5 Hz, 1H), 2.73 (t, J=5.6 Hz, 1H), 2.37 (dd, J=16.2, 5.9 Hz, 1H), 2.22 (dd, J=12.2, 6.6 Hz, 1H), 2.15-2.10 (m, 1H), 1.70 (d, J=1.8 Hz, 3H), 1.47 (s, 3H), 1.37 (s, 3H), 1.22 (s, 3H) ppm. ¹³C NMR (150 MHz, CDCl₃): δ 200.83, 174.95, 172.97, 149.64, 144.81, 143.19, 138.90, 136.46, 131.06, 126.55, 110.34, 88.47, 82.92, 73.44, 51.81, 50.33, 49.51, 47.74, 45.29, 43.67, 41.24, 41.15, 32.18, 18.59, 17.23, 15.21, 12.93 ppm. HRMS (ESI-TOF): calc'd for C₂₇H₃₀O₇[M+H]⁺: 467.2064, found: 467.2060. TLC:R_{f}=0.47 (1:1 hexanes:ethyl acetate).

Compound S-29 characterization data: [α]_{D}²³=+153.47 (c 0.043, CHCl₃). ¹H NMR (600 MHz, CDCl₃): δ 7.34 (t, J=1.7 Hz, 1H), 7.20 (s, 1H), 6.84 (d, J=11.5 Hz, 1H), 6.21 (dd, J=1.9, 0.9 Hz, 1H), 6.05 (dd, J=11.6, 1.1 Hz, 1H), 5.46-5.42 (m, 1H), 4.28 (dd, J=11.6, 3.1 Hz, 1H), 4.24 (d, J=3.0 Hz, 1H), 3.77-3.62 (m, 4H), 2.92 (dd, J=13.3, 7.7 Hz, 1H), 2.69 (t, J=11.6 Hz, 1H), 2.57 (ddd, J=13.2, 3.2, 1.2 Hz, 1H), 2.50 (dd, J=15.7, 4.1 Hz, 1H), 2.41 (dd, J=15.8, 6.4 Hz, 1H), 2.22 (ddt, J=13.6, 7.3, 3.8 Hz, 2H), 2.17 (ddt, J=11.4, 7.6, 3.6 Hz, 1H), 2.11 (dt, J=12.0, 8.4 Hz, 1H), 1.77 (d, J=1.8 Hz, 3H), 1.40 (s, 3H), 1.30 (s, 3H) ppm. ¹³C NMR (150 MHz, CDCl₃): δ 200.71, 176.59, 173.21, 143.94, 143.79, 143.47, 138.91, 136.42, 132.91, 126.63, 110.22, 88.36, 83.49, 77.43, 52.36, 49.82, 49.75, 48.30, 48.29, 45.49, 42.11, 41.57, 35.12, 32.90, 16.03, 14.94, 13.03 ppm. HRMS (ESI-TOF): not detected. TLC:R_{f}=0.45 (1:1 hexanes:ethyl acetate).

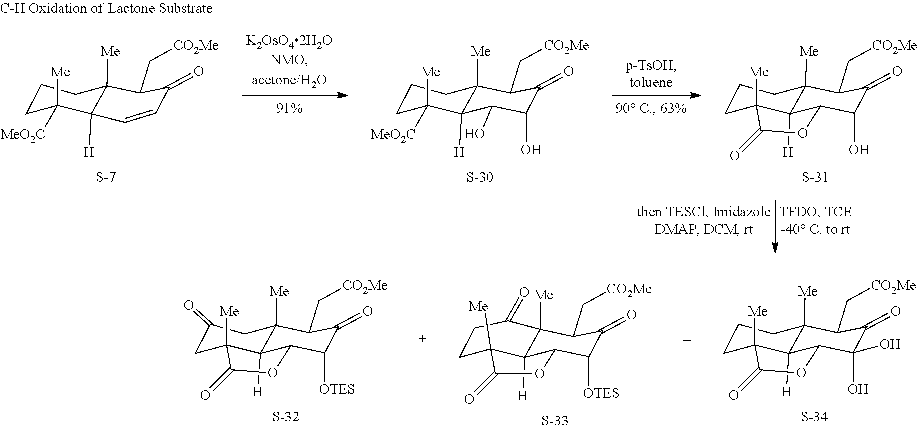

Compound 30: To compound S-7 (848.8 mg, 2.75 mmol) dissolved in 20 mL acetone and 10 mL H₂O at rt was added NMO (1.04 g, 8.85 mmol, 3.0 eq) followed by the K₂OsO₄·2H₂O (21.8 mg, 0.059 mmol, 0.02 eq). The mixture was stirred at rt for 24 hours then quenched by saturated aq. Na₂S₂O₃ (100 mL). It was then extracted with ethyl acetate (80 mL×5), dried over Na₂SO₄, then concentrated via rotary evaporation to give the crude which purified on the silica gel chromatography (SiO₂, hexane:ethyl acetate=1:2) afford the product S-30 as white solid (858.2 mg, 91%).

[α]_{D}²⁷=−45.26 (c 1.1, CHCl₃). ¹H NMR (600 MHz, CDCl₃): δ 4.10 (s, 1H), 3.87 (s, 1H), 3.66 (d, J=12.2 Hz, 3H), 3.65 (s, 3H), 3.42 (dd, J=10.0, 3.7 Hz, 1H), 3.01 (d, J=11.7 Hz, 1H), 2.65 (dd, J=16.9, 10.1 Hz, 2H), 2.26 (dd, J=16.8, 3.6 Hz, 1H), 1.71-1.51 (m, 5H), 1.38-1.33 (m, 1H), 1.31 (s, 3H), 0.69 (s, 3H) ppm. ¹³C NMR (150 MHz, CDCl₃): δ 208.80, 180.48, 173.64, 76.80, 70.43, 54.28, 52.37, 52.10, 46.77, 44.51, 38.04, 37.84, 37.19, 26.95, 18.05, 16.44, 15.51 ppm. HRMS (ESI-TOF): calc'd for C17H2607 [M+H]⁺: 343.1751, found: 343.1755. TLC:R_{f}=0.53 (1:2 hexanes:ethyl acetate).

Compound S-31: The compound S-30 (352.1 mg, 1.03 mmol) was dissolved in 50 mL toluene and the p-TsOH-H₂O (98.3 mg, 0.5 eq) was added, then the mixture was warmed to 90° C. After stirring for 40 minutes. The reaction was cooled to rt, saturated aq. NaHCO₃ (50 mL) was added to quench the reaction, extraction with ethyl acetate (50 ml×5), dried over Na₂SO₄, then concentrated via rotary evaporation to give the crude, purified on the silica gel chromatography (SiO₂, hexane:ethyl acetate=2:1) afford the product S-31 as white solid (201.0 mg 63%).

[α]_{D}²⁵=−14.78 (c 0.257, CHCl₃). ¹H NMR (600 MHz, CDCl₃): δ 4.56 (d, J=3.4 Hz, 1H), 4.27 (dd, J=12.4, 3.4 Hz, 1H), 3.66 (s, 3H), 3.39 (dd, J=10.8, 3.1 Hz, 1H), 2.78-2.69 (m, 2H), 2.21 (dd, J=16.8, 3.1 Hz, 1H), 1.92 (dt, J=12.7, 3.2 Hz, 1H), 1.81 (ddq, J=11.9, 4.9, 2.8 Hz, 1H), 1.77-1.67 (m, 2H), 1.63 (td, J=12.8, 3.9 Hz, 1H), 1.37 (td, J=13.0, 4.4 Hz, 1H), 1.23 (s, 3H), 0.78 (s, 3H) ppm. ¹³C NMR (150 MHz, CDCl₃): δ 207.26, 179.85, 172.79, 76.34, 74.94, 53.93, 52.21, 49.44, 41.90, 38.25, 37.81, 32.39, 27.17, 19.46, 15.87, 14.62 ppm.

HRMS (ESI-TOF): not detected. TLC:R_{f}=0.54 (1:1 hexanes:ethyl acetate).

Compounds S-32/S-33/S-34: The compound S-31 (1.4 g, 4.5 mmol) was dissolved in 45 mL tetrachloroethane (TCE) in sealed tube covered with aluminum foil, then cool to −40° C. and the TFDO solution (about 1.1 M, 10 mL, fresh distilled) was added to the reaction tube. The reaction was allowed to slowly warm to rt, and stir at rt for 12 hours until the reaction was completed. The solvent was removed via rotary evaporation to afford the crude. The crude was redissolved in 45 mL CH₂Cl₂, and stirred at rt. Imidazole (1.3 g, 19.15 mmol, 5.0 eq), TESCl (1.73 g, 1.9 ml, 3.0 eq) and DMAP (467.9 mg, 3.83 mmol, 1.0 eq) were added to the reaction tube and stirred at rt for 20 minutes, the reaction was quenched by saturated aq. NaHCO₃ (100 mL), then extraction with CH₂Cl₂ (60 mL×5), dried over Na₂SO₄, concentrated via rotary evaporation to give the crude, purified on the silica gel chromatography (SiO₂, hexane:ethyl acetate=6:1-4:1) afford the product S-32 as colorless oil (334.0 mg 23%) and product S-33 as colorless oil (78.2 mg, 6%). Note: compound S-34 decomposed during the protection step. The NMR yield: S-34 34%; S-22 27%; S-33 7%.

Compound S-34 characterization data: ¹H NMR (600 MHz, C6D6): δ4.54 (s, 1H, OH), 3.93 (dd, J=12.4, 1.4 Hz, 1H), 3.35 (s, 3H), 3.23-3.16 (m, 1H), 2.62 (ddd, J=16.7, 10.5, 1.4 Hz, 1H), 2.14 (d, J=12.4 Hz, 1H), 1.85 (dt, J=16.7, 2.1 Hz, 1H), 1.54 (dd, J=13.0, 3.5 Hz, 1H), 1.17 (td, J=13.1, 4.1 Hz, 1H), 1.06-1.00 (m, 1H), 1.00-0.91 (m, 1H), 0.85 (dd, J=13.3, 3.6 Hz, 1H), 0.64 (s, 3H), 0.48 (dd, J=13.3, 4.7 Hz, 1H), 0.06 (s, 3H) ppm. ¹³C NMR (150 MHz, C6D6): δ205.14, 178.63, 171.92, 94.04, 79.50, 55.41, 52.61, 51.61, 41.51, 37.44, 37.01, 32.16, 27.75, 18.94, 15.27, 14.55 ppm.

Compound S-33 characterization data: [α]_{D}²⁵=−14.21 (c 0.183, CHCl₃). ¹H NMR (600 MHz, CDCl₃): δ 4.47 (s, 1H), 4.29 (d, J=11.1 Hz, 1H), 3.79 (d, J=10.9 Hz, 1H), 3.65 (s, 3H), 3.06 (d, J=12.0 Hz, 1H), 2.90 (dd, J=16.6, 2.7 Hz, 1H), 2.85-2.75 (m, 1H), 2.69 (dd, J=16.7, 10.9 Hz, 1H), 2.49 (d, J=16.4 Hz, 1H), 2.22-2.12 (m, 2H), 1.49 (s, 3H), 1.05 (s, 3H), 0.94 (td, J=8.0, 1.7 Hz, 9H), 0.68-0.60 (m, 6H) ppm. ¹³C NMR (150 MHz, CDCl₃): δ 209.26, 207.16, 177.64, 171.96, 75.34, 74.61, 51.93, 49.29, 47.70, 41.32, 35.58, 33.69, 28.92, 16.06, 13.95, 6.66, 4.65 ppm. HRMS (ESI-TOF): calc'd for C₂₂H₃₄O₇Si [M+H]⁺: 439.2147, found: 437.2143. TLC:R_{f}=0.44 (3:1 hexanes:ethyl acetate).

Compound S-32 characterization data: [α]_{D}²⁵=−45.73 (c 0.293, CHCl₃). ¹H NMR (600 MHz, CDCl₃): δ 4.55 (d, J=3.2 Hz, 1H), 4.32 (dd, J=12.0, 3.2 Hz, 1H), 3.66 (s, 3H), 3.59 (s, 1H), 3.33 (d, J=12.1 Hz, 1H), 2.77-2.68 (m, 3H), 2.49-2.41 (m, 2H), 2.13 (dd, J=16.7, 3.2 Hz, 1H), 1.22 (s, 3H), 0.97 (t, J=7.9 Hz, 9H), 0.82 (s, 3H), 0.73-0.63 (m, 6H) ppm. 13C NMR (150 MHz, CDCl₃): δ 205.95, 205.73, 177.27, 171.76, 75.73, 75.40, 53.98, 53.61, 52.18, 50.52, 48.61, 43.31, 38.23, 27.29, 17.50, 16.35, 6.66, 4.64 ppm. HRMS (ESI-TOF): calc'd for C₂₂H₃₄O₇Si [M+H]⁺: 439.2147, found: 437.2158. TLC:R_{f}=0.51 (3:1 hexanes:ethyl acetate).

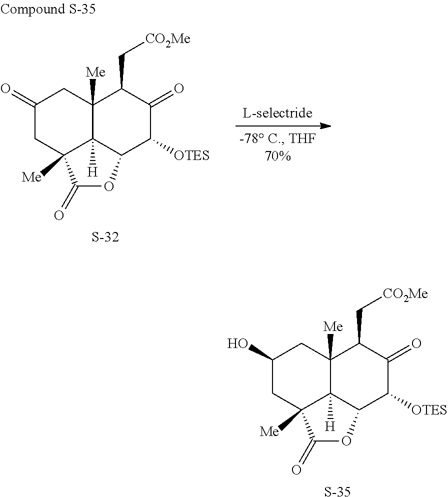

Under Ar_{(g)}, the ketone S-32 (334.0 mg, 0.76 mmol) was dissolved in 15 mL THF, and cooled to −78° C., the L-selectride solution (1 M in THF, 1.1 mL, 1.5 equiv.) was added dropwise. After stirring for 30 minutes, the reaction was quenched with 20 mL saturated NH₄Cl (aq.) and warmed to rt. Additional 30 mL H₂O was added then extraction with ethyl acetate (50 mL×4), dried over Na₂SO₄, then concentrated via rotary evaporation to give the crude mixture. Purification on the silica gel chromatography (SiO₂, hexanes:EA=2:1) afforded the product, which was then purified again on the silica gel chromatography (SiO₂, Et₂O:hexanes=1:1) to afford the clean product S-35 as colorless oil (233.7 mg) in 70% yield.

[α]_{D}¹=−41.22 (c 0.101, CHCl₃). ¹H NMR (600 MHz, CDCl₃): δ 4.48 (dd, J=6.4, 3.0 Hz, 2H), 4.31 (dd, J=12.2, 3.3 Hz, 1H), 3.65 (s, 3H), 3.35 (dd, J=10.6, 3.1 Hz, 1H), 2.74 (dd, J=16.6, 10.6 Hz, 1H), 2.68 (d, J=12.2 Hz, 1H), 2.25-2.18 (m, 2H), 1.99 (dt, J=14.3, 1.7 Hz, 1H), 1.82 (dd, J=14.3, 3.7 Hz, 1H), 1.59-1.56 (m, 1H), 1.50 (s, 3H), 1.00 (s, 3H), 0.94 (t, J=7.9 Hz, 9H), 0.64 (qd, J=7.9, 2.6 Hz, 6H) ppm. ¹³C NMR (150 MHz, CDCl₃): δ 207.39, 179.53, 172.37, 76.36, 75.66, 69.62, 54.13, 52.03, 49.32, 45.08, 41.35, 39.03, 37.18, 27.47, 17.37, 16.08, 6.69, 4.68 ppm. HRMS (ESI-TOF): not detected. TLC:R_{f}=0.45 (1:1 hexanes:ethyl acetate).

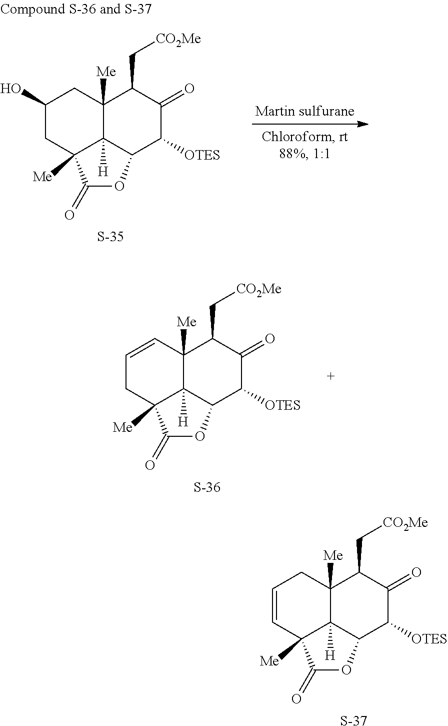

Under Ar(g), the alcohol S-35 (126.2 mg, 0.28 mmol) and Martin sulfurane (481.6 mg, 0.72 mmol, 2.5 equiv.) was dissolved in 15 mL CHCl₃ stirring at rt. After 20 minutes, the reaction was complete. Removal the solvent via rotary evaporation afforded the crude mixture, which was purified on the silica gel chromatography (SiO₂, hexanes:ethyl acetate=7:1) to obtain the mixture products (S-36/S-37) as colorless oil (106.6 mg, ratio 1:1) in 88% yield.

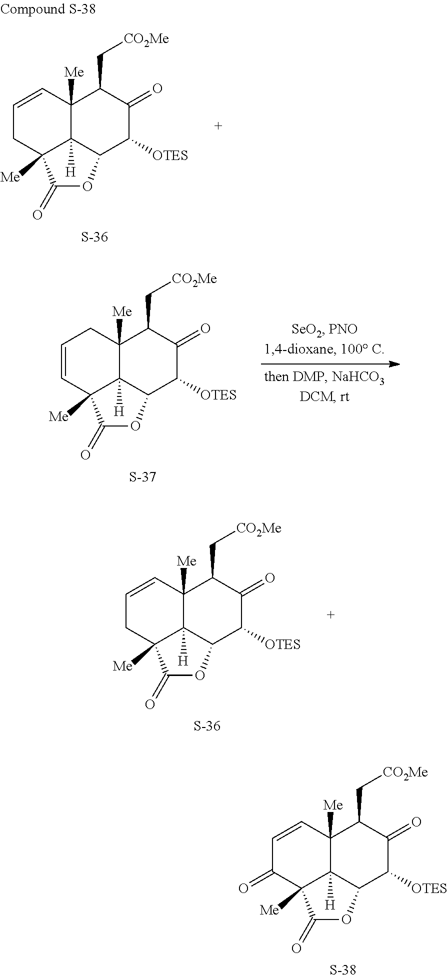

Under argon atmosphere, the above mixture (13.1 mg, 0.031 mmol), SeO₂ (17.2 mg, 0.155 mmol, 5.0 eq) and PNO (dry with P₂O₅ under vacuo for 24 hours) (29.5 mg, 0.31 mmol, 10.0 eq) were dissolved in 4 mL dioxane then warmed to 100° C. After 14 hours, the reaction was completed, and cooled to rt. Saturated Na₂S₂O₃ aq. (15 mL) was added to quench the reaction, which was then extracted with EA (20 ml×5), dried over Na₂SO₄, and concentrated via rotary evaporation to give the crude. The crude was re-dissolved in 3 mL CH₂Cl₂. NaHCO₃ (13.2 mg, 0.155 mmol, 5.0 eq) was added followed by DMP (39.4 mg, 0.093 mmol, 3.0 eq). After stirring for 30 minutes, the reaction was quenched by saturated aq. NaHCO₃ (20 mL) and saturated aq. Na₂S₂O₃ (20 mL). Extraction with CH₂Cl₂ (30 mL×4), drying over Na₂SO₄, remove of the solvent give the crude, which was purified on the silica gel chromatography (SiO₂, hexanes:ethyl acetate=5:1) affording the product S-36 as colorless (5.2 mg, 40%) and S38 as colorless oil (5.8 mg, 43%).

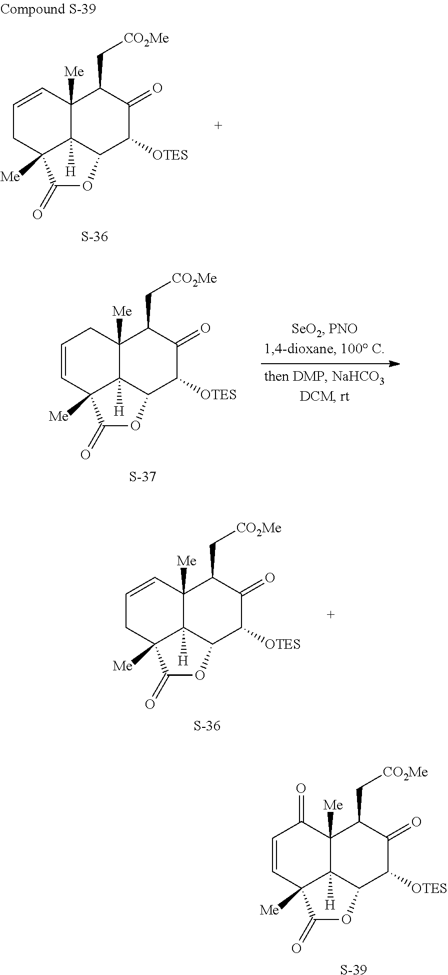

Under argon atmosphere, the above mixture (18.1 mg, 0.043 mmol), SeO₂ (24.2 mg, 0.215 mmol, 5.0 eq) and PNO (dry with P₂O₅ under vacuo for 24 hours) (40.8 mg, 0.43 mmol, 10.0 eq) were dissolved in 4 mL dioxane then warmed to 100° C. After 4.5 hours, the reaction was completed, and cooled to rt. Saturated Na₂S₂O₃ aq. (20 mL) was added to quench the reaction, which was then extracted with EA (20 ml×5), dried over Na₂SO₄, and concentrated via rotary evaporation to give the crude. The crude was re-dissolved in 5 mL CH₂Cl₂. NaHCO₃ (18.1 mg, 0.215 mmol, 5.0 eq) was added followed by DMP (54.7 mg, 0.129 mmol, 3.0 eq). After stirring for 30 minutes, the reaction was quenched by saturated aq. NaHCO₃ (20 mL) and saturated aq. Na₂S₂O₃ (20 mL). Extraction with CH₂Cl₂ (30 mL×4), drying over Na₂SO₄, remove of the solvent give the crude, which was purified on the silica gel chromatography (SiO₂, hexanes:ethyl acetate=5:1) affording the product S-36 as colorless (7.8 mg, 42%) and S39 as colorless oil (8.3 mg, 46%).

Compound S-36: [α]_{D}²⁴=−99.98 (c 0.220, CHCl₃). ¹H NMR (600 MHz, CDCl₃): δ 5.74-5.67 (m, 2H), 4.50 (d, J=3.2 Hz, 1H), 4.33 (dd, J=12.6, 3.3 Hz, 1H), 3.65 (s, 3H), 3.39 (dd, J=10.7, 2.9 Hz, 1H), 3.12 (d, J=12.6 Hz, 1H), 2.74 (dd, J=16.6, 10.8 Hz, 1H), 2.50 (d, J=17.3 Hz, 1H), 2.29 (ddd, J=28.9, 17.4, 3.4 Hz, 2H), 1.22 (s, 3H), 0.94 (t, J=7.9 Hz, 9H), 0.83 (s, 3H), 0.63 (qd, J=8.0, 2.1 Hz, 6H) ppm. ¹³C NMR (151 MHz, CDCl₃): δ 206.82, 179.89, 172.16, 132.09, 126.55, 75.93, 75.47, 52.56, 52.04, 47.35, 39.93, 37.94, 35.06, 27.39, 17.31, 16.03, 6.66, 4.68 ppm. HRMS (ESI-TOF): not detected. TLC:R_{f}=0.51 (4:1 hexanes:ethyl acetate).

Compound S-39: [α]_{D}²¹=−12.00 (c 0.10, CHCl₃). ¹H NMR (600 MHz, CDCl₃): δ 7.35 (d, J=9.7 Hz, 1H), 6.01 (d, J=9.7 Hz, 1H), 4.54 (d, J=3.3 Hz, 1H), 4.37 (dd, J=12.4, 3.4 Hz, 1H), 3.79 (dd, J=11.2, 2.7 Hz, 1H), 3.65 (s, 3H), 3.57 (d, J=12.5 Hz, 1H), 3.22 (dd, J=17.0, 2.7 Hz, 1H), 2.81 (dd, J=17.0, 11.2 Hz, 1H), 1.47 (s, 3H), 1.00 (s, 3H), 0.94 (t, J=8.0 Hz, 9H), 0.69-0.60 (m, 6H) ppm. ¹³C NMR (150 MHz, CDCl₃): δ 206.13, 199.58, 174.76, 172.09, 150.00, 131.24, 75.57, 74.91, 51.95, 48.30, 46.65, 45.21, 44.11, 28.54, 18.75, 14.23, 6.64, 4.63 ppm. HRMS (ESI-TOF): not detected. TLC:R_{f}=0.36 (3:1 hexanes:ethyl acetate).

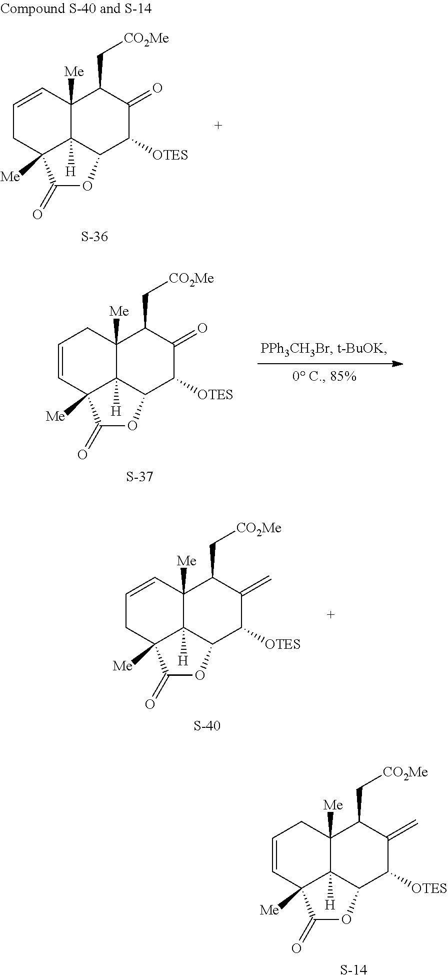

Under Ar(g), the PPH₃CH₃Br (334.4 mg, 0.94 mmol, 5.2 equiv.) was dissolved in 15 mL THF at 0° C. The t-BuOK solution (1 M in THF, 0.9 mL, 0.9 mmol, 5.0 equiv.) was added dropwise. 45 minutes later, the starting material S-36/S-37 (76.1 mg, 0.18 mmol) in 4 mL THF was added dropwise, then the reaction warmed to rt. After additional 30 minutes stirring, the reaction was quenched with 30 mL saturated NH₄Cl (aq.) and 30 mL H₂O, extracted with ethyl acetate (40 mL×4). The combined organic phase was dried over Na₂SO₄, then concentrated via rotary evaporation to give the crude mixture. Purification on the silica gel chromatography (SiO₂, hexane:ethyl acetate=8:1) afforded the products (S-40/S-14) as colorless oil (64.4 mg, ratio 1:1) in 85% yield.

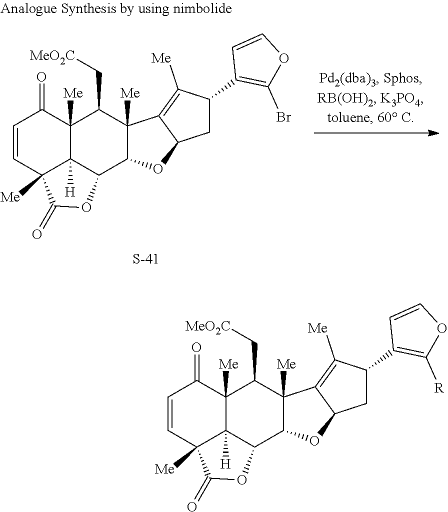

Under Ar(g), the bromo-nimbolide, RB(OH)₂ (5.0 equiv.), Pd₂(dba)₃ (0.5 equiv.), K₃PO₄ (7.0 equiv.) and Sphos (1.0 equiv.) were dissolved in toluene (0.02M) then warm to 60° C. or 80° C. after stir for 12 hour to 48 hours. When the reaction completed, filtered by a short silica gel plug then concentrated via rotary evaporation to give the crude, purified on the PTLC afford the target compounds.

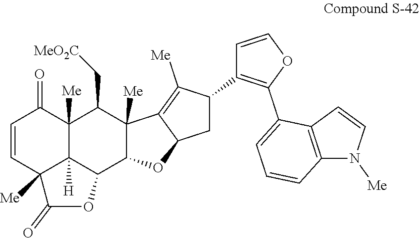

The compound was synthesized following the general procedure on 5.1 μmol scale to afford the product in 2.5 mg, yield 83%.

¹H NMR (400 MHz, CDCl₃): δ 7.74 (s, 1H), 7.42 (dd, J=8.6, 1.5 Hz, 1H), 7.37-7.32 (m, 2H), 7.29 (d, J=9.7 Hz, 1H), 7.08 (d, J=3.1 Hz, 1H), 6.51 (d, J=3.0 Hz, 1H), 6.33 (d, J=1.9 Hz, 1H), 5.94 (d, J=9.7 Hz, 1H), 5.64 (t, J=7.3 Hz, 1H), 4.63 (dd, J=12.5, 3.7 Hz, 1H), 4.29 (d, J=3.6 Hz, 1H), 4.17-4.12 (m, 1H), 3.82 (s, 3H), 3.69 (s, 3H), 3.24 (t, J=3.0 Hz, 1H), 3.20 (d, J=6.6 Hz, 1H), 2.81 (t, J=5.4 Hz, 1H), 2.42 (dd, J=16.4, 4.9 Hz, 1H), 2.34-2.28 (m, 1H), 2.24 (dt, J=12.0, 8.4 Hz, 1H), 1.67 (d, J=1.4 Hz, 3H), 1.48 (s, 3H), 1.36 (s, 3H), 1.25 (d, J=7.4 Hz, 3H) ppm. ¹³C NMR (151 MHz, CDCl₃): δ 200.92, 175.12, 173.21, 151.08, 149.79, 145.05, 140.94, 137.25, 136.17, 131.18, 129.76, 128.57, 122.98, 121.03, 120.78, 119.38, 111.59, 109.51, 101.54, 88.69, 83.11, 73.62, 52.11, 50.68, 49.95, 47.92, 45.51, 43.81, 41.47, 41.25, 33.10, 32.45, 18.77, 17.52, 15.31, 13.31 ppm. [α]_{D}²⁶+39.86 (c 0.08, CHCl₃).

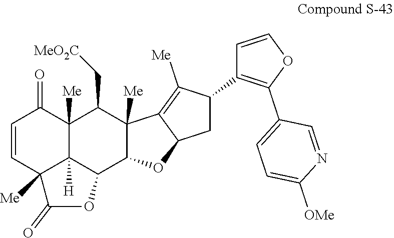

The compound was synthesized following the general procedure on 7.5 μmol scale to afford the product in 3.7 mg, yield 86%.

¹H NMR (400 MHz, CDCl₃): δ 8.31 (d, J=2.2 Hz, 1H), 7.75 (dd, J=8.7, 2.4 Hz, 1H), 7.35 (d, J=1.8 Hz, 1H), 7.29 (d, J=9.6 Hz, 1H), 6.81 (d, J=8.7 Hz, 1H), 6.34 (d, J=1.8 Hz, 1H), 5.93 (d, J=9.7 Hz, 1H), 5.60 (t, J=7.1 Hz, 1H), 4.63 (dd, J=12.5, 3.6 Hz, 1H), 4.29 (d, J=3.6 Hz, 1H), 3.97 (s, 3H), 3.68 (s, 3H), 3.26-3.14 (m, 2H), 2.79 (t, J=5.4 Hz, 1H), 2.40 (dd, J=16.4, 4.9 Hz, 1H), 2.30-2.15 (m, 2H), 1.67 (s, 3H), 1.48 (s, 3H), 1.37 (s, 3H), 1.24 (s, 3H) ppm. ¹³C NMR (151 MHz, CDCl₃): δ 200.88, 175.04, 173.25, 163.35, 149.81, 146.93, 145.68, 144.60, 142.02, 137.09, 136.51, 131.15, 122.57, 121.18, 111.76, 111.16, 88.48, 83.13, 73.51, 53.74, 52.10, 50.67, 49.76, 47.91, 45.49, 43.78, 41.44, 41.25, 32.43, 18.75, 17.48, 15.29, 13.29 ppm. [α]_{D}²⁶+34.48 (c 0.12, CHCl₃).

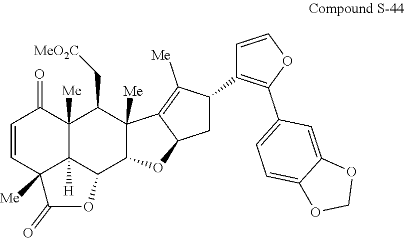

The compound was synthesized following the general procedure on 5.3 μmol scale to afford the product in 2.8 mg, yield 91%.

¹H NMR (400 MHz, CDCl₃): δ 7.31-7.27 (m, 2H), 7.03-6.90 (m, 2H), 6.89-6.82 (m, 1H), 6.30 (d, J=1.9 Hz, 1H), 5.99 (s, 2H), 5.93 (d, J=9.7 Hz, 1H), 5.60 (t, J=7.3 Hz, 1H), 4.63 (dd, J=12.5, 3.7 Hz, 1H), 4.28 (d, J=3.6 Hz, 1H), 4.03 (d, J=7.7 Hz, 1H), 3.67 (s, 3H), 3.28-3.15 (m, 2H), 2.79 (t, J=5.4 Hz, 1H), 2.39 (dt, J=16.5, 5.2 Hz, 1H), 1.69-1.64 (m, 4H), 1.48 (s, 3H), 1.37 (s, 3H), 1.24 (s, 3H) ppm. ¹³C NMR (151 MHz, CDCl₃): δ 200.89, 175.07, 173.22, 149.80, 148.01, 145.47, 141.23, 136.77, 131.16, 125.58, 121.69, 120.40, 111.75, 108.63, 107.21, 101.32, 88.55, 83.14, 73.55, 52.10, 50.68, 49.82, 47.91, 45.50, 43.79, 41.34, 41.24, 32.44, 18.76, 17.51, 15.30, 13.31 ppm. [α]_{D}²⁶+18.44 (c 0.08, CHCl₃).

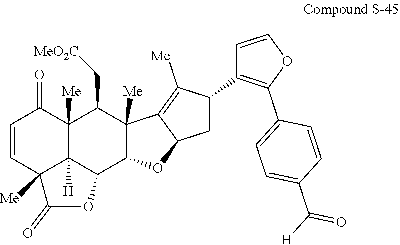

The compound was synthesized following the general procedure on 7.2 μmol scale to afford the product in 3.5 mg, yield 87%.

¹H NMR (600 MHz, CDCl₃): δ 10.01 (s, 1H), 7.92 (d, J=7.9 Hz, 2H), 7.71 (d, J=7.8 Hz, 2H), 7.42 (s, 1H), 7.29 (d, J=9.7 Hz, 1H), 6.39 (s, 1H), 5.93 (d, J=9.7 Hz, 1H), 5.60 (s, 1H), 4.64 (dd, J=12.5, 2.8 Hz, 1H), 4.32 (d, J=2.5 Hz, 1H), 4.17-4.10 (m, 1H), 3.69 (s, 3H), 3.26-3.17 (m, 2H), 2.78 (t, J=5.1 Hz, 1H), 2.41 (dd, J=16.4, 4.5 Hz, 1H), 2.32-2.27 (m, 2H), 1.69 (s, 3H), 1.49 (s, 3H), 1.38 (s, 3H), 1.25 (s, 3H) ppm. ¹³C NMR (151 MHz, CDCl₃): δ 200.85, 191.77, 175.02, 173.28, 149.82, 147.90, 146.25, 143.07, 136.96, 136.15, 134.75, 131.13, 130.31, 126.13, 125.99, 112.56, 88.42, 83.21, 73.45, 52.11, 50.73, 50.00, 47.90, 45.50, 43.79, 41.26, 41.24, 32.48, 18.76, 17.49, 15.28, 13.37 ppm. [α]_{D}²⁵+50.38 (c 0.11, CHCl₃).

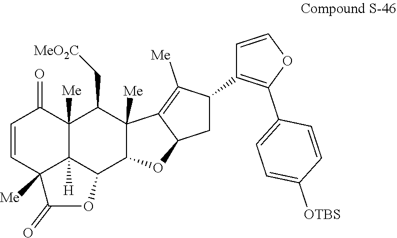

The compound was synthesized following the general procedure on 5.3 μmol scale to afford the product in 3.3 mg, yield 92%.

¹H NMR (600 MHz, CDCl₃): δ 7.39 (d, J=7.8 Hz, 2H), 7.30 (d, J=6.0 Hz, 2H), 6.88 (d, J=7.6 Hz, 2H), 6.29 (s, 1H), 5.93 (d, J=9.7 Hz, 1H), 5.59 (t, J=6.3 Hz, 1H), 4.63 (d, J=12.5 Hz, 1H), 4.29 (s, 1H), 4.04 (d, J=8.5 Hz, 1H), 3.67 (s, 3H), 3.21 (d, J=13.6 Hz, 2H), 2.79 (t, J=5.1 Hz, 1H), 2.40 (dd, J=16.2, 4.6 Hz, 1H), 2.29-2.17 (m, 2H), 1.67 (s, 3H), 1.48 (s, 3H), 1.37 (s, 3H), 1.24 (s, 3H), 0.99 (s, 9H), 0.22 (s, 6H) ppm. ¹³C NMR (151 MHz, CDCl₃): δ 200.89, 175.08, 173.19, 155.26, 149.79, 149.52, 145.35, 141.13, 136.92, 131.17, 127.77, 124.82, 121.32, 120.40, 111.68, 88.59, 83.14, 73.57, 52.09, 50.68, 49.83, 47.92, 45.51, 43.80, 41.40, 41.26, 32.45, 29.85, 25.82, 18.77, 18.38, 17.52, 15.31, 13.32, −4.24 ppm. [α]_{D}²⁶+16.76 (c 0.11, CHCl₃).

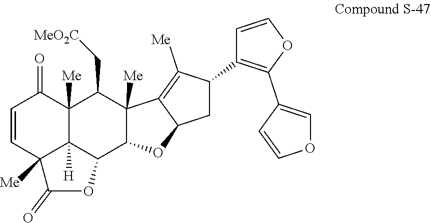

The compound was synthesized following the general procedure on 9.5 μmol scale to afford the product in 4.7 mg, yield 93%.

¹H NMR (600 MHz, CDCl₃) δ 7.63 (s, 1H), 7.46 (s, 1H), 7.30-7.27 (m, 2H), 6.65 (s, 1H), 6.27 (s, 1H), 5.93 (d, J=9.7 Hz, 1H), 5.58 (t, J=6.7 Hz, 2H), 4.63 (dd, J=12.5, 3.4 Hz, 1H), 4.29 (d, J=3.1 Hz, 1H), 3.93 (d, J=7.0 Hz, 1H), 3.64 (s, 3H), 3.26-3.14 (m, 2H), 2.77 (t, J=5.3 Hz, 1H), 2.39 (dd, J=16.3, 5.0 Hz, 1H), 2.20 (t, J=7.6 Hz, 2H), 1.69 (s, 3H), 1.48 (s, 3H), 1.38 (s, 3H), 1.24 (s, 3H) ppm. ¹³C NMR (151 MHz, CDCl₃): δ 200.88, 175.05, 173.17, 149.79, 145.50, 143.44, 143.32, 141.34, 138.49, 136.54, 131.17, 122.18, 117.44, 111.47, 108.62, 88.60, 83.14, 73.53, 52.07, 50.64, 49.65, 47.91, 45.47, 43.79, 41.26, 41.15, 32.39, 18.75, 17.46, 15.32, 13.28 ppm. [α]_{D}²⁵+25.23 (c 0.15, CHCl₃).

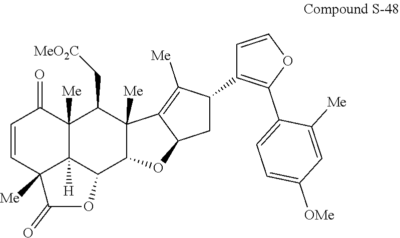

The compound was synthesized following the general procedure on 6.8 μmol scale to afford the product in 3.3 mg, yield 82%.

¹H NMR (600 MHz, CDCl₃): δ 7.34 (s, 1H), 7.29 (d, J=9.7 Hz, 1H), 7.11 (t, J=7.9 Hz, 1H), 6.81 (d, J=2.5 Hz, 1H), 6.75 (dd, J=8.4, 2.7 Hz, 1H), 6.32 (d, J=2.0 Hz, 1H), 5.94 (d, J=9.7 Hz, 1H), 5.67-5.62 (m, 1H), 4.61 (dd, J=12.8, 3.7 Hz, 1H), 4.23 (d, J=3.6 Hz, 1H), 3.83 (s, 4H), 3.66 (s, 3H), 3.62 (d, J=8.9 Hz, 1H), 3.25-3.18 (m, 2H), 2.80 (t, J=5.5 Hz, 1H), 2.38 (dd, J=16.3, 5.3 Hz, 1H), 2.27-2.18 (m, 5H), 2.10 (dt, J=11.9, 8.6 Hz, 1H), 1.47 (s, 3H), 1.33 (s, 3H), 1.23 (s, 3H) ppm. ¹³C NMR (151 MHz, CDCl₃): δ 200.94, 175.09, 173.24, 159.92, 150.28, 149.79, 144.73, 141.54, 140.21, 137.19, 131.83, 131.20, 123.06, 122.93, 116.04, 110.87, 110.58, 88.72, 83.07, 73.61, 55.38, 52.09, 50.59, 49.76, 47.94, 45.45, 43.79, 41.25, 41.17, 32.31, 20.58, 18.73, 17.47, 15.33, 15.30, 13.02 ppm. [α]_{D}²⁵+26.56 (c 0.11, CHCl₃).

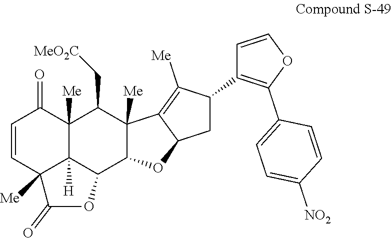

The compound was synthesized following the general procedure on 5.5 μmol scale to afford the product in 1.0 mg, yield 31%.

¹H NMR (600 MHz, CDCl₃): δ 8.30-8.22 (m, 2H), 7.73-7.67 (m, 2H), 7.44 (d, J=1.9 Hz, 1H), 7.29 (d, J=9.7 Hz, 1H), 6.41 (d, J=1.9 Hz, 1H), 5.94 (d, J=9.7 Hz, 1H), 5.59 (tt, J=7.4, 1.9 Hz, 1H), 4.64 (dd, J=12.6, 3.7 Hz, 1H), 4.32 (d, J=3.6 Hz, 1H), 4.13-4.09 (m, 1H), 3.69 (s, 3H), 3.23 (dd, J=16.4, 6.0 Hz, 1H), 3.19 (d, J=12.5 Hz, 1H), 2.78 (t, J=5.5 Hz, 1H), 2.40 (dd, J=16.4, 4.9 Hz, 1H), 2.33-2.27 (m, 2H), 1.69 (d, J=1.9 Hz, 3H), 1.49 (s, 3H), 1.38 (s, 3H), 1.25 (s, 3H) ppm. ¹³C NMR (151 MHz, CDCl₃): δ 200.82, 174.98, 173.30, 149.83, 147.04, 146.56, 146.30, 143.55, 137.32, 135.85, 131.12, 126.93, 126.06, 124.32, 112.77, 88.35, 83.24, 73.40, 52.11, 50.75, 50.01, 47.90, 45.49, 43.79, 41.24, 41.22, 32.49, 18.76, 17.49, 15.28, 13.38 ppm. [α]_{D}²⁶=−4.78 (c 1.2, CHCl₃).

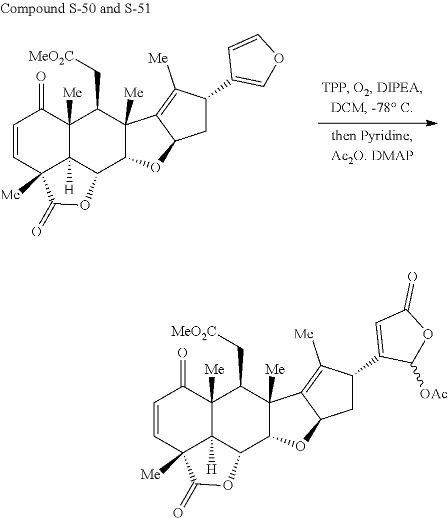

Oxygen was bubbled through of the starting material (6.3 mg, 0.0135 mmol) in CH₂Cl₂ containing a catalytic amount of TPP (1.2 mg, 0.002 mmol) and DIPEA (3 ul, 0.0169 mmol) at −78° C., the mixture was irradiated with the visible-light lamp, after 10 minutes later sparing with oxygen and irradiation was discontinued, the cool bath was removed. Then warm to rt 0.1 mL pyridine and 0.05 mL Ac₂O was added followed by DMAP. 15 minutes later, 10 mL saturated NaHCO₃ was added to quench the reaction, extraction with ethyl acetate (25 mL×5). The combined organic phase was washed with 1 N HCl (5 mL×2), saturated brine (10 mL×2) and H₂O (10 mL×2), dried over Na₂SO₄, then concentrated via rotary evaporation to give the crude, purified on the PTLC (CH₂Cl₂:MeOH=20:1) afford the diastereomer 1 (1.5 mg) as colorless oil and diastereomer 2 (2.6 mg) as colorless oil.

Diastereomer 1: ¹H NMR (400 MHz, CDCl₃): δ 7.28 (d, 1H), 6.83 (d, J=1.1 Hz, 1H), 6.02 (d, J=1.2 Hz, 1H), 5.92 (dd, J=9.8, 1.7 Hz, 1H), 5.41 (td, J=6.2, 3.0 Hz, 1H), 4.60 (dd, J=12.5, 3.7 Hz, 1H), 4.28 (d, J=3.6 Hz, 1H), 3.69 (d, J=1.9 Hz, 3H), 3.38 (d, J=8.9 Hz, 1H), 3.28 (dd, J=16.6, 5.2 Hz, 1H), 3.13 (dd, J=12.5, 1.7 Hz, 1H), 2.69 (t, J=5.4 Hz, 1H), 2.42-2.29 (m, 2H), 2.17 (d, J=1.7 Hz, 3H), 2.07 (dt, J=12.2, 8.7 Hz, 1H), 1.77 (d, J=1.9 Hz, 3H), 1.48 (s, 3H), 1.37 (d, J=2.0 Hz, 3H), 1.22 (d, J=1.9 Hz, 3H) ppm.

Diastereomer 2: ¹H NMR (400 MHz, CDCl₃): δ 7.22 (d, 1H), 6.77 (d, J=0.9 Hz, 1H), 5.90 (d, J=1.0 Hz, 1H), 5.85 (d, J=9.7 Hz, 1H), 5.29 (ddt, J=8.3, 6.4, 1.8 Hz, 1H), 4.53 (dd, J=12.5, 3.7 Hz, 1H), 4.21 (d, J=3.6 Hz, 1H), 3.64 (s, 3H), 3.38 (d, J=9.1 Hz, 1H), 3.21 (dd, J=16.7, 5.2 Hz, 1H), 3.06 (d, J=12.5 Hz, 1H), 2.61 (t, J=5.4 Hz, 1H), 2.29 (dd, J=16.7, 5.5 Hz, 1H), 2.20 (dd, J=12.4, 6.5 Hz, 1H), 2.11 (s, 3H), 2.09-1.95 (m, 1H), 1.70 (d, J=1.8 Hz, 3H), 1.40 (s, 3H), 1.31 (s, 3H), 1.19 (d, J=1.4 Hz, 3H), 1.16 (s, 3H) ppm.

### General Procedure to Access Amide Analogues

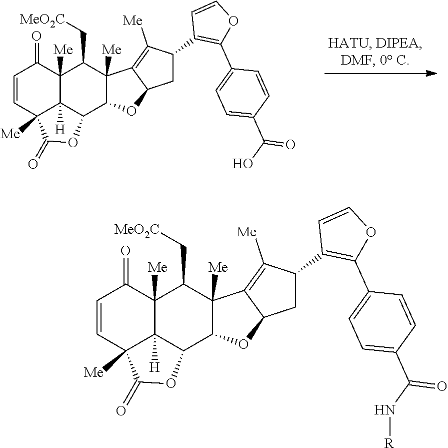

The starting material acid, HATU (3.0 equiv.) were dissolved in 0.5 mL DMF stir at 0° C., and the DIPEA (3.0 equiv.) was added. After 10 minutes stirring, the amine (2.0 equiv.) was added then stir at 0° C. for 6 hours the reaction was quenched by 10 mL saturated NH₄Cl, and the aqueous phase was extracted by EtOAc (10 mL×4). The combined organic layer was washed with H₂O (20 mL), brine (20 mL), dried over Na₂SO₄, and concentrated via rotary evaporation to give the crude, purified on the PTLC (hexanes:EA=1:1).

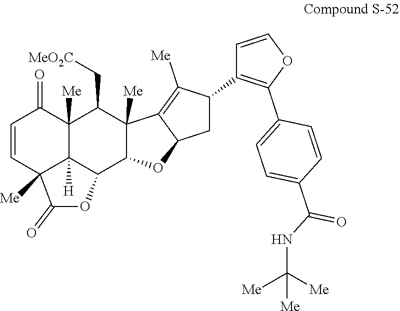

The compound was synthesized following the general procedure on 7.7 μmol scale to afford the product in 4.0 mg, yield 82%.

¹H NMR (400 MHz, CDCl₃): δ 7.76 (d, J=8.1 Hz, 2H), 7.58 (d, J=8.0 Hz, 2H), 7.38 (d, J=1.8 Hz, 1H), 7.29 (d, J=9.6 Hz, 1H), 6.35 (t, J=1.6 Hz, 1H), 5.93 (d, J=10.0 Hz, 2H), 5.60 (s, 1H), 4.63 (dd, J=12.6, 3.6 Hz, 1H), 4.31 (d, J=3.6 Hz, 1H), 4.12 (m, 2H), 3.68 (d, J=1.2 Hz, 2H), 3.29-3.14 (m, 2H), 2.89 (d, J=5.0 Hz, 1H), 2.78 (t, J=5.3 Hz, 1H), 2.40 (dd, J=16.4, 4.9 Hz, 1H), 2.26 (t, J=7.7 Hz, 2H), 1.66 (s, 3H), 1.48 (s, 12H), 1.37 (s, 3H), 1.24 (s, 3H) ppm.

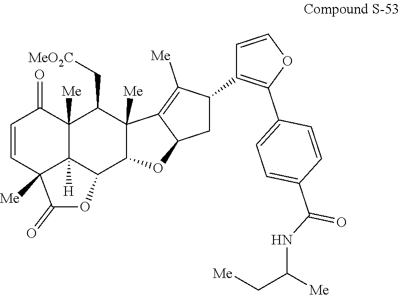

The compound was synthesized following the general procedure on 7.7 μmol scale to afford the product in 3.0 mg, yield 61%.

¹H NMR (400 MHz, CDCl₃): δ 7.80 (d, J=8.4 Hz, 2H), 7.59 (d, J=8.5 Hz, 2H), 7.38 (d, J=1.9 Hz, 1H), 7.29 (d, J=9.7 Hz, 1H), 6.35 (d, J=1.9 Hz, 1H), 5.93 (d, J=9.7 Hz, 1H), 5.88 (d, J=8.4 Hz, 1H), 5.60 (s, 1H), 4.64 (dd, J=12.5, 3.7 Hz, 1H), 4.31 (d, J=3.6 Hz, 1H), 4.12 (q, J=7.1, 6.3 Hz, 2H), 3.68 (s, 3H), 3.27-3.19 (m, 1H), 3.23-3.16 (m, 1H), 2.78 (t, J=5.4 Hz, 1H), 2.40 (dd, J=16.4, 4.9 Hz, 1H), 2.33-2.19 (m, 2H), 1.67 (d, J=1.8 Hz, 3H), 1.63-1.55 (m, 2H), 1.37 (s, 3H), 1.26-1.23 (m, 8H), 0.98 (t, J=7.4 Hz, 3H) ppm.

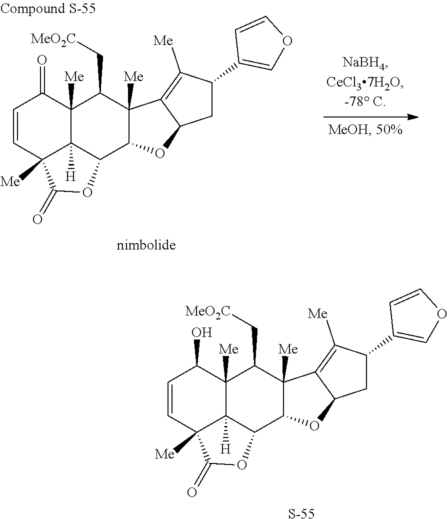

Nimbolide (25 mg, 0.054 mmol) was dissolved in 3 ml MeOH. The mixture was cooled to −78° C. and CeCl₃·7H₂O (40.3 mg, 0.108 mmol, 2 eq) was added followed by NaBH₄ (4.9 mg, 0.108 mmol, 2 eq). After stirring at −78° C. for 30 min, the reaction was quenched by 30 ml saturated NH₄Cl then warm to rt another 30 ml H₂O was added. The layers were separated and the aqueous layer was extracted with EtOAc (30 mL×6). The combined organic extracts were washed with brine, dried over MgSO₄, and concentrated give the crude. Purification by silica gel chromatography (hexane:EA 1:1.5) afforded the product S-55 as white soild 12 mg (yield 50%).

[α]_{D}²⁶=+38.01 (c 0.1, CHCl₃). ¹H NMR (400 MHz, CDCl₃): δ 7.34 (t, J=1.7 Hz, 1H), 7.21 (s, 1H), 6.24 (d, J=1.6 Hz, 1H), 6.16 (dd, J=9.9, 2.4 Hz, 1H), 5.50 (dd, J=9.9, 2.4 Hz, 1H), 5.48-5.41 (m, 1H), 4.51 (dd, J=12.3, 3.7 Hz, 1H), 4.24 (dd, J=8.0, 3.1 Hz, 2H), 3.67 (d, J=8.6 Hz, 1H), 3.54 (s, 3H), 2.82 (dd, J=15.5, 6.3 Hz, 1H), 2.56 (d, J=12.3 Hz, 1H), 2.40 (dd, J=15.5, 5.2 Hz, 1H), 2.30 (t, J=5.7 Hz, 1H), 2.23 (dd, J=12.1, 6.6 Hz, 1H), 2.17-2.10 (m, 1H), 1.74 (d, J=1.9 Hz, 3H), 1.35 (s, 3H), 1.32 (s, 3H), 1.06 (s, 3H) ppm. ¹³C NMR (100 MHz, CDCl₃): δ 176.74, 174.75, 145.54, 143.29, 138.95, 136.16, 133.14, 130.66, 126.84, 110.53, 88.33, 83.06, 74.48, 52.14, 50.29, 49.60, 47.55, 46.73, 43.61, 41.41, 40.75, 32.20, 29.85, 19.15, 16.64, 13.09, 12.81 ppm. HRMS (ESI-TOF): calc'd for C₂₇H₃₂07 [M+H]⁺: 469.2221, found: 469.2221. TLC:R_{f}=0.4 (EA:hexane 1:1).

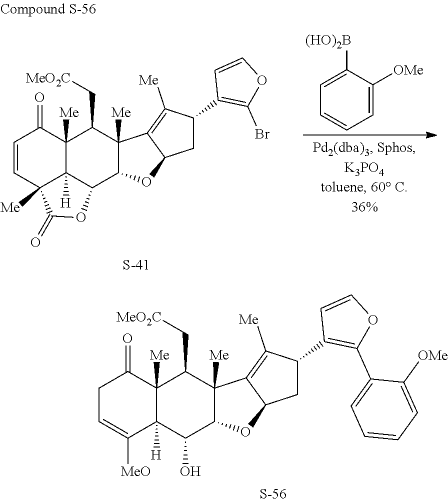

Under Ar(g), the starting material S-41 (7.0 mg, 0.0128 mmol), (2-Methoxyphenyl)boric acid (9.7 mg, 0.064 mmol, 5.0 equiv.), Pd₂(dba)₃ (6.2 mg, 0.006 mmol, 0.5 equiv.), K₃PO₄ (20.4 mg, 0.096 mmol, 7.0 equiv.) and Sphos (5.3 mg, 0.013 mmol, 1.0 equiv.) were dissolved in 2 mL toluene then warm to 80° C. after stir for 48 hours when the reaction completed, filtered by a short silica gel plug then concentrated via rotary evaporation to give the crude, purified on the PTLC afford the target compounds S-56 as colorless oil (2.5 mg) in 36% yield.

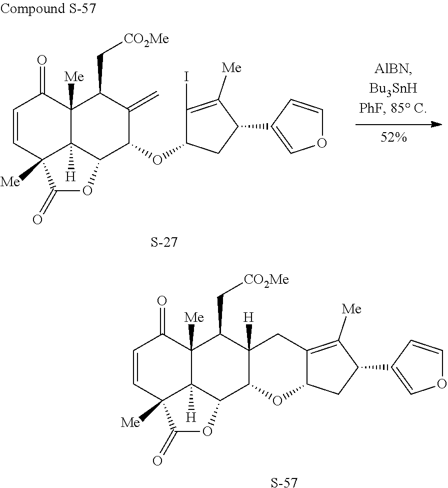

Under Ar (g), compound S-27 (11.4 mg, 0.0192 mmol), AIBN (6.3 mg, 0.0385 mmol, 2.0 eq) and Bu₃SnH (19.1 mg, 0.0768 mmol, 4.0 eq) were dissolved in 2 ml hexafluorobenzene. Then the mixture was warmed to 80° C. After 4.5 minutes the reaction was completed and cooled to 0° C. stop the reaction. After that, removal of the solvent via rotary evaporation afforded the crude mixture. Purification on PTLC (hexanes:ethyl acetate=2:1) afforded the compound S-57 as colorless oil (5.6 mg, 62%).

[α]_{D}²³=+51.99 (c 0.05, CHCl₃). ¹H NMR (600 MHz, CDCl₃): δ 7.35 (t, J=1.7 Hz, 1H), 7.29 (s, 1H), 7.28 (s, 1H), 6.37 (d, J=0.9 Hz, 1H), 5.96 (d, J=9.7 Hz, 1H), 4.52 (dd, J=12.4, 3.8 Hz, 1H), 4.49-4.44 (m, 1H), 4.24 (d, J=2.2 Hz, 1H), 3.63 (s, 3H), 3.53-3.45 (m, 1H), 3.26-3.15 (m, 2H), 2.70 (ddd, J=11.7, 5.9, 3.6 Hz, 1H), 2.62 (dt, J=13.0, 7.4 Hz, 1H), 2.55 (dd, J=15.2, 1.9 Hz, 1H), 2.40 (dd, J=17.2, 5.8 Hz, 1H), 2.27 (d, J=13.6 Hz, 1H), 1.94-1.88 (m, 1H), 1.69-1.62 (m, 1H), 1.45 (s, 3H), 1.35 (q, J=1.6 Hz, 3H), 1.17 (s, 3H) ppm. ¹³C NMR (150 MHz, CDCl₃): δ 202.13, 175.61, 172.91, 149.66, 143.36, 139.39, 136.60, 131.88, 129.85, 127.56, 110.24, 83.38, 74.85, 73.53, 51.86, 47.64, 46.07, 43.99, 42.64, 39.12, 37.67, 37.26, 33.15, 26.61, 18.79, 13.68, 12.14 ppm. HRMS (ESI-TOF): not detected. TLC:R_{f}=0.45 (1:1 hexanes:ethyl acetate).

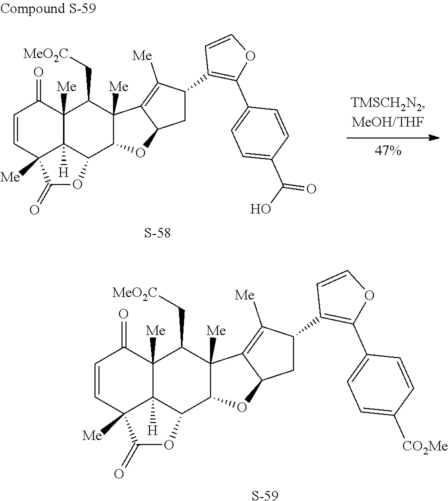

Under Ar(g), the starting material S-58 (7.0 mg, 0.012 mmol) was dissolved in 1.5 mL THF and 1.5 mL MeOH, stir at 0° C., the TMSCH₂N₂ (2 M in hexane, 12 ul, 0.024 mmol, 2.0 equiv.) was added, after 30 minutes later the reaction completed, remove the solvent via rotary evaporation to give the crude, purified on PTLC (hexanes:EA=2:1) afford the product S-59 as colorless oil (3.4 mg) in 47% yield (this compound is not stable).

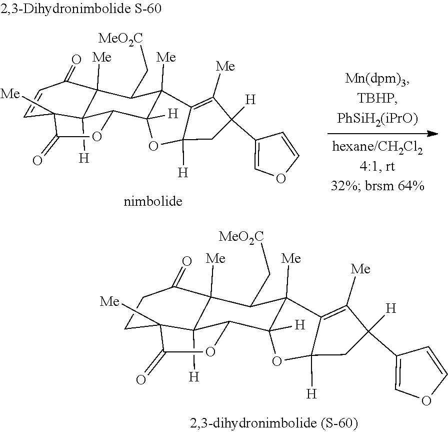

Nimbolide (20 mg, 0.0429 mmol) and Mn(dpm)₃ (1.3 mg, 0.0021 mmol, 5%) was dissolved in 2 ml hexane and 0.5 ml CH₂Cl₂ under Ar. PhSiH₂ (ⁱPrOH) (15 μl, 0.0858 mmol, 2.0 eq) and TBHP (5.5 M in decane, 16 μL, 0.0858 mmol, 2.0 eq) were added in sequence. The mixture was stirred at room temperature for 50 min. Saturated Na₂S₂O₃ were added. The layers were separated and the aqueous layer was extracted several times with EtOAc. The combined organic extracts were washed with brine, dried over MgSO₄, and concentrated. Purification by silica gel chromatography (hexane:EA=2:1) afforded the product 2,3-dihydronimbolide (S-60) as white solid (6.4 mg, yield 32%, brsm: 62%) and recovered nimbolide (5) (9.6 mg, yield 48%).

[α]_{D}²⁵=+119.22 (c 0.253, MeOH); literature reported [α]_{D}²⁵=+122.2 (c 0.1, MeOH). ¹H NMR (400 MHz, CDCl₃): δ 7.33 (t, J=1.7 Hz, 1H), 7.26-7.24 (m, 1H), 6.32 (dd, J=1.9, 0.9 Hz, 1H), 5.53 (ddt, J=8.4, 6.6, 1.9 Hz, 1H), 4.56 (dd, J=12.1, 3.5 Hz, 1H), 4.21 (d, J=3.5 Hz, 1H), 3.67 (dd, J=8.5, 1.8 Hz, 1H), 3.56 (s, 3H), 2.86 (dd, J=15.7, 5.2 Hz, 1H), 2.81 (ddd, J=16.2, 11.6, 8.5 Hz, 1H), 2.71-2.67 (m, 2H), 2.40-2.35 (m, 1H), 2.32 (dd, J=15.7, 5.8 Hz, 1H), 2.22 (dd, J=12.1, 6.7 Hz, 1H), 2.14-2.08 (m, 3H), 1.70 (d, J=1.8 Hz, 3H), 1.50 (s, 3H), 1.33 (s, 3H), 1.28 (s, 3H) ppm. ¹³C NMR (100 MHz, CDCl₃): δ 210.42, 177.70, 172.90, 144.99, 143.01, 138.89, 135.97, 126.52, 110.38, 88.31, 82.77, 72.71, 51.64, 50.02, 49.60, 49.48, 49.33, 41.16, 40.79 (2C), 34.37, 33.22, 32.87, 17.06, 15.72, 15.09, 12.81 ppm. HRMS (ESI-TOF): calc'd for C₂₇H₃₂O₇[M+H]⁺: 469.2220, found: 469.2216. TLC:R_{f}=0.4 (1:1 hexanes:ethyl acetate).

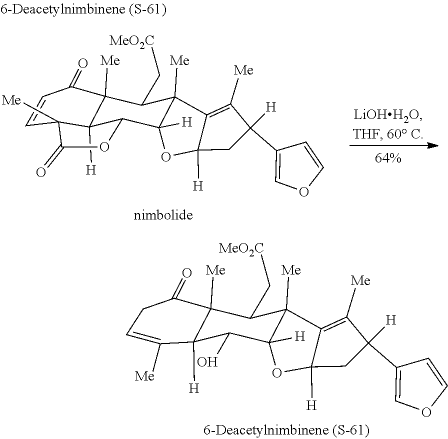

Under Ar (g), the nimbolide (26.8 mg, 0.057 mmol) and LiOH. H₂O (4.8 mg, 0.0857 mmol, 2.0 eq) were dissolved in 2 ml THF and stirred at 70° C. for 7 hours. Then remove the solvent via rotary evaporation to give the crude. Purification on the silica gel chromatography (SiO₂, hexane:EA=5:1) afforded the 6-deacetylnimbinene (S-61) as colorless oil (16.3 mg, 64%).

[α]_{D}²⁴=+124.45 (c 0.143, CHCl₃), (literature [α]_{D}²⁰=+132 (c 1, CHCl₃). ¹H NMR (600 MHz, CDCl₃): δ 7.30 (t, J=1.7 Hz, 1H), 7.20 (s, 1H), 6.27 (t, J=1.3 Hz, 1H), 5.56 (s, 1H), 5.44 (ddt, J=8.5, 6.6, 2.0 Hz, 1H), 4.02 (q, J=8.6, 6.0 Hz, 2H), 3.65 (d, J=7.8 Hz, 1H), 3.57 (s, 3H), 2.96 (ddq, J=19.3, 5.4, 2.9 Hz, 1H), 2.87 (dd, J=19.3, 5.7 Hz, 1H), 2.83-2.76 (m, 2H), 2.64 (dd, J=6.1, 4.4 Hz, 1H), 2.35-2.26 (m, 1H), 2.25-2.19 (m, 1H), 2.16 (dd, J=11.8, 6.5 Hz, 1H), 2.07-1.99 (m, 4H), 1.68 (d, J=1.9 Hz, 3H), 1.31 (s, 3H), 1.04 (s, 3H) ppm. ¹³C NMR (150 MHz, CDCl₃): δ 213.27, 173.71, 147.31, 143.15, 138.98, 138.69, 134.91, 126.85, 119.19, 110.44, 88.02, 86.43, 66.44, 51.71, 49.57, 49.44, 47.33, 47.30, 41.38, 40.01, 37.19, 33.69, 21.81, 17.49, 14.08, 12.94 ppm. HRMS (ESI-TOF): calc'd for C₂₆H₃₂06 [M+H]⁺: 463.2091, found: 463.2081. TLC:R_{f}=0.65 (1:1 hexanes:ethyl acetate).

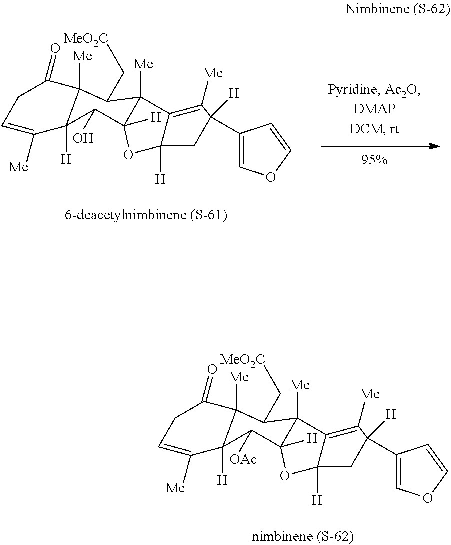

To 6-deacetylnimbinene S-61 (16.3 mg, 0.037 mmol) was dissolved in 2 ml CH₂Cl₂ at rt was added pyridine (12 ul, 0.148 mmol, 4.0 eq) followed by Ac₂O (7 ul, 0.074 mmol, 2.0 eq) and DMAP (2.7 mg, 0.185 mmol, 0.5 eq). After stirring at rt for 24 hours, the reaction mixture was quenched by saturated aq. NaHCO₃ (20 ml). The mixture was extract with CH₂Cl₂ (30 ml×4), dried over Na₂SO₄, then concentrated via rotary evaporation to give the crude which was purified on the silica gel chromatography (SiO₂, hexane:EA=8:1) to afford the product nimbinene (S-62) as colorless oil (16.9 mg, 95%).

[α]_{D}²⁴=+162.01 (c 0.143, CHCl₃), (literature [α]_{D}²⁰=+168 (c 1, CHCl₃). ¹H NMR (600 MHz, CDCl₃): δ 7.32 (t, J=1.7 Hz, 1H), 7.22 (t, J=1.1 Hz, 1H), 6.29 (dd, J=1.9, 0.9 Hz, 1H), 5.60 (ddq, J=5.8, 2.8, 1.5 Hz, 1H), 5.50 (tq, J=6.4, 1.9 Hz, 1H), 5.36 (dd, J=12.3, 3.2 Hz, 1H), 4.08 (d, J=3.2 Hz, 1H), 3.68-3.62 (m, 1H), 3.57 (s, 3H), 3.30 (d, J=12.3 Hz, 1H), 3.01 (dq, J=19.2, 2.6 Hz, 1H), 2.95-2.88 (m, 1H), 2.84 (dd, J=16.2, 5.7 Hz, 1H), 2.74 (t, J=5.2 Hz, 1H), 2.26 (dd, J=16.2, 4.8 Hz, 1H), 2.19 (dd, J=12.0, 6.6 Hz, 1H), 2.16 (s, 3H), 2.05 (dt, J=11.9, 8.5 Hz, 1H), 1.82-1.75 (m, 3H), 1.68 (d, J=1.8 Hz, 3H), 1.39 (s, 3H), 1.12 (s, 3H) ppm. ¹³C NMR (150 MHz, CDCl₃): δ 212.35, 173.54, 170.73, 146.37, 143.08, 138.90, 136.72, 135.21, 126.82, 120.10, 110.42, 86.87, 85.16, 69.15, 51.69, 49.43, 49.40, 47.81, 43.02, 41.37, 39.89, 36.94, 33.53, 21.70, 21.16, 17.16, 14.23, 12.89 ppm. HRMS (ESI-TOF): calc'd for C₂₈H₃₄O₇[M+H]⁺: 483.2378, found: 483.2374. TLC:R_{f}=0.62 (3:1 hexanes:ethyl acetate).

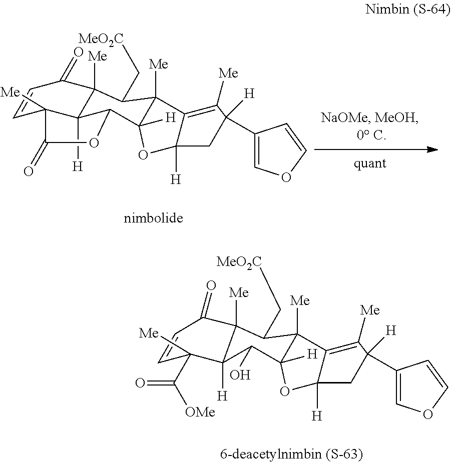

Under Ar(g), nimbolide (20.8 mg, 0.0448 mmol) and MeONa (7.2 mg, 0.134 mmol, 3.0 eq) were dissolved in 4 ml MeOH. After stirring at 0° C. for 1 hour, the solvent was removed via rotary evaporation to give the crude, which was purified on the silica gel chromatography (SiO₂, hexane:EA=1.5:1) afford the product 6-deacetylnimbin (S-63) as white solid (22.3 mg, quant).

[α]_{D}²⁴=+101.16 (c 0.17, CHCl₃), (literature [α]_{D}²⁰=+110 (c 1, CHCl₃). ¹H NMR (600 MHz, CDCl₃): δ 7.33 (t, J=1.7 Hz, 1H), 7.24 (d, J=1.1 Hz, 1H), 6.41 (d, J=10.1 Hz, 1H), 6.33 (dd, J=1.9, 0.9 Hz, 1H), 5.85 (d, J=10.1 Hz, 1H), 5.55 (ddt, J=8.4, 6.6, 2.0 Hz, 1H), 4.02 (d, J=3.3 Hz, 1H), 3.92 (dd, J=11.7, 3.3 Hz, 1H), 3.70 (s, 3H), 3.67 (s, 1H), 3.66 (s, 3H), 3.40 (d, J=11.7 Hz, 1H), 2.90 (dd, J=16.4, 5.7 Hz, 1H), 2.76 (dd, J=5.7, 3.8 Hz, 1H), 2.26-2.20 (m, 1H), 2.20-2.17 (m, 1H), 2.04 (dt, J=11.9, 8.5 Hz, 1H), 1.68 (d, J=1.9 Hz, 3H), 1.59 (s, 3H), 1.29 (s, 3H), 1.21 (s, 3H) ppm. ¹³C NMR (150 MHz, CDCl₃): δ 202.25, 175.61, 173.73, 148.14, 146.84, 143.12, 139.05, 134.98, 126.86, 126.47, 110.48, 87.44, 86.97, 66.25, 53.08, 51.73, 49.65, 47.79, 47.51, 47.37, 43.68, 41.48, 39.10, 34.42, 17.58, 17.19, 16.45, 12.91 ppm. HRMS (ESI-TOF): calc'd for C₂₈H₃₄08 [M+H]⁺: 499.2319, found: 499.2326. TLC:R_{f}=0.42 (6:1 dichloromethane:ethyl acetate).

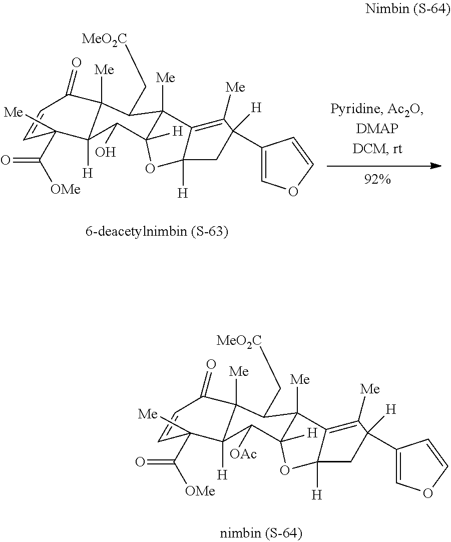

To 6-deacetylnimbin (S-63) (21.8 mg, 0.0437 mmol) in 2 ml CH₂Cl₂ at rt was added pyridine (22 ul, 0.276 mmol, 6.3 eq) followed by Ac20 (13 ul, 0.138 mmol, 3.2 eq) and DMAP (4.2 mg, 0.0345 mmol, 0.79 eq). After stirring at rt for 24 hours, the reaction mixture was quenched by saturated aq. NaHCO₃ (20 ml). The mixture was extracted with CH₂Cl₂ (30 ml×4), dried over Na₂SO₄, then concentrated via rotary evaporation to give the crude which was then purified on the silica gel chromatography (SiO₂, hexane:EA=1:1) to afford the product nimbin (S-64) as colorless oil (21.7 mg, 92%).

[α]_{D}²⁴=+115.27 (c 0.17, CHCl₃), (literature [α]_{D}²⁴=+170 (c 1, CHCl₃). ¹H NMR (600 MHz, CDCl₃): δ 7.32 (t, J=1.7 Hz, 1H), 7.23 (t, J=1.2 Hz, 1H), 6.35-6.33 (m, 1H), 6.33-6.31 (m, 1H), 5.88 (d, J=10.1 Hz, 1H), 5.56 (tq, J=6.6, 1.9 Hz, 1H), 5.21 (dd, J=12.4, 3.0 Hz, 1H), 4.04 (d, J=3.0 Hz, 1H), 3.73 (s, 3H), 3.67 (d, J=12.4 Hz, 4H), 3.64-3.61 (m, 1H), 2.90 (dd, J=16.4, 5.4 Hz, 1H), 2.84 (dd, J=5.4, 4.0 Hz, 1H), 2.22 (dd, J=16.4, 4.0 Hz, 1H), 2.19 (dd, J=11.9, 6.6 Hz, 1H), 2.04 (s, 3H), 2.03-1.99 (m, 1H), 1.66 (d, J=2.0 Hz, 3H), 1.35 (s, 3H), 1.34 (s, 3H), 1.28 (s, 3H). ¹³C NMR (150 MHz, CDCl₃): δ 201.58, 174.57, 173.62, 170.61, 147.52, 146.02, 142.95, 138.94, 135.06, 126.73, 125.90, 110.40, 87.03, 84.47, 68.60, 53.04, 51.65, 49.38, 47.94, 47.85, 47.00, 41.51, 41.41, 38.49, 34.19, 20.93, 17.13, 16.65, 16.58, 12.78 ppm. HRMS (ESI-TOF): calc'd for C₃₀H₃₆O₉[M+H]⁺: 541.2432, found: 541.2410. TLC:R_{f}=0.50 (6:1 dichloromethane:ethyl acetate).

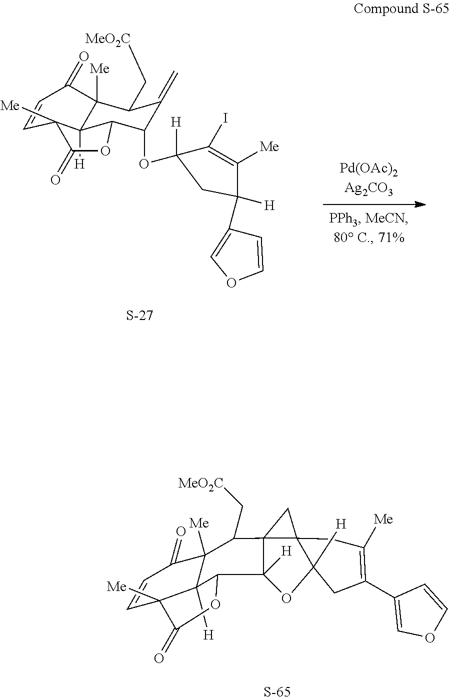

Under Ar (g), compound S-27 (11.8 mg, 0.02 mmol), Pd(OAc)₂ (6.7 mg, 0.03 mmol, 1.5 eq), Ag₂CO₃ (55.2 mg, 0.2 mmol, 10.0 eq) and PPh₃ (31.5 mg, 0.12 mmol, 6.0 eq) were dissolved in 2 ml MeCN. The mixture was stirred at 80° C. 30 minutes. After cooling to rt, the reaction mixture was quenched by saturated aq. NaHCO₃ (20 ml). The mixture was extracted with EA (30 ml×4), dried over Na₂SO₄ and concentrated via rotary evaporation to give the crude which was purified on the silica gel chromatography (SiO₂, hexane:EA=2:1) to afford the product S-65 as white solid (5.6 mg, 71%).

[α]_{D}²³=+117.23 (c 0.087, CHCl₃). ¹H NMR (600 MHz, CDCl₃): δ 7.36 (t, J=1.7 Hz, 1H), 7.32 (s, 1H), 7.00 (d, J=9.7 Hz, 1H), 6.40 (dd, J=1.9, 0.9 Hz, 1H), 5.63 (d, J=9.7 Hz, 1H), 4.77 (dd, J=12.9, 5.5 Hz, 1H), 4.73 (dd, J=8.8, 5.6 Hz, 1H), 4.67 (d, J=5.5 Hz, 1H), 3.73 (s, 3H), 2.79 (ddd, J=17.2, 8.8, 1.9 Hz, 1H), 2.68-2.62 (m, 2H), 2.60 (d, J=12.9 Hz, 1H), 2.52-2.46 (m, 1H), 2.13 (ddq, J=17.2, 5.2, 2.5 Hz, 1H), 1.67 (t, J=2.2 Hz, 3H), 1.45 (s, 3H), 1.33 (d, J=5.9 Hz, 1H), 1.10 (s, 3H), 1.00 (d, J=5.8 Hz, 1H) ppm. ¹³C NMR (150 MHz, CDCl₃): δ 199.46, 175.82, 173.36, 148.41, 142.96, 140.25, 129.81, 128.63, 127.88, 122.13, 109.49, 87.33, 81.26, 73.35, 54.03, 52.19, 48.41, 46.12, 44.57, 41.39, 40.87, 37.16, 35.74, 20.40, 17.79, 17.21, 13.19 ppm. HRMS (ESI-TOF): calc'd for C₂₇H₂₈O₇[M+H]⁺: 465.1908, found: 465.1908. TLC:R_{f}=0.25 (1.5:1 hexanes:ethyl acetate).

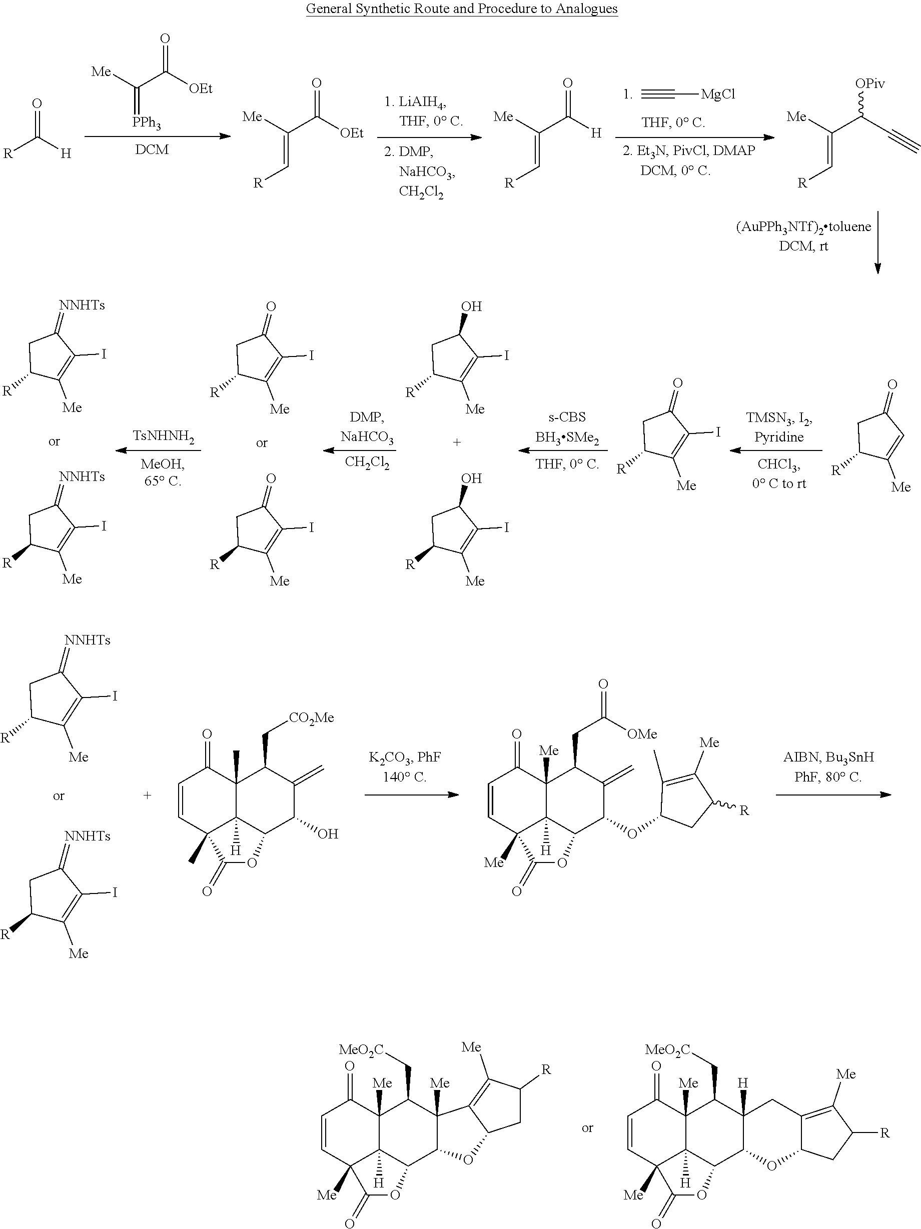

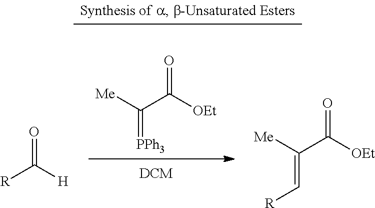

Aldehyde (10.00 mmol) was added to a stirred mixture of ethyl 2-(triphenyl-15-phosphanylidene) propanoate (5.43 g, 15.00 mmol, 1.5 eq) in dry CH₂Cl₂ (100 mL). The mixture was stirred at room temperature until TLC indicated full consumption of aldehyde. The solvent was removed under reduced pressure. The product was purified by column chromatography.

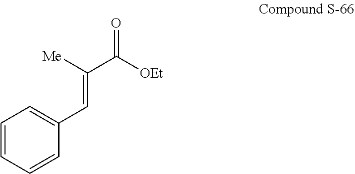 [SMILES: CCOC(=O)/C(C)=C/c1ccccc1]

Yield: quant. (1.920 g). Physical State: colorless oil. ¹H NMR (600 MHz, CDCl₃) δ 7.70 (q, J=1.5 Hz, 1H), 7.41-7.37 (m, 4H), 7.32 (ddt, J=8.6, 5.9, 3.0 Hz, 1H), 4.28 (q, J=7.1 Hz, 2H), 2.12 (d, J=1.5 Hz, 3H), 1.35 (t, J=7.1 Hz, 3H) ppm. ¹³C NMR (150 MHz, CDCl₃): δ 168.82, 138.76, 136.06, 129.74 (2 C), 128.73, 128.46 (2 C), 128.35, 60.99, 60.71, 14.44, 14.17 ppm. HRMS (m/z): calculated for C₁₂H₁₄O₂[M+H]⁺ 191.1067, found 191.1067. TLC:R_{f}=0.65 (20:1 hexanes:ethyl acetate).

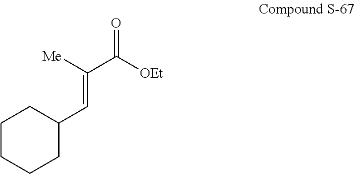 [SMILES: CCOC(=O)/C(C)=C/C1CCCCC1]

Yield: quant. (1.972 g). Physical State: colorless oil. ¹H NMR (600 MHz, CDCl₃): δ 6.57 (dq, J=9.6, 1.5 Hz, 1H), 4.17 (q, J=7.1 Hz, 2H), 2.30 (tdt, J=11.1, 9.6, 3.7 Hz, 1H), 1.83 (d, J=1.4 Hz, 3H), 1.73 (dq, J=10.6, 3.5 Hz, 2H), 1.70-1.60 (m, 4H), 1.29 (t, J=7.2 Hz, 4H), 1.21 (tt, J=12.5, 3.3 Hz, 1H), 1.18-1.10 (m, 2H) ppm. ¹³C NMR (150 MHz, CDCl₃): δ 168.80, 147.43, 126.03, 60.54, 37.87, 32.04 (2 C), 26.01, 25.77, 14.44, 12.54 ppm. HRMS (m/z): calculated for C₁₂H₂₀O₂[M+H]⁺ 197.1533, found 197.1536. TLC:R_{f}=0.67 (20:1 hexanes:ethyl acetate).

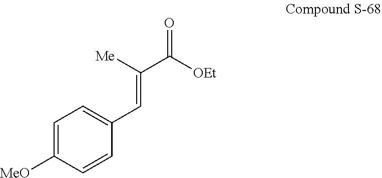 [SMILES: CCOC(=O)/C(C)=C/c1ccc(OC)cc1]

Yield: quant. (2.212 g). Physical State: colorless oil. ¹H NMR (600 MHz, CDCl₃): δ 7.64 (d, J=1.9 Hz, 1H), 7.41-7.35 (m, 2H), 6.96-6.88 (m, 2H), 4.26 (q, J=7.1 Hz, 2H), 3.83 (s, 3H), 2.13 (d, J=1.4 Hz, 3H), 1.34 (t, J=7.1 Hz, 3H) ppm. ¹³C NMR (150 MHz, CDCl₃): δ 169.10, 159.74, 138.48, 131.54, 128.65, 126.53, 113.95, 60.90, 55.43, 14.49, 14.22 ppm. HRMS (m/z): calculated for C₁₃H₁₆O₃[M+H]⁺ 221.1172, found 221.1172. TLC:R_{f}=0.32 (20:1 hexanes:ethyl acetate).

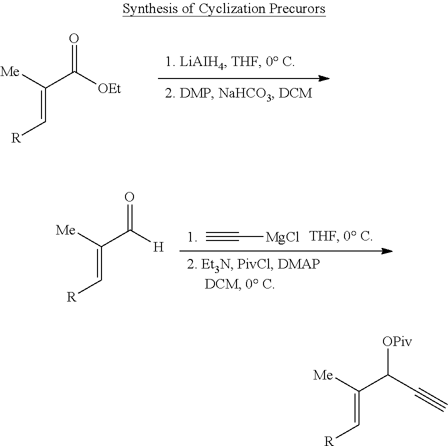

The LiAlH₄ (760.0 mg, 20.00 mmol, 2.0 eq) was added to 50 ml dry THF at 0° C. Then the α,β-unsaturated ester (10.00 mmol) in 100 ml dry THF was added dropwise. The mixture was stirred at 0° C. until TLC showed complete consumption of starting material. The reaction was quenched by 60 ml THF/H₂O (v/v=5/1). Then 10 mL 1 N NaOH was added to the mixture. Filtered the white solid, then concentrated via rotary evaporation to give the crude product used for next step without purified.

To the crude product was dissolved in 200 mL CH₂Cl₂ stirring at 0° C., NaHCO₃ (4.20 g, 50.00 mmol, 5.0 eq) was added followed by DMP (8.48 g, 20.00 mmol, 2.0 eq). The mixture was stirred at room temperature until TLC showed complete consumption of starting material (1 to 3 hours). The reaction was quenched by saturated aq. NaHCO₃ (150 ml) and Na₂S204 (150 ml). The layers were separated and the aqueous layer was extract with CH₂Cl₂ (100 ml×4). The combined organic extracts were washed with brine, dried over MgSO₄, and concentrated via rotary evaporation to give the crude product used for next step without purified.

To the crude aldehyde dissolved in 100 ml dry THF, ethynylmagnesium chloride (0.5 mol/in THF, 40.0 ml, 20.00 mmol, 2.0 eq) was added at 0° C. The mixture was stirred at 0° C. until TLC showed complete consumption of starting material. The reaction was quenched by saturated aq. NH₄Cl (150 ml). The layers were separated and the aqueous layer was extracted with EA (100 ml×5). The combined organic extracts were washed with brine, dried over MgSO₄, and concentrated via rotary evaporation to give the crude product used for next step without purified.

A flask equipped with a magnetic stir bar was charged with the crude propargyl alcohol in anhydrous dichloromethane (100 ml). Then triethylamine (4.18 ml, 30.00 mmol, 3.0 eq) and pivaloyl chloride (1.47 ml, 12.00 mmol, 1.2 eq) were added at 0° C. followed by catalytic amounts of DMAP (610.0 mg, 5.00 mmol, 0.5 eq). The reaction was stirred at 0° C. for 30 min. Quenched by saturated aq. NaHCO₃ (150 ml) and extracted with CH₂Cl₂ (80 ml×4). Combined organic layers were dried over anhydrous MgSO₄, filtered and concentrate via rotary evaporation to give the crude product. purified by column chromatography.

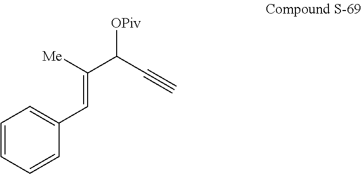

Yield: 81%. (2.261 g). Physical State: colorless oil. ¹H NMR (600 MHz, CDCl₃): δ 7.31 (t, J=7.6 Hz, 2H), 7.25 (d, J=6.9 Hz, 2H), 7.20 (dd, J=7.9, 2.1 Hz, 1H), 6.71 (s, 1H), 5.88 (d, J=2.2 Hz, 1H), 2.52 (d, J=2.2 Hz, 1H), 1.93 (d, J=1.3 Hz, 3H), 1.21 (s, 9H) ppm. ¹³C NMR (150 MHz, CDCl₃): δ 177.22, 136.76, 132.93, 129.61, 129.18, 128.32, 127.23, 79.91, 74.79, 68.87, 39.03, 27.16, 14.32 ppm. HRMS (m/z): calculated for C₁₇H₂₀O₂[M+H]⁺ 279.1348, found 279.1356. TLC:R_{f}=0.64 (20:1 hexanes:ethyl acetate)

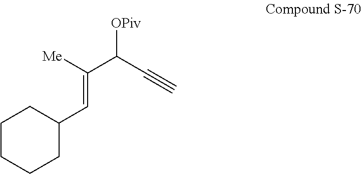

Yield: 85%. (2.237 g). Physical State: colorless oil. ¹H NMR (600 MHz, CDCl₃): δ 5.71 (d, J=2.2 Hz, 1H), 5.51 (dt, J=9.1, 1.3 Hz, 1H), 2.48 (d, J=2.2 Hz, 1H), 2.31-2.07 (m, 1H), 1.72 (d, J=1.3 Hz, 3H), 1.72-1.58 (m, 5H), 1.31-1.21 (m, 3H), 1.20 (s, 9H), 1.11-1.03 (m, 2H) ppm. ¹³C NMR (150 MHz, CDCl₃): δ 177.21, 136.69, 128.66, 80.37, 74.21, 68.79, 38.93, 36.96, 32.72, 27.12, 26.11, 25.95, 12.62 ppm. HRMS (m/z): calculated for C₁₇H₂₆O₂[M+H]⁺ 263.2007, found 263.2001. TLC:R_{f}=0.64 (20:1 hexanes:ethyl acetate)

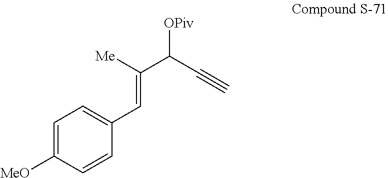

Yield: 74%. (2.116 g). Physical State: yellow oil. H NMR (600 MHz, CDCl₃): δ 7.28-7.22 (m, 2H), 6.91-6.86 (m, 2H), 6.69 (s, 1H), 5.91 (dd, J=2.2, 1.0 Hz, 1H), 3.82 (s, 3H), 2.56 (d, J=2.2 Hz, 1H), 1.97 (d, J=1.4 Hz, 3H), 1.24 (s, 9H) ppm. ¹³C NMR (150 MHz, CDCl₃¹³C NMR (151 MHz, CDCl₃) δ 177.27, 158.75, 131.18, 130.50, 129.32, 129.30, 113.74, 80.06, 74.69, 69.18, 55.40, 39.03, 27.16, 14.32 ppm. HRMS (ESI-TOF): not detected. TLC:R _{f}=0.3 (20:1 hexanes:ethyl acetate).

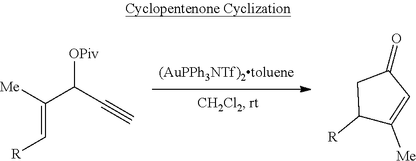

(PPh₃AuNTf)₂·toluene (157.3 mg, 0.10 mmol, 2 mol %), CH₂Cl₂ (250 mL) and water (0.09 ml, 5.00 mmol, 1.0 eq) was added to a 1000 ml flask. The starting material (5.00 mmol) in CH₂Cl₂ (250 mL, 0.02 M) was added dropwise over 1 h through an addition funnel. After addition, solvent was removed via rotary evaporation to give the crude product. Purification by column chromatography.

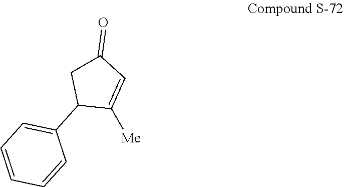 [SMILES: CC1=CC(=O)CC1c1ccccc1]

Yield: 87%. (753.0 mg). Physical State: yellow oil. ¹H NMR (600 MHz, CDCl₃): δ 7.36-7.30 (m, 2H), 7.29-7.23 (m, 1H), 7.14-7.09 (m, 2H), 6.09 (p, J=1.4 Hz, 1H), 3.93-3.88 (m, 1H), 2.93 (dd, J=18.9, 7.2 Hz, 1H), 2.41 (dd, J=18.9, 2.4 Hz, 1H), 1.91 (d, J=1.2 Hz, 3H) ppm. ¹³C NMR (150 MHz, CDCl₃): δ 209.22, 180.34, 141.33, 131.42, 129.17, 127.41, 127.38, 50.80, 45.91, 17.85 ppm. HRMS (m/z): calculated for C₁₂H₁₂0 [M+H]⁺ 173.0961, found 173.0963. TLC:R_{f}=0.34 (4:1 hexanes:ethyl acetate).

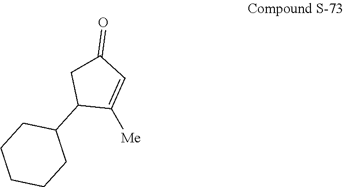 [SMILES: CC1=CC(=O)CC1C1CCCCC1]

Yield: 92%. (925.2 mg). Physical State: pale yellow solid. ¹H NMR (600 MHz, CDCl₃): δ 5.93 (t, J=1.5 Hz, 1H), 2.77 (s, 1H), 2.31 (dd, J=18.8, 6.6 Hz, 1H), 2.22 (dd, J=18.8, 2.4 Hz, 1H), 2.06 (s, 3H), 1.79-1.76 (m, 1H), 1.74-1.60 (m, 4H), 1.35-1.26 (m, 1H), 1.16 (dt, J=12.6, 3.4 Hz, 2H), 1.10 (dddt, J=17.2, 12.9, 9.0, 4.3 Hz, 2H), 0.84 (qd, J=13.5, 13.0, 4.2 Hz, 1H) ppm. ¹³C NMR (150 MHz, CDCl₃): δ 209.65, 180.96, 131.68, 49.78, 38.21, 37.50, 32.36, 26.82, 26.43, 26.23, 25.36, 17.70. ppm. HRMS (m/z): calculated for C₁₂H₁₈O [M+Na]+201.1250, found 201.1250. TLC:R_{f}=0.35 (4:1 hexanes:ethyl acetate)

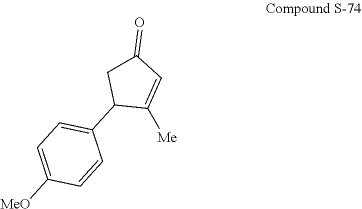 [SMILES: COc1ccc(C2CC(=O)C=C2C)cc1]

Yield: 76%. (774.8 mg). Physical State: yellow solid. ¹H NMR (600 MHz, CDCl₃): δ 7.05-7.01 (m, 2H), 6.89-6.83 (m, 2H), 6.06 (p, J=1.4 Hz, 1H), 3.86 (d, J=7.2 Hz, 1H), 3.79 (s, 3H), 2.90 (dd, J=18.9, 7.1 Hz, 1H), 2.36 (dd, J=18.8, 2.3 Hz, 1H), 1.90 (s, 3H) ppm. ¹³C NMR (150 MHz, CDCl₃): δ 209.34, 180.64, 158.84, 133.23, 131.15, 128.38 (2 C), 114.49 (2 C), 55.41, 50.00, 45.99, 17.78 ppm. HRMS (m/z): calculated for C₁₃H₁₄O₂[M+H]⁺203.1067, found 203.1065. TLC:R_{f}=0.23 (3:1 hexanes:ethyl acetate).

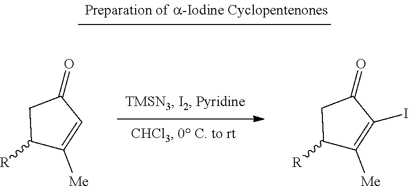

Cyclopentenone (4.00 mmol) was dissolved in CHCl₃ (40 mL), TMSN₃ (1.58 mL, 11.90 mmol, 3.0 eq) was added dropwise at 0° C. After stirring at 0° C. for 2 hours, I₂ (2.03 g, 8.00 mmol, 2.0 eq) was dissolved in CHCl₃ (3.0 ml) and pyridine (3.0 ml) was added dropwise. The mixture was stirred for 24 hours before quenched by saturated aq. NaHCO₃ and Na₂S₂O₃. Extracted with CH₂Cl₂ (80 mL×4). Combined organic layers were dried over anhydrous MgSO₄, filtered and concentrated. The product was purified by column chromatography.

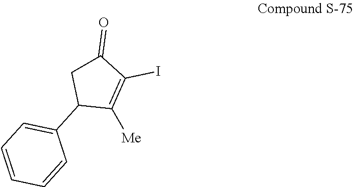 [SMILES: CC1=C(I)C(=O)CC1c1ccccc1]

Yield: 42% rbsm: 71%. (502.3 mg). Physical State: orange oil. H NMR (600 MHz, CDCl₃): δ 7.37-7.32 (m, 2H), 7.32-7.26 (m, 1H), 7.12-7.07 (m, 2H), 4.04 (dd, J=7.2, 1.2 Hz, 1H), 3.09 (dd, J=19.0, 7.1 Hz, 1H), 2.59 (dd, J=18.9, 2.3 Hz, 1H), 2.02 (s, 3H) ppm. ¹³C NMR (150 MHz, CDCl₃): δ 202.94, 181.48, 140.61, 129.34, 127.77, 127.31, 103.82, 52.07, 43.19, 20.85 ppm. HRMS (m/z): calculated for C₁₂H₁₁IO [M+H]⁺ 298.9927, found 298.9927. TLC:R_{f}=0.44 (4:1 hexanes:ethyl acetate).

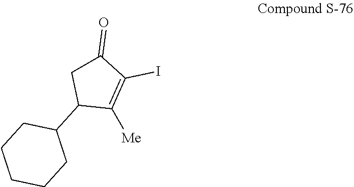 [SMILES: CC1=C(I)C(=O)CC1C1CCCCC1]

Yield: 38%, rbsm: 69%. (463.7 mg). Physical State: yellow oil. ¹H NMR (600 MHz, CDCl₃): δ 2.94 (dt, J=6.3, 2.8 Hz, 1H), 2.48 (dd, J=18.8, 6.6 Hz, 1H), 2.40 (dd, J=18.8, 2.3 Hz, 1H), 2.18 (s, 3H), 1.89-1.82 (m, 1H), 1.81-1.61 (m, 4H), 1.30 (qt, J=13.0, 3.6 Hz, 1H), 1.23-1.14 (m, 2H), 1.14-1.04 (m, 2H), 0.82 (qd, J=13.0, 4.1 Hz, 1H) ppm. ¹³C NMR (150 MHz, CDCl₃): δ 203.19, 182.43, 103.31, 51.41, 38.90, 34.95, 31.86, 26.66, 26.31, 26.12, 25.54, 20.79 ppm. HRMS (m/z): calculated for C12H1710 [M+H]⁺ 305.0397, found 305.0993. TLC:R _{f}=0.52 (4:1 hexanes:ethyl acetate).

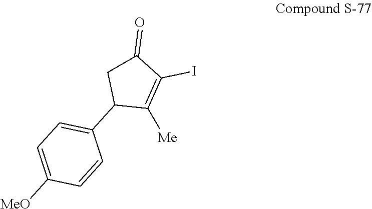 [SMILES: COc1ccc(C2CC(=O)C(I)=C2C)cc1]

Yield: 33%, rbsm: 67%. (434.3 mg). Physical State: orange oil. ¹H NMR (600 MHz, CDCl₃): δ 7.07-6.98 (m, 2H), 6.92-6.79 (m, 2H), 4.00 (d, J=7.0 Hz, 1H), 3.80 (s, 3H), 3.07 (dd, J=18.9, 7.1 Hz, 1H), 2.55 (dd, J=18.9, 2.3 Hz, 1H), 2.01 (d, J=0.9 Hz, 3H) ppm. 13C NMR (150 MHz, CDCl₃): δ 203.05, 181.84, 159.14, 132.52, 128.36, 114.67, 103.54, 55.47, 51.34, 43.31, 20.82. HRMS (m/z): calculated for C₁₃H₁₃IO₂ [M+H]⁺ 329.0033, found 329.0027. TLC:R_{f}=0.36 (3:1 hexanes:ethyl acetate).

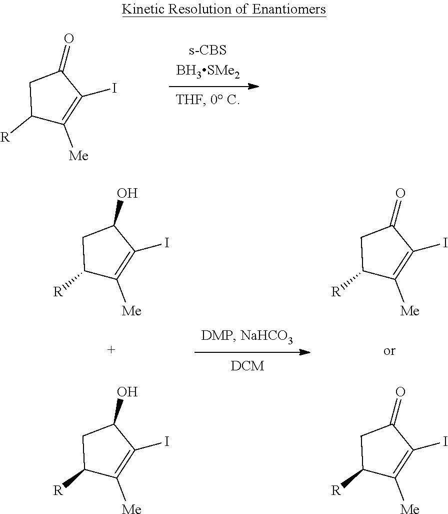

(S)-CBS (277.6 mg, 1.00 mmol, 0.5 eq) was added to the solution of ketone (2.00 mmol) in THF (20 mL) at 0° C. under an argon atmosphere. After the mixture was stirred for 10 min, BH₃·Me₂S (0.19 ml, 2.00 mmol, 1.0 eq) was added dropwise and stirred for 2 hours. Quenched by saturated aq. NH₄Cl. The mixture was extracted by EA (60 mL×4), combined organic layers were dried over anhydrous MgSO₄, filtered and concentrated. The product was purified by column chromatography afford the alcohol.

To the alcohol (1.0 mmol) dissolved in CH₂Cl₂ (10 mL) was added NaHCO₃ (420.4 mg, 5.00 mmol, 5.0 eq) and DMP (868.2 mg, 2.05 mmol, 2.0 eq). The mixture was stirred at room temperature until TLC showed complete consumption of starting material. The reaction was quenched by saturated aq. NaHCO₃ and aq. Na₂S₂O₃. The layers were separated and the aqueous layer was extracted several times with CH₂Cl₂. The combined organic extracts were washed with brine, dried over MgSO₄, and concentrated to get the crude. The product was purified by column chromatography afford the ketone. Stereo-chemistry was assigned according to rotation and

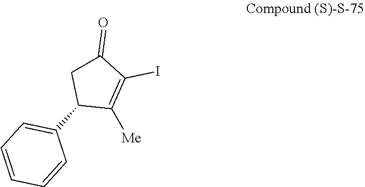 [SMILES: CC1=C(I)C(=O)C[C@H]1c1ccccc1]

94% ee. [α]_{D}²²=+127.28 (c 1.114, CHCl₃).

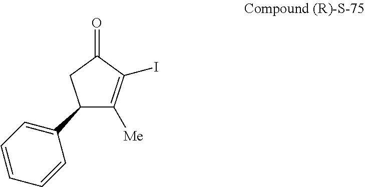 [SMILES: CC1=C(I)C(=O)C[C@@H]1c1ccccc1]

75% ee. [α]_{D}²²=−102.66 (c 0.637, CHCl₃).

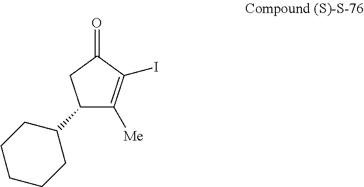 [SMILES: CC1=C(I)C(=O)C[C@H]1C1CCCCC1]

87% ee. Minimal optical rotation was observed for this compound. The structure assignment is relied on analogy of related analogues.

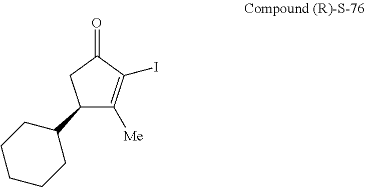 [SMILES: CC1=C(I)C(=O)C[C@@H]1C1CCCCC1]

73% ee. Minimal optical rotation was observed for this compound. The structure assignment is relied on analogy of related analogues.

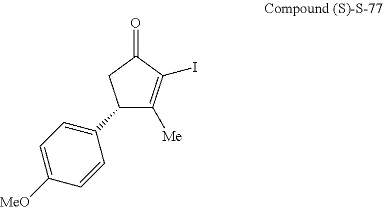 [SMILES: COc1ccc([C@@H]2CC(=O)C(I)=C2C)cc1]

79% ee. [α]_{D}²⁴=+102.66 (c 0.637, CHCl₃).

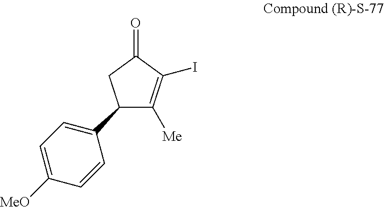 [SMILES: COc1ccc([C@H]2CC(=O)C(I)=C2C)cc1]

80% ee. [α]_{D}²⁴=−107.02 (c 0.213, CHCl₃).

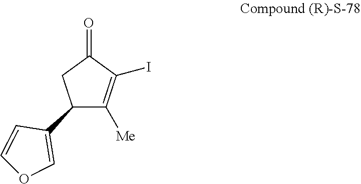 [SMILES: CC1=C(I)C(=O)C[C@@H]1c1ccoc1]

83% ee. [α]_{D}²²=−86.71 (c 0.362, CHCl₃).

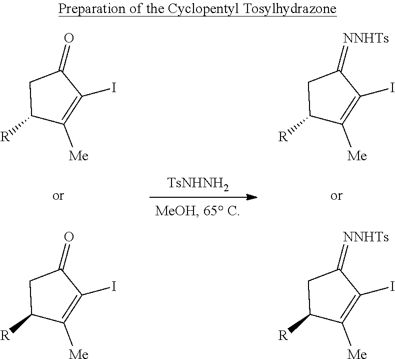

The cyclopentenone (0.5 mmol) and TsNHNH₂ (447.0 mg, 2.4 mmol, 6.0 eq) were dissolved in 5 ml MeOH then warm to 65° C. After 12-24 hours the reaction was completed, remove the solvent via rotary evaporation to give the crude, which was purified on the silica gel chromatography to afford the product tosyl-hydrazone.

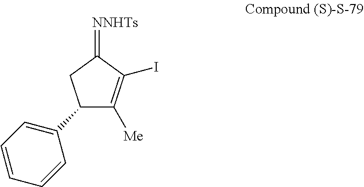

Yield: 73%. (170.4 mg). Physical State: yellow oil. [α]_{D}²⁴=+135.32 (c 0.130, CHCl₃).

The NMR spectra matched with (R)-S-79.

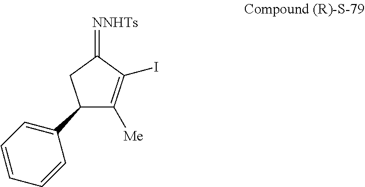

Yield: 69%. (161.1 mg). Physical State: yellow oil. [α]_{D}²⁴=−101.89 (c 0.263, CHCl₃). ¹H NMR (600 MHz, CDCl₃): δ 7.96 (s, 1H), 7.94 (s, 1H), 7.33 (d, J=8.1 Hz, 2H), 7.29 (t, J=7.3 Hz, 2H), 7.26-7.23 (m, 1H), 7.21 (s, 1H), 7.01 (dd, J=6.8, 1.6 Hz, 2H), 3.91 (d, J=8.0 Hz, 1H), 3.01 (dd, J=17.8, 7.9 Hz, 1H), 2.47-2.42 (m, 4H), 1.82 (d, J=1.2 Hz, 3H) ppm. 13C NMR (150 MHz, CDCl₃): δ 165.27, 163.34, 144.27, 141.45, 135.06, 129.56, 129.18, 128.60, 127.62, 127.29, 95.98, 53.28, 35.36, 21.82, 19.24 ppm. HRMS (m/z): calculated for C₁₉H₁₉1N₂02S [M+H]⁺=467.0285, found 467.0279. TLC:R_{f}=0.42 (2:1 hexanes:ethyl acetate).

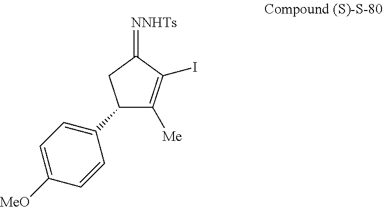

Yield: 66%. (164.0 mg). Physical State: yellow foam. [α]_{D}²⁴=+107.48 (c 0.08, CHCl₃).

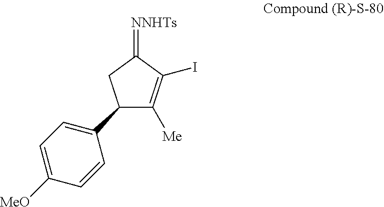

Yield: 61%. (151.6 mg). Physical State: yellow foam. [α]_{D}²⁴=−105.98 (c 0.10, CHCl₃). ¹H NMR (600 MHz, CDCl₃): δ 7.95 (s, 1H), 7.93 (s, 1H), 7.36-7.30 (m, 3H), 6.95-6.90 (m, 2H), 6.84-6.79 (m, 2H), 3.86 (d, J=7.9 Hz, 1H), 3.78 (s, 3H), 2.43 (s, 3H), 2.41 (dd, J=17.9, 2.6 Hz, 1H), 1.80 (d, J=1.3 Hz, 3H) ppm. ¹³C NMR (150 MHz, CDCl₃): δ 165.60, 163.41, 158.97, 144.22, 135.06, 133.44, 129.54, 128.58, 128.33, 114.47, 95.64, 55.42, 52.55, 35.48, 21.80, 19.20 ppm. HRMS (m/z): calculated for C₂₀H₂₁IN₂O₃S [M+H]⁺=497.0390, found 497.0387. TLC:R_{f}=0.21 (2:1 hexanes:ethyl acetate).

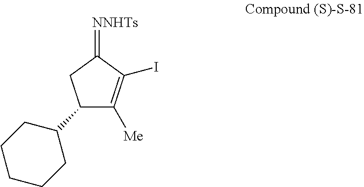

Yield: 68%. (160.8 mg). Physical State: yellow oil. [α]_{D}²⁴=+20.65 (c 0.213, CHCl₃).

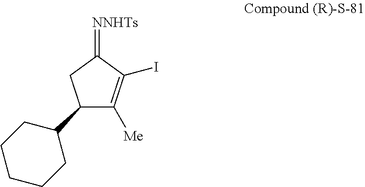

Yield: 68%. (160.8 mg). Physical State: yellow oil. [α]_{D}²⁴=−12.99 (c 0.277, CHCl₃). ¹H NMR (600 MHz, Acetone-D6): δ 8.89 (s, 1H), 8.01-7.75 (m, 2H), 7.37 (d, J=8.0 Hz, 2H), 2.94-2.90 (m, 1H), 2.83 (s, 1H), 2.56-2.49 (m, 2H), 2.41 (s, 3H), 1.99 (d, J=1.2 Hz, 3H), 1.79 (tq, J=12.1, 3.3 Hz, 1H), 1.76-1.71 (m, 1H), 1.67-1.62 (m, 1H), 1.62-1.56 (m, 1H), 1.29 (tdd, J=13.0, 9.4, 3.6 Hz, 1H), 1.22-1.03 (m, 4H), 0.72 (qd, J=12.7, 3.7 Hz, 1H) ppm. ¹³C NMR (150 MHz, Acetone-D6): δ 166.14, 164.39, 144.37, 137.40, 130.02, 129.14, 95.70, 53.18, 39.61, 31.94, 27.74, 27.27, 27.05, 26.72, 25.98, 21.49, 19.17 ppm. HRMS (m/z): calculated for C₁₉H₂₅IN₂02S [M+H]⁺ 473.0754, found 473.0758. TLC:R_{f}=0.42 (3:1 hexanes:ethyl acetate).

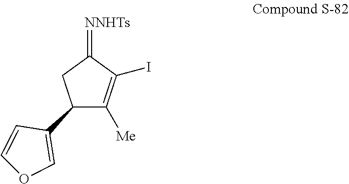

Yield: 65%. (126.8 mg). Physical State: yellow oil. [α]_{D}²³=−95.37 (c 0.26, CHCl₃). ¹H NMR (600 MHz, CD₃CN): δ8.13 (s, 1H), 7.90-7.81 (m, 2H), 7.42 (t, J=1.8 Hz, 1H), 7.39 (dd, J=7.4, 1.5 Hz, 3H), 6.15 (dd, J=1.9, 0.9 Hz, 1H), 3.99 (d, J=7.9 Hz, 1H), 3.01 (dd, J=18.1, 7.9 Hz, 1H), 2.44 (m, 4H), 1.84 (d, J=1.3 Hz, 3H) ppm. ¹³C NMR (150 MHz, CD₃CN): δ166.10, 164.25, 145.20, 144.95, 140.75, 136.57, 130.31, 129.06, 126.50, 109.70, 95.39, 43.91, 35.18, 21.51, 19.14 ppm. HRMS (ESI-TOF): calc'd for C₁₉H₂₃N₃O₄S [M+H]⁺: 390.1482, found: 390.1491.

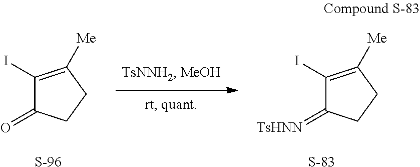

Compound S-96 (1.0 g, 4.5 mmol) and TsNNH₂ (922.5 mg, 4.95 mmol, 1.1 eq) were dissolved in 20 ml MeOH and stirred at rt for 24 hours, during which the product crush out as white solids. The solvent was removed by filtration and the resulting solid was dried via vacuo to afford the product S-83 as white solid (1.39 g, quant). ¹H NMR (600 MHz, CDCl₃): δ 7.94 (d, J=8.3 Hz, 2H), 7.31 (d, J=8.2 Hz, 2H), 7.09 (s, 1H), 2.67-2.62 (m, 2H), 2.53-2.48 (m, 2H), 2.42 (s, 3H), 2.11-1.96 (m, 3H) ppm. ¹³C NMR (150 MHz, CDCl₃): δ 165.19, 163.54, 144.22, 135.07, 129.53, 128.57, 94.51, 35.46, 25.42, 21.79, 20.42 ppm. HRMS (ESI-TOF): calc'd for C₁₃H₁₅IN₂02S [M+H]⁺: 390.1482, found: 390.1491. TLC:R_{f}=0.50 (3:1 hexanes ethyl acetate).

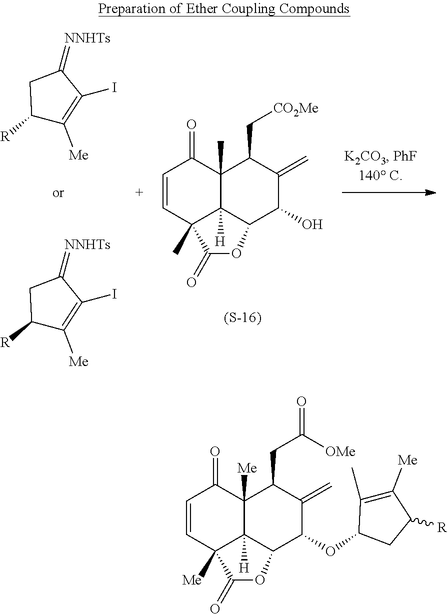

The compound S-16 (10.0 mg, 0.0312 mmol), tosyl-hydrazone (0.937 mmol, 3.0 eq) and K₂CO₃ (17.2 mg, 0.125 mmol, 4.0 eq) were dissolved in 1 ml PhF. The mixture was stirred at 140° C. (0.5-1 hour), then cooled to rt. Removal of the solvent via rotary evaporation gave the crude. Purified on the silica gel chromatography afforded the coupling compounds.

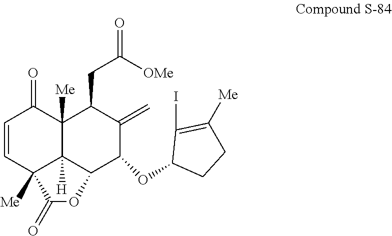

Yield: 45%. (7.6 mg). Physical State: colorless oil. [α]_{D}²⁵=+78.51 (c 0.433, CHCl₃). ¹H NMR (600 MHz, CD₃CN): δ7.28 (d, J=9.7 Hz, 1H), 5.95 (d, J=9.7 Hz, 1H), 5.40 (d, J=1.4 Hz, 1H), 5.10 (d, J=1.7 Hz, 1H), 4.72 (d, J=3.7 Hz, 1H), 4.54 (m, 1H), 4.36 (dd, J=12.4, 3.7 Hz, 1H), 3.65 (s, 3H), 3.61 (dd, J=16.5, 3.2 Hz, 1H), 3.39 (d, J=12.4 Hz, 1H), 3.28 (d, J=11.7 Hz, 1H), 2.58 (dd, J=16.4, 11.6 Hz, 1H), 2.52-2.44 (m, 1H), 2.29-2.23 (m, 1H), 2.20 (m, 1H), 1.94-1.87 (m, 1H), 1.78 (d, J=1.3 Hz, 3H), 1.42 (s, 3H), 1.02 (s, 3H) ppm. ¹³C NMR (150 MHz, CD₃CN): δ202.02, 175.58, 172.54, 150.03, 149.39, 144.76, 131.69, 118.00, 94.07, 88.97, 79.53, 75.90, 51.82, 47.62, 46.14, 44.10, 41.07, 35.35, 31.44, 30.81, 19.28, 18.69, 13.05 ppm. HRMS (ESI-TOF): calc'd for C₂₃H₂₇IO₆ [M+Na]⁺: 549.0745, found: 549.0737. TLC:R_{f}=0.21 (2:1 hexanes:ethyl acetate).

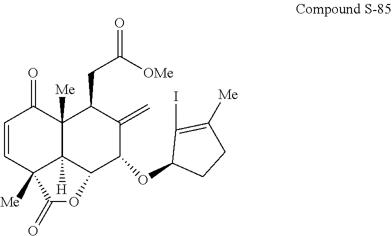

Yield: 43%. (7.2 mg). Physical State: colorless oil. [α]_{D}²⁵=−12.01 (c 0.333, CHCl₃). ¹H NMR (600 MHz, CDCl₃): δ 7.28 (d, J=9.7 Hz, 1H), 5.97 (d, J=9.7 Hz, 1H), 5.39 (d, J=1.3 Hz, 1H), 5.16 (d, J=1.7 Hz, 1H), 4.54 (s, 1H), 4.48 (d, J=3.6 Hz, 1H), 4.37 (dd, J=12.3, 3.7 Hz, 1H), 3.65 (dd, J=16.3, 3.4 Hz, 1H), 3.65 (s, 3H), 3.58 (d, J=11.7 Hz, 1H), 3.53 (d, J=12.3 Hz, 1H), 2.60 (dd, J=16.4, 11.7 Hz, 1H), 2.52-2.44 (m, 1H), 2.29-2.22 (m, 1H), 2.17 (m, 1H), 1.91-1.85 (m, 1H), 1.78 (d, J=1.5 Hz, 3H), 1.42 (s, 3H), 1.03 (s, 3H) ppm. ¹³C NMR (150 MHz, CDCl₃): δ 201.93, 175.79, 172.52, 149.91, 149.07, 143.81, 131.66, 118.91, 94.67, 86.19, 77.19, 75.73, 51.86, 47.52, 46.24, 44.12, 40.89, 35.39, 31.68, 28.74, 19.24, 18.66, 13.05 ppm. HRMS (ESI-TOF): calc'd for C₂₃H₂₇IO₆ [M+Na]⁺: 549.0745, found: 549.0743. TLC:R_{f}=0.21 (2:1 hexanes:ethyl acetate).

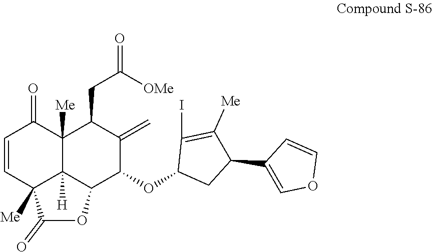

Yield: 47%. (8.9 mg). Physical State: colorless oil. [α]_{D}²⁹=−50.16 (c 0.223, CHCl₃). ¹H NMR (600 MHz, CDCl₃): δ 7.34 (t, J=1.8 Hz, 1H), 7.29 (d, J=9.6 Hz, 1H), 7.24 (s, 1H), 6.12 (s, 1H), 5.98 (d, J=9.7 Hz, 1H), 5.39 (d, J=1.3 Hz, 1H), 5.18 (d, J=1.7 Hz, 1H), 4.69 (ddt, J=7.3, 3.7, 1.8 Hz, 1H), 4.48 (d, J=3.6 Hz, 1H), 4.38 (dd, J=12.3, 3.6 Hz, 1H), 3.80 (s, 1H), 3.68-3.64 (m, 4H), 3.59 (d, J=12.5 Hz, 1H), 3.54 (d, J=12.3 Hz, 1H), 2.61 (dd, J=16.4, 11.7 Hz, 1H), 2.33 (ddd, J=13.4, 8.5, 3.5 Hz, 1H), 2.10-2.03 (m, 1H), 1.66 (s, 3H), 1.43 (s, 3H), 1.04 (s, 3H) ppm. ¹³C NMR (150 MHz, CDCl₃): δ 201.87, 175.77, 172.47, 151.27, 149.90, 143.66, 143.61, 139.21, 131.67, 126.86, 119.16, 109.34, 96.31, 84.80, 77.37, 75.65, 51.85, 47.58, 46.24, 44.14, 43.46, 40.94, 38.01, 31.65, 18.64, 17.88, 13.05.ppm.

HRMS (ESI-TOF): calc'd for C₂₇H₂₉IO₇ [M+H]⁺: 615.0850, found: 615.0833. TLC:R_{f}=0.20 (2:1 hexanes:ethyl acetate).

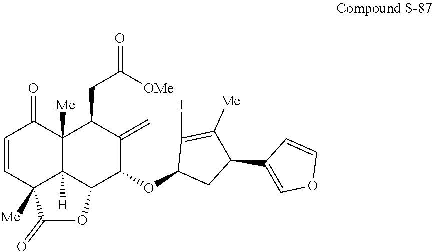

Yield: 41%. (7.8 mg). Physical State: colorless oil. [α]_{D}²⁹=−46.71 (c 0.107, CHCl₃). ¹H NMR (600 MHz, CDCl₃): δ 7.32 (t, J=1.8 Hz, 1H), 7.30 (d, J=9.7 Hz, 1H), 7.27 (s, 1H), 6.36 (dd, J=1.9, 0.9 Hz, 1H), 5.96 (d, J=9.7 Hz, 1H), 5.41 (d, J=1.4 Hz, 1H), 5.11 (d, J=1.8 Hz, 1H), 4.80 (d, J=3.5 Hz, 1H), 4.63-4.59 (m, 1H), 4.40 (dd, J=12.4, 3.6 Hz, 1H), 3.61 (s, 4H), 3.51-3.45 (m, 2H), 3.29 (d, J=11.9 Hz, 1H), 2.66 (ddd, J=13.7, 8.6, 7.2 Hz, 1H), 2.58 (dd, J=16.4, 11.9 Hz, 1H), 1.87 (ddd, J=13.7, 4.5, 3.6 Hz, 1H), 1.64 (s, 3H), 1.44 (s, 3H), 1.02 (s, 3H) ppm. ¹³C NMR (150 MHz, CDCl₃): δ 201.92, 175.54, 172.48, 150.90, 149.97, 144.32, 143.53, 139.19, 131.76, 126.91, 118.21, 110.35, 95.35, 87.05, 78.88, 76.24, 51.76, 47.71, 46.10, 44.03, 43.46, 41.21, 39.28, 31.37, 18.77, 17.77, 13.09 ppm. HRMS (ESI-TOF): calc'd for C₂₇H₂₉IO₇ [M+H]⁺: 615.0850, found: 615.0840. TLC:R_{f}=0.24 (2:1 hexanes ethyl acetate).

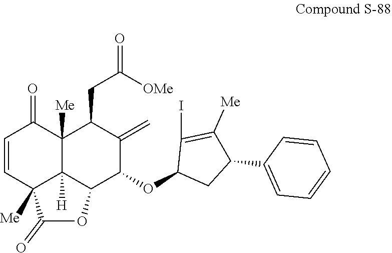

Yield: 46%. (8.9 mg). Physical State: colorless oil. [α]_{D}²⁵=+28.66 (c 0.3, CHCl₃). ¹H NMR (600 MHz, CDCl₃): δ 7.30 (d, J=9.7 Hz, 1H), 7.28 (t, J=7.7 Hz, 2H), 7.22-7.18 (m, 1H), 7.04 (d, J=6.8 Hz, 2H), 5.96 (d, J=9.7 Hz, 1H), 5.40 (d, J=1.3 Hz, 1H), 5.10 (d, J=1.7 Hz, 1H), 4.75 (ddd, J=7.2, 3.1, 1.6 Hz, 1H), 4.73 (d, J=3.7 Hz, 1H), 4.38 (dd, J=12.4, 3.7 Hz, 1H), 3.89-3.84 (m, 1H), 3.61 (dd, J=16.5, 3.3 Hz, 1H), 3.58 (s, 3H), 3.42 (d, J=12.3 Hz, 1H), 3.30 (d, J=11.7 Hz, 1H), 2.61-2.55 (m, 1H), 2.44 (ddd, J=13.9, 8.5, 3.3 Hz, 1H), 2.17 (ddd, J=13.9, 7.2, 4.7 Hz, 1H), 1.61 (s, 3H), 1.43 (s, 3H), 1.02 (s, 3H) ppm. ¹³C NMR (150 MHz, CDCl₃): δ 201.97, 175.61, 172.45, 152.10, 149.99, 144.68, 143.55, 131.73, 128.80, 127.51, 126.77, 118.06, 96.17, 88.15, 79.59, 75.95, 53.66, 51.78, 47.69, 46.15, 44.11, 41.57, 41.13, 31.44, 18.69, 18.06, 13.04 ppm. HRMS (ESI-TOF): calc'd for C₂₉H₃₁IO₆ [M+Na]⁺: 625.1058, found: 625.1049. TLC:R_{f}=0.31 (2:1 hexanes:ethyl acetate).

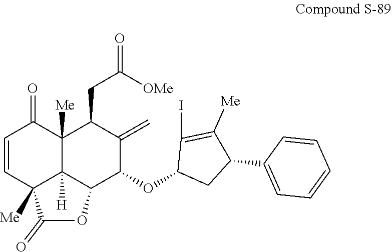

Yield: 41%. (8.9 mg). Physical State: colorless oil. [α]_{D}²⁵=+31.49 (c 0.127, CHCl₃). ¹H NMR (600 MHz, CDCl₃): δ 7.35-7.33 (m, 1H), 7.33-7.28 (m, 2H), 7.26-7.24 (m, 2H), 7.24-7.20 (m, 1H), 5.99 (d, J=9.7 Hz, 1H), 5.39 (d, J=1.2 Hz, 1H), 5.19 (d, J=1.6 Hz, 1H), 4.57 (ddd, J=7.2, 3.5, 1.8 Hz, 1H), 4.50 (d, J=3.7 Hz, 1H), 4.39 (dd, J=12.3, 3.7 Hz, 1H), 3.70-3.64 (m, 5H), 3.60-3.55 (m, 2H), 2.71-2.59 (m, 2H), 1.96-1.92 (m, 1H), 1.57 (s, 3H), 1.44 (s, 3H), 1.05 (s, 3H) ppm. ¹³C NMR (150 MHz, CDCl₃): δ 201.96, 175.71, 172.54, 151.38, 150.02, 143.59, 143.11, 131.65, 128.76, 128.17, 126.93, 119.16, 96.27, 84.57, 77.04, 75.63, 53.95, 51.87, 47.60, 46.30, 44.19, 40.94, 38.31, 31.71, 18.64, 17.94, 13.06 ppm. HRMS (ESI-TOF): calc'd for C₂₉H₃₁IO₆ [M+Na]⁺: 625.1058, found: 625.1035. TLC:R_{f}=0.31 (2:1 hexanes:ethyl acetate). Note: compound S35 and S36 were separated by the PTLC (hexane:EA=3:1 (3 times)).

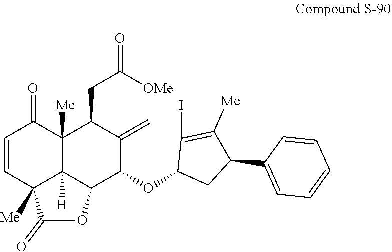

Yield: 44%. (8.2 mg). Physical State: colorless oil. [α]_{D}²⁵=−58.20 (c 0.213, CHCl₃). ¹H NMR (600 MHz, CDCl₃): δ 7.31-7.26 (m, 3H), 7.23-7.19 (m, 1H), 7.05 (d, J=6.8 Hz, 2H), 5.99 (d, J=9.7 Hz, 1H), 5.37 (d, J=1.3 Hz, 1H), 5.18 (d, J=1.7 Hz, 1H), 4.76-4.74 (m, 1H), 4.48 (d, J=3.6 Hz, 1H), 4.39 (dd, J=12.3, 3.7 Hz, 1H), 3.89-3.83 (m, 1H), 3.71-3.65 (m, 4H), 3.62 (d, J=11.6 Hz, 1H), 3.56 (d, J=12.2 Hz, 1H), 2.62 (dd, J=16.2, 11.6 Hz, 1H), 2.43 (ddd, J=13.7, 8.8, 3.5 Hz, 1H), 2.10 (ddd, J=13.7, 7.0, 4.3 Hz, 1H), 1.61 (s, 3H), 1.43 (s, 3H), 1.04 (s, 3H) ppm. ¹³C NMR (150 MHz, CDCl₃): δ 201.90, 175.78, 172.47, 151.80, 149.92, 143.64, 143.57, 131.68, 128.84, 127.49, 126.82, 119.21, 96.88, 85.10, 77.35, 75.66, 53.87, 51.87, 47.60, 46.26, 44.15, 40.97, 39.34, 31.72, 18.66, 18.05, 13.06 ppm. HRMS (ESI-TOF): calc'd for C₂₉H₃₁IO₆ [M+Na]⁺: 625.1058, found: 625.1050. TLC:R_{f}=0.31 (2:1 hexanes:ethyl acetate).

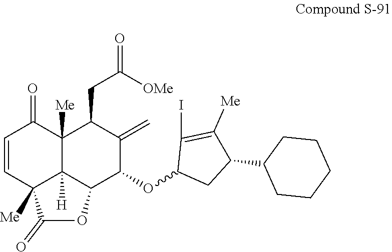

Yield: 83%. (16.2 mg). Physical State: colorless oil. The ratio is 1.4:1. HRMS (ESI-TOF): calc'd for C₂₉H₃₇IO₆ [M+Na]⁺: 631.1527, found: 631.1521. TLC:R_{f}=0.41 (2:1 hexanes:ethyl acetate). ¹H NMR (600 MHz, CDCl₃): δ 7.28 (dd, J=9.7, 1.2 Hz, 1H), 5.95 (dd, J=11.6, 9.7 Hz, 1H), 5.39 (dd, J=8.9, 1.4 Hz, 1H), 5.14 (dd, J=24.9, 1.7 Hz, 1H), 4.71 (d, J=3.7 Hz, 1H), 4.51-4.46 (m, 1H), 4.42-4.33 (m, 1H), 3.65 (s, 2H), 3.64 (s, 1H), 3.61 (dd, J=16.1, 3.1 Hz, 1H), 3.40 (d, J=12.4 Hz, 1H), 3.31-3.26 (m, 1H), 2.66 (dt, J=9.0, 3.3 Hz, 1H), 2.59 (ddd, J=16.2, 11.6, 8.5 Hz, 1H), 2.14-2.07 (m, 1H), 1.82 (ddd, J=13.6, 8.9, 4.4 Hz, 1H), 1.73 (s, 2H), 1.72 (s, 2H), 1.70-1.63 (m, 2H), 1.52 (dddd, J=17.4, 11.0, 5.7, 2.6 Hz, 2H), 1.42 (s, 1H), 1.41 (s, 2H), 1.24 (dh, J=12.4, 3.5 Hz, 2H), 1.19-1.04 (m, 4H), 1.03 (s, 1H), 1.01 (s, 2H) ppm. ¹³C NMR (150 MHz, CDCl₃): δ 202.01, 175.73, 175.59, 172.52, 172.48, 151.20, 150.55, 150.02, 149.99, 144.79, 143.83, 131.69, 131.63, 118.81, 118.03, 95.89, 95.44, 87.90, 84.11, 79.45, 75.97, 75.67, 52.84, 52.64, 51.82, 47.61, 47.45, 46.14, 44.08, 41.17, 40.89, 39.31, 38.93, 32.99, 31.75, 31.72, 31.66, 31.49, 29.97, 26.99, 26.87, 26.67, 26.62, 26.61, 26.43, 26.38, 26.25, 18.71, 18.02, 17.95, 13.04 ppm.

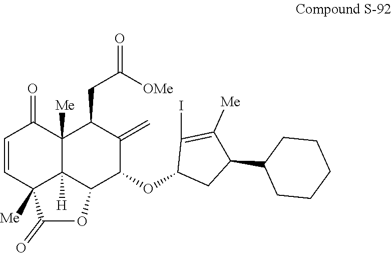

Yield: 43%. (8.4 mg). Physical State: colorless oil. [α]_{D}²⁵=−14.28 (c 0.210, CHCl₃). ¹H NMR (600 MHz, CDCl₃): δ 7.28 (d, J=9.7 Hz, 1H), 5.96 (d, J=9.8 Hz, 1H), 5.40 (s, 1H), 5.20 (d, J=1.7 Hz, 1H), 4.47 (d, J=3.6 Hz, 1H), 4.45-4.41 (m, 1H), 4.37 (dd, J=12.3, 3.6 Hz, 1H), 3.65 (dd, J=16.0, 3.4 Hz, 1H), 3.62 (s, 3H), 3.58 (d, J=11.6 Hz, 1H), 3.52 (d, J=12.2 Hz, 1H), 2.69 (d, J=6.0 Hz, 1H), 2.60 (dd, J=16.1, 11.7 Hz, 1H), 1.95 (ddd, J=13.6, 7.2, 3.5 Hz, 1H), 1.83 (ddd, J=13.4, 9.0, 4.2 Hz, 1H), 1.72 (t, J=1.3 Hz, 3H), 1.69-1.63 (m, 2H), 1.56-1.48 (m, 2H), 1.42 (s, 3H), 1.25 (tdd, J=9.4, 8.0, 6.7, 3.4 Hz, 2H), 1.21-1.17 (m, 1H), 1.13 (ddd, J=15.0, 12.3, 2.7 Hz, 1H), 1.06 (td, J=12.7, 3.7 Hz, 2H), 1.03 (s, 3H), 0.73 (qd, J=12.2, 3.4 Hz, 1H) ppm. ¹³C NMR (150 MHz, CDCl₃): δ 201.94, 175.76, 172.45, 151.03, 149.92, 143.81, 131.65, 119.01, 95.81, 85.10, 77.19, 75.72, 52.96, 51.78, 47.53, 46.25, 44.12, 40.96, 39.27, 31.89, 31.66, 31.02, 26.83, 26.61, 26.40, 26.32, 18.66, 18.01, 13.01 ppm. HRMS (ESI-TOF): calc'd for C₂₉H₃₇IO₆ [M+Na]⁺: 631.1527, found: 631.1520. TLC:R_{f}=0.41 (2:1 hexanes:ethyl acetate).

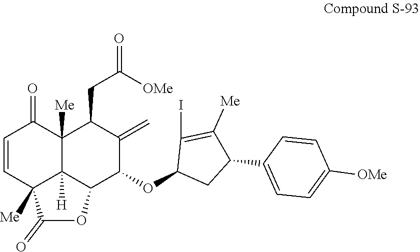

Yield: 44%. (8.9 mg). Physical State: colorless oil. [α]_{D}²⁴=+55.36 (c 0.177, CHCl₃). ¹H NMR (600 MHz, CDCl₃): δ 7.30 (d, J=9.7 Hz, 1H), 6.98-6.93 (m, 2H), 6.84-6.79 (m, 2H), 5.96 (d, J=9.7 Hz, 1H), 5.40 (d, J=1.3 Hz, 1H), 5.10 (d, J=1.7 Hz, 1H), 4.73 (dd, J=8.4, 3.2 Hz, 2H), 4.38 (dd, J=12.4, 3.7 Hz, 1H), 3.84-3.80 (m, 1H), 3.78 (s, 3H), 3.62-3.59 (m, 4H), 3.42 (d, J=12.4 Hz, 1H), 3.30 (ddt, J=11.7, 3.2, 1.6 Hz, 1H), 2.58 (dd, J=16.4, 11.7 Hz, 1H), 2.41 (ddd, J=13.9, 8.5, 3.3 Hz, 1H), 2.12 (ddd, J=13.9, 7.2, 4.8 Hz, 1H), 1.59 (s, 3H), 1.43 (s, 3H), 1.02 (s, 3H) ppm. ¹³C NMR (150 MHz, CDCl₃): δ 201.97, 175.61, 172.45, 158.43, 152.49, 149.99, 144.69, 135.55, 131.73, 128.48, 118.02, 114.13, 95.79, 88.13, 79.54, 75.96, 55.39, 52.82, 51.79, 47.68, 46.14, 44.10, 41.64, 41.13, 31.45, 18.69, 18.03, 13.04 ppm. HRMS (ESI-TOF): calc'd for C₃₀H₃₃IO₇ [M+Na]⁺: 655.1163, found: 655.1167. TLC:R _{f}=0.36 (2:1 hexanes:ethyl acetate).

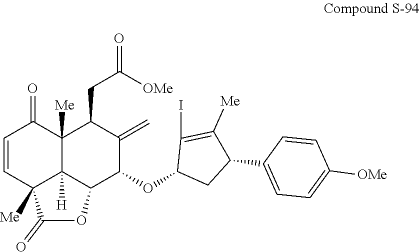

Yield: 39%. (7.9 mg). Physical State: colorless oil. [α]_{D}²⁴=+34.28 (c 0.07, CHCl₃). ¹H NMR (600 MHz, CDCl₃): δ 7.30 (d, J=9.7 Hz, 1H), 7.17 (d, J=8.6 Hz, 2H), 6.88 (d, J=8.6 Hz, 2H), 5.99 (d, J=9.7 Hz, 1H), 5.39 (s, 1H), 5.19 (s, 1H), 4.54 (d, J=5.4 Hz, 1H), 4.49 (d, J=3.7 Hz, 1H), 4.39 (dd, J=12.3, 3.6 Hz, 1H), 3.80 (s, 3H), 3.67 (d, J=6.5 Hz, 5H), 3.58 (d, J=12.3 Hz, 1H), 3.52 (dd, J=8.8, 4.3 Hz, 1H), 2.70-2.58 (m, 2H), 1.96-1.88 (m, 1H), 1.57 (s, 3H), 1.44 (s, 3H), 1.05 (s, 3H) ppm. ¹³C NMR (150 MHz, CDCl₃): δ 201.96, 175.73, 172.54, 158.59, 151.78, 149.99, 143.58, 135.25, 131.67, 129.19, 119.17, 114.07, 95.83, 84.53, 76.99, 75.63, 55.39, 53.16, 51.87, 47.59, 46.30, 44.20, 40.94, 38.34, 31.72, 18.64, 17.91, 13.06 ppm.

HRMS (ESI-TOF): calc'd for C₃₀H₃₃IO₇ [M+Na]⁺: 655.1163, found: 655.1167. TLC:R_{f}=0.36 (2:1 hexanes:ethyl acetate). Note: compound S-93 and S-94 were separated by the PTLC (hexanes:EA=3:1 (3 times)).

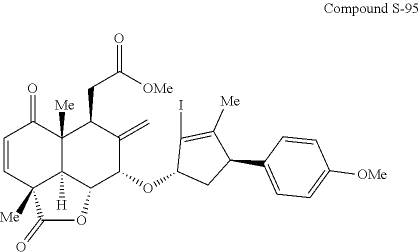

Yield: 46%. (9.3 mg). Physical State: colorless oil. [α]_{D}²⁴=−75.99 (c 0.20, CHCl₃). ¹H NMR (600 MHz, CDCl₃): δ 7.30 (d, J=9.7 Hz, 1H), 6.98-6.94 (m, 2H), 6.84-6.80 (m, 2H), 5.98 (d, J=9.7 Hz, 1H), 5.37 (d, J=1.3 Hz, 1H), 5.18 (d, J=1.8 Hz, 1H), 4.74-4.72 (m, 1H), 4.48 (d, J=3.7 Hz, 1H), 4.38 (dd, J=12.3, 3.7 Hz, 1H), 3.83-3.80 (m, 1H), 3.78 (s, 3H), 3.68 (s, 3H), 3.67 (dd, J=13.1, 3.1 Hz, 1H), 3.62 (d, J=11.6 Hz, 1H), 3.55 (d, J=12.3 Hz, 1H), 2.62 (dd, J=16.3, 11.6 Hz, 1H), 2.40 (ddd, J=13.6, 8.7, 3.4 Hz, 1H), 2.08-2.03 (m, 1H), 1.59 (s, 3H), 1.43 (s, 3H), 1.04 (s, 3H) ppm. ¹³C NMR (150 MHz, CDCl₃): δ 201.91, 175.79, 172.47, 158.46, 152.19, 149.92, 143.66, 135.56, 131.67, 128.46, 119.17, 114.16, 114.07, 96.51, 85.05, 77.32, 75.67, 55.40, 53.02, 51.86, 47.58, 46.25, 44.15, 40.95, 39.39, 31.71, 18.66, 18.01, 13.05 ppm. HRMS (ESI-TOF): calc'd for C₃₀H₃₃IO₇ [M+Na]⁺: 655.1163, found: 655.1167. TLC:R_{f}=0.33 (2:1 hexanes:ethyl acetate).

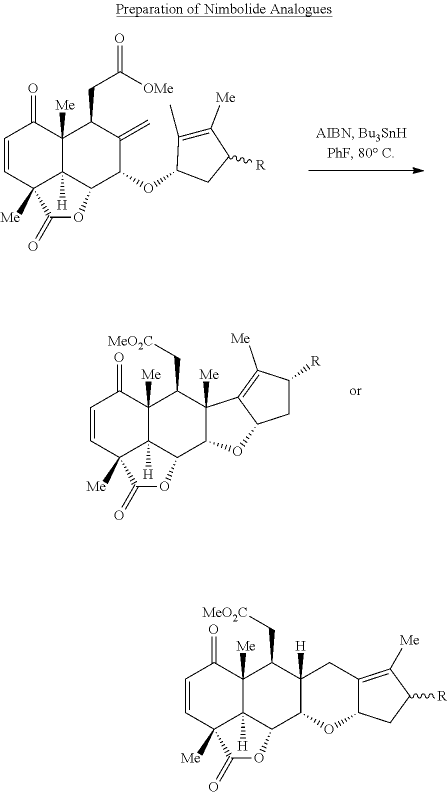

Under Ar (g), the ether compound (0.025 mmol), AIBN (8.2 mg, 0.05 mmol, 2.0 eq) and Bu₃SnH (24.9 mg, 0.1 mmol, 4.0 eq) were dissolved in 2.5 ml hexafluorobenzene, then the mixture was warmed to 80° C. After 4.5 minutes the reaction was completed then cool to 0° C. stop this reaction after that removal the solvent via rotary evaporation afford the crude mixture. Purification on PTLC afforded the target compound (41-75%).

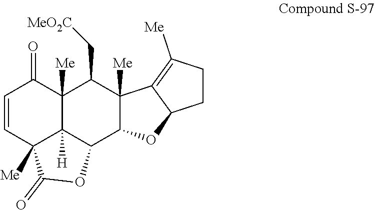

Yield: 47% (4.7 mg). Physical State: colorless oil. [α]_{D}²⁴=+159.97 (c 0.04, CHCl₃). ¹H NMR (600 MHz, CDCl₃): δ 7.27 (d, J=9.6 Hz, 1H), 5.92 (d, J=9.7 Hz, 1H), 5.18 (ddt, J=8.4, 6.6, 3.0 Hz, 1H), 4.59 (dd, J=12.5, 3.7 Hz, 1H), 4.19 (d, J=3.6 Hz, 1H), 3.60 (s, 3H), 3.35 (dd, J=15.9, 4.9 Hz, 1H), 3.18 (d, J=12.5 Hz, 1H), 2.63 (dd, J=7.3, 4.8 Hz, 1H), 2.51-2.44 (m, 1H), 2.39 (dd, J=15.8, 7.3 Hz, 1H), 2.29-2.18 (m, 2H), 1.75 (t, J=1.6 Hz, 3H), 1.67 (ddt, J=11.8, 10.3, 8.6 Hz, 1H), 1.46 (s, 3H), 1.34 (s, 3H), 1.19 (s, 3H) ppm. ¹³C NMR (150 MHz, CDCl₃): δ 201.21, 175.14, 173.03, 149.81, 144.03, 134.50, 131.22, 89.56, 82.81, 73.71, 51.76, 50.17, 47.80, 45.36, 43.82, 41.37, 39.78, 34.01, 32.34, 18.66, 17.06, 15.33, 14.65 ppm. HRMS (ESI-TOF): calc'd for C₂₃H₂₈06 [M+H]⁺: 401.1959, found: 401.1958. TLC:R_{f}=0.33 (1:1 hexanes:ethyl acetate).

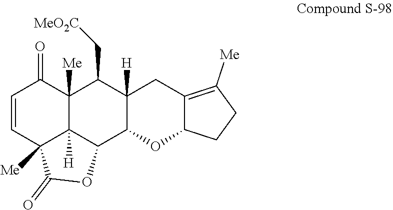

Yield: 74% (7.2 mg). Physical State: colorless oil. [α]_{D}²⁴=+39.99 (c 0.07, CHCl₃). ¹H NMR (600 MHz, CDCl₃): δ 7.24 (d, J=9.7 Hz, 1H), 5.90 (d, J=9.7 Hz, 1H), 4.50 (dt, J=14.9, 5.8 Hz, 2H), 4.20 (dd, J=3.9, 1.7 Hz, 1H), 3.69 (s, 3H), 3.14 (d, J=12.5 Hz, 1H), 3.04 (dd, J=17.3, 4.2 Hz, 1H), 2.60 (dd, J=17.3, 5.0 Hz, 1H), 2.52 (ddd, J=14.5, 7.1, 3.3 Hz, 2H), 2.31-2.17 (m, 4H), 1.98 (ddt, J=11.6, 5.3, 1.9 Hz, 1H), 1.72-1.64 (m, 1H), 1.54 (d, J=1.8 Hz, 3H), 1.44 (s, 3H), 1.15 (s, 3H) ppm. ¹³C NMR (150 MHz, CDCl₃): δ 202.34, 175.75, 173.16, 149.60, 135.21, 131.79, 128.20, 85.57, 74.95, 73.41, 51.79, 47.41, 46.27, 44.01, 37.27, 37.14, 35.35, 32.73, 29.22, 26.47, 18.81, 13.91, 13.82 ppm. HRMS (ESI-TOF): calc'd for C₁₉H₂₃N₃O₄S [M+H]⁺: 390.1482, found: 390.1491. TLC:R_{f}=0.35 (1:1 hexanes:ethyl acetate).

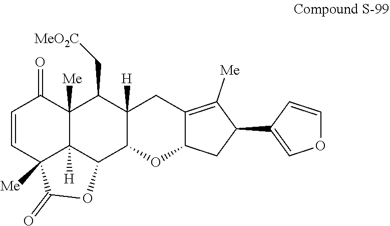

Yield: 75% (8.7 mg). Physical State: colorless oil. [α]_{D}²⁴=+39.99 (c 0.07, CHCl₃). ¹H NMR (600 MHz, CDCl₃): δ 7.31 (t, J=1.7 Hz, 1H), 7.25 (d, J=8.6 Hz, 1H), 7.15 (s, 1H), 6.07 (s, 1H), 5.92 (d, J=9.7 Hz, 1H), 4.73-4.66 (m, 1H), 4.52 (dd, J=12.5, 3.8 Hz, 1H), 4.23 (dd, J=3.9, 1.7 Hz, 1H), 3.69 (s, 3H), 3.62 (d, J=8.3 Hz, 1H), 3.15 (d, J=12.4 Hz, 1H), 3.05 (dd, J=17.3, 4.2 Hz, 1H), 2.61 (dd, J=17.2, 5.0 Hz, 1H), 2.56 (ddd, J=15.3, 8.1, 3.1 Hz, 2H), 2.28 (d, J=15.5 Hz, 1H), 2.22-2.12 (m, 2H), 2.02 (ddt, J=11.5, 3.7, 2.4 Hz, 1H), 1.46 (s, 3H), 1.45 (s, 3H), 1.16 (s, 3H) ppm. ¹³C NMR (150 MHz, CDCl₃): δ 202.25, 175.69, 173.13, 149.62, 143.31, 138.62, 137.53, 131.79, 129.17, 128.30, 109.74, 84.48, 74.83, 73.56, 51.85, 47.45, 46.28, 44.01, 43.93, 38.17, 37.32, 37.16, 32.70, 26.67, 18.81, 13.80, 12.44 ppm. HRMS (ESI-TOF): calc'd for C₂₇H₃₀O₇[M+H]⁺: 467.2064, found: 467.2062. TLC:R_{f}=0.36 (1:1 hexanes:ethyl acetate).

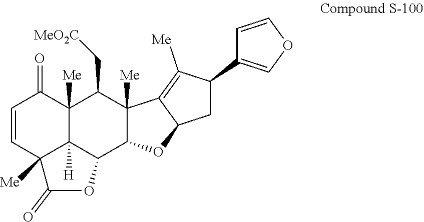

Yield: 27% (3.15 mg). Physical State: colorless oil. [α]_{D}²⁴=+86.95 (c 0.023, CHCl₃). ¹H NMR (600 MHz, CDCl₃): δ 7.38 (t, J=1.7 Hz, 1H), 7.29 (d, J=9.7 Hz, 1H), 7.26 (s, 1H), 6.21 (dd, J=1.9, 0.9 Hz, 1H), 5.93 (d, J=9.7 Hz, 1H), 5.15 (tq, J=6.5, 2.0 Hz, 1H), 4.63 (dd, J=12.6, 3.7 Hz, 1H), 4.27 (d, J=3.6 Hz, 1H), 3.73 (dd, J=10.7, 5.5 Hz, 1H), 3.64 (s, 3H), 3.35 (dd, J=16.1, 4.9 Hz, 1H), 3.20 (d, J=12.5 Hz, 1H), 2.69 (dd, J=6.9, 4.9 Hz, 1H), 2.58 (dt, J=12.0, 6.1 Hz, 1H), 2.41 (dd, J=16.1, 7.0 Hz, 1H), 1.74 (ddd, J=11.8, 10.1, 8.3 Hz, 1H), 1.54 (t, J=1.7 Hz, 3H), 1.48 (s, 3H), 1.36 (s, 3H) ppm. ¹³C NMR (150 MHz, CDCl₃): δ 201.11, 175.08, 173.03, 149.84, 145.60, 143.57, 139.61, 135.70, 131.22, 126.23, 109.74, 86.76, 82.92, 73.63, 51.87, 50.36, 47.88, 46.37, 45.36, 43.82, 42.39, 41.42, 32.24, 18.66, 17.27, 15.35, 12.67 ppm. HRMS (ESI-TOF): calc'd for C₂₇H₃₀O₇[M+H]⁺: 467.2064, found: 467.2075. TLC:R_{f}=0.36 (1:1 hexanes:ethyl acetate).

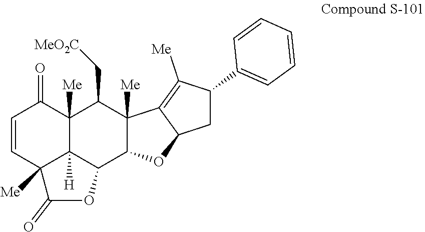

Yield: 41% (4.8 mg). Physical State: colorless oil. [α]_{D}²³=+168.24 (c 0.063, CHCl₃). ¹H NMR (600 MHz, CDCl₃): δ 7.29-7.25 (m, 3H), 7.18 (d, J=7.4 Hz, 1H), 7.14-7.06 (m, 2H), 5.93 (d, J=9.7 Hz, 1H), 5.62 (t, J=7.3 Hz, 1H), 4.63 (dd, J=12.6, 3.7 Hz, 1H), 4.31 (d, J=3.7 Hz, 1H), 3.82-3.76 (m, 1H), 3.50 (s, 3H), 3.25 (dd, J=16.2, 5.5 Hz, 1H), 3.20 (d, J=12.5 Hz, 1H), 2.80 (t, J=5.6 Hz, 1H), 2.41 (dd, J=16.2, 5.6 Hz, 1H), 2.29-2.17 (m, 2H), 1.67 (d, J=1.9 Hz, 3H), 1.48 (s, 3H), 1.39 (s, 3H), 1.23 (s, 3H) ppm. ¹³C NMR (150 MHz, CDCl₃): δ 200.96, 175.10, 173.04, 149.78, 145.39, 143.04, 137.13, 131.18, 128.72, 127.76, 126.62, 88.85, 83.18, 73.61, 60.31, 51.97, 50.56, 47.93, 45.44, 43.80, 41.72, 41.37, 32.42, 18.75, 17.44, 15.33, 13.23 ppm. HRMS (ESI-TOF): calc'd for C₂₉H₃₂06 [M+H]⁺: 477.2272, found: 477.2272. TLC:R_{f}=0.43 (1:1 hexanes:ethyl acetate).

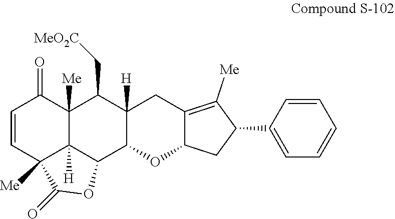

Yield: 68% (8.1 mg). Physical State: colorless oil. [α]_{D}²³=+59.81 (c 0.107, CHCl₃). ¹H NMR (600 MHz, CDCl₃): δ 7.34-7.27 (m, 3H), 7.20 (dd, J=8.3, 6.7 Hz, 3H), 5.98 (d, J=9.7 Hz, 1H), 4.54 (dd, J=12.5, 3.8 Hz, 1H), 4.51-4.46 (m, 1H), 4.27 (dd, J=3.9, 1.6 Hz, 1H), 3.61 (s, 3H), 3.55-3.49 (m, 1H), 3.29-3.20 (m, 2H), 2.78-2.68 (m, 2H), 2.59 (dd, J=15.3, 2.0 Hz, 1H), 2.45 (dd, J=17.1, 5.9 Hz, 1H), 2.31 (d, J=15.4 Hz, 1H), 1.96 (dd, J=11.6, 5.4 Hz, 1H), 1.68 (ddd, J=13.1, 8.5, 7.1 Hz, 1H), 1.46 (s, 3H), 1.29 (q, J=1.6 Hz, 3H), 1.18 (s, 3H) ppm. ¹³C NMR (150 MHz, CDCl₃): δ 202.21, 175.60, 172.99, 149.70, 144.07, 137.25, 131.89, 130.50, 128.59, 128.46, 126.50, 83.66, 74.84, 73.62, 53.18, 51.90, 47.64, 46.13, 44.01, 40.60, 37.70, 37.45, 33.35, 26.64, 18.81, 13.70, 12.23 ppm. TLC:R_{f}=0.42 (1:1 hexanes ethyl acetate). HRMS (ESI-TOF): calc'd for C₂₉H₃₂O₆ [M+H]⁺: 477.2272, found: 477.2263.

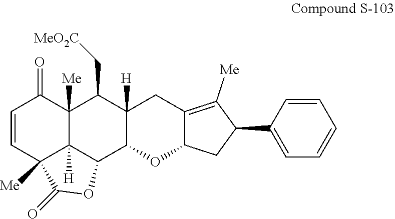

Yield: 68% (8.1 mg). Physical State: colorless oil. [α]_{D}²³=−76.82 (c 0.083, CHCl₃). ¹H NMR (600 MHz, CDCl₃): δ 7.27 (d, J=2.8 Hz, 1H), 7.26-7.23 (m, 2H), 7.17 (t, J=7.4 Hz, 1H), 7.05-6.94 (m, 2H), 5.93 (d, J=9.7 Hz, 1H), 4.81-4.75 (m, 1H), 4.54 (dd, J=12.4, 3.8 Hz, 1H), 4.27 (dd, J=3.8, 1.7 Hz, 1H), 3.74-3.71 (m, 1H), 3.69 (s, 3H), 3.17 (d, J=12.4 Hz, 1H), 3.04 (dd, J=17.4, 4.5 Hz, 1H), 2.65 (dd, J=17.4, 4.8 Hz, 1H), 2.63-2.55 (m, 2H), 2.35 (d, J=16.6 Hz, 1H), 2.29-2.19 (m, 2H), 2.09-2.04 (m, 1H), 1.45 (s, 3H), 1.39 (s, 3H), 1.18 (s, 3H) ppm. ¹³C NMR (150 MHz, CDCl₃): δ 202.26, 175.70, 173.14, 149.62, 145.11, 138.37, 131.80, 129.62, 128.67, 127.40, 126.31, 84.94, 74.85, 73.65, 54.54, 51.85, 47.48, 46.32, 44.02, 39.05, 37.34, 37.17, 32.60, 26.75, 18.83, 13.84, 12.56 ppm. HRMS (ESI-TOF): calc'd for C₂₉H₃₂06 [M+H]⁺: 477.2272, found: 477.2265. TLC:R_{f}=0.42 (1:1 hexanes:ethyl acetate).

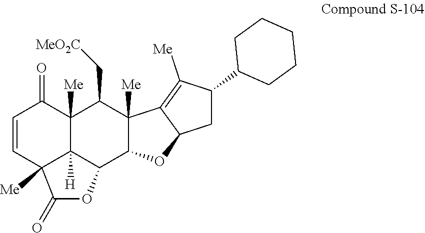

Yield: 32% (8.3 mg). Physical State: colorless oil. [α]_{D}²⁴=+103.74 (c 0.13, CHCl₃). ¹H NMR (600 MHz, CDCl₃): δ 7.26 (d, J=9.7 Hz, 1H), 5.89 (d, J=9.7 Hz, 1H), 5.27 (tt, J=7.1, 2.0 Hz, 1H), 4.59 (dd, J=12.5, 3.7 Hz, 1H), 4.21 (d, J=3.7 Hz, 1H), 3.64 (s, 3H), 3.19-3.07 (m, 2H), 2.62 (dd, J=6.5, 4.3 Hz, 1H), 2.55 (d, J=9.1 Hz, 1H), 2.34 (dd, J=16.3, 4.3 Hz, 1H), 2.11 (dd, J=12.6, 6.8 Hz, 1H), 1.81-1.66 (m, 6H), 1.66-1.61 (m, 1H), 1.52-1.47 (m, 1H), 1.45 (s, 4H), 1.41-1.34 (m, 1H), 1.32 (s, 3H), 1.30-1.22 (m, 2H), 1.21 (s, 3H), 1.15-1.04 (m, 3H) ppm. ¹³C NMR (150 MHz, CDCl₃): δ 200.87, 175.18, 172.74, 149.76, 144.17, 136.55, 131.13, 89.03, 82.74, 73.77, 60.23, 51.88, 50.68, 47.99, 45.51, 43.80, 41.24, 39.99, 35.85, 33.29, 32.64, 28.30, 27.04, 26.70, 26.62, 18.78, 17.55, 15.26, 13.36 ppm. HRMS (ESI-TOF): calc'd for C₂₉H₃₈O₆[M+H]⁺: 483.2741, found: 483.2733. TLC:R_{f}=0.54 (1:1 hexanes ethyl acetate).

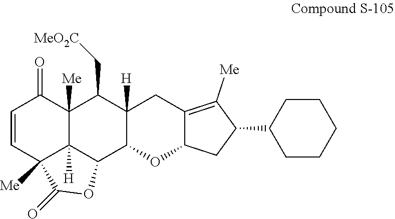

Yield: 66% (8.0 mg). Physical State: colorless oil. [α]_{D}²⁴=+18.39 (c 0.087, CHCl₃). ¹H NMR (600 MHz, CDCl₃): δ 7.24 (s, 1H), 5.91 (d, J=9.7 Hz, 1H), 4.50 (dd, J=12.4, 3.8 Hz, 1H), 4.35 (d, J=7.2 Hz, 1H), 4.21 (dd, J=3.9, 1.7 Hz, 1H), 3.66 (s, 3H), 3.11 (d, J=12.4 Hz, 1H), 2.96 (dd, J=17.1, 4.7 Hz, 1H), 2.66 (dd, J=17.1, 4.8 Hz, 1H), 2.54 (dd, J=14.9, 2.0 Hz, 1H), 2.48-2.39 (m, 2H), 2.21 (d, J=15.6 Hz, 1H), 2.11 (dt, J=13.3, 7.9 Hz, 1H), 2.01 (dd, J=11.6, 7.2 Hz, 1H), 1.72 (s, 2H), 1.64 (d, J=12.9 Hz, 1H), 1.49 (s, 3H), 1.44 (s, 4H), 1.34-1.28 (m, 2H), 1.20 (dt, J=14.7, 3.4 Hz, 1H), 1.15 (s, 3H), 1.12-0.99 (m, 2H), 0.97-0.76 (m, 3H). ppm. ¹³C NMR (150 MHz, CDCl₃): δ 202.23, 175.75, 173.09, 149.68, 137.15, 131.66, 129.44, 83.91, 74.96, 73.54, 51.99, 51.78, 47.55, 46.38, 44.05, 38.27, 37.44, 37.22, 32.76, 32.05, 30.09, 27.18, 26.77 (2 C), 26.60, 26.03, 18.82, 13.85, 12.15. HRMS (ESI-TOF): calc'd for C₂₉H₃₈O₆[M+H]⁺: 483.2741, found: 483.2733. TLC:R_{f}=0.54 (1:1 hexanes:ethyl acetate).

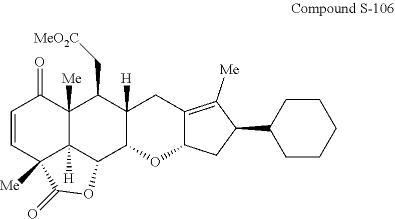

Yield: 66% (8.0 mg). Physical State: colorless oil. [α]_{D}²⁴=−9.71 (c 0.103, CHCl₃). ¹H NMR (600 MHz, CDCl₃): δ 7.23 (d, J=9.7 Hz, 1H), 5.90 (d, J=9.7 Hz, 1H), 4.49 (dd, J=12.4, 3.8 Hz, 1H), 4.46-4.40 (m, 1H), 4.17 (dd, J=3.9, 1.6 Hz, 1H), 3.68 (s, 3H), 3.13 (d, J=12.4 Hz, 1H), 2.99 (dd, J=17.4, 4.7 Hz, 1H), 2.65 (dd, J=17.4, 4.6 Hz, 1H), 2.52 (ddd, J=16.3, 7.0, 3.3 Hz, 3H), 2.20 (d, J=15.3 Hz, 1H), 2.07-1.97 (m, 2H), 1.76-1.61 (m, 5H), 1.48 (s, 3H), 1.43 (s, 3H), 1.35 (q, J=7.4 Hz, 1H), 1.31-1.26 (m, 1H), 1.15 (s, 3H), 1.09 (ddd, J=14.2, 10.7, 6.4 Hz, 3H), 0.92 (t, J=7.3 Hz, 1H), 0.71 (qd, J=12.3, 3.7 Hz, 1H) ppm. ¹³C NMR (150 MHz, CDCl₃): δ 202.36, 175.77, 173.18, 149.60, 137.02, 131.79, 128.79, 85.14, 74.98, 73.44, 53.64, 51.80, 47.40, 46.35, 44.02, 39.55, 37.21, 37.13, 32.57, 32.45, 31.83, 27.01, 26.83, 26.78, 26.63, 26.43, 18.81, 13.85, 12.44 ppm. HRMS (ESI-TOF): calc'd for C₂₉H₃₈O₆ [M+H]⁺: 483.2741, found: 483.2733. TLC:R_{f}=0.54 (1:1 hexanes:ethyl acetate).

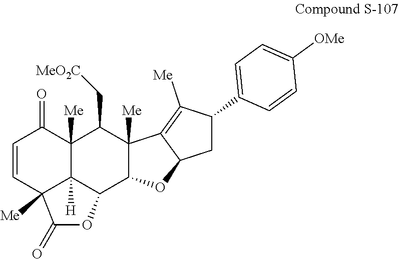

Yield: 39% (4.9 mg). Physical State: colorless oil. [α]_{D}²⁴=+114.98 (c 0.16, CHCl₃). ¹H NMR (600 MHz, CDCl₃): δ 7.28 (d, J=9.7 Hz, 1H), 7.03 (d, J=8.6 Hz, 2H), 6.80 (d, J=8.6 Hz, 2H), 5.93 (d, J=9.7 Hz, 1H), 5.60 (tt, J=7.1, 1.9 Hz, 1H), 4.63 (dd, J=12.5, 3.7 Hz, 1H), 4.30 (d, J=3.7 Hz, 1H), 3.77 (s, 3H), 3.74 (d, J=8.0 Hz, 1H), 3.53 (s, 3H), 3.25 (dd, J=16.2, 5.5 Hz, 1H), 3.19 (d, J=12.5 Hz, 1H), 2.79 (t, J=5.6 Hz, 1H), 2.41 (dd, J=16.2, 5.6 Hz, 1H), 2.25-2.17 (m, 2H), 1.66 (d, J=1.9 Hz, 3H), 1.48 (s, 3H), 1.38 (s, 3H), 1.23 (s, 3H) ppm. ¹³C NMR (150 MHz, CDCl₃): δ 200.97, 175.09, 173.12, 158.35, 149.79, 145.11, 137.35, 135.15, 131.17, 128.76, 114.02, 88.82, 83.16, 73.62, 59.53, 55.40, 52.01, 50.54, 47.92, 45.45, 43.80, 41.92, 41.37, 32.42, 18.75, 17.45, 15.32, 13.18 ppm. HRMS (ESI-TOF): calc'd for C₃₀H₃₄O₇ [M+H]⁺: 507.2377, found: 507.2369. TLC:R_{f}=0.35 (1:1 hexanes:ethyl acetate).

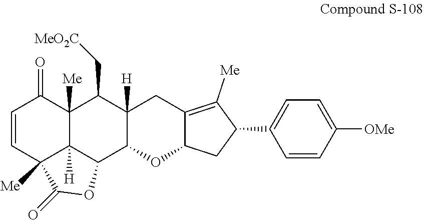

Yield: 72% (9.1 mg). Physical State: colorless oil. [α]_{D}²⁴=+62.76 (c 0.137, CHCl₃). ¹H NMR (600 MHz, CDCl₃): δ 7.28 (d, J=9.7 Hz, 1H), 7.12 (d, J=8.5 Hz, 2H), 6.86 (d, J=8.6 Hz, 2H), 5.98 (d, J=9.7 Hz, 1H), 4.54 (dd, J=12.5, 3.8 Hz, 1H), 4.47 (dt, J=8.5, 4.1 Hz, 1H), 4.26 (dd, J=3.8, 1.6 Hz, 1H), 3.79 (s, 3H), 3.62 (s, 3H), 3.47 (d, J=5.1 Hz, 1H), 3.28-3.20 (m, 2H), 2.75 (ddd, J=11.7, 5.9, 3.5 Hz, 1H), 2.69 (dt, J=13.2, 7.4 Hz, 1H), 2.57 (dd, J=15.3, 2.0 Hz, 1H), 2.44 (dd, J=17.1, 5.9 Hz, 1H), 2.33-2.27 (m, 1H), 1.94 (ddt, J=11.7, 5.6, 1.8 Hz, 1H), 1.65-1.61 (m, 1H), 1.46 (s, 3H), 1.29-1.27 (m, 3H), 1.18 (s, 3H) ppm. 13C NMR (150 MHz, CDCl₃): δ 202.23, 175.60, 172.99, 158.26, 149.71, 137.47, 136.20, 131.90, 130.21, 129.39, 113.96, 83.57, 74.86, 73.59, 55.37, 52.33, 51.91, 47.64, 46.11, 44.00, 40.76, 37.73, 37.41, 33.34, 26.63, 18.81, 13.70, 12.21 ppm. HRMS (ESI-TOF): calc'd for C₃₀H₃₄07 [M+H]⁺: 507.2377, found: 507.2371. TLC:R_{f}=0.35 (1:1 hexanes:ethyl acetate).

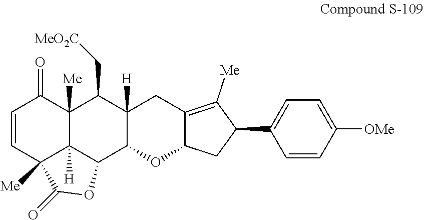

Yield: 72% (9.1 mg). Physical State: colorless oil. [α]_{D}²⁴=−58.34 (c 0.137, CHCl₃). ¹H NMR (600 MHz, CDCl₃): δ 7.25 (d, J=9.4 Hz, 1H), 6.96-6.90 (m, 2H), 6.82-6.75 (m, 2H), 5.93 (d, J=9.7 Hz, 1H), 4.80-4.73 (m, 1H), 4.53 (dd, J=12.4, 3.8 Hz, 1H), 4.26 (dd, J=3.8, 1.7 Hz, 1H), 3.77 (s, 3H), 3.69 (s, 3H), 3.67 (d, J=9.2 Hz, 1H), 3.16 (d, J=12.4 Hz, 1H), 3.03 (dd, J=17.4, 4.5 Hz, 1H), 2.65 (dd, J=17.4, 4.7 Hz, 1H), 2.61-2.55 (m, 2H), 2.33 (d, J=15.0 Hz, 1H), 2.26-2.15 (m, 2H), 2.08-2.03 (m, 1H), 1.45 (s, 3H), 1.39 (d, J=1.2 Hz, 3H), 1.17 (s, 3H) ppm. ¹³C NMR (150 MHz, CDCl₃): δ 202.28, 175.71, 173.14, 158.09, 149.62, 138.61, 137.20, 131.80, 129.29, 128.30, 114.01, 84.91, 74.87, 73.64, 55.38, 53.68, 51.84, 47.47, 46.33, 44.02, 39.19, 37.33, 37.17, 32.59, 26.76, 18.83, 13.84, 12.52 ppm. HRMS (ESI-TOF): calc'd for C₃₀H₃₄O₇[M+H]⁺: 507.2377, found: 507.2374. TLC:R_{f}=0.35 (1:1 hexanes:ethyl acetate).

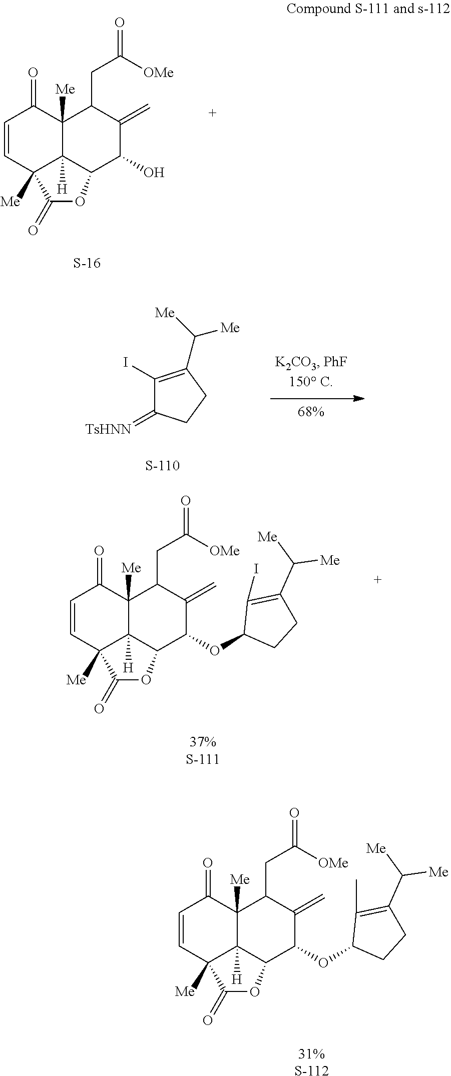

The S-16 (5.0 mg) and hydrazone (20.3 mg, 3.0 eq) were dissolved in 0.5 ml fluorobenzene, stirring at rt. After K₂CO₃ (9.0 mg, 4.0 eq) was added, the mixture was warmed to 150° C. After 1 hour the reaction was complete, then cooled to rt, purified on PTLC (hexane:EA=2:1, twice) afford the product as colorless oil (3.3 mg) in 37% yield and the other product as colorless oil (2.8 mg) in 31% yield.

Product 1 (S-111). ¹H NMR (400 MHz, CDCl₃): δ 7.29 (d, J=9.7 Hz, 1H), 5.95 (d, J=9.7 Hz, 1H), 5.39 (d, J=1.3 Hz, 1H), 5.11 (d, J=1.7 Hz, 1H), 4.73 (d, J=3.7 Hz, 1H), 4.36 (dd, J=12.4, 3.7 Hz, 1H), 3.65 (s, 3H), 3.61 (dd, J=16.4, 3.2 Hz, 1H), 3.41 (d, J=12.4 Hz, 1H), 3.29 (dd, J=11.9, 2.6 Hz, 1H), 2.74 (h, J=6.9 Hz, 1H), 2.58 (dd, J=16.4, 11.6 Hz, 1H), 2.50-2.36 (m, 2H), 2.33-2.10 (m, 2H), 1.86 (ddt, J=13.1, 8.8, 4.5 Hz, 1H), 1.42 (s, 3H), 1.10-0.99 (m, 6H), 0.95 (d, J=6.9 Hz, 3H).

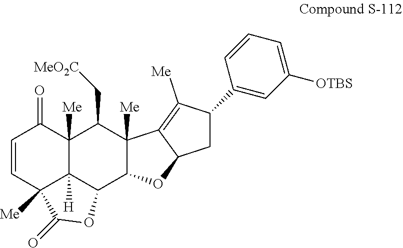

¹H NMR (600 MHz, CDCl₃): δ 7.28 (d, J=9.7 Hz, 1H), 7.11 (t, J=7.8 Hz, 1H), 6.70 (dt, J=7.8, 1.3 Hz, 1H), 6.65 (ddd, J=8.0, 2.4, 1.0 Hz, 1H), 6.60 (t, J=2.0 Hz, 1H), 5.93 (d, J=9.7 Hz, 1H), 5.60 (tt, J=6.9, 2.0 Hz, 1H), 4.63 (dd, J=12.5, 3.7 Hz, 1H), 4.28 (d, J=3.7 Hz, 1H), 3.71 (d, J=8.5 Hz, 1H), 3.47 (s, 3H), 3.26 (dd, J=16.1, 5.3 Hz, 1H), 3.20 (d, J=12.6 Hz, 1H), 2.78 (t, J=5.6 Hz, 1H), 2.40 (dd, J=16.1, 6.0 Hz, 1H), 2.27 (dd, J=12.5, 6.9 Hz, 1H), 2.20 (ddd, J=12.5, 9.1, 7.7 Hz, 1H), 1.67 (d, J=1.8 Hz, 3H), 1.47 (s, 3H), 1.38 (s, 3H), 1.22 (s, 3H), 0.97 (s, 9H), 0.18 (d, J=5.7 Hz, 6H) ppm. ¹³C NMR (150 MHz, CDCl₃): δ 200.96, 175.10, 172.82, 155.82, 149.73, 145.18, 144.75, 137.19, 131.23, 129.64, 120.26, 120.18, 118.13, 88.89, 83.11, 73.62, 60.28, 51.97, 50.62, 47.90, 45.43, 43.80, 41.48, 41.31, 32.40, 25.86, 18.74, 17.45, 15.38, 13.27, −4.23, −4.28 ppm.

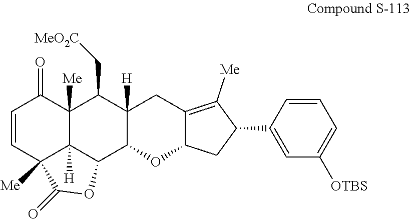

¹H NMR (600 MHz, CDCl₃): δ 7.13 (t, J=7.8 Hz, 1H), 6.84 (d, J=11.5 Hz, 1H), 6.71-6.66 (m, 2H), 6.60 (t, J=2.1 Hz, 1H), 6.05 (dd, J=11.5, 1.1 Hz, 1H), 5.51-5.45 (m, 1H), 4.29 (dd, J=11.6, 3.0 Hz, 1H), 4.26 (d, J=3.1 Hz, 1H), 3.78 (d, J=8.7 Hz, 1H), 3.67 (s, 3H), 2.94 (dd, J=13.2, 7.5 Hz, 1H), 2.70 (t, J=11.6 Hz, 1H), 2.61-2.56 (m, 2H), 2.46 (dd, J=15.9, 5.8 Hz, 1H), 2.32-2.18 (m, 5H), 1.74 (d, J=1.8 Hz, 3H), 1.41 (s, 3H), 1.32 (s, 3H), 0.98 (s, 9H), 0.19 (d, J=3.3 Hz, 6H) ppm.

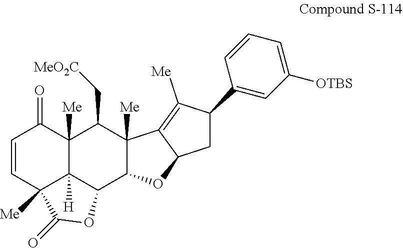

¹H NMR (400 MHz, CDCl₃): δ 7.29 (d, J=9.7 Hz, 1H), 7.15 (t, J=7.8 Hz, 1H), 6.71 (dd, J=8.0, 2.6 Hz, 2H), 6.58 (t, J=2.1 Hz, 1H), 5.94 (d, J=9.7 Hz, 1H), 5.22-5.14 (m, 1H), 4.65 (dd, J=12.5, 3.7 Hz, 1H), 4.32 (d, J=3.6 Hz, 1H), 3.70 (t, J=8.1 Hz, 1H), 3.65 (s, 3H), 3.36 (dd, J=16.1, 4.9 Hz, 1H), 3.22 (d, J=12.5 Hz, 1H), 2.73 (dd, J=6.8, 4.9 Hz, 1H), 2.66 (dt, J=12.2, 6.2 Hz, 1H), 2.42 (dd, J=16.1, 6.9 Hz, 1H), 1.80 (ddd, J=12.1, 9.9, 8.1 Hz, 1H), 1.48 (s, 3H), 1.46 (d, J=1.6 Hz, 3H), 1.38 (s, 3H), 1.22 (s, 3H), 0.98 (s, 9H), 0.18 (s, 6H) ppm.

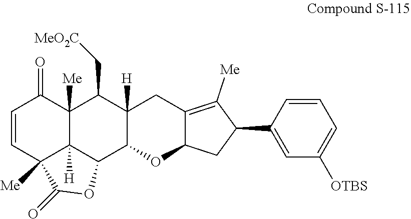

¹H NMR (600 MHz, CDCl₃): δ 7.16 (t, J=7.8 Hz, 1H), 6.85 (d, J=11.5 Hz, 1H), 6.78-6.66 (m, 2H), 6.60 (t, J=2.0 Hz, 1H), 6.06 (dd, J=11.4, 1.0 Hz, 1H), 5.17-5.11 (m, 1H), 4.32 (dd, J=11.6, 3.1 Hz, 1H), 4.28 (d, J=3.0 Hz, 1H), 3.77 (t, J=8.0 Hz, 1H), 3.71 (s, 3H), 2.92 (dd, J=13.3, 7.7 Hz, 1H), 2.72 (t, J=11.7 Hz, 1H), 2.67 (dt, J=12.3, 6.2 Hz, 1H), 2.56 (dd, J=13.5, 3.8 Hz, 1H), 2.54-2.43 (m, 2H), 2.29-2.18 (m, 2H), 1.81 (ddd, J=12.1, 9.8, 8.1 Hz, 1H), 1.51 (t, J=1.6 Hz, 3H), 1.42 (s, 3H), 1.30 (s, 3H), 0.98 (s, 9H), 0.19 (s, 6H) ppm.

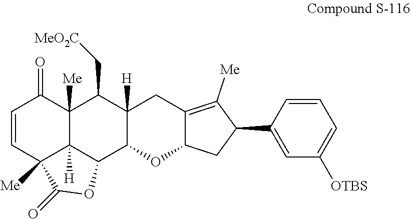

¹H NMR (400 MHz, CDCl₃): δ 7.25 (d, J=9.7 Hz, 1H), 7.10 (t, J=7.8 Hz, 1H), 6.64 (ddt, J=11.5, 7.6, 1.2 Hz, 2H), 6.47 (t, J=2.0 Hz, 1H), 5.93 (d, J=9.7 Hz, 1H), 4.75 (t, J=7.2 Hz, 1H), 4.53 (dd, J=12.5, 3.8 Hz, 1H), 4.26 (dd, J=3.9, 1.6 Hz, 1H), 3.69 (s, 3H), 3.66 (d, J=5.2 Hz, 1H), 3.16 (d, J=12.4 Hz, 1H), 3.04 (dd, J=17.3, 4.4 Hz, 1H), 2.65 (dd, J=17.3, 4.7 Hz, 1H), 2.61-2.54 (m, 2H), 2.38-2.30 (m, 1H), 2.22 (dd, J=7.3, 5.4 Hz, 2H), 2.06 (d, J=13.1 Hz, 1H), 1.45 (s, 3H), 1.39 (d, J=1.8 Hz, 3H), 1.17 (s, 3H), 0.96 (s, 9H), 0.17 (s, 6H) ppm.

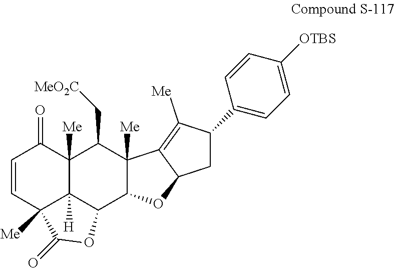

¹H NMR (400 MHz, CDCl₃); δ7.28 (d, J=9.7 Hz, 1H), 6.95 (d, J=8.5 Hz, 2H), 6.72 (d, J=8.5 Hz, 2H), 5.93 (d, J=9.7 Hz, 1H), 5.66-5.56 (m, 1H), 4.62 (dd, J=12.5, 3.7 Hz, 1H), 4.29 (d, J=3.6 Hz, 1H), 3.72 (d, J=7.7 Hz, 1H), 3.48 (s, 3H), 3.27 (dd, J=16.2, 5.3 Hz, 1H), 3.20 (d, J=12.5 Hz, 1H), 2.79 (t, J=5.6 Hz, 1H), 2.42 (dd, J=16.2, 6.0 Hz, 1H), 2.25-2.15 (m, 2H), 1.65 (d, J=1.8 Hz, 3H), 1.47 (s, 3H), 1.38 (s, 3H), 1.23 (s, 3H), 0.96 (s, 9H), 0.16 (s, 6H) ppm.

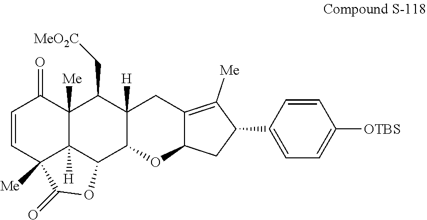

¹H NMR (400 MHz, CDCl₃): δ 6.95 (d, J=8.5 Hz, 2H), 6.84 (d, J=11.5 Hz, 1H), 6.73 (d, J=8.5 Hz, 2H), 6.05 (dd, J=11.5, 1.1 Hz, 1H), 5.52-5.44 (m, 1H), 4.34-4.23 (m, 2H), 3.78 (d, J=7.7 Hz, 1H), 3.69 (s, 3H), 2.94 (dd, J=13.3, 7.5 Hz, 1H), 2.70 (t, J=11.4 Hz, 1H), 2.58 (dt, J=15.9, 3.7 Hz, 2H), 2.45 (dd, J=15.7, 5.9 Hz, 1H), 2.28-2.14 (m, 4H), 1.72 (d, J=1.8 Hz, 3H), 1.41 (s, 3H), 1.32 (s, 3H), 0.97 (s, 9H), 0.18 (s, 6H) ppm.

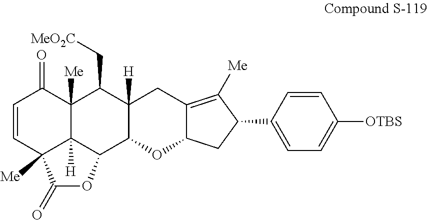

¹H NMR (400 MHz, CDCl₃): δ 7.28 (d, J=9.8 Hz, 1H), 7.03 (d, J=8.4 Hz, 2H), 6.78 (d, J=8.4 Hz, 2H), 5.98 (d, J=9.7 Hz, 1H), 4.53 (dd, J=12.4, 3.8 Hz, 1H), 4.50-4.42 (m, 1H), 4.26 (dd, J=3.7, 1.6 Hz, 1H), 3.61 (s, 3H), 3.45 (s, 1H), 3.27-3.16 (m, 2H), 2.80-2.64 (m, 2H), 2.58 (dd, J=15.4, 1.9 Hz, 1H), 2.45 (dd, J=17.0, 5.8 Hz, 1H), 2.29 (d, J=15.6 Hz, 1H), 1.95 (dd, J=11.5, 5.1 Hz, 1H), 1.65 (ddd, J=13.3, 8.7, 7.3 Hz, 2H), 1.46 (s, 3H), 1.26 (q, J=1.6 Hz, 3H), 1.18 (s, 3H), 0.97 (s, 9H), 0.18 (d, J=0.9 Hz, 6H) ppm.

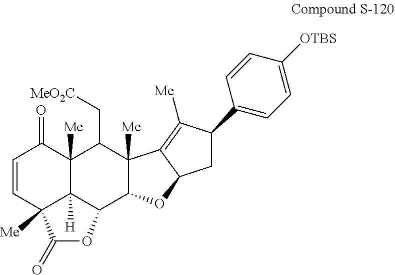

¹H NMR (400 MHz, CDCl₃): δ 7.29 (d, J=9.8 Hz, 1H), 6.96 (d, J=8.3 Hz, 2H), 6.77 (d, J=8.2 Hz, 2H), 5.94 (d, J=9.7 Hz, 1H), 5.17 (s, 1H), 4.65 (dd, J=12.6, 3.6 Hz, 1H), 4.31 (d, J=3.6 Hz, 1H), 3.70 (d, J=5.7 Hz, 1H), 3.65 (d, J=0.8 Hz, 3H), 3.36 (dd, J=16.2, 4.9 Hz, 1H), 3.22 (d, J=12.5 Hz, 1H), 2.72 (dd, J=6.9, 5.0 Hz, 1H), 2.64 (dt, J=12.1, 6.1 Hz, 1H), 2.42 (dd, J=16.1, 6.8 Hz, 1H), 1.82-1.71 (m, 2H), 1.48 (s, 3H), 1.43 (d, J=2.1 Hz, 3H), 1.38 (s, 3H), 1.22 (s, 3H), 0.98 (d, J=0.9 Hz, 9H), 0.19 (d, J=0.9 Hz, 6H) ppm.

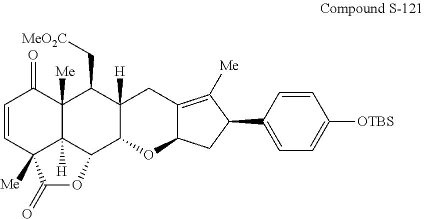

¹H NMR (400 MHz, CDCl₃): δ 6.98 (d, J=8.4 Hz, 2H), 6.85 (d, J=11.5 Hz, 1H), 6.81-6.74 (m, 3H), 6.05 (d, J=11.5 Hz, 1H), 5.13 (s, 1H), 4.34-4.26 (m, 2H), 3.76 (s, 1H), 3.71 (s, 3H), 2.94-2.88 (m, 1H), 2.71 (d, J=11.5 Hz, 1H), 2.68-2.63 (m, 1H), 2.56 (d, J=13.4 Hz, 1H), 2.49 (dd, J=11.7, 5.1 Hz, 2H), 2.35 (t, J=7.5 Hz, 1H), 1.79 (dd, J=20.1, 10.0 Hz, 2H), 1.49 (d, J=1.6 Hz, 3H), 1.41 (s, 3H), 1.30 (s, 3H), 0.98 (s, 9H), 0.19 (s, 6H) ppm.

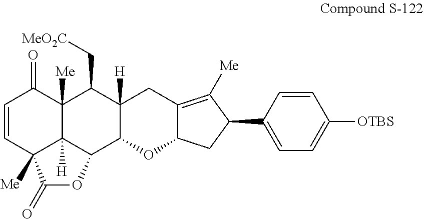

¹H NMR (600 MHz, CDCl₃): δ 7.24 (s, 1H), 6.85 (d, J=8.5 Hz, 2H), 6.71 (d, J=8.5 Hz, 2H), 5.92 (d, J=9.7 Hz, 1H), 4.75 (t, J=7.1 Hz, 1H), 4.53 (dd, J=12.4, 3.8 Hz, 1H), 4.25 (dd, J=3.8, 1.7 Hz, 1H), 3.69 (s, 3H), 3.65 (d, J=7.8 Hz, 1H), 3.16 (d, J=12.4 Hz, 1H), 3.03 (dd, J=17.4, 4.6 Hz, 1H), 2.65 (dd, J=17.4, 4.7 Hz, 1H), 2.61-2.53 (m, 2H), 2.37-2.30 (m, 1H), 2.26-2.16 (m, 2H), 2.07-2.03 (m, 1H), 1.45 (s, 3H), 1.37 (d, J=1.7 Hz, 3H), 1.17 (s, 3H), 0.96 (s, 9H), 0.17 (s, 6H) ppm. ¹³C NMR (150 MHz, CDCl₃): δ 202.28, 175.72, 173.14, 154.00, 149.61, 138.73, 137.75, 131.80, 129.14, 128.24, 120.08, 84.94, 74.88, 73.64, 53.77, 51.83, 47.47, 46.33, 44.02, 39.13, 37.33, 37.17, 32.58, 26.77, 25.81, 18.83, 13.84, 12.51, −4.29 ppm.

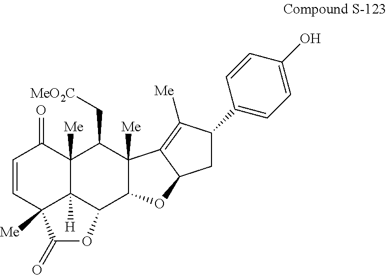

¹H NMR (400 MHz, CDCl₃): δ 7.28 (d, J=9.7 Hz, 1H), 6.98 (d, J=8.5 Hz, 2H), 6.73 (d, J=8.5 Hz, 2H), 5.93 (d, J=9.7 Hz, 1H), 5.64-5.54 (m, 1H), 4.65 (d, J=5.3 Hz, 1H), 4.65-4.59 (m, 1H), 4.29 (d, J=3.7 Hz, 1H), 3.73 (d, J=7.2 Hz, 1H), 3.53 (s, 3H), 3.25 (dd, J=16.2, 5.4 Hz, 1H), 3.19 (d, J=12.5 Hz, 1H), 2.79 (t, J=5.6 Hz, 1H), 2.41 (dd, J=16.2, 5.6 Hz, 1H), 2.25-2.15 (m, 2H), 1.66 (d, J=1.8 Hz, 3H), 1.48 (s, 3H), 1.38 (s, 3H), 1.23 (s, 3H) ppm.

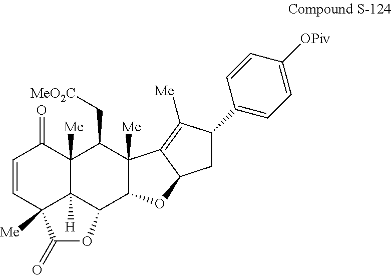

¹H NMR (400 MHz, CDCl₃): δ 7.30-7.27 (m, 1H), 7.12 (d, J=8.5 Hz, 2H), 6.94 (d, J=8.5 Hz, 2H), 5.93 (d, J=9.7 Hz, 1H), 5.66-5.57 (m, 1H), 4.63 (dd, J=12.5, 3.7 Hz, 1H), 4.30 (d, J=3.6 Hz, 1H), 3.79 (d, J=7.9 Hz, 1H), 3.51 (s, 3H), 3.26 (dd, J=16.2, 5.3 Hz, 1H), 3.19 (d, J=12.5 Hz, 1H), 2.79 (t, J=5.5 Hz, 1H), 2.40 (dd, J=16.2, 5.8 Hz, 1H), 2.29-2.18 (m, 2H), 1.67 (d, J=1.8 Hz, 3H), 1.48 (s, 3H), 1.39 (s, 3H), 1.33 (s, 9H), 1.23 (s, 3H) ppm.

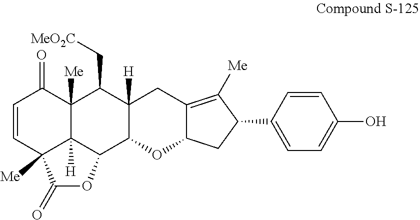

¹H NMR (400 MHz, CDCl₃): δ 7.28 (d, J=9.7 Hz, 1H), 7.07 (d, J=8.5 Hz, 2H), 6.78 (d, J=8.6 Hz, 2H), 5.98 (d, J=9.7 Hz, 1H), 4.62 (s, 1H), 4.54 (dd, J=12.4, 3.8 Hz, 1H), 4.45 (d, J=7.6 Hz, 1H), 4.26 (dd, J=3.8, 1.6 Hz, 1H), 3.61 (s, 3H), 3.44 (d, J=10.0 Hz, 1H), 3.28-3.19 (m, 2H), 2.79-2.64 (m, 2H), 2.60-2.53 (m, 1H), 2.43 (dd, J=17.1, 5.9 Hz, 1H), 2.29 (d, J=15.7 Hz, 1H), 1.94 (dd, J=11.6, 5.3 Hz, 1H), 1.68-1.57 (m, 2H), 1.46 (s, 3H), 1.27 (d, J=1.7 Hz, 3H), 1.18 (s, 3H) ppm.

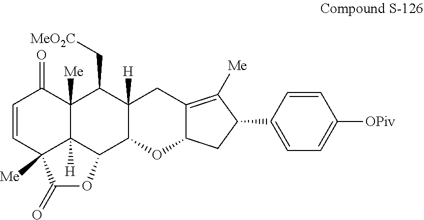

¹H NMR (400 MHz, CDCl₃): δ 7.28 (d, J=10.1 Hz, 1H), 7.20 (d, J=8.4 Hz, 2H), 7.00 (d, J=8.3 Hz, 2H), 5.98 (dd, J=9.7, 0.8 Hz, 1H), 4.53 (dd, J=12.5, 3.8 Hz, 1H), 4.47 (d, J=8.1 Hz, 1H), 4.26 (d, J=3.8 Hz, 1H), 3.62 (s, 3H), 3.52 (s, 1H), 3.31-3.17 (m, 2H), 2.80-2.65 (m, 2H), 2.57 (d, J=15.3 Hz, 1H), 2.40 (dd, J=17.1, 6.0 Hz, 1H), 2.30 (d, J=15.7 Hz, 1H), 1.93 (dd, J=11.5, 5.1 Hz, 1H), 1.64 (dt, J=13.1, 7.9 Hz, 2H), 1.46 (s, 3H), 1.34 (s, 9H), 1.29 (s, 3H), 1.18 (s, 3H) ppm.

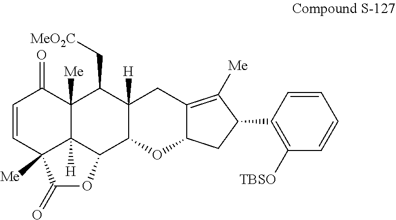

¹H NMR (600 MHz, CDCl₃): δ 7.28 (s, 1H), 7.14 (dd, J=7.6, 1.8 Hz, 1H), 7.05 (td, J=7.6, 1.8 Hz, 1H), 6.96 (td, J=7.4, 1.3 Hz, 1H), 6.77 (dd, J=8.0, 1.3 Hz, 1H), 5.97 (d, J=9.7 Hz, 1H), 4.53 (dd, J=12.4, 3.8 Hz, 1H), 4.49 (d, J=8.1 Hz, 1H), 4.27 (dd, J=4.0, 1.6 Hz, 1H), 4.16-4.10 (m, 1H), 3.63 (s, 3H), 3.27-3.17 (m, 2H), 2.77-2.66 (m, 2H), 2.60 (dd, J=15.3, 1.9 Hz, 1H), 2.48 (dd, J=17.1, 5.8 Hz, 1H), 2.38-2.27 (m, 2H), 1.99-1.93 (m, 1H), 1.45 (s, 3H), 1.32 (q, J=1.6 Hz, 3H), 1.18 (s, 3H), 0.99 (s, 9H), 0.21 (d, J=7.4 Hz, 6H) ppm. 13C NMR (150 MHz, CDCl₃): δ 202.23, 175.60, 173.03, 153.43, 149.70, 137.36, 134.51, 131.88, 130.23, 129.38, 126.91, 121.95, 118.43, 83.91, 74.86, 73.65, 51.92, 47.62, 46.15, 44.32, 44.01, 39.75, 37.73, 37.47, 33.37, 29.85, 26.71, 25.97, 18.84, 13.73, 12.22, −3.87, −4.07 ppm.

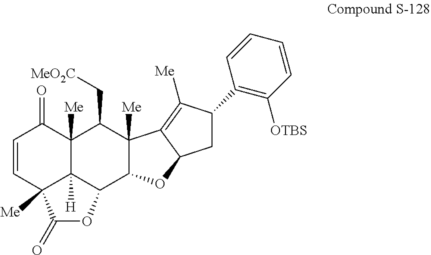

¹H NMR (400 MHz, CDCl₃): δ 7.28 (s, 1H), 7.06 (td, J=7.6, 1.9 Hz, 1H), 6.93 (dd, J=7.7, 1.8 Hz, 1H), 6.86-6.80 (m, 1H), 6.77 (dd, J=8.1, 1.2 Hz, 1H), 5.91 (d, J=9.7 Hz, 1H), 5.47-5.36 (m, 1H), 4.63 (dd, J=12.5, 3.7 Hz, 1H), 4.32 (d, J=3.6 Hz, 1H), 4.24 (d, J=8.1 Hz, 1H), 3.67 (s, 3H), 3.24-3.14 (m, 2H), 2.80-2.73 (m, 1H), 2.46 (dd, J=16.3, 4.5 Hz, 1H), 2.20-2.07 (m, 2H), 1.71 (d, J=1.8 Hz, 3H), 1.47 (s, 3H), 1.40 (s, 3H), 1.24 (s, 3H), 1.01 (s, 9H), 0.23 (d, J=10.6 Hz, 6H) ppm.

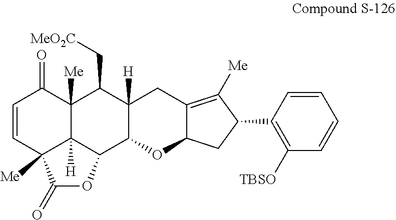

¹H NMR (400 MHz, CDCl₃): δ 7.09 (td, J=7.7, 1.8 Hz, 1H), 6.93 (dd, J=7.6, 1.8 Hz, 1H), 6.88-6.77 (m, 3H), 6.07-6.00 (m, 1H), 5.29 (d, J=6.1 Hz, 1H), 4.33-4.20 (m, 3H), 3.78 (s, 3H), 2.93 (dd, J=13.1, 6.5 Hz, 1H), 2.72-2.63 (m, 2H), 2.59 (d, J=13.2 Hz, 1H), 2.47 (dd, J=15.8, 5.3 Hz, 1H), 2.35 (t, J=7.5 Hz, 1H), 2.24 (s, 2H), 2.18-2.10 (m, 2H), 1.77 (d, J=1.8 Hz, 3H), 1.41 (s, 3H), 1.33 (s, 3H), 1.01 (s, 9H), 0.25 (d, J=7.0 Hz, 6H) ppm.

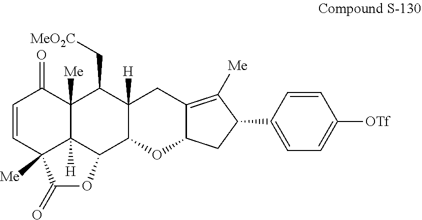

¹H NMR (600 MHz, CDCl₃): δ 7.27 (d, J=1.8 Hz, 1H), 7.17 (d, J=8.7 Hz, 2H), 7.09 (d, J=8.7 Hz, 2H), 5.94 (d, J=9.7 Hz, 1H), 4.75 (d, J=7.3 Hz, 1H), 4.55 (dd, J=12.5, 3.8 Hz, 1H), 4.27 (dd, J=3.8, 1.7 Hz, 1H), 3.81-3.74 (m, 1H), 3.71 (s, 3H), 3.16 (d, J=12.5 Hz, 1H), 3.12-3.02 (m, 1H), 2.67-2.54 (m, 3H), 2.40-2.33 (m, 1H), 2.29 (ddd, J=13.9, 9.6, 6.3 Hz, 1H), 2.19 (ddd, J=13.9, 7.6, 1.9 Hz, 1H), 2.10-2.06 (m, 1H), 1.47 (s, 3H), 1.40 (d, J=1.8 Hz, 3H), 1.19 (s, 3H) ppm. ¹³C NMR (150 MHz, CDCl₃): δ 202.15, 175.63, 173.09, 149.60, 148.04, 145.68, 137.56, 131.78, 130.61, 129.12, 121.50, 84.57, 74.71, 73.66, 53.87, 51.86, 47.48, 46.25, 43.99, 38.89, 37.37, 37.13, 32.64, 26.70, 18.80, 13.78, 12.53 ppm.

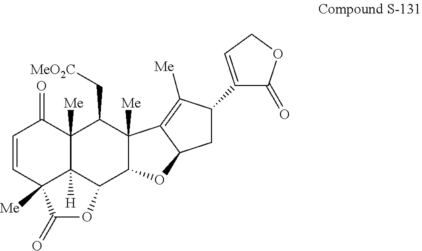

¹H NMR (600 MHz, CDCl₃): δ 7.27 (d, J=9.8 Hz, 1H), 7.19 (q, J=1.6 Hz, 1H), 5.90 (d, J=9.7 Hz, 1H), 5.37 (tq, J=6.4, 1.9 Hz, 1H), 4.80-4.70 (m, 2H), 4.61 (dd, J=12.5, 3.7 Hz, 1H), 4.29 (d, J=3.6 Hz, 1H), 3.64 (s, 3H), 3.59 (d, J=8.7 Hz, 1H), 3.24 (dd, J=16.4, 5.5 Hz, 1H), 3.12 (d, J=12.5 Hz, 1H), 2.62 (t, J=5.5 Hz, 1H), 2.38 (dd, J=16.4, 5.5 Hz, 1H), 2.17 (dd, J=12.2, 6.4 Hz, 1H), 2.07 (dt, J=12.2, 8.5 Hz, 1H), 1.75 (d, J=1.8 Hz, 3H), 1.47 (s, 3H), 1.37 (s, 3H), 1.22 (s, 3H) ppm. ¹³C NMR (150 MHz, CDCl₃): δ 200.95, 174.89, 174.29, 173.62, 149.90, 147.29, 145.02, 135.04, 134.35, 130.97, 87.98, 83.12, 73.27, 70.41, 51.92, 50.41, 48.80, 47.73, 45.44, 43.75, 41.28, 39.99, 32.40, 18.71, 17.20, 15.16, 13.26 ppm.

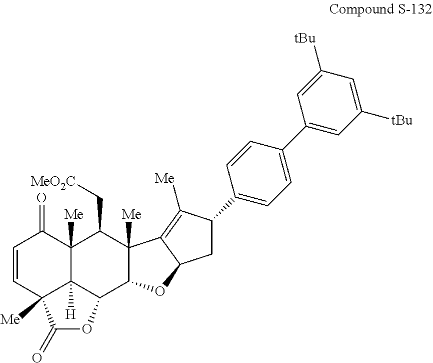

¹H NMR (600 MHz, CDCl₃): δ 7.48 (d, J=8.2 Hz, 2H), 7.41 (d, J=1.8 Hz, 1H), 7.37 (d, J=1.8 Hz, 2H), 7.29 (d, J=9.7 Hz, 1H), 7.18 (d, J=8.2 Hz, 2H), 5.94 (d, J=9.7 Hz, 1H), 5.68-5.64 (m, 1H), 4.64 (dd, J=12.5, 3.7 Hz, 1H), 4.33 (d, J=3.7 Hz, 1H), 3.85-3.82 (m, 1H), 3.52 (s, 3H), 3.27 (dd, J=16.3, 5.4 Hz, 1H), 3.21 (d, J=12.5 Hz, 1H), 2.82 (t, J=5.6 Hz, 1H), 2.44 (dd, J=16.3, 5.7 Hz, 1H), 2.31 (dd, J=12.5, 6.8 Hz, 1H), 2.27-2.23 (m, 1H), 1.72 (d, J=1.8 Hz, 4H), 1.49 (s, 3H), 1.41 (s, 3H), 1.36 (s, 18H), 1.24 (s, 3H) ppm.

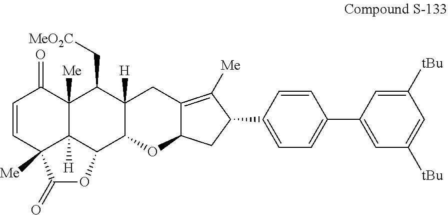

¹H NMR (600 MHz, CDCl₃): δ 7.52-7.46 (m, 1H), 7.46-7.41 (m, 1H), 7.38 (d, J=1.9 Hz, 1H), 7.18 (d, J=8.0 Hz, 1H), 6.85 (d, J=11.5 Hz, 1H), 6.06 (d, J=11.5 Hz, 1H), 5.54 (s, 1H), 5.37-5.32 (m, 1H), 4.31 (d, J=10.3 Hz, 2H), 3.90 (d, J=8.4 Hz, 1H), 3.71 (s, 3H), 2.95 (dd, J=13.4, 7.8 Hz, 1H), 2.71 (t, J=11.3 Hz, 1H), 2.64-2.58 (m, 1H), 2.48 (dd, J=15.9, 6.3 Hz, 1H), 2.35 (t, J=7.6 Hz, 2H), 2.31-2.23 (m, 2H), 1.78 (d, J=1.8 Hz, 3H), 1.42 (s, 3H), 1.34 (s, 3H), 1.25 (s, 18H) ppm.

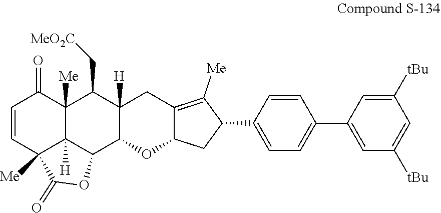

¹H NMR (600 MHz, CDCl₃): δ 7.57-7.53 (m, 2H), 7.41 (q, J=1.5 Hz, 3H), 7.30-7.27 (m, 2H), 5.99 (d, J=9.7 Hz, 1H), 4.55 (dd, J=12.4, 3.8 Hz, 1H), 4.51 (t, J=7.5 Hz, 1H), 4.28 (dd, J=3.9, 1.6 Hz, 1H), 3.63 (s, 3H), 3.57 (s, 1H), 3.29-3.22 (m, 2H), 2.81-2.71 (m, 2H), 2.61 (dd, J=15.2, 2.0 Hz, 1H), 2.46 (dd, J=17.1, 5.9 Hz, 1H), 2.33 (d, J=17.0 Hz, 1H), 1.97 (dd, J=11.6, 5.3 Hz, 1H), 1.71 (ddd, J=13.1, 8.6, 7.2 Hz, 1H), 1.47 (s, 3H), 1.37 (s, 18H), 1.34 (q, J=1.5 Hz, 3H), 1.19 (s, 3H) ppm.

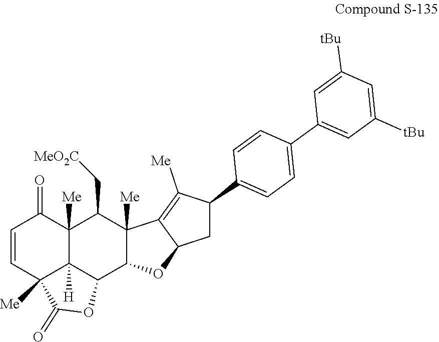

¹H NMR (400 MHz, CDCl₃): δ 7.53 (d, J=8.1 Hz, 2H), 7.41 (dd, J=10.7, 1.8 Hz, 3H), 7.30 (d, J=9.7 Hz, 1H), 7.18 (d, J=8.1 Hz, 2H), 5.95 (d, J=9.7 Hz, 1H), 5.28-5.16 (m, 1H), 4.67 (dd, J=12.5, 3.7 Hz, 1H), 4.36 (d, J=3.6 Hz, 1H), 3.82 (t, J=7.9 Hz, 1H), 3.67 (s, 3H), 3.38 (dd, J=16.2, 4.9 Hz, 1H), 3.23 (d, J=12.5 Hz, 1H), 2.72 (ddt, J=18.2, 12.2, 5.6 Hz, 2H), 2.44 (dd, J=16.1, 6.9 Hz, 1H), 2.35 (t, J=7.5 Hz, 1H), 1.86 (ddd, J=12.1, 9.8, 8.1 Hz, 1H), 1.52 (d, J=1.6 Hz, 3H), 1.49 (s, 3H), 1.42 (s, 3H), 1.37 (s, 18H) ppm.

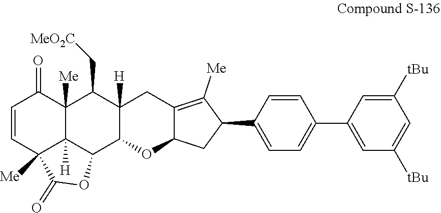

¹H NMR (400 MHz, CDCl₃): δ 7.56-7.52 (m, 2H), 7.43 (t, J=1.8 Hz, 1H), 7.40 (d, J=1.8 Hz, 2H), 7.20 (d, J=8.1 Hz, 2H), 6.86 (d, J=11.5 Hz, 1H), 6.06 (dd, J=11.4, 1.0 Hz 5.18 (s, 1H), 4.34 (d, J=10.3 Hz, 2H), 3.92-3.85 (m, 1H), 3.73 (s, 3H), 2.93 (dd, J=13.3, 7.2 Hz, 1H), 2.79-2.68 (m, 2H), 2.54 (ddd, J=23.3, 11.9, 5.1 Hz, 3H), 2.30-2.20 (m, 2H), 1.91-1.84 (m, 1H), 1.57 (t, J=1.6 Hz, 3H), 1.42 (s, 3H), 1.38 (s, 18H), 1.33 (s, 3H) ppm.

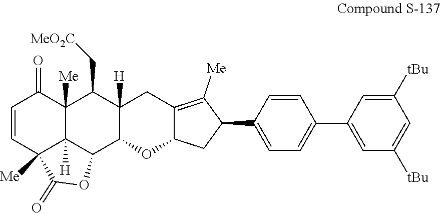

¹H NMR (400 MHz, CDCl₃): δ 7.51-7.45 (m, 2H), 7.41 (t, J=1.8 Hz, 1H), 7.38 (d, J=1.8 Hz, 2H), 7.26 (d, J=9.7 Hz, 2H), 7.15-7.03 (m, 2H), 5.94 (d, J=9.7 Hz, 1H), 4.81 (t, J=7.1 Hz, 1H), 4.55 (dd, J=12.4, 3.8 Hz, 1H), 4.28 (dd, J=3.9, 1.6 Hz, 1H), 3.77 (d, J=4.6 Hz, 1H), 3.71 (s, 3H), 3.18 (d, J=12.4 Hz, 1H), 3.05 (dd, J=17.2, 4.3 Hz, 1H), 2.74-2.56 (m, 3H), 2.41-2.33 (m, 1H), 2.28 (dd, J=7.4, 5.3 Hz, 2H), 2.08 (dd, J=11.5, 5.3 Hz, 1H), 1.46 (s, 3H), 1.44 (d, J=1.8 Hz, 3H), 1.37 (s, 18H), 1.19 (s, 3H) ppm.

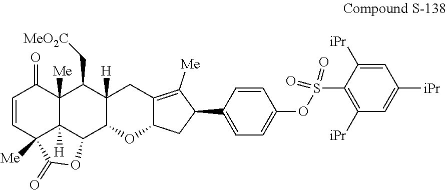

¹H NMR (400 MHz, CDCl₃): δ 7.25 (d, J=8.7 Hz, 1H), 7.17 (s, 2H), 6.95-6.84 (m, 4H), 5.92 (d, J=9.7 Hz, 1H), 4.70 (s, 1H), 4.52 (dd, J=12.4, 3.8 Hz, 1H), 4.23 (dd, J=3.9, 1.6 Hz, 1H), 4.01 (p, J=6.8 Hz, 2H), 3.69 (s, 4H), 3.14 (d, J=12.4 Hz, 1H), 3.04 (dd, J=17.1, 4.1 Hz, 1H), 2.96-2.89 (m, 1H), 2.65-2.52 (m, 3H), 2.35-2.28 (m, 1H), 2.22 (ddd, J=13.8, 9.5, 6.3 Hz, 1H), 2.15-2.08 (m, 1H), 2.08-2.01 (m, 1H), 1.45 (s, 3H), 1.34 (s, 3H), 1.28 (s, 3H), 1.26 (s, 3H), 1.17 (s, 3H), 1.15 (d, J=6.7 Hz, 13H) ppm.

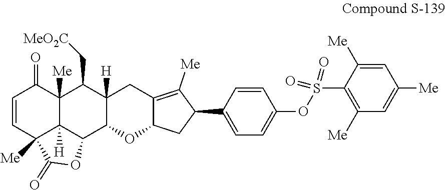

¹H NMR (400 MHz, CDCl₃): δ 7.25 (d, J=9.7 Hz, 1H), 6.96 (d, J=3.6 Hz, 2H), 6.93-6.81 (m, 4H), 5.92 (d, J=9.7 Hz, 1H), 4.71 (s, 1H), 4.52 (dd, J=12.4, 3.8 Hz, 1H), 4.27-4.21 (m, 1H), 3.68 (d, J=9.4 Hz, 4H), 3.14 (d, J=12.4 Hz, 1H), 3.10-2.99 (m, 1H), 2.52 (s, 6H), 2.32 (s, 3H), 2.26-2.09 (m, 2H), 2.04 (q, J=5.1 Hz, 1H), 1.45 (s, 3H), 1.33 (s, 3H), 1.17 (s, 3H) ppm.

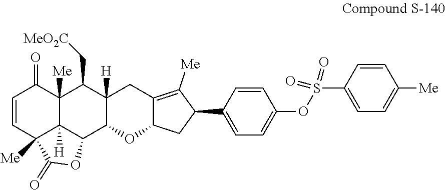

¹H NMR (400 MHz, CDCl₃): δ 7.69 (d, J=8.3 Hz, 2H), 7.30 (d, J=8.1 Hz, 2H), 7.25 (d, J=6.8 Hz, 1H), 6.95-6.83 (m, 4H), 5.92 (d, J=9.7 Hz, 1H), 4.72 (s, 1H), 4.53 (dd, J=12.4, 3.8 Hz, 1H), 4.24 (dd, J=3.9, 1.6 Hz, 1H), 3.69 (s, 4H), 3.14 (d, J=12.4 Hz, 1H), 3.04 (dd, J=17.0, 3.9 Hz, 1H), 2.65-2.53 (m, 3H), 2.45 (s, 3H), 2.32 (d, J=14.7 Hz, 1H), 2.23 (ddd, J=13.8, 9.4, 6.3 Hz, 1H), 2.14 (ddd, J=13.8, 7.6, 2.1 Hz, 1H), 2.04 (t, J=8.1 Hz, 1H), 1.45 (s, 3H), 1.35 (s, 3H), 1.17 (s, 3H) ppm.

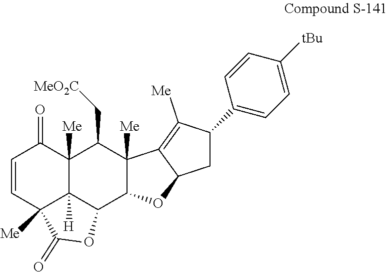

¹H NMR (600 MHz, CDCl₃): δ 7.30-7.26 (m, 3H), 7.03 (d, J=8.3 Hz, 2H), 5.93 (d, J=9.7 Hz, 1H), 5.64-5.57 (m, 1H), 4.63 (dd, J=12.5, 3.7 Hz, 1H), 4.30 (d, J=3.7 Hz, 1H), 3.80-3.72 (m, 1H), 3.47 (s, 3H), 3.24 (dd, J=16.2, 5.6 Hz, 1H), 3.20 (d, J=12.5 Hz, 1H), 2.78 (t, J=5.6 Hz, 1H), 2.41 (dd, J=16.2, 5.6 Hz, 1H), 2.28 (dd, J=12.4, 6.7 Hz, 1H), 2.20 (ddd, J=12.4, 9.0, 7.8 Hz, 1H), 1.68 (d, J=1.8 Hz, 3H), 1.48 (s, 3H), 1.38 (s, 3H), 1.27 (s, 9H), 1.23 (s, 3H) ppm. ¹³C NMR (150 MHz, CDCl₃): δ 200.96, 175.11, 173.01, 149.79, 149.39, 145.07, 139.84, 137.34, 131.18, 127.34, 125.52, 88.89, 83.14, 73.64, 59.68, 51.90, 50.54, 47.93, 45.45, 43.80, 41.73, 41.43, 32.44, 31.49, 18.75, 17.44, 15.33, 13.29 ppm.

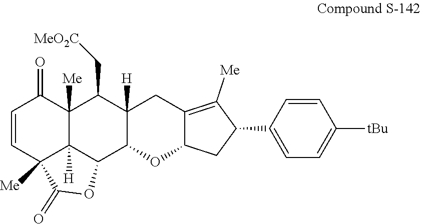

¹H NMR (600 MHz, CDCl₃): δ 7.34 (d, J=8.2 Hz, 2H), 7.30 (d, J=9.6 Hz, 1H), 7.13 (d, J=8.2 Hz, 2H), 6.01 (d, J=9.7 Hz, 1H), 4.56 (dd, J=12.4, 3.8 Hz, 1H), 4.50 (t, J=7.5 Hz, 1H), 4.29 (dd, J=3.8, 1.5 Hz, 1H), 3.65 (s, 3H), 3.55-3.50 (m, 1H), 3.27-3.21 (m, 2H), 2.76 (ddt, J=11.2, 5.4, 2.8 Hz, 1H), 2.74-2.69 (m, 1H), 2.62 (dd, J=15.3, 1.9 Hz, 1H), 2.52 (dd, J=17.1, 5.7 Hz, 1H), 2.36-2.31 (m, 1H), 2.00 (dd, J=11.4, 5.2 Hz, 1H), 1.68 (ddd, J=13.1, 8.5, 7.1 Hz, 1H), 1.48 (s, 3H), 1.33 (s, 13H), 1.21 (s, 3H) ppm. ¹³C NMR (150 MHz, CDCl₃): δ 202.21, 175.60, 173.05, 149.68, 149.18, 140.99, 137.44, 131.90, 130.30, 127.96, 125.46, 83.67, 74.86, 73.59, 52.67, 51.91, 47.62, 46.17, 44.01, 40.64, 37.65, 37.47, 33.31, 31.56, 26.70, 18.82, 13.73, 12.34 ppm.

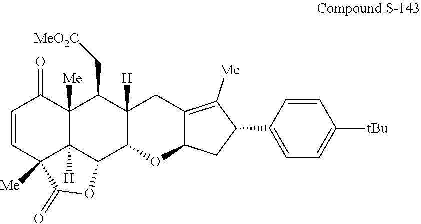

¹H NMR (400 MHz, CDCl₃): δ 7.30 (dd, J=10.9, 8.9 Hz, 3H), 7.04 (d, J=8.2 Hz, 2H), 5.94 (d, J=9.7 Hz, 1H), 5.22-5.14 (m, 1H), 4.65 (dd, J=12.5, 3.7 Hz, 1H), 4.33 (d, J=3.6 Hz, 1H), 3.74 (t, J=7.9 Hz, 1H), 3.65 (s, 3H), 3.36 (dd, J=16.0, 5.1 Hz, 1H), 3.22 (d, J=12.5 Hz, 1H), 2.73 (dd, J=6.9, 5.0 Hz, 1H), 2.65 (dt, J=12.2, 6.2 Hz, 1H), 2.42 (dd, J=16.1, 6.8 Hz, 1H), 1.81 (ddd, J=12.0, 9.8, 8.0 Hz, 1H), 1.48 (s, 3H), 1.46 (s, 3H), 1.39 (s, 3H), 1.31 (s, 9H), 1.22 (s, 3H) ppm.

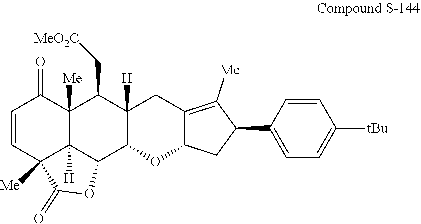

¹H NMR (600 MHz, CDCl₃): δ 7.28-7.24 (m, 2H), 6.97-6.92 (m, 2H), 5.93 (d, J=9.7 Hz, 1H), 4.78 (tt, J=5.6, 3.7 Hz, 1H), 4.53 (dd, J=12.4, 3.8 Hz, 1H), 4.26 (dd, J=3.9, 1.6 Hz, 1H), 3.69 (s, 4H), 3.17 (d, J=12.4 Hz, 1H), 3.03 (dd, J=17.5, 4.6 Hz, 1H), 2.65 (dd, J=17.4, 4.7 Hz, 1H), 2.58 (dt, J=14.9, 3.5 Hz, 2H), 2.37-2.30 (m, 1H), 2.24 (td, J=6.8, 2.2 Hz, 2H), 2.08-2.04 (m, 1H), 1.45 (s, 3H), 1.41-1.39 (m, 3H), 1.29 (s, 9H), 1.17 (s, 3H) ppm. ¹³C NMR (150 MHz, CDCl₃): δ 202.28, 175.71, 173.14, 149.62, 149.03, 141.95, 138.58, 131.80, 129.31, 127.00, 125.50, 84.97, 74.88, 73.62, 53.94, 51.83, 47.47, 46.33, 44.02, 39.00, 37.31, 37.18, 34.48, 32.57, 31.51, 26.76, 18.82, 13.84, 12.62 ppm.

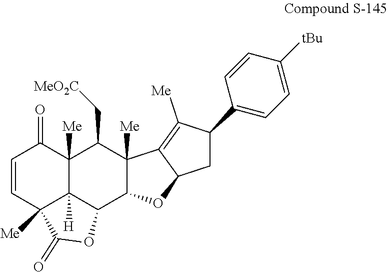

¹H NMR (400 MHz, CDCl₃): δ 7.30 (dd, J=10.9, 8.9 Hz, 3H), 7.04 (d, J=8.2 Hz, 2H), 5.94 (d, J=9.7 Hz, 1H), 5.23-5.13 (m, 1H), 4.65 (dd, J=12.5, 3.7 Hz, 1H), 4.33 (d, J=3.6 Hz, 1H), 3.74 (t, J=7.9 Hz, 1H), 3.65 (s, 3H), 3.36 (dd, J=16.0, 5.1 Hz, 1H), 3.22 (d, J=12.5 Hz, 1H), 2.73 (dd, J=6.9, 5.0 Hz, 1H), 2.65 (dt, J=12.2, 6.2 Hz, 1H), 2.42 (dd, J=16.1, 6.8 Hz, 1H), 1.81 (ddd, J=12.0, 9.8, 8.0 Hz, 1H), 1.48 (s, 3H), 1.46 (d, J=1.7 Hz, 3H), 1.39 (s, 3H), 1.31 (s, 9H), 1.22 (s, 3H) ppm.

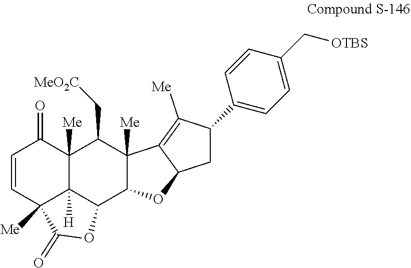

¹H NMR (600 MHz, CDCl₃): δ 7.28 (d, J=9.7 Hz, 1H), 7.21 (d, J=7.8 Hz, 2H), 7.07 (d, J=7.8 Hz, 2H), 5.93 (d, J=9.8 Hz, 1H), 5.61 (tt, J=7.1, 2.0 Hz, 1H), 4.68 (s, 2H), 4.63 (dd, J=12.5, 3.7 Hz, 1H), 4.30 (d, J=3.6 Hz, 1H), 3.78 (d, J=8.4 Hz, 1H), 3.52 (s, 3H), 3.24 (dd, J=16.3, 5.5 Hz, 1H), 3.19 (d, J=12.5 Hz, 1H), 2.80 (t, J=5.5 Hz, 1H), 2.41 (dd, J=16.3, 5.5 Hz, 1H), 2.27-2.18 (m, 2H), 1.66 (d, J=1.9 Hz, 3H), 1.48 (s, 3H), 1.39 (s, 3H), 1.23 (s, 3H), 0.92 (s, 9H), 0.18-0.01 (m, 6H) ppm. ¹³C NMR (150 MHz, CDCl₃): δ 200.96, 175.10, 173.03, 149.78, 145.31, 141.66, 139.79, 137.21, 131.17, 127.62, 126.47, 88.87, 83.17, 73.62, 64.85, 60.03, 51.98, 50.57, 47.94, 45.45, 43.80, 41.82, 41.35, 32.44, 26.07, 18.75, 18.55, 17.44, 15.32, 13.22, −5.13 ppm.

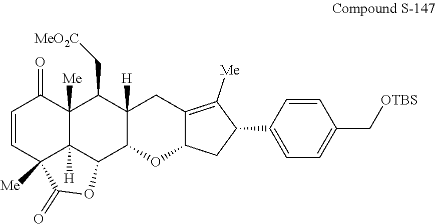

¹H NMR (600 MHz, CDCl₃): δ 7.28 (d, J=9.7 Hz, 1H), 7.26 (d, J=7.8 Hz, 2H), 7.19-7.12 (m, 2H), 5.98 (d, J=9.7 Hz, 1H), 4.72 (s, 2H), 4.54 (dd, J=12.4, 3.8 Hz, 1H), 4.48 (tt, J=6.3, 3.3 Hz, 1H), 4.26 (dd, J=3.8, 1.6 Hz, 1H), 3.61 (s, 3H), 3.53-3.47 (m, 1H), 3.27-3.19 (m, 2H), 2.75 (ddd, J=11.6, 5.9, 3.5 Hz, 1H), 2.69 (dt, J=13.1, 7.4 Hz, 1H), 2.59 (dd, J=15.4, 1.9 Hz, 1H), 2.45 (dd, J=17.1, 5.9 Hz, 1H), 2.34-2.27 (m, 1H), 1.96 (ddt, J=11.5, 5.4, 1.7 Hz, 1H), 1.67 (ddd, J=13.1, 8.6, 7.2 Hz, 1H), 1.46 (s, 3H), 1.28 (d, J=1.5 Hz, 3H), 1.18 (s, 3H), 0.93 (s, 9H), 0.09 (d, J=1.6 Hz, 6H) ppm. ¹³C NMR (150 MHz, CDCl₃): δ 202.23, 175.60, 173.00, 149.69, 142.69, 139.58, 137.33, 131.91, 130.39, 128.32, 126.42, 83.64, 74.85, 73.62, 65.02, 52.86, 51.91, 47.63, 46.13, 44.00, 40.64, 37.69, 37.45, 33.37, 26.64, 26.12, 18.81, 18.58, 13.70, 12.21, −5.09 ppm.

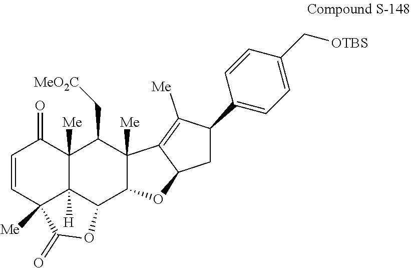

¹H NMR (600 MHz, CDCl₃): δ 7.29 (d, J=9.7 Hz, 1H), 7.27 (s, 2H), 7.10-7.06 (m, 2H), 5.94 (d, J=9.7 Hz, 1H), 5.18 (ddt, J=8.4, 6.6, 1.9 Hz, 1H), 4.72 (s, 2H), 4.65 (dd, J=12.5, 3.7 Hz, 1H), 4.33 (d, J=3.6 Hz, 1H), 3.76 (ddt, J=7.9, 5.8, 3.2 Hz, 1H), 3.65 (s, 3H), 3.36 (dd, J=16.1, 4.9 Hz, 1H), 3.22 (d, J=12.5 Hz, 1H), 2.73 (dd, J=6.9, 5.0 Hz, 1H), 2.65 (dt, J=12.2, 6.2 Hz, 1H), 2.42 (dd, J=16.1, 6.9 Hz, 1H), 1.80 (ddd, J=12.1, 9.9, 8.1 Hz, 1H), 1.48 (s, 3H), 1.45 (t, J=1.6 Hz, 3H), 1.39 (s, 3H), 1.22 (s, 3H), 0.94 (s, 9H), 0.10 (s, 6H) ppm. ¹³C NMR (150 MHz, CDCl₃): δ 201.14, 175.09, 173.05, 149.84, 146.28, 141.40, 140.15, 136.19, 131.23, 128.23, 126.40, 87.04, 83.16, 73.69, 64.85, 56.46, 51.88, 50.44, 47.91, 45.37, 43.95, 43.83, 41.41, 32.24, 26.10, 18.67, 18.58, 17.38, 15.37, 12.86, −5.11 ppm.

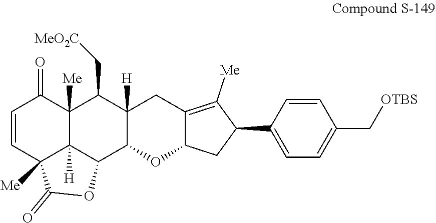

¹H NMR (600 MHz, CDCl₃): δ 7.26 (d, J=9.6 Hz, 1H), 7.21 (d, J=7.8 Hz, 2H), 6.97 (d, J=8.2 Hz, 2H), 5.93 (d, J=9.7 Hz, 1H), 4.77 (t, J=7.1 Hz, 1H), 4.69 (s, 2H), 4.53 (dd, J=12.5, 3.8 Hz, 1H), 4.26 (dd, J=3.8, 1.6 Hz, 1H), 3.69 (s, 4H), 3.17 (d, J=12.4 Hz, 1H), 3.04 (dd, J=17.4, 4.5 Hz, 1H), 2.65 (dd, J=17.4, 4.7 Hz, 1H), 2.58 (dt, J=14.8, 3.5 Hz, 2H), 2.34 (dd, J=15.0, 4.7 Hz, 1H), 2.28-2.18 (m, 2H), 2.08-2.04 (m, 1H), 1.45 (s, 3H), 1.38 (d, J=2.0 Hz, 3H), 1.17 (s, 3H), 0.93 (s, 10H), 0.08 (s, 6H) ppm. ¹³C NMR (150 MHz, CDCl₃): δ 202.28, 175.70, 173.14, 149.62, 143.75, 139.42, 138.45, 131.81, 129.50, 127.25, 126.45, 84.96, 74.86, 73.65, 64.92, 54.22, 51.85, 47.48, 46.32, 44.02, 39.11, 37.34, 37.17, 32.61, 26.76, 26.11, 18.83, 13.84, 12.55, −5.10 ppm.

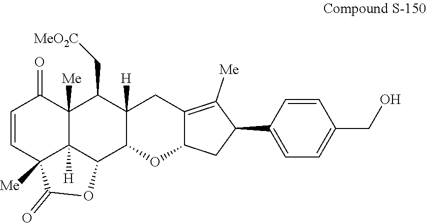

¹H NMR (600 MHz, CDCl₃): δ 7.28-7.24 (m, 3H), 7.01 (d, J=8.1 Hz, 2H), 5.93 (d, J=9.7 Hz, 1H), 4.80-4.74 (m, 1H), 4.66-4.62 (m, 2H), 4.54 (dd, J=12.4, 3.8 Hz, 1H), 4.26 (dd, J=3.9, 1.7 Hz, 1H), 3.73 (d, J=9.3 Hz, 1H), 3.69 (s, 3H), 3.16 (d, J=12.4 Hz, 1H), 3.04 (dd, J=17.4, 4.4 Hz, 1H), 2.64 (dd, J=17.3, 4.8 Hz, 1H), 2.61-2.55 (m, 2H), 2.35 (ddq, J=15.0, 5.6, 1.8 Hz, 1H), 2.29-2.17 (m, 2H), 2.08-2.04 (m, 1H), 1.45 (s, 3H), 1.39 (d, J=1.8 Hz, 3H), 1.17 (s, 3H) ppm. ¹³C NMR (150 MHz, CDCl₃): δ 202.25, 175.69, 173.13, 149.61, 144.69, 138.89, 138.23, 131.79, 129.76, 127.62, 127.55, 127.53, 84.87, 74.83, 73.64, 65.33, 54.22, 51.85, 47.47, 46.30, 44.01, 39.07, 37.34, 37.16, 32.62, 26.74, 18.81, 13.82, 12.54 ppm.

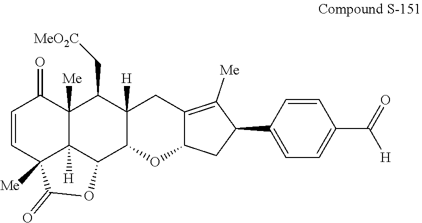

¹H NMR (600 MHz, CDCl₃): δ 9.96 (s, 1H), 7.79 (d, J=7.8 Hz, 2H), 7.27 (s, 1H), 7.18 (d, J=7.9 Hz, 2H), 5.93 (d, J=9.8 Hz, 1H), 4.80-4.76 (m, 1H), 4.54 (dd, J=12.4, 3.8 Hz, 1H), 4.28 (dd, J=3.8, 1.7 Hz, 1H), 3.82 (d, J=9.5 Hz, 1H), 3.70 (s, 3H), 3.16 (d, J=12.4 Hz, 1H), 3.09-3.04 (m, 1H), 2.65-2.57 (m, 3H), 2.37 (dd, J=16.1, 5.1 Hz, 1H), 2.30 (ddd, J=13.7, 9.5, 6.2 Hz, 1H), 2.22 (ddd, J=14.0, 7.7, 1.9 Hz, 1H), 2.09-2.04 (m, 1H), 1.46 (s, 3H), 1.39 (s, 3H), 1.18 (s, 3H) ppm. ¹³C NMR (150 MHz, CDCl₃): δ 202.18, 192.05, 175.64, 173.11, 152.56, 149.62, 137.46, 135.00, 131.80, 130.77, 130.36, 128.11, 84.71, 74.72, 73.69, 54.71, 51.88, 47.49, 46.27, 44.01, 38.78, 37.40, 37.15, 32.67, 26.74, 18.82, 13.80, 12.59 ppm.

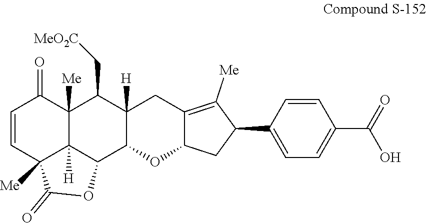

¹H NMR (600 MHz, CDCl₃): δ 7.99 (d, J=8.3 Hz, 2H), δ7.26 (d, J=9.7 Hz, 1H), 7.11 (d, J=8.3 Hz, 2H), 5.93 (d, J=9.7 Hz, 1H), 4.81-4.76 (m, 1H), 4.54 (dd, J=12.4, 3.8 Hz, 1H), 4.28 (dd, J=3.9, 1.6 Hz, 1H), 3.80 (d, J=8.9 Hz, 1H), 3.70 (s, 3H), 3.16 (d, J=12.3 Hz, 1H), 3.10-3.03 (m, 1H), 2.66-2.56 (m, 3H), 2.40-2.33 (m, 1H), 2.29 (ddd, J=13.8, 9.6, 6.3 Hz, 1H), 2.22 (ddd, J=13.9, 7.6, 1.9 Hz, 1H), 2.09-2.05 (m, 1H), 1.39 (d, J=1.9 Hz, 3H), 1.25 (s, 3H), 1.18 (s, 3H) ppm. ¹³C NMR (150 MHz, CDCl₃) δ 202.21, 175.67, 173.13, 151.59, 149.63, 137.61, 131.79, 130.73, 130.56, 127.59, 127.44, 84.75, 74.75, 73.67, 54.59, 51.87, 47.48, 46.27, 44.01, 38.79, 37.39, 37.15, 32.66, 29.84, 26.73, 18.82, 13.80, 12.57 ppm.

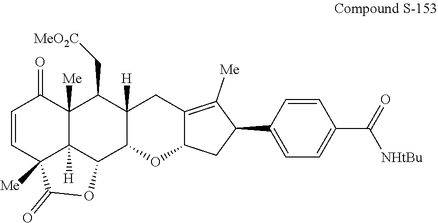

¹H NMR (600 MHz, CDCl₃): δ 7.61 (d, J=8.2 Hz, 2H), δ7.25 (d, J=6.8 Hz, 1H), 7.05 (d, J=8.2 Hz, 2H), 5.93 (d, J=9.7 Hz, 1H), 5.89 (s, 1H), 4.80-4.74 (m, 1H), 4.54 (dd, J=12.4, 3.8 Hz, 1H), 4.27 (dd, J=3.8, 1.6 Hz, 1H), 3.76 (d, J=9.5 Hz, 1H), 3.69 (s, 3H), 3.16 (d, J=12.4 Hz, 1H), 3.06 (dd, J=17.0, 3.9 Hz, 1H), 2.65-2.55 (m, 3H), 2.39-2.33 (m, 1H), 2.27 (ddd, J=13.7, 9.5, 6.3 Hz, 1H), 2.18 (ddd, J=13.8, 7.6, 1.9 Hz, 1H), 2.08-2.03 (m, 1H), 1.45 (s, 9H), 1.37 (s, 3H), 1.25 (s, 3H), 1.18 (s, 3H) ppm. ¹³C NMR (150 MHz, CDCl₃): δ 202.21, 175.66, 173.11, 166.92, 149.61, 148.56, 137.82, 134.11, 131.80, 130.23, 127.51, 127.19, 84.78, 74.77, 73.66, 54.32, 51.86, 51.69, 47.48, 46.28, 44.01, 38.92, 37.38, 37.15, 32.66, 29.02, 26.72, 18.82, 13.81, 12.51 ppm.

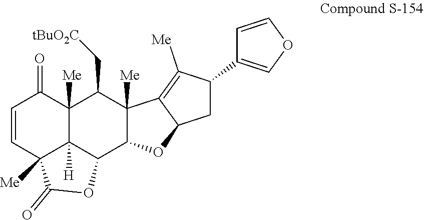

¹H NMR (600 MHz, CDCl₃): δ 7.27 (s, 1H), 7.25 (s, 2H), 7.17 (s, 1H), 6.23 (t, J=1.3 Hz, 1H), 5.90 (d, J=9.7 Hz, 1H), 5.56 (tq, J=6.7, 1.9 Hz, 1H), 4.62 (dd, J=12.5, 3.7 Hz, 1H), 4.29 (d, J=3.7 Hz, 1H), 3.67 (d, J=8.5 Hz, 1H), 3.18 (d, J=12.6 Hz, 1H), 2.95 (dd, J=17.3, 6.8 Hz, 1H), 2.76 (dd, J=6.8, 2.8 Hz, 1H), 2.23-2.15 (m, 2H), 2.10 (dt, J=11.8, 8.3 Hz, 1H), 1.72 (d, J=1.8 Hz, 3H), 1.46 (s, 3H), 1.45 (s, 9H), 1.33 (s, 3H), 1.22 (s, 3H) ppm.

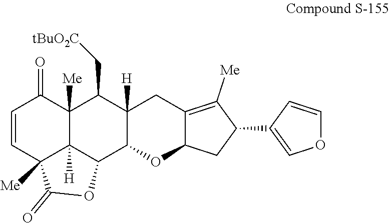

¹H NMR (600 MHz, CDCl₃): δ 7.33 (s, 1H), 7.17 (s, 1H), 6.82 (d, J=11.6 Hz, 1H), 6.19 (t, J=1.2 Hz, 1H), 6.04 (d, J=11.6 Hz, 1H), 5.46-5.40 (m, 1H), 4.28 (dd, J=11.6, 3.1 Hz, 1H), 4.24 (d, J=3.0 Hz, 1H), 3.71 (d, J=8.4 Hz, 1H), 2.91 (dd, J=13.3, 7.5 Hz, 1H), 2.69 (t, J=11.7 Hz, 1H), 2.53 (dd, J=13.3, 4.4 Hz, 1H), 2.48 (dd, J=16.4, 3.2 Hz, 1H), 2.31-2.25 (m, 2H), 2.20 (td, J=11.4, 11.0, 7.1 Hz, 2H), 2.11 (dt, J=11.9, 8.3 Hz, 1H), 1.79 (d, J=2.0 Hz, 3H), 1.48 (s, 9H), 1.38 (s, 3H), 1.29 (s, 3H) ppm.

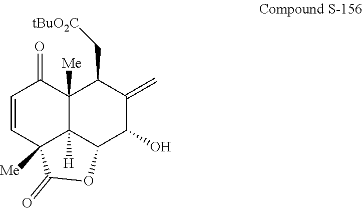

¹H NMR (600 MHz, CDCl₃): δ 7.27 (d, J=9.9 Hz, 1H), 5.97 (d, J=9.7 Hz, 1H), 5.39 (d, J=1.4 Hz, 1H), 5.07 (d, J=1.7 Hz, 1H), 4.75 (d, J=3.5 Hz, 1H), 4.37 (dd, J=12.3, 3.5 Hz, 1H), 3.53 (dd, J=16.7, 3.1 Hz, 1H), 3.40 (d, J=12.3 Hz, 1H), 3.34 (ddd, J=10.6, 3.2, 1.6 Hz, 1H), 2.48 (dd, J=16.7, 12.0 Hz, 2H), 1.43 (s, 3H), 1.41 (s, 9H), 1.01 (s, 3H) ppm.

### C. Biological Activity of Nimbolide Derivatives

Nimbolide and its analogs represent the 2nd generation PARP1 inhibitors. Compared to regular PARPi, nimbolide and its analogs have the following attributes: (1) Nimbolide inhibits RNF114, leading to the trapping of PARP1 and PAR-dependent DNA repair factors. In contrast, regular PARPi only trap PARP1. Nimbolide therefore is a “super trapper”. (2) Nimbolide and its analogs are able to kill cancers with defects in genes in the homologous recombination pathway. (3) Nimbolide and its analogs are able to kill cancers with intrinsic and acquired resistance to regular PARPi. (4) Nimbolide and its analogs synergize with other anti-cancer agents, including immune check point inhibitors and agents targeting the DNA repair enzymes (e.g., ATM, ATR and CHK).

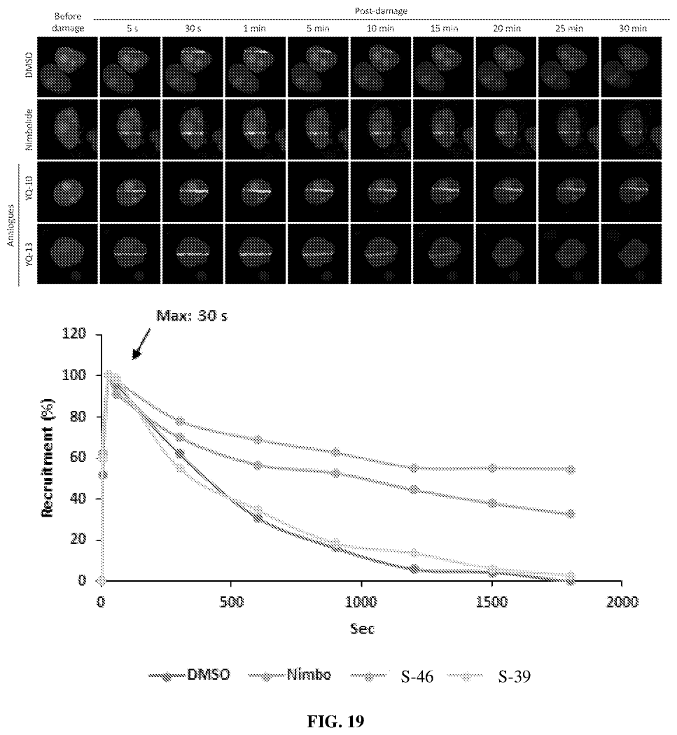

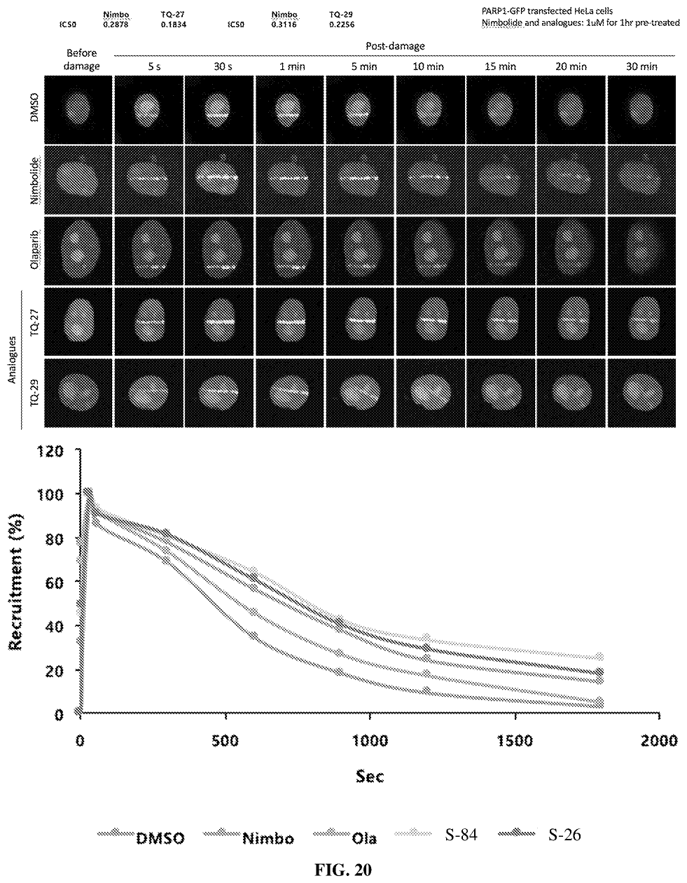

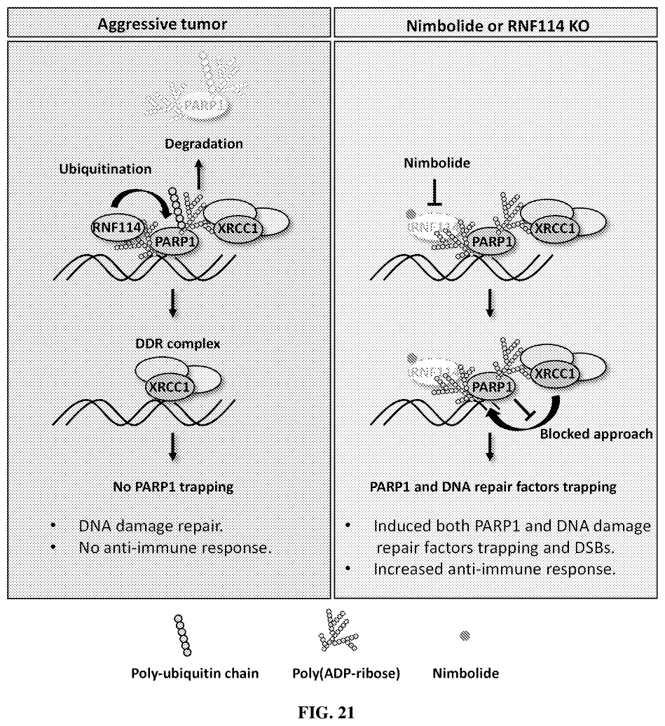

UWB1 cell viability upon treatment with nimbolide and derivatives are show below in Table 2. Furthermore, we measured the PARP1 trapping activity of several nimbolide analogs. See FIG. 19. In particular, S-46 displayed more potent trapping activity, compared to nimbolide. This compound also demonstrated excellent cytotoxicity against UWB1 cells (Table 2).

TABLE 2
UWB1 cell viability upon treatment
with nimbolide and derivatives.

| | IC₅₀ (μM) | | IC₅₀ (μM) |
|---|---|---|---|
| Compound ID | UWB1 cells | Compound ID | UWB1 cells |
| Nimbolide | 0.332 | S-57 | 0.591 |
| (S-28) | | S-58 | 1.794 |
| S-16 | >10 | S-59 | 0.203 |
| S-16-a | >10 | S-60 | >10 |
| S-16-b | >10 | S-61 | >10 |
| S-26 | 0.226 | S-62 | >10 |
| S-26-1 | 0.265 | S-63 | >10 |
| S-27 | 0.415 | S-64 | >10 |
| S-38 | >10 | S-65 | >10 |
| S-39 | 1.179 | S-84 | 0.183 |
| S-41 | 0.465 | S-97 | 1.45 |
| S-42 | 0.144 | S-98 | 0.83 |
| S-43 | 0.208 | S-99 | 0.88 |
| S-44 | 0.383 | S-100 | 0.98 |
| S-45 | 0.251 | S-101 | 0.30 |
| S-46 | 0.089 | S-102 | 0.17 |
| S-47 | 0.296 | S-103 | 0.16 |
| S-48 | 0.212 | S-104 | 0.21 |
| S-49 | 0.194 | S-105 | 0.20 |
| S-50 | 1.041 | S-106 | 0.16 |
| S-51 | 0.954 | S-107 | 0.27 |
| S-52 | 0.199 | S-108 | 0.11 |
| S-53 | 0.170 | S-109 | 0.17 |
| S-54 | 0.265 | S-111 | 0.255 |
| S-55 | >10 | S-112a | 0.198 |
| S-56 | >10 | S-112 | 0.2193 |
| S-113 | 0.2164 | S-140 | 0.1186 |
| S-114 | 0.2425 | S-141 | 0.2477 |
| S-115 | 0.1956 | S-142 | 0.1451 |
| S-116 | 0.1498 | S-143 | 0.2384 |
| S-117 | 0.0788 | S-144 | 0.1395 |
| S-118 | 0.0815 | S-145 | 0.1567 |
| S-119 | 0.1882 | S-146 | 0.1799 |
| S-120 | 0.1651 | S-147 | 0.1915 |
| S-121 | 0.1904 | S-148 | 0.1606 |
| S-122 | 0.0534 | S-149 | 0.1625 |
| S-123 | 0.4643 | S-150 | 0.3514 |
| S-124 | 0.5637 | S-151 | 0.7664 |
| S-126 | 0.1408 | S-152 | 2.882 |
| S-127 | 0.9275 | S-153 | 0.2454 |
| S-128 | 0.0908 | S-154 | 0.4525 |
| S-129 | 0.1631 | S-155 | 0.3062 |
| S-130 | 0.2250 | S-156 | 3.646 |
| S-131 | 1.1839 | | |
| S-132 | 0.1418 | | |
| S-133 | 0.1864 | | |
| S-134 | 0.0910 | | |
| S-135 | 0.1852 | | |
| S-136 | 0.0934 | | |
| S-137 | 0.1692 | | |
| S-138 | 0.1882 | | |
| S-139 | 0.1402 | | |

All the compounds, formulations, and methods disclosed and claimed herein can be made and executed without undue experimentation in light of the present disclosure. While the compounds, formulations, and methods of this disclosure have been described in terms of preferred embodiments, it will be apparent to those of skill in the art that variations may be applied to the compounds, formulations, and methods, as well as in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references to the extent that they provide exemplary procedural or other details supplementary to those set forth herein, are specifically incorporated herein by reference.
Anderson, Practical Process Research & Development—A Guide for Organic Chemists, 2^{nd} ed., Academic Press, New York, 2012.
Babykutty et al., Molecular carcinogenesis, 51:475-490, 2012.
Berger et al., British journal of pharmacology, 175:192-222, 2018.
Bijlmakers et al., Human molecular genetics, 20:3129-3137, 2011.
Bryant et al., Nature, 434:913-917, 2005.
Chabanon et al., The Journal of clinical investigation, 129:1211-1228, 2019.
Dawson and Dawson, Annual review of pharmacology and toxicology, 57:437-454, 2017.
Farmer et al., Nature, 434:917-921, 2005.
Giannini et al., The Biochemical journal, 410:101-111, 2008.
Handbook of Pharmaceutical Salts: Properties, and Use, Stahl and Wermuth Eds., Verlag Helvetica Chimica Acta, 2002.
Hamulić et al., J. Nat. Prod., 82:823-831, 2019.
Hu et al., Journal of proteome research, 15:477-486, 2016.
Kim et al., eLife, 9:e60637, 2020.
Kraus, Molecular cell, 58:902-910, 2015.
Ledermann et al. N. Engl. J. Med., 366:1382-1392, 2012.
Lehmann et al., The Journal of clinical investigation, 121:2750-2767, 2011.
Li et al., Chem. Comm., 52:12426, 2016.
Litton et al., N. Engl. J. Med., 379:753-763, 2018.
Lord and Ashworth, Molecular and cellular biology, 27:5597-5605, 2007.
Mahapatra et al., Evidence-based complementary and alternative medicine: eCAM, 2012:303019, 2012.
Masson et al., Molecular and cellular biology, 18:3563-3571, 1998.
Mirza et al., N. Engl. J. Med., 375:2154-2164, 2016.
Murai, International journal of clinical oncology, 22:619-628, 2017.
Murai et al., Cancer research, 72:5588-5599, 2012.
Murai et al., Molecular cancer therapeutics, 13:433-443, 2014.
Pommier et al., Science translational medicine, 8:362ps317, 2016.
Raja Singh et al., Cell biochemistry and function, 32:217-228, 2014.
Reagan-Shaw et al., FASEB J., 22(3):659-661, 2008
Rouleau et al., Nature Rev. Cancer, 10:293-301, 2010.
Roy et al., Phytotherapy research: PTR, 21:245-250, 2007.
Shen et al., Cancer research, 79:311-319, 2019.
Spradlin et al., Nature chemical biology, 15:747-755, 2019.
Sun et al., Science, 339:786-791, 2013.
Swisher et al., The Lancet Oncology, 18:75-87, 2017.
Tsai and Carstens, Nature protocols, 1:2820-2827, 2006.
Wang et al., Oncotarget, 7:44790-44802, 2016.
Wang et al., Nature chemical biology, 15:1223-1231, 2019.
Wang et al., Nature communications, 10:3201, 2019.
Yang et al., EMBO reports, 18:205-216, 2017.
Yazinski et al., Genes & development, 31:318-332, 2017.
Zhao et al., Journal of immunology, 174:5288-5297, 2005.
Zhen and Yu, Biochemistry, 57:429-440, 2018.
Zhen et al., Cell reports, 21:2326-2337, 2017.

What is claimed is:

1. A compound of the formula: 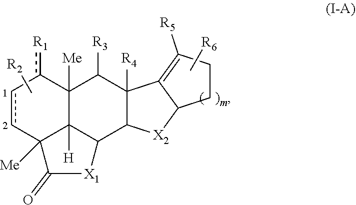
wherein:
the bond between atoms 1 and 2 is a single bond or a double bond;
m is 1 or 2;
X₁ and X₂ are each independently —O— or —NRₐ—, wherein:
Rₐ is hydrogen, alkyl_{(C≤8)}, or substituted alkyl_{(C≤8)};
R₁ is hydroxy or oxo;
R₂ is hydrogen, amino, halo, hydroxy; or
alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
Y₁ is alkanediyl_{(C≤8)} or substituted alkanediyl_{(C≤8)};
R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₄ is hydrogen, alkyl_{(C≤8)}, or substituted alkyl_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
A₁-R₇, wherein:
A₁ is heteroarenediyl_{(C≤12)} or substituted heteroarenediyl_{(C≤12)};
R₇ is hydrogen, amino, hydroxy, or an amino protecting group; or
alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 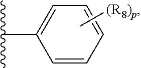
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
—C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 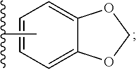
or
-A₂-R₉, wherein:
A₂ is cycloalkanediyl_{(C≤8)}, heterocycloalkanediyl_{(C≤8)}, arenediyl_{(C≤12)} or a substituted version of any of these groups;
R₉ is hydrogen, amino, halo, hydroxy, or an amino protecting group; or
alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤8)}, or a substituted version of any of these groups;
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 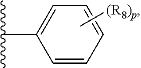
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 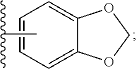
or
R₆ is a group of the formula: 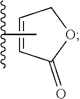
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
provided the compound is not: 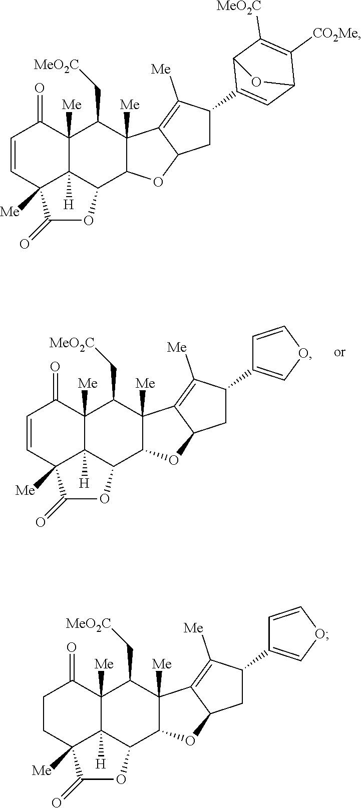
or
a compound of the formula: 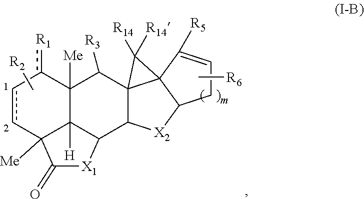
wherein:
the bond between atoms 1 and 2 is a single bond or a double bond;
m is 1 or 2;
X₁ and X₂ are each independently —O— or —NRₐ—, wherein:
Rₐ is hydrogen, alkyl_{(C≤8)}, or substituted alkyl_{(C≤8)};
R₁ is hydroxy or oxo;
R₂ is hydrogen, amino, halo, hydroxy; or
alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
Y₁ is alkanediyl_{(C≤8)} or substituted alkanediyl_{(C≤8)};
R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₅ is hydrogen, alkyl_{(C≤8)}, substituted alkyl_{(C≤8)}, or R₅ is taken together with R₆ as defined below;
R₆ is hydrogen, amino, halo, or hydroxy; or
-A₁-R₇, wherein:
A₁ is heteroarenediyl_{(C≤12)} or substituted heteroarenediyl_{(C≤12)}; and
R₇ is hydrogen, amino, hydroxy, or an amino protecting group; or
alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 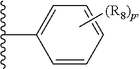
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
—C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 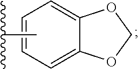
or
-A₂-R₉, wherein:
A₂ is cycloalkanediyl_{(C≤8)}, heterocycloalkanediyl_{(C≤8)}, arenediyl_{(C≤12)} or a substituted version of any of these groups; and
R₉ is hydrogen, amino, halo, hydroxy, or an amino protecting group; or
alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤18)}, or a substituted version of any of these groups;
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 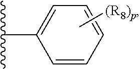
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 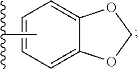
or
R₆ is a group of the formula: 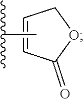
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)}; and
R₁₄ and R_{14′} are each independently hydrogen, alkyl_{(C≤8)}, or substituted alkyl_{(C≤8)}; or
a compound of the formula: 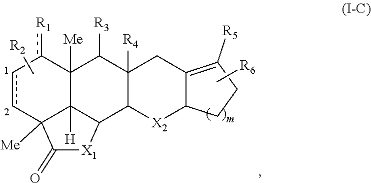
wherein:
the bond between atoms 1 and 2 is a single bond or a double bond;
m is 1 or 2;
X₁ and X₂ are each independently —O— or —NRₐ—, wherein:
Rₐ is hydrogen, alkyl_{(C≤8)}, or substituted alkyl_{(C≤8)};
R₁ is hydroxy or oxo;
R₂ is hydrogen, amino, halo, hydroxy; or
alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
Y₁ is alkanediyl_{(C≤8)} or substituted alkanediyl_{(C≤8)};
R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₄ is hydrogen, alkyl_{(C≤8)}, or substituted alkyl_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
-A₁-R₇, wherein:
A₁ is heteroarenediyl_{(C≤12)} or substituted heteroarenediyl_{(C≤12)};
R₇ is hydrogen, amino, hydroxy, or an amino protecting group; or
alkyl_{(C≤8)}, cycloalkyl_{(C≤8)}, heterocycloalkyl_{(C≤8)}, heteroaryl_{(C≤12)}, arylsulfonyloxy_{(C≤8)}, or a substituted version of any of these groups; or
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 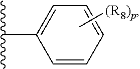
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
—C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 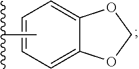
or
-A₂-R₉, wherein:
A₂ is cycloalkanediyl_{(C≤8)}, heterocycloalkanediyl arenediyl_{(C≤12)} or a substituted version of any of these groups;
R₉ is hydrogen, amino, halo, hydroxy, or an amino protecting group; or
alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤8)}, or a substituted version of any of these groups;
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 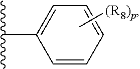
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 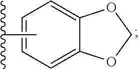
or
R₆ is a group of the formula: 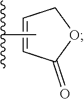
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)}; or
a compound of the formula: 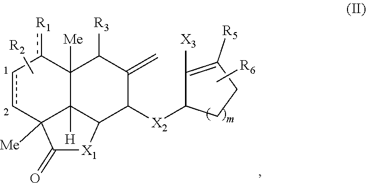
wherein:
the bond between atoms 1 and 2 is a single bond or a double bond;
m is 1 or 2;
n is 0 or 1;
X₁ and X₂ are each independently —O— or —NRₐ—, wherein:
Rₐ is hydrogen, alkyl_{(C≤8)}, or substituted alkyl_{(C≤8)};
X₃ is hydrogen, amino, halo, or hydroxy;
R₁ is hydroxy or oxo;
R₂ is hydrogen, amino, halo, hydroxy; or
alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
Y₁ is alkanediyl_{(C≤8)} or substituted alkanediyl_{(C≤8)};
R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
-A₃-R₁₀, wherein:
A₃ is cycloalkanediyl_{(C≤8)}, heterocycloalkanediyl_{(C≤8)}, arenediyl_{(C≤12)} heteroarenediyl_{(C≤12)}, or a substituted version of any of these groups; and
R₁₀ is hydrogen, amino, hydroxy, or an amino protecting group; or
alkyl_{(C≤8)}, cycloalkyl_{(C≤8)}, heterocycloalkyl_{(C≤8)}, heteroaryl_{(C≤12)}, arylsulfonyloxy_{(C≤8)}, or a substituted version of any of these groups; or
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 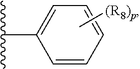
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
—C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 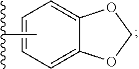
or
R₆ is a group of the formula: 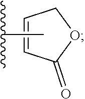
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically accepted salt of any of these formulae.

2. The compound of claim 1, wherein the compound is further defined as: 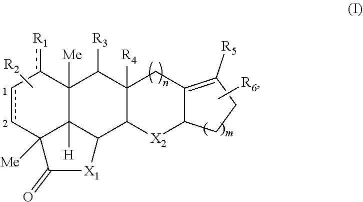
wherein:
the bond between atoms 1 and 2 is a single bond or a double bond;
m is 1 or 2;
n is 0 or 1;
X₁ and X₂ are each independently —O— or —NRₐ—, wherein:
Rₐ is hydrogen, alkyl_{(C≤8)}, or substituted alkyl_{(C≤8)};
R₁ is hydroxy or oxo;
R₂ is hydrogen, amino, halo, hydroxy; or
alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
Y₁ is alkanediyl_{(C≤8)} or substituted alkanediyl_{(C≤8)};
R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₄ is hydrogen, alkyl_{(C≤8)}, or substituted alkyl_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
-A₁-R₇, wherein:
A₁ is heteroarenediyl_{(C≤12)} or substituted heteroarenediyl_{(C≤12)};
R₇ is hydrogen, amino, hydroxy, or an amino protecting group; or
alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 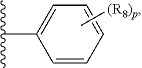
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
—C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 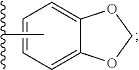
or
-A₂-R₉, wherein:
A₂ is cycloalkanediyl_{(C≤8)}, heterocycloalkanediyl_{(C≤8)}, arenediyl_{(C≤12)} or a substituted version of any of these groups;
R₉ is hydrogen, amino, halo, hydroxy, or an amino protecting group; or
alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤8)}, or a substituted version of any of these groups;
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 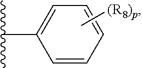
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 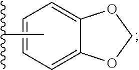
or R₅ is a group of the formula: 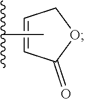
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
provided the compound is not: 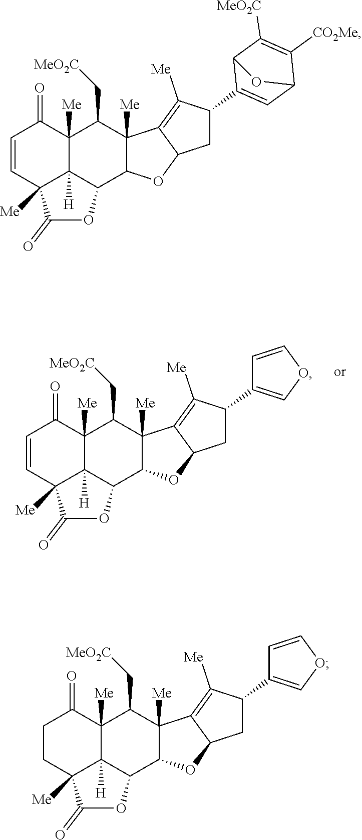
or
a compound of the formula: 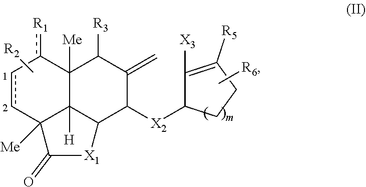
wherein:
the bond between atoms 1 and 2 is a single bond or a double bond;
m is 1 or 2;
X₁ and X₂ are each independently —O— or —NRₐ—, wherein:
Rₐ is hydrogen, alkyl_{(C≤8)}, or substituted alkyl_{(C≤8)};
X₃ is hydrogen, amino, halo, or hydroxy;
R₁ is hydroxy or oxo;
R₂ is hydrogen, amino, halo, hydroxy; or
alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
Y₁ is alkanediyl_{(C≤8)} or substituted alkanediyl_{(C≤8)};
R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
-A₃-R₁₀, wherein:
A₃ is cycloalkanediyl_{(C≤8)}, heterocycloalkanediyl_{(C≤8)}, arenediyl_{(C≤12)} heteroarenediyl_{(C≤12)}, or a substituted version of any of these groups;
R₁₀ is hydrogen, amino, halo, hydroxy, or an amino protecting group; or
alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤8)}, or a substituted version of any of these groups;
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 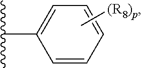
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 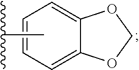
or
R₆ is a group of the formula: 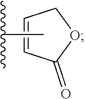
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically accepted salt of either of these formulae.

3. The compound of either claim 1 or claim 2, wherein the compound is further defined as: 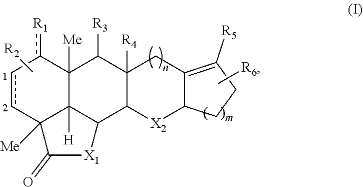
wherein:
the bond between atoms 1 and 2 is a single bond or a double bond;
m is 1 or 2;
n is 0 or 1;
X₁ and X₂ are each independently —O— or —NRₐ—, wherein:
Rₐ is hydrogen, alkyl_{(C≤8)}, or substituted alkyl_{(C≤8)};
R₁ is hydroxy or oxo;
R₂ is hydrogen, amino, halo, hydroxy; or
alkyl_{(C≤8)} or substituted alkyl_{(C≤8)};
R₃ hydrogen, alkyl_{(C≤8)}, substituted alkyl_{(C≤8)}, or —Y₁C(O)R_{b}, wherein:
Y₁ is alkanediyl_{(C≤8)} or substituted alkanediyl_{(C≤8)};
R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₄ is hydrogen, alkyl_{(C≤8)}, or substituted alkyl_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
-A₁-R₇, wherein:
A₁ is heteroarenediyl_{(C≤12)} or substituted heteroarenediyl_{(C≤12)};
R₇ is hydrogen, amino, hydroxy, or an amino protecting group; or
alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 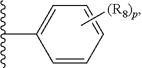
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
—C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 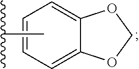
or
-A₂-R₉, wherein:
A₂ is cycloalkanediyl_{(C≤8)}, heterocycloalkanediyl_{(C≤8)}, arenediyl_{(C≤12)} or a substituted version of any of these groups;
R₉ is hydrogen, amino, halo, hydroxy, or an amino protecting group; or
alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤8)}, or a substituted version of any of these groups;
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 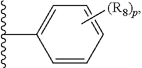
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 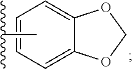
or
R₆ is a group of the formula: 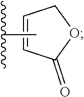
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
provided the compound is not: 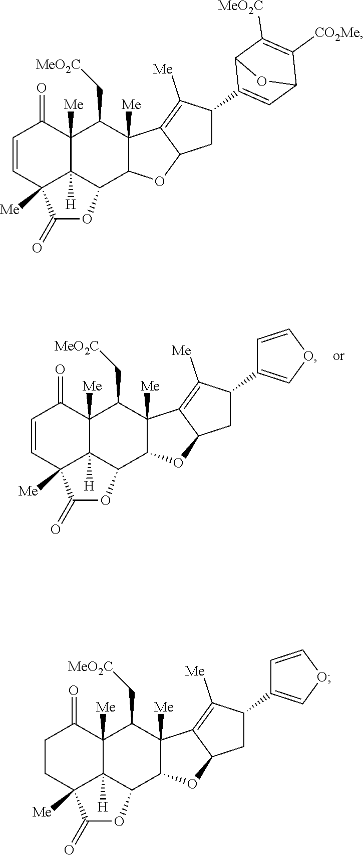
or a pharmaceutically acceptable salt thereof.

4. The compound of either claim 1 or claim 2, wherein the compound is further defined as: 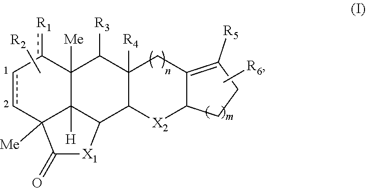
wherein:
the bond between atoms 1 and 2 is a single bond or a double bond;
m is 1 or 2;
n is 0 or 1;
X₁ and X₂ are each independently —O— or —NRₐ—, wherein:
Rₐ is hydrogen, alkyl_{(C≤8)}, or substituted alkyl_{(C≤8)};
R₁ is hydroxy or oxo;
R₂ is hydrogen, amino, halo, hydroxy; or
alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
Y₁ is alkanediyl_{(C≤8)} or substituted alkanediyl_{(C≤8)};
R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₄ is hydrogen, alkyl_{(C≤8)}, or substituted alkyl_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
-A₁-R₇, wherein:
A₁ is an oxygen-containing heteroarenediyl_{(C≤12)} or a substituted oxygen-containing heteroarenediyl_{(C≤12)};
R₇ is hydrogen, amino, hydroxy, or an amino protecting group; or
alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 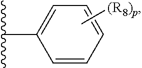
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
—C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 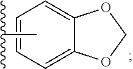
or
-A₂-R₉, wherein:
A₂ is a nitrogen-containing heteroarenediyl_{(C≤12)}, cycloalkanediyl_{(C≤8)}, heterocycloalkanediyl_{(C≤8)}, arenediyl_{(C≤12)} or a substituted version of any of these groups;
R₉ is hydrogen, amino, halo, hydroxy, or an amino protecting group; or
alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤8)}, or a substituted version of any of these groups;
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 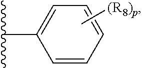
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, acyloxy_{(C≤8)}, alkylsilyloxy_{(C≤8)}, alkylamino_{(C≤8)}, or
dialkylamino_{(C≤8)}, or a substituted version of any of these groups; or
C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 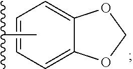
or
R₆ is a group of the formula: 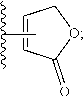
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically accepted salt thereof.

5. The compound according to any one of claims 1-4, wherein the compound is further defined as: 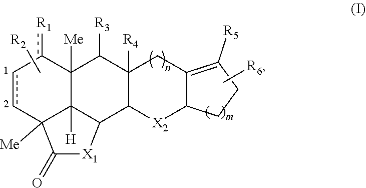
wherein:
the bond between atoms 1 and 2 is a single bond or a double bond;
m is 1 or 2;
n is 0 or 1;
X₁ and X₂ are each independently —O— or —NRₐ—, wherein:
Rₐ is hydrogen, alkyl_{(C≤8)}, or substituted alkyl_{(C≤8)};
R₁ is hydroxy or oxo;
R₂ is hydrogen, amino, halo, hydroxy; or
alkyl_{(C≤8)} or substituted alkyl_{(C≤8)};
R₃ hydrogen, alkyl_{(C≤8)}, substituted alkyl_{(C≤8)}, or —Y₁C(O)R_{b}, wherein:
Y₁ is alkanediyl_{(C≤8)} or substituted alkanediyl_{(C≤8)};
R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₄ is hydrogen, alkyl_{(C≤8)}, or substituted alkyl_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
-A₁-R₇, wherein:
A₁ is heteroarenediyl_{(C≤12)} or substituted heteroarenediyl_{(C≤12)};
R₇ is hydrogen, amino, hydroxy, or an amino protecting group; or
alkyl_{(C≤8)}, cycloalkyl_{(C≤8)}, heterocycloalkyl_{(C≤8)}, heteroaryl_{(C≤12)}, arylsulfonyloxy_{(C≤8)}, or a substituted version of any of these groups; or
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 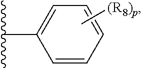
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
—C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 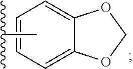
or
-A₂-R₉, wherein:
A₂ is cycloalkanediyl_{(C≤8)}, heterocycloalkanediyl_{(C≤8)}, arenediyl_{(C≤12)} or a substituted version of any of these groups;
R₉ is hydrogen, amino, halo, hydroxy, or an amino protecting group; or
alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤8)}, or a substituted version of any of these groups;
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 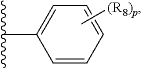
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 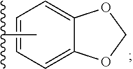
or
R₆ is a group of the formula: 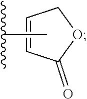
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically acceptable salt thereof.

6. The compound according to any one of claims 1-3, and 5, wherein the compound is further defined as: 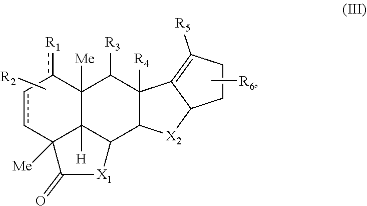
wherein:
the bond between atoms 1 and 2 is a single bond or a double bond;
X₁ and X₂ are each independently —O— or —NRₐ—, wherein:
Rₐ is hydrogen, alkyl_{(C≤8)}, or substituted alkyl_{(C≤8)};
R₁ is hydroxy or oxo;
R₂ is hydrogen, amino, halo, hydroxy; or
alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
Y₁ is alkanediyl_{(C≤8)} or substituted alkanediyl_{(C≤8)};
R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₄ is hydrogen, alkyl_{(C≤8)}, or substituted alkyl_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
-A₁-R₇, wherein:
A₁ is heteroarenediyl_{(C≤12)} or substituted heteroarenediyl_{(C≤12)};
R₇ is hydrogen, amino, hydroxy, or an amino protecting group; or
alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 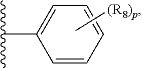
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy(cs), alkylamino_{(C≤8)}, or dialkylamino_{(C≤8)}, or a substituted version of any of these groups; or
—C(O)R₁₃, wherein:
R₁₃ is amino; or
alkyl_{(C≤8)}, alkoxy(c s), alkylamino_{(C≤8)}, or
dialkylamino_{(C≤8)}, or a substituted version of any of these groups; or
a group of the formula: 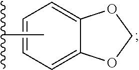
or
-A₂-R₉, wherein:
A₂ is cycloalkanediyl_{(C≤8)}, heterocycloalkanediyl_{(C≤8)}, arenediyl_{(C≤12)} or a substituted version of any of these groups;
R₉ is hydrogen, amino, halo, hydroxy, or an amino protecting group; or
alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤8)}, or a substituted version of any of these groups;
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 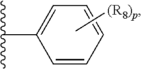
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 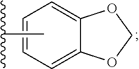
or
R₆ is a group of the formula: 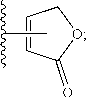
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically acceptable salt thereof.

7. The compound according to any one of claims 1-3, 5, and 6, wherein the compound is further defined as: 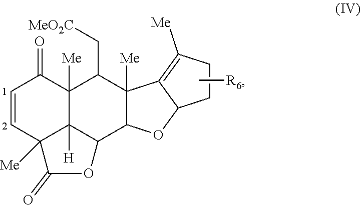
wherein:
R₆ is hydrogen, amino, halo, or hydroxy; or
-A₁-R₇, wherein:
A₁ is heteroarenediyl_{(C≤12)} or substituted heteroarenediyl_{(C≤12)};
R₇ is hydrogen, amino, hydroxy, or an amino protecting group; or
alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 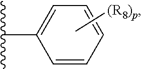
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
—C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 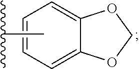
or
-A₂-R₉, wherein:
A₂ is cycloalkanediyl_{(C≤8)}, heterocycloalkanediyl_{(C≤8)}, arenediyl_{(C≤12)} or a substituted version of any of these groups;
R₉ is hydrogen, amino, halo, hydroxy, or an amino protecting group; or
alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤8)}, or a substituted version of any of these groups;
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 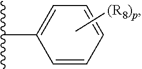
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 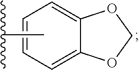
or
R₆ is a group of the formula: 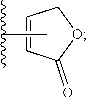
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically acceptable salt thereof.

8. The compound according to any one of claims 1-3, and 5-7, wherein the compound is further defined as: 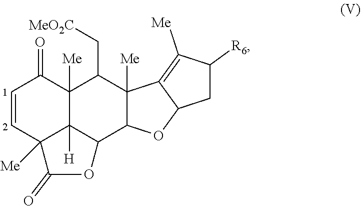
wherein:
R₆ is hydrogen, amino, halo, or hydroxy; or
-A₁-R₇, wherein:
A₁ is heteroarenediyl_{(C≤12)} or substituted heteroarenediyl_{(C≤12)};
R₇ is hydrogen, amino, hydroxy, or an amino protecting group; or
alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 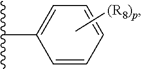
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
—C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 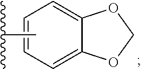
or
-A₂-R₉, wherein:
A₂ is cycloalkanediyl_{(C≤8)}, heterocycloalkanediyl_{(C≤8)}, arenediyl_{(C≤12)} or a substituted version of any of these groups;
R₉ is hydrogen, amino, halo, hydroxy, or an amino protecting group; or
alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤8)}, or a substituted version of any of these groups;
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 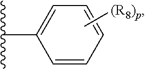
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 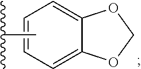
or
R₆ is a group of the formula: 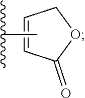
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically acceptable salt thereof.

9. The compound according to any one of claims 1-3, and 5-7, wherein the compound is further defined as: 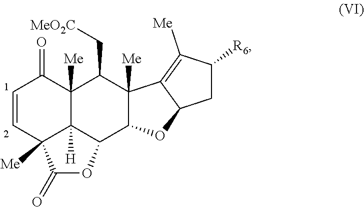
wherein:
R₆ is hydrogen, amino, halo, or hydroxy; or
-A₁-R₇, wherein:
A₁ is heteroarenediyl_{(C≤12)} or substituted heteroarenediyl_{(C≤12)};
R₇ is hydrogen, amino, hydroxy, or an amino protecting group; or
alkyl_{(C≤8)}, cycloalkyl_{(C≤8)}, heterocycloalkyl_{(C≤8)}, heteroaryl_{(C≤12)}, or a substituted version of any of these groups; or
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 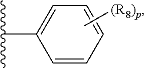
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
—C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 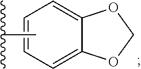
or
-A₂-R₉, wherein:
A₂ is cycloalkanediyl_{(C≤8)}, heterocycloalkanediyl_{(C≤8)}, arenediyl_{(C≤12)} or a substituted version of any of these groups;
R₉ is hydrogen, amino, halo, hydroxy, or an amino protecting group; or
alkyl_{(C≤12)}, cycloalkyl_{(C≤12)}, heterocycloalkyl_{(C≤12)}, aryl_{(C≤18)}, heteroaryl_{(C≤12)}, arylsulfonyloxy_{(C≤8)}, or a substituted version of any of these groups;
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 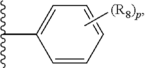
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, acyloxy_{(C≤8)}, alkylsilyloxy_{(C≤8)}, alkylamino_{(C≤8)}, or dialkylamino_{(C≤8)}, or a substituted version of any of these groups; or
C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 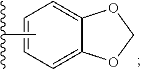
or
R₆ is a group of the formula: 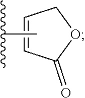
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically acceptable salt thereof.

10. The compound of claim 1, wherein the compound is of formula (I-A).

11. The compound of claim 1, wherein the compound is of formula (I-B).

12. The compound of claim 1, wherein the compound is of formula (I-C).

13. The compound of either claim 1 or claim 2, wherein the compound is further defined as: 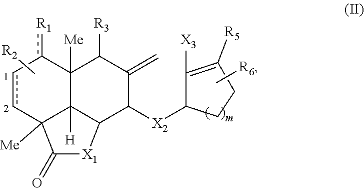
wherein:
the bond between atoms 1 and 2 is a single bond or a double bond;
m is 1 or 2;
X₁ and X₂ are each independently —O— or —NRₐ—, wherein:
Rₐ is hydrogen, alkyl_{(C≤8)}, or substituted alkyl_{(C≤8)};
X₃ is hydrogen, amino, halo, or hydroxy;
R₁ is hydroxy or oxo;
R₂ is hydrogen, amino, halo, hydroxy; or
alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
Y₁ is alkanediyl_{(C≤8)} or substituted alkanediyl_{(C≤8)};
R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
-A₃-R₁₀, wherein:
A₃ is cycloalkanediyl_{(C≤8)}, heterocycloalkanediyl_{(C≤8)}, arenediyl_{(C≤12)} heteroarenediyl_{(C≤12)}, or a substituted version of any of these groups;
R₁₀ is hydrogen, amino, hydroxy, or an amino protecting group; or
alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 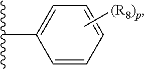
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
—C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 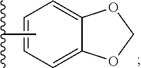
or
R₆ is a group of the formula: 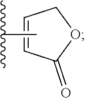
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically accepted salt thereof.

14. The compound according to any one of claims 1, 2, and 13, wherein the compound is further defined as: 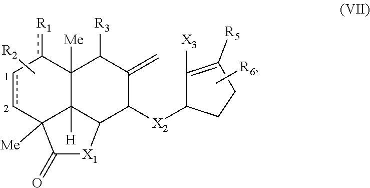
wherein:
the bond between atoms 1 and 2 is a single bond or a double bond;
X₁ and X₂ are each independently —O— or —NRₐ—, wherein:
Rₐ is hydrogen, alkyl_{(C≤8)}, or substituted alkyl_{(C≤8)};
X₃ is hydrogen, amino, halo, or hydroxy;
R₁ is hydroxy or oxo;
R₂ is hydrogen, amino, halo, hydroxy; or
alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
Y₁ is alkanediyl_{(C≤8)} or substituted alkanediyl_{(C≤8)};
R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
-A₃-R₁₀, wherein:
A₃ is cycloalkanediyl_{(C≤8)}, heterocycloalkanediyl_{(C≤8)}, arenediyl_{(C≤12)} heteroarenediyl_{(C≤12)}, or a substituted version of any of these groups;
R₁₀ is hydrogen, amino, hydroxy, or an amino protecting group; or
alkyl_{(C≤8)}, cycloalkyl_{(C≤8)}, heterocycloalkyl_{(C≤8)}, heteroaryl_{(C≤12)}, arylsulfonyloxy_{(C≤8)}, or a substituted version of any of these groups; or
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 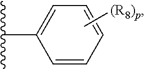
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
—C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 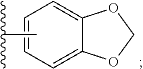
or
R₆ is a group of the formula: 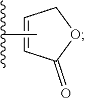
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically accepted salt thereof.

15. The compound according to any one of claims 1, 2, 13, and 14, wherein the compound is further defined as: 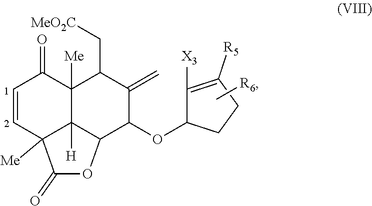
wherein:
X₃ is hydrogen, amino, halo, or hydroxy;
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
-A₃-R₁₀, wherein:
A₃ is cycloalkanediyl_{(C≤8)}, heterocycloalkanediyl_{(C≤8)}, arenediyl_{(C≤12)} heteroarenediyl_{(C≤12)}, or a substituted version of any of these groups;
R₁₀ is hydrogen, amino, hydroxy, or an amino protecting group; or
alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 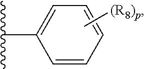
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
—C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 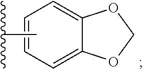
or
R₆ is a group of the formula: 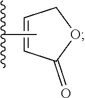
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically accepted salt thereof.

16. The compound according to any one of claims 1, 2, and 13-15, wherein the compound is further defined as: 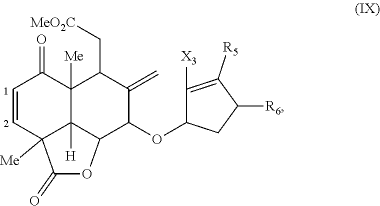
wherein:
X₃ is hydrogen, amino, halo, or hydroxy;
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
-A₃-R₁₀, wherein:
A₃ is cycloalkanediyl_{(C≤8)}, heterocycloalkanediyl_{(C≤8)}, arenediyl_{(C≤12)} heteroarenediyl_{(C≤12)}, or a substituted version of any of these groups;
R₁₀ is hydrogen, amino, hydroxy, or an amino protecting group; or
alkyl_{(C≤8)}, cycloalkyl_{(C≤8)}, heterocycloalkyl_{(C≤8)}, heteroaryl_{(C≤12)}, arylsulfonyloxy_{(C≤18)}, or a substituted version of any of these groups; or
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 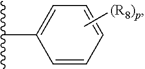
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
—C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 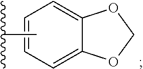
or R₆ is a group of the formula: 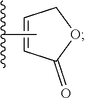
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically accepted salt thereof.

17. The compound according to any one of claims 1, 2, and 13-16, wherein the compound is further defined as: 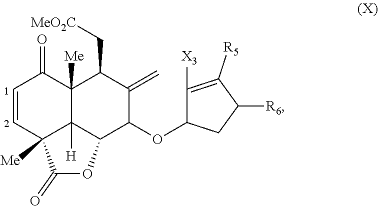
wherein:
X₃ is hydrogen, amino, halo, or hydroxy;
R₅ is hydrogen, alkyl_{(C≤8)}, substituted alkyl_{(C≤8)}, or R₅ is taken together with R₆ as defined below; and
R₆ is hydrogen, amino, halo, or hydroxy; or
-A₃-R₁₀, wherein:
A₃ is cycloalkanediyl_{(C≤8)}, heterocycloalkanediyl_{(C≤8)}, arenediyl_{(C≤12)} heteroarenediyl_{(C≤12)}, or a substituted version of any of these groups;
R₁₀ is hydrogen, amino, hydroxy, or an amino protecting group; or
alkyl_{(C≤8)}, cycloalkyl_{(C≤8)}, heterocycloalkyl_{(C≤8)}, heteroaryl_{(C≤12)}, arylsulfonyloxy_{(C≤8)}, or a substituted version of any of these groups; or
a group of the formula:
—(CH₂)ₓC(O)Rₐ
wherein:
x is 0, 1, or 2;
Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)};
a group of the formula:
—(CH₂)_{y}OR_{b}
or
—(CH₂)_{y}NR_{c}R_{d}
wherein:
y is 0, 1, or 2
R_{b} is a hydroxy protecting group;
R_{c} and R_{d} are each independently a monovalent amino protecting group, or R_{c} and R_{d} are taken together and are a divalent amino protecting group;
a group of the formula: 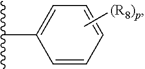
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
—C(O)R₁₃, wherein:
R₁₃ is amino; or
alkyl_{(C≤8)}, alkoxy_{(C≤8)}, alkylamino_{(C≤8)}, or
dialkylamino_{(C≤8)}, or a substituted version of any of these groups; or
a group of the formula: 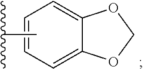
or
R₆ is a group of the formula: 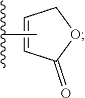
or
R₅ and R₆ are taken together with the atoms to which they are attached and are cycloalkyl_{(C≤8)}, substituted cycloalkyl_{(C≤8)}, heterocycloalkyl_{(C≤8)}, or substituted heterocycloalkyl_{(C≤8)};
or a pharmaceutically accepted salt thereof.

18. The compound of according to any one of claims 1-5, and 10-13, wherein m is 1.

19. The compound according to any one of claims 1-5, and 18, wherein n is 0.

20. The compound according to any one of claims 1-5, and 18, wherein n is 1.

21. The compound according to any one of claims 1-6, 10-12, and 18-20, wherein the bond between atoms 1 and 2 is a single bond.

22. The compound according to any one of claims 1-6, 10-12, 14, and 18-20, wherein the bond between atoms 1 and 2 is a double bond.

23. The compound according to any one of claims 1-6, 10-13, 14, and 18-22, wherein R₁ is hydroxy.

24. The compound according to any one of claims 1-6, 10-13, 14, and 18-22, wherein R₁ is oxo.

25. The compound according to any one of claims 1-6, 10-13, 14, and 18-24, wherein R₂ is hydrogen.

26. The compound according to any one of claims 1-6, 10-13, 14, and 18-25, wherein R₃ is alkyl_{(C≤8)} or substituted alkyl_{(C≤8)}.

27. The compound according to any one of claims 1-6, 10-13, 14, and 18-26, wherein R₃ is substituted alkyl_{(C≤8)}.

28. The compound according to any one of claims 1-6, 10-13, 14, and 18-26, wherein R₃ is (methoxycarbonyl)methyl.

29. The compound according to any one of claims 1-6, 10-13, 14, and 18-25, wherein R₃ is —Y₁C(O)R_{b}.

30. The compound of claim 29, wherein Y₁ is alkanediyl_{(C≤8)}.

31. The compound of claim 30, wherein Y₁ is —CH₂—.

32. The compound according to any one of claims 29-31, wherein R_{b} is alkoxy_{(C≤8)}.

33. The compound of claim 32, wherein R_{b} is methoxy or t-butoxy.

34. The compound according to any one of claims 1-6, 10, 12, and 18-33, wherein R₄ is alkyl_{(C≤8)} or substituted alkyl_{(C≤8)}.

35. The compound according to any one of claims 1-6, 10, 12, and 18-34, wherein R₄ is alkyl_{(C≤8)}.

36. The compound according to any one of claims 1-6, 10, 12, and 18-35, wherein R₄ is methyl.

37. The compound according to any one of claims 1-6 and 10-36, wherein R₅ is alkyl_{(C≤8)} or substituted alkyl_{(C≤8)}.

38. The compound according to any one of claims 1-6 and 10-37, wherein R₅ is alkyl_{(C≤8)}.

39. The compound according to any one of claims 1-6 and 10-38, wherein R₅ is methyl or isopropyl.

40. The compound according to any one of claims 1-39, wherein R₆ is hydrogen.

41. The compound according to any one of claims 1-12 and 18-39, wherein A₂ is heterocycloalkanediyl_{(C≤8)} or substituted heterocycloalkanediyl_{(C≤8)}.

42. The compound of claim 41, wherein A₂ is substituted heterocycloalkanediyl_{(C≤8)}.

43. The compound of claim 42, wherein A₂ is 2-acetoxy-5-oxo-2,5-dihydrofuran-2,3-diyl.

44. The compound according to any one of claims 1-12 and 18-39, wherein A₂ is cycloalkanediyl_{(C≤8)} or substituted cycloalkanediyl_{(C≤8)}.

45. The compound of claim 44, wherein A₂ is cycloalkanediyl_{(C≤8)}.

46. The compound of claim 45, wherein A₂ is cyclopentanediyl or cyclohexanediyl.

47. The compound according to any one of claims 1-12 and 18-39, wherein A₂ is arenediyl_{(C≤12)} or substituted arenediyl_{(C≤12)}.

48. The compound of claim 47, wherein A₂ is arenediyl_{(C≤12)}.

49. The compound of claim 48, wherein A₂ is benzenediyl.

50. The compound of claim 47, wherein A₂ is substituted arenediyl_{(C≤12)}.

51. The compound of claim 50, wherein A₂ is 4-methoxybenzen-1,3-diyl.

52. The compound according to any one of claims 1-12 and 18-51, wherein R₉ is hydrogen.

53. The compound according to any one of claims 1-12, and 18-39, wherein A₁ is heteroarenediyl_{(C≤12)} or substituted heteroarenediyl_{(C≤12)}.

54. The compound according to any one of claims 1-12, 18-39, and 53, wherein A₁ is heteroarenediyl_{(C≤12)}.

55. The compound according to any one of claims 1-12, 18-39, 53, and 54, wherein A₁ is furan-2,3-diyl.

56. The compound according to any one of claims 1-3, 10-12, 18-39, and 53-55, wherein R₇ is hydrogen.

57. The compound according to any one of claims 1, 11, 12, 18-39, and 53-55, wherein R₇ is halo.

58. The compound according to any one of claims 1, 11, 12, 18-39, 53-55, and 57, wherein R₇ is bromo.

59. The compound according to any one of claims 1, 11, 12, 18-39, and 53-55, wherein R₇ is aryl_{(C≤12)} or substituted aryl_{(C≤12)}.

60. The compound according to any one of claims 1, 11, 12, 18-39, 53-55, and 59, wherein R₇ is substituted aryl_{(C≤12)}.

61. The compound according to any one of claims 1, 11, 12, 18-39, 53-55, 59, and 60, wherein R₇ is 4-nitrophenyl, 4-(methoxycarbonyl)phenyl, or 4-methoxy-3-methylphenyl.

62. The compound according to any one of claims 1-12, 18-39 and 53-55, wherein R₇ is heteroaryl_{(C≤12)} or substituted heteroaryl_{(C≤12)}.

63. The compound according to any one of claims 1-12, 18-39, 53-55, and 62, wherein R₇ is heteroaryl_{(C≤12)}.

64. The compound according to any one of claims 1-12, 18-39, 53-55, 62, and 63, wherein R₇ is furan-3-yl or 1-methyl-1H-indol-4-yl.

65. The compound according to any one of claims 1-12, 18-39, 53-55, and 62, wherein R₇ is substituted heteroaryl_{(C≤12)}.

66. The compound according to any one of claims 1-12, 18-39, 53-55, 62, and 65, wherein R₇ is 2-methoxypyridin-5-yl.

67. The compound according to any one of claims 1-12, 18-39 and 53-55, wherein R₇ is a group of the formula:

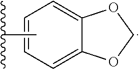

68. The compound according to any one of claims 1-12, 18-39, 53-55, and 66, wherein R₇ is a group of the formula:

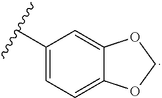 [SMILES: *c1ccc2c(c1)OCO2]

69. The compound according to any one of claims 1-12, 18-39, and 53-55, wherein R₇ is a group of the formula: 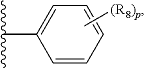
wherein:
p is 0, 1, 2, 3, 4, or 5; and
R₈ is, in each instance independently, hydrogen, hydroxy, halo, amino, cyano, or nitro; or
alkyl_{(C≤8)}, alkoxy_{(C≤8)}, alkylsilyloxy_{(C≤8)}, alkylamino_{(C≤8)}, or dialkylamino_{(C≤8)}, or a substituted version of any of these groups; or
C(O)R₁₃, wherein:
R₁₃ is amino; or
alkyl_{(C≤8)}, alkoxy_{(C≤8)}, alkylamino_{(C≤8)}, or dialkylamino_{(C≤8)}, or a substituted version of any of these groups.

70. The compound according to any one of claims 1-12 and 18-55, wherein R₇ or R₉ are —(CH₂)ₓC(O)Rₐ; wherein: x is 0, 1, or 2; Rₐ is hydrogen, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)}.

71. The compound of claim 70, wherein x is 0.

72. The compound of claim 70, wherein x is 1.

73. The compound according to any one of claims 70-72, wherein Rₐ is hydrogen.

74. The compound according to any one of claims 70-72, wherein Rₐ is hydroxy.

75. The compound according to any one of claims 70-72, wherein Rₐ is alkoxy_{(C≤8)} or substituted alkoxy_{(C≤8)}.

76. The compound according to any one of claims 70-72, wherein Rₐ is alkylamino_{(C≤8)} or substituted alkylamino_{(C≤8)}.

77. The compound of claim 76, wherein Rₐ is t-butylamino.

78. The compound according to any one of claims 1-12 and 18-55, wherein R₇ or R₉ is —(CH₂)_{y}OR_{b}; wherein: y is 0, 1, or 2; and R_{b} is a hydroxy protecting group.

79. The compound of claim 78, wherein y is 0.

80. The compound of claim 78, wherein y is 1.

81. The compound according to any one of claims 78-80, wherein the hydroxy protecting group is an acyl_{(C≤8)} or a alkylsilyl_{(C≤12)} group.

82. The compound of claim 81, wherein the protecting group is pivaloyl group or a tbutyldimethylsilyl group.

83. The compound according to any one of claims 1-12, 18-39, 53-55, and 69, wherein p is 0, 1, or 2.

84. The compound of claim 83, wherein p is 0.

85. The compound of claim 83, wherein p is 1.

86. The compound of claim 83, wherein p is 2.

87. The compound according to any one of claims 1-12, 18-39, 53-55, and 69-86, wherein R₈ is alkyl_{(C≤8)} or substituted alkyl_{(C≤8)}.

88. The compound of claim 87, wherein R₈ is alkyl_{(C≤8)}.

89. The compound of claim 88, wherein R₈ is methyl.

90. The compound according to any one of claims 1-12, 18-39, 53-55, and 69-86, wherein R₈ is alkoxy_{(C≤8)} or substituted alkoxy_{(C≤8)}.

91. The compound of claim 90, wherein R₈ is alkoxy_{(C≤8)}.

92. The compound of claim 91, wherein R₈ is methoxy.

93. The compound according to any one of claims 1-12, 18-39, 53-55, and 69-86, wherein R₈ is alkylsilyloxy_{(C≤8)} or substituted alkylsilyloxy_{(C≤8)}.

94. The compound of claim 93, wherein R₈ is alkylsilyloxy_{(C≤8)}.

95. The compound of claim 94, wherein R₈ is t-butylsilyloxy.

96. The compound according to any one of claims 1-12, 18-39, 53-55, and 69-86, wherein R₈ is nitro.

97. The compound according to any one of claims 1-12, 18-39, 53-55, and 69-86, wherein R₁₃ is alkoxy_{(C≤8)} or substituted alkoxy_{(C≤8)}.

98. The compound of claim 97, wherein R₁₃ is alkoxy_{(C≤8)}.

99. The compound of claim 98, wherein R₁₃ is methoxy.

100. The compound according to any one of claims 1-12, 18-39, 53-55, and 69-86, wherein R₁₃ is alkylamino_{(C≤8)} or substituted alkylamino_{(C≤8)}.

101. The compound of claim 100, wherein R₁₃ is alkylamino_{(C≤8)}.

102. The compound of claim 101, wherein R₁₃ is s-butylamino or t-butylamino.

103. The compound of either claim 1 or claim 11, wherein R₁₄ is hydrogen.

104. The compound according to any one of claims 1, 11, and 103, wherein R_{14′} is hydrogen.

105. The compound according to any one of claims 1, 2, and 13-39, wherein A₃ is heteroarenediyl_{(C≤12)} or substituted heteroarenediyl_{(C≤12)}.

106. The compound according to any one of claims 1, 2, 13-39, and 105, wherein A₃ is heteroarenediyl_{(C≤12)}.

107. The compound according to any one of claims 1, 2, 13-39, 105, and 106, wherein A₃ is furan-2,3-diyl.

108. The compound according to any one of claims 1, 2, 13-39, and 105-107, wherein R₁₀ is hydrogen.

109. The compound according to any one of claims 1, 2, 13-28, 37-39, and 105-108, wherein X₃ is hydrogen.

110. The compound according to any one of claims 1, 2, 13-28, 37-39, and 105-108, wherein X₃ is halo.

111. The compound according to any one of claims 1, 2, 13-28, 37-39, 105-108, and 110, wherein X₃ is iodo.

112. The compound according to any one of claims 1-111, wherein the compound is further defined as: 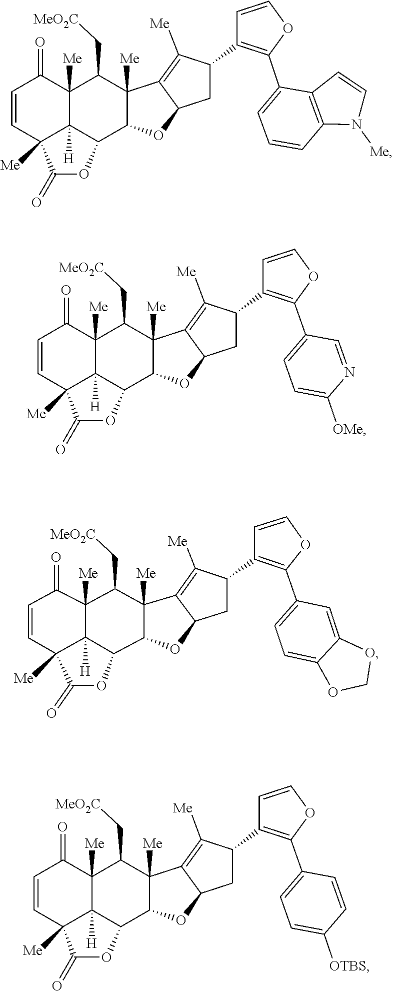 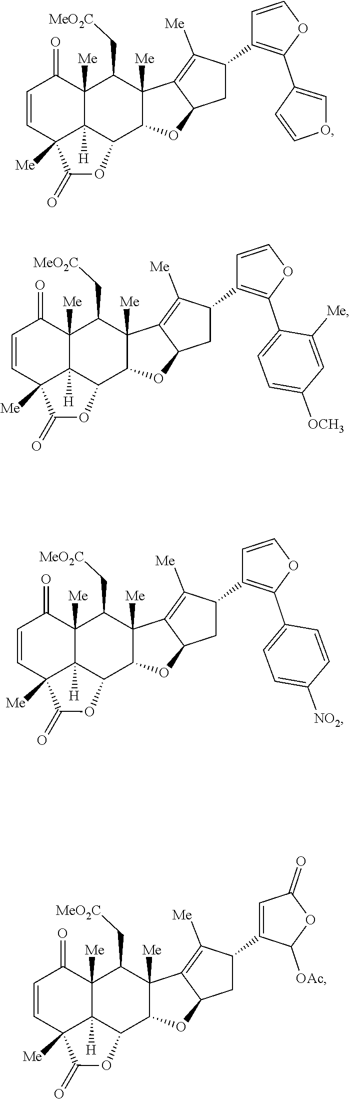 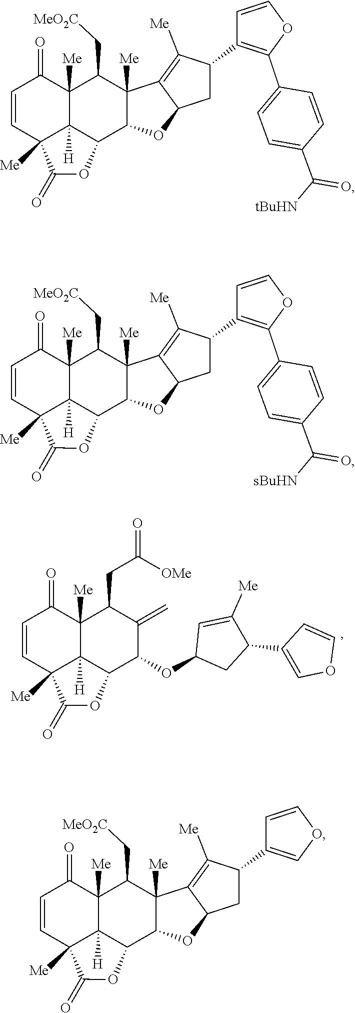 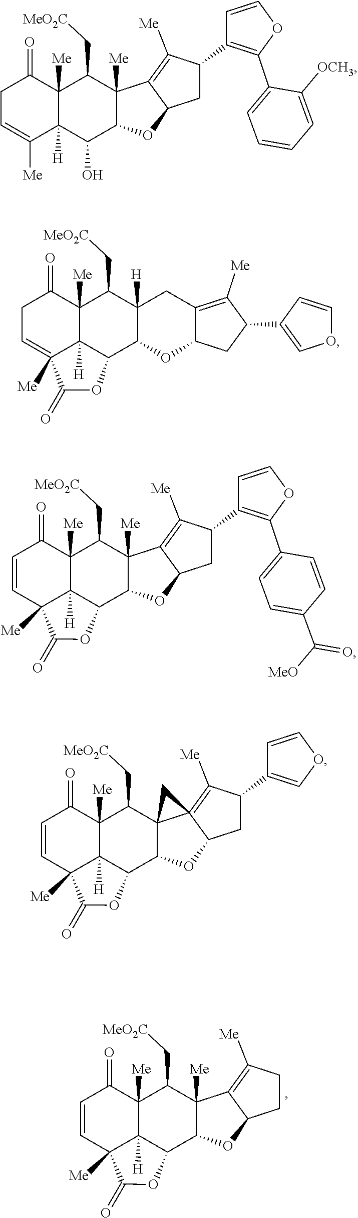 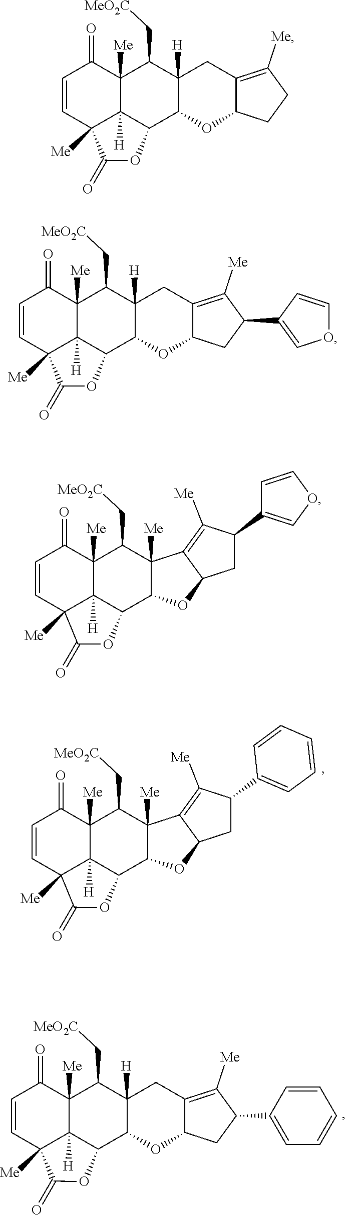 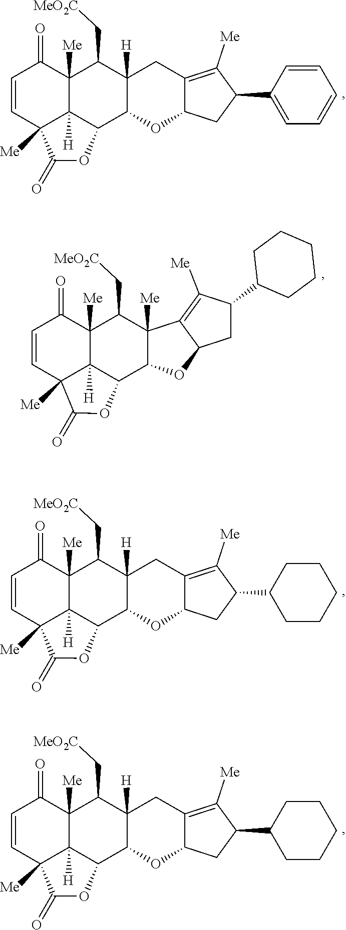 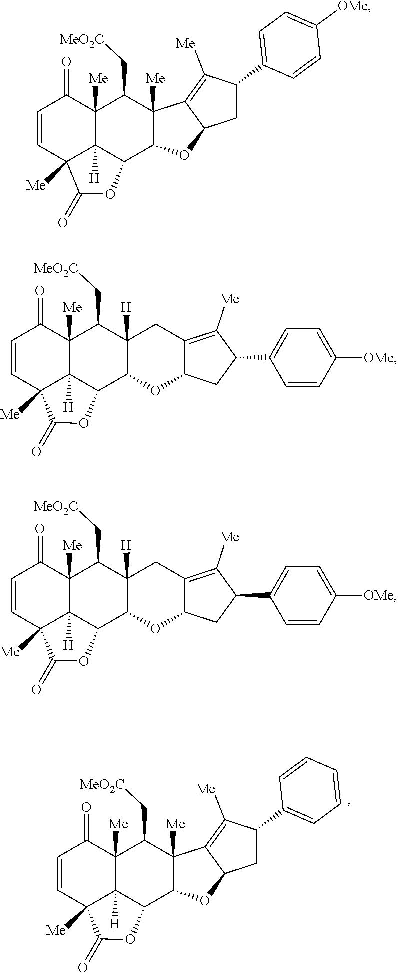 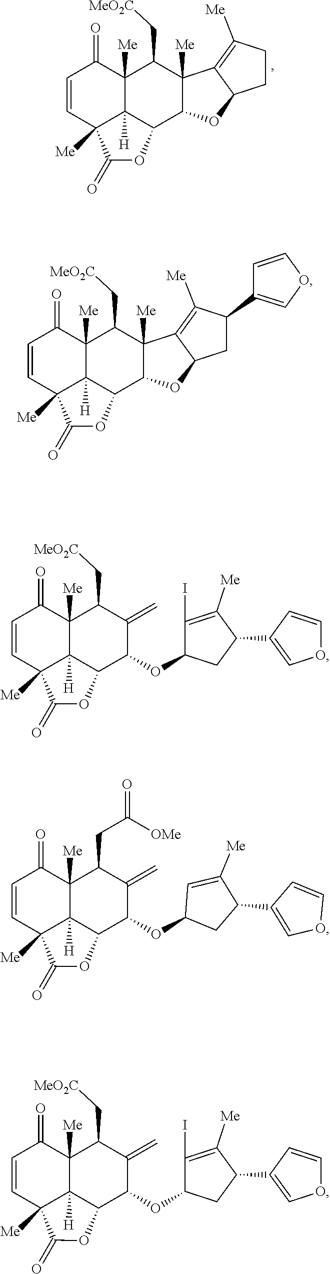 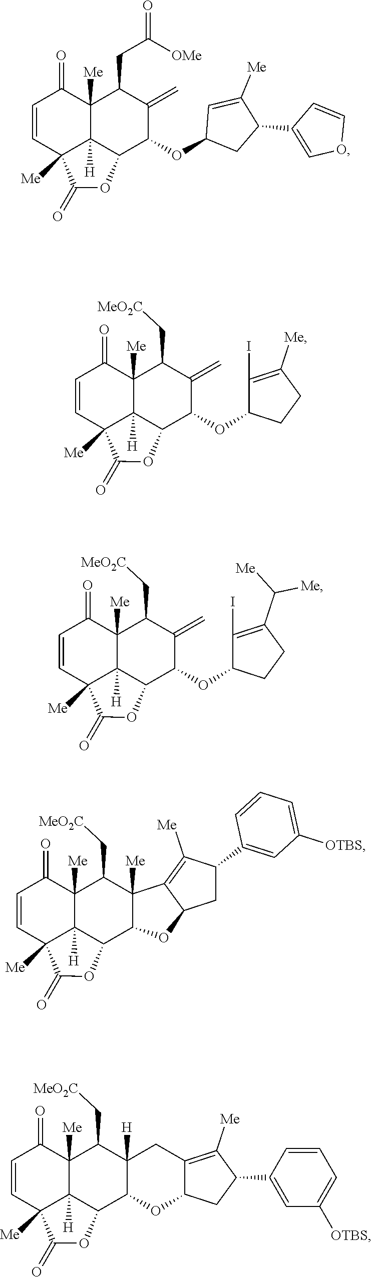 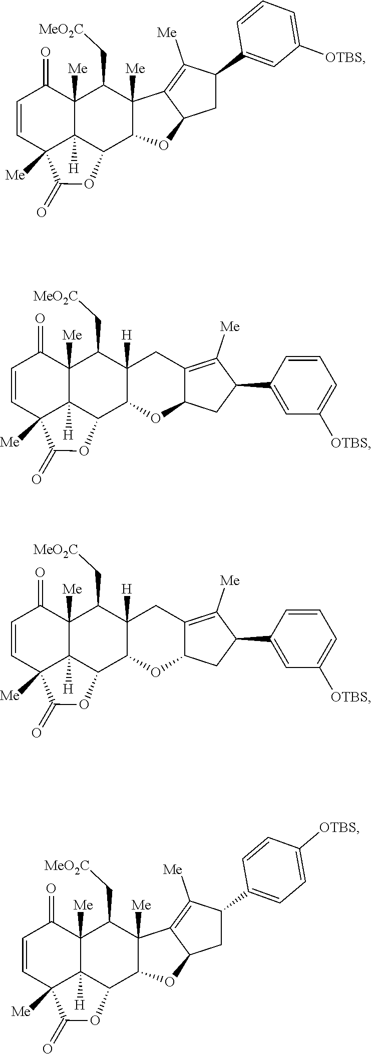 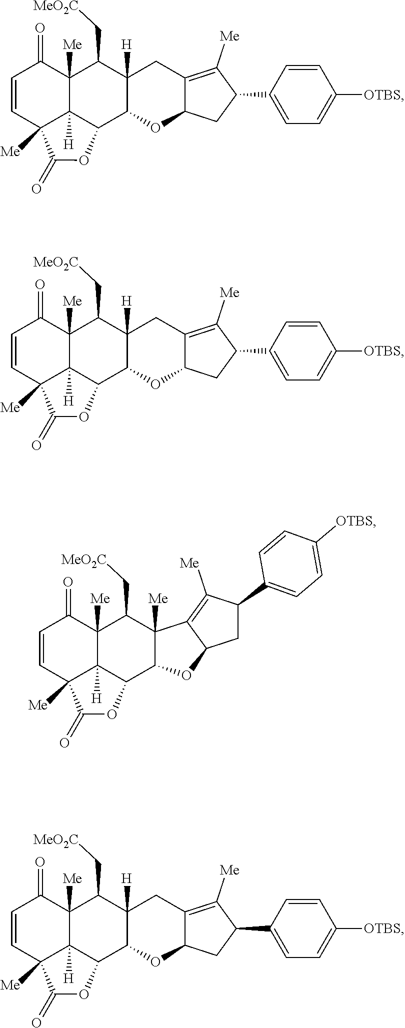 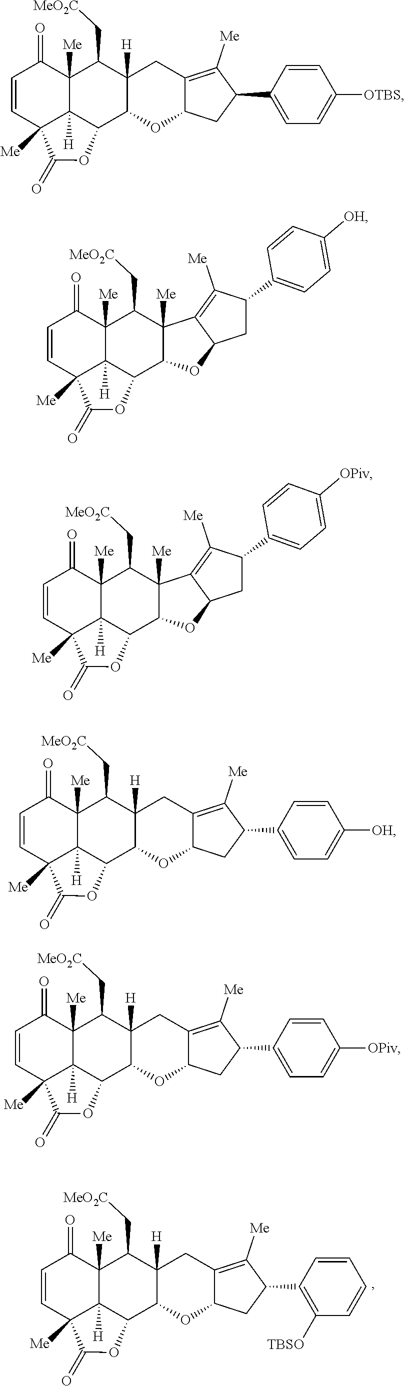 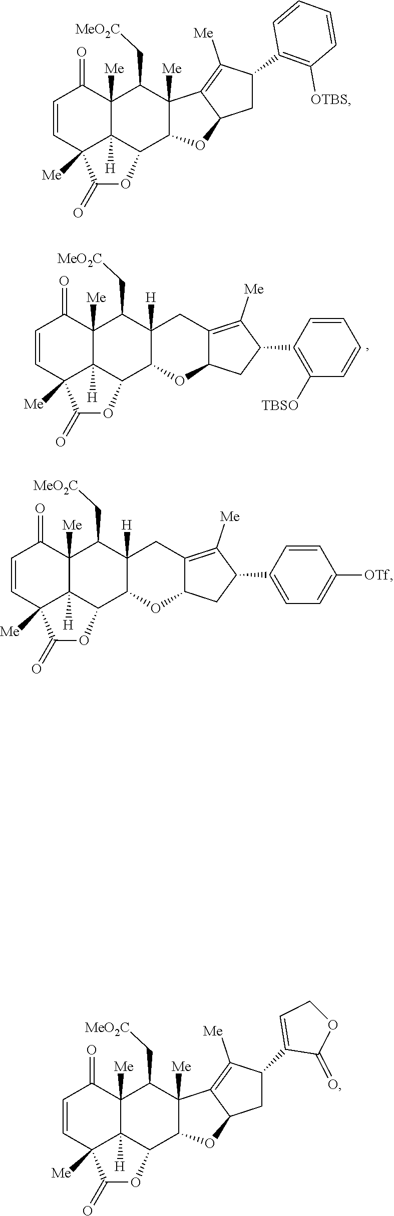 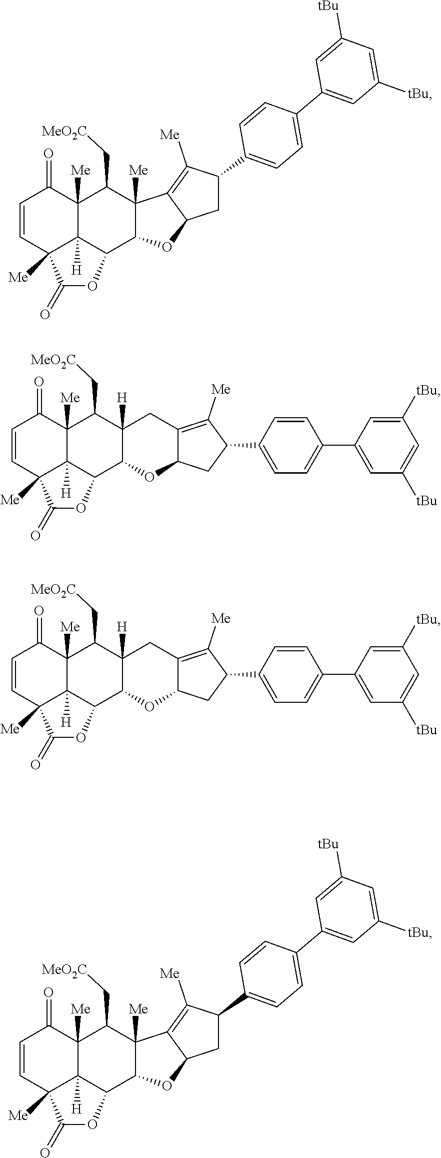 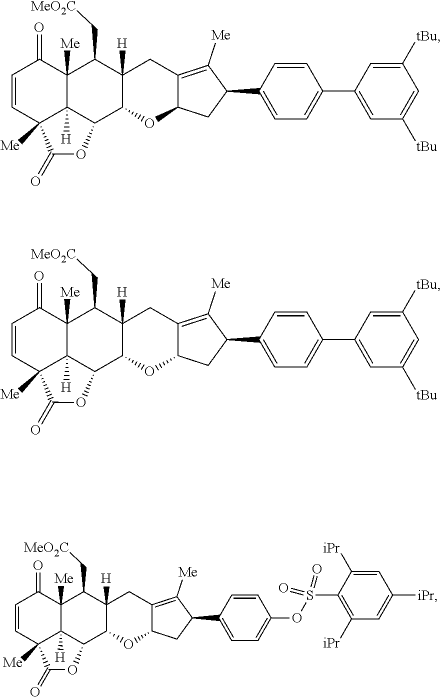 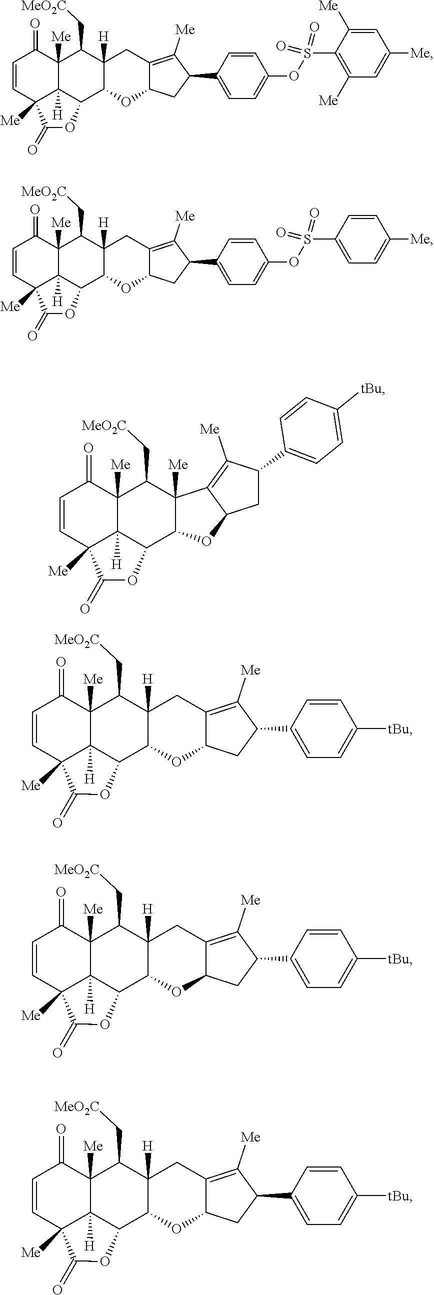 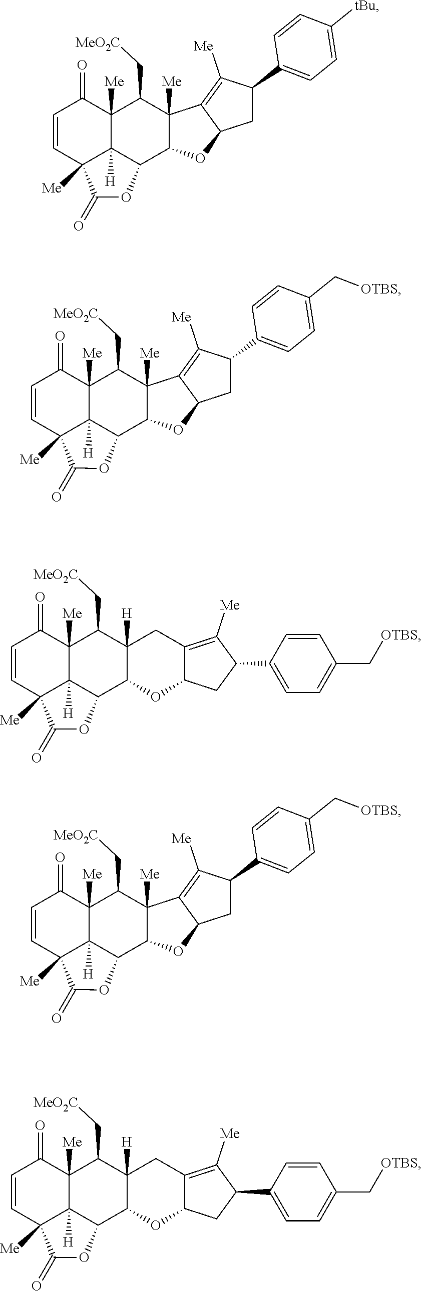 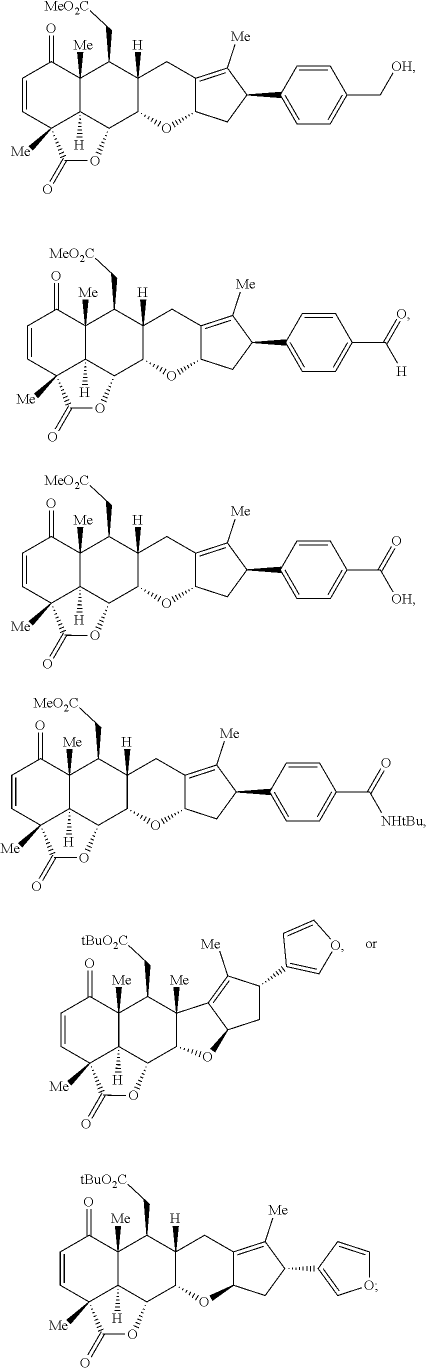
or a pharmaceutically acceptable salt thereof.

113. The compound according to any one of claims 1-86, wherein the compound is further defined as: 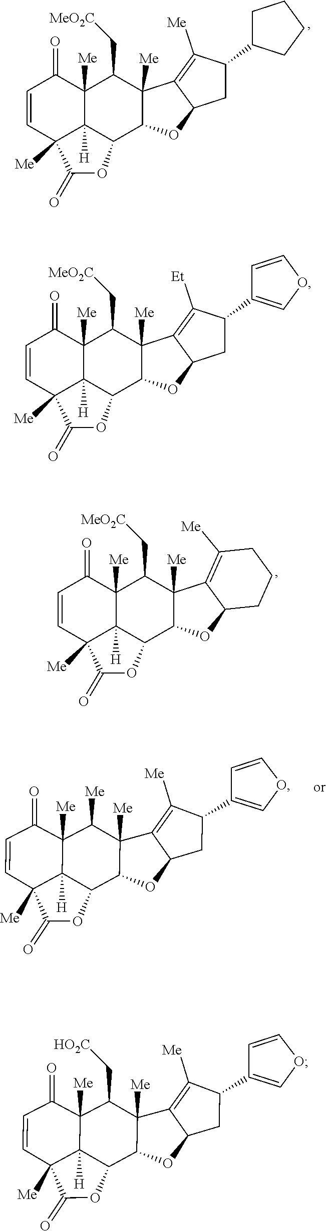
or a pharmaceutical salt thereof.

114. A compound of the formula: 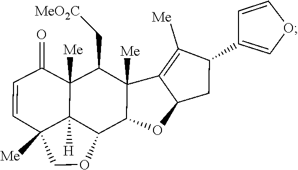
or a pharmaceutical salt thereof.

115. A pharmaceutical composition comprising:
(A) a compound according to any one of claims 1-114; and
(B) an excipient.

116. The pharmaceutical composition of claim 115, wherein the pharmaceutical composition is formulated for administration orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crémes, in lipid compositions, via a catheter, via a lavage, via continuous infusion, via infusion, via inhalation, via injection, via local delivery, or via localized perfusion.

117. The pharmaceutical composition of either claim 115 or claim 116, wherein the pharmaceutical composition is formulated as a unit dose.

118. A method of treating or preventing a disease or disorder in a patient in need thereof comprising administering to the patient a pharmaceutically effective amount of a compound or composition according to any one of claims 1-117.

119. The method of claim 118, wherein the disease or disorder is a cancer or proliferative disease.

120. A method of treating a cancer in a patient in need thereof, the method comprising administering a therapeutically effective amount of a compound that induces the trapping of both PARylated-PARP1 and PAR-binding proteins at DNA lesions.

121. A method of treating a cancer in a patient in need thereof, the method comprising administering a therapeutically effective amount of a compound that inhibits RNF114.

122. The method of claim 121, wherein said compound is not nimbolide.

123. The method of claim 120 or 121, wherein said compound is the compound according to any one of claims 1-114.

124. The method of claim 120 or 121, wherein said cancer is a PARP inhibitor resistant cancer.

125. The method of claim 124, wherein said compound is nimbolide or an analogue or derivative thereof.

126. The method of claim 125, wherein said compound is the compound according to any one of claims 1-114.

127. The method of any one of claims 119-125, wherein said cancer is a lung cancer, abreast cancer, a liver cancer, a kidney cancer, a brain cancer, a head and neck cancer, a testicular cancer, a prostate cancer, an ovarian cancer, a breast cancer, a uterine cancer, a bladder cancer, a skin cancer, an esophageal cancer, a stomach cancer, a pancreatic cancer, a colon cancer, a bone cancer, a Ewing's sarcoma, a thyroid cancer, an endometrial, or a leukemia.

128. The method of any one of claims 121-127, wherein said cancer is deficient in a homologous recombination (HR) dependent deoxyribonucleic acid (DNA) double strand break (DSB) repair pathway.

129. The method of claim 128, wherein said cancer is deficient in breast cancer 1 (BRCA1) and/or breast cancer 2 (BRCA2).

130. The method of claim 129, wherein said cancer is deficient in ATM, ATR, CHK1, CHK2, Rad51, RPA, XRCC3, Fanconi anemia complementation group A (FANCA), Fanconi anemia complementation group (FANCC), Fanconi anemia complementation group D2 (FANCD2), Fanconi anemia complementation group F (FANCF), Fanconi anemia complementation group G (FANCG) or Fanconi anemia complementation group M (FANCM).

131. The method of claim 119-130, wherein said cancer is a PARP inhibitor resistant cancer.

132. The method of claim 131, wherein said PARP inhibitor resistant cancer is intrinsically resistant to PARP inhibitor therapy.

133. The method of claim 131, wherein said PARP inhibitor resistant cancer has acquired resistance to PARP inhibitor therapy.

134. The method of any one of claims 118-133, wherein said compound or composition traps PARP1 at sites of DNA damage.

135. The method of any one of claims 118-133, wherein said compound or composition traps a PAR-binding protein at sites of DNA damage.

136. The method of claim 135, wherein said compound or composition traps a PAR-binding DNA repair factor at sites of DNA damage.

137. The method of claim 135, wherein said compound or composition traps XRCC1 at sites of DNA damage.

138. The method of any one of claims 118-133, wherein said compound or composition traps PARylated-PARP1 at sites of DNA damage.

139. The method of any one of claims 118-133, wherein said compound or composition prevents the degradation of PARylated PARP1.

140. The method of claim 139, wherein said compound or composition inhibits the function of a ubiquitin E3 ligase.

141. The method of claim 140, wherein said compound or composition inhibits the E3 ligase activity of RNF114.

142. The method of any one of claims 118-141, further comprising administering the patient multiple doses of said compound or composition.

143. The method of claim 142, wherein said compound or composition is administered daily, every other day, twice weekly, weekly, every two weeks, monthly or every other month.

144. The method of any one of claims 118-143, wherein administering comprises oral, intravenous, intra-arterial, or subcutaneous administration.

145. The method of any one of claims 118-144, wherein said patient is a human patient.

146. The method of any one of claims 118-144, wherein said patient is a non-human animal patient.

147. The method of any one of claims 118-146, further comprising administering to said patient at least one additional therapeutic.

148. The method of claim 147, wherein said at least one additional therapeutic is an anti-cancer therapy.

149. The method of claim 147, wherein said at least one additional therapeutic is a chemotherapy, a radiation therapy, a hormonal therapy, a toxin therapy, a surgical therapy, a cytokine therapy, or an immunotherapy.

150. The method of claim 149, wherein said immunotherapy is an immune checkpoint inhibitor therapy.

151. The method of claim 150, wherein said immune checkpoint inhibitor therapy targets PD1, PD-L1, CTLA4, STING, cGAS, BTLA, VISTA, TIM-3, LAG3, CD47, CD137, CD40L, ICOS, CD27, KIR, 4-1BB, CD28, TCR, TIGIT, OX40, or GITR.

152. The method of claim 149, wherein said chemotherapy is a DNA damaging agent or an inhibitor of homologous recombination (HR) dependent DNA DSB repair.

153. The method of claim 152, wherein said chemotherapy is an ATM/ATR inhibitor.

154. The method of claim 153, wherein said chemotherapy is a CHK inhibitor.

155. The method of claim 154, wherein said radiation therapy is an ionizing radiation therapy.

156. The method of any one of claims 118-155, wherein the cancer comprises cancer cells defective in homologous recombination.

157. The method of any one of claims 118-156, wherein said cancer has previously been identified as a cancer that is deficient in a homologous recombination (HR) dependent deoxyribonucleic acid (DNA) double strand break (DSB) repair pathway.

158. The method of any one of claims 118-157, wherein the method comprises (a) determining or having determined whether the cancer is defective in homologous recombination; (b) selecting or having selected the patient for treatment with the compound or composition when the cancer is defective in homologous recombination; and (c) administering or having administered to the selected patient the compound or composition.

159. The method of claim 158, wherein step (a) comprises (i) obtaining or having obtained a biological sample from the patient; and (ii) performing or having performed an assay on the biological sample to determine whether the cancer is defective in homologous recombination.

160. The method of any one of claims 118-157, wherein said cancer comprises one or more cancer cells having a reduced or abrogated ability to repair DNA DSB by HR.

161. The method of claim 160, wherein said one or more cancer cells have a reduced or abrogated ability to repair DNA DSB by HR relative to normal cells.

162. The method of any one of claims 118-161, further comprising identifying a cancer cell obtained from the patient as deficient in homologous recombination (HR) dependent deoxyribonucleic acid (DNA) double strand break (DSB) repair relative to normal cells.

163. The method of any one of claims 155-162, wherein said cancer cells are deficient in breast cancer 1 (BRCA1), breast cancer 2 (BRCA2), ATM, ATR, CHK1, CHK2, Rad51, RPA, XRCC3, Fanconi anemia complementation group A (FANCA), Fanconi anemia complementation group (FANCC), Fanconi anemia complementation group D2 (FANCD2), Fanconi anemia complementation group F (FANCF), Fanconi anemia complementation group G (FANCG) and Fanconi anemia complementation group M (FANCM).

164. The method of claim 163, wherein said cancer cells are homozygous for a mutation in BRCA1, BRCA2, ATM, ATR, CHK1, CHK2, Rad51, RPA, XRCC3, FANCA, FANCC, FANCD2, FANCF, FANCG, and FANCM.

165. The method of any one of claims 118-164, wherein said cancer is identified as a HR dependent DNA DSB repair deficient cancer by determining the HR dependent DNA DSB repair activity of cancer cells from the individual relative to normal cells.

166. The method of any one of claims 118-165, wherein said cancer is identified as an HR dependent DSB repair deficient cancer by determining the presence in cancer cells from the individual of one or more mutations or polymorphisms in a nucleic acid sequence encoding a component of the HR dependent DNA DSB repair pathway.

167. The method of any one of claims 118-166, wherein the patient is heterozygous for a mutation in a gene encoding a component of the HR dependent DNA DSB repair pathway.

168. The method of claim 167, wherein the individual is heterozygous for a mutation in ATM, ATR, CHK1, CHK2, Rad51, RPA, XRCC3, BRCA1, and/or BRCA2.

169. An intermediate of the formula: 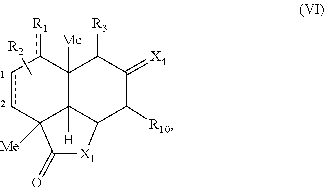
wherein:
the bond between atoms 1 and 2 is a single bond or a double bond;
X₁ is —O— or —NRₐ—, wherein:
Rₐ is hydrogen, alkyl_{(C≤8)}, or substituted alkyl_{(C≤8)};
X₄ is ═O or ═CH₂;
R₁ is hydroxy or oxo;
R₂ is hydrogen, amino, halo, hydroxy; or
alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
Y₁ is alkanediyl_{(C≤8)} or substituted alkanediyl_{(C≤8)};
R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)}; and
R₁₀ is amino or hydroxy; or
alkoxy_{(C≤8)}, acyloxy_{(C≤8)}, alkylsilyloxy_{(C≤8)}, alkylamino_{(C≤8)}, dialkylamino_{(C≤8)}, amido_{(C≤8)}, or a substituted version of any of these groups; or
an intermediate of the formula: 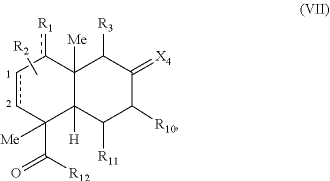
wherein:
the bond between atoms 1 and 2 is a single bond or a double bond;
X₄ is ═O or ═CH₂;
R₁ is hydroxy or oxo;
R₂ is hydrogen, amino, halo, hydroxy; or
alkyl_{(C≤8)} or substituted alkyl_{(C≤8)};
R₃ is hydrogen, alkyl_{(C≤8)}, or substituted alkyl_{(C≤8)}; and
R₁₀ and R₁₁ are each independently amino or hydroxy; or
alkoxy_{(C≤8)}, acyloxy_{(C≤8)}, alkylsilyloxy_{(C≤8)}, alkylamino_{(C≤8)}, dialkylamino_{(C≤8)}, amido_{(C≤8)}, or a substituted version of any of these groups; or
R₁₂ is hydroxy or amino; or
alkoxy_{(C≤8)}, alkylamino_{(C≤8)}, dialkylamino_{(C≤8)}, or a substituted version of any of these groups;
or
an intermediate of the formula: 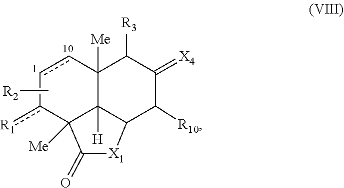
wherein:
the bond between atoms 1 and 10 is a single bond or a double bond;
X₁ is —O— or —NRₐ—, wherein:
Rₐ is hydrogen, alkyl_{(C≤8)}, or substituted alkyl_{(C≤8)};
X₄ is ═O or ═CH₂;
R₁ is hydroxy or oxo;
R₂ is hydrogen, amino, halo, hydroxy; or
alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
Y₁ is alkanediyl_{(C≤8)} or substituted alkanediyl_{(C≤8)};
R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)}; and
R₁₀ is amino or hydroxy; or
alkoxy_{(C≤8)}, acyloxy_{(C≤8)}, alkylsilyloxy_{(C≤8)}, alkylamino_{(C≤8)}, dialkylamino_{(C≤8)}, amido_{(C≤8)}, or a substituted version of any of these groups; or
or a salt thereof.

170. The intermediate of claim 169, wherein the intermediate is further defined as: 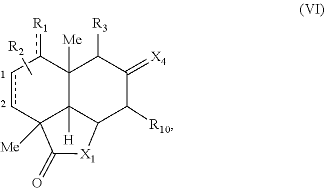
wherein:
the bond between atoms 1 and 2 is a single bond or a double bond;
X₁ is —O— or —NRₐ—, wherein:
Rₐ is hydrogen, alkyl_{(C≤8)}, or substituted alkyl_{(C≤8)};
X₄ is ═O or ═CH₂;
R₁ is hydroxy or oxo;
R₂ is hydrogen, amino, halo, hydroxy; or
alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
Y₁ is alkanediyl_{(C≤8)} or substituted alkanediyl_{(C≤8)};
R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)}; and
R₁₀ is amino or hydroxy; or
alkoxy_{(C≤8)}, acyloxy_{(C≤8)}, alkylsilyloxy_{(C≤8)}, alkylamino_{(C≤8)}, dialkylamino_{(C≤8)}, amido_{(C≤8)}, or a substituted version of any of these groups;
or a salt thereof.

171. The intermediate of claim 169 or claim 170, wherein the intermediate is further defined as: 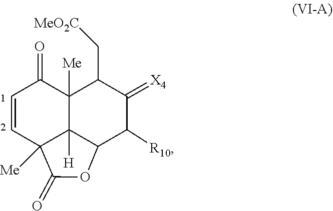
wherein:
X₄ is ═O or ═CH₂; and
R₁₀ is amino or hydroxy; or
alkoxy_{(C≤8)}, acyloxy_{(C≤8)}, alkylsilyloxy_{(C≤8)}, alkylamino_{(C≤8)}, dialkylamino_{(C≤8)}, amido_{(C≤8)}, or a substituted version of any of these groups;
or a salt thereof.

172. The intermediate of claim 169, wherein the intermediate is further defined as: 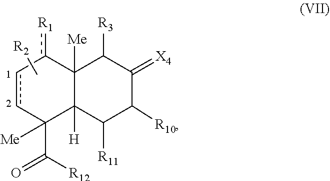
wherein:
the bond between atoms 1 and 2 is a single bond or a double bond;
X₄ is ═O or ═CH₂;
R₁ is hydroxy or oxo;
R₂ is hydrogen, amino, halo, hydroxy; or
alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
Y₁ is alkanediyl_{(C≤8)} or substituted alkanediyl_{(C≤8)};
R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)}; and
R₁₀ and R₁₁ are each independently amino or hydroxy; or
alkoxy_{(C≤8)}, acyloxy_{(C≤8)}, alkylsilyloxy_{(C≤8)}, alkylamino_{(C≤8)}, dialkylamino_{(C≤8)}, amido(c8), or a substituted version of any of these groups; or
R₁₂ is hydroxy or amino; or
alkoxy_{(C≤8)}, alkylamino_{(C≤8)}, dialkylamino_{(C≤8)}, or a substituted version of any of these groups;
or a salt thereof.

173. The intermediate of either claim 169 or claim 172, wherein the intermediate is further defined as: 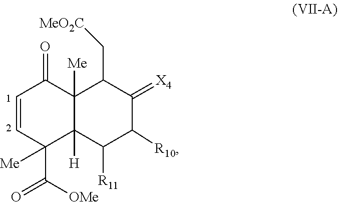
wherein:
the bond between atoms 1 and 2 is a single bond or a double bond;
X₄ is ═O or ═CH₂;
R₁₀ and Rn are each independently amino or hydroxy; or
alkoxy_{(C≤8)}, acyloxy_{(C≤8)}, alkylsilyloxy_{(C≤8)}, alkylamino_{(C≤8)}, dialkylamino_{(C≤8)}, amido_{(C≤8)}, or a substituted version of any of these groups;
or a salt thereof.

174. The intermediate of claim 169, wherein the intermediate is further defined as: 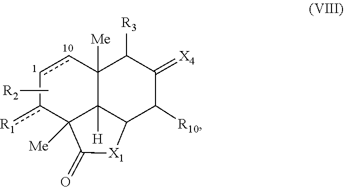
wherein:
the bond between atoms 1 and 10 is a single bond or a double bond;
X₁ is —O— or —NRₐ—, wherein:
Rₐ is hydrogen, alkyl_{(C≤8)}, or substituted alkyl_{(C≤8)};
X₄ is ═O or ═CH₂;
R₁ is hydroxy or oxo;
R₂ is hydrogen, amino, halo, hydroxy; or
alkyl_{(C≤8)} or substituted alkyl_{(C≤8)};
R₃ is hydrogen, alkyl_{(C≤8)}, substituted alkyl_{(C≤8)}, or —Y₁C(O)R_{b}, wherein:
Y₁ is alkanediyl_{(C≤8)} or substituted alkanediyl_{(C≤8)};
R_{b} is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, or substituted dialkylamino_{(C≤8)}; and
R₁₀ is amino or hydroxy; or
alkoxy_{(C≤8)}, acyloxy_{(C≤8)}, alkylsilyloxy_{(C≤8)}, alkylamino_{(C≤8)}, dialkylamino_{(C≤8)}, amido_{(C≤8)}, or a substituted version of any of these groups; or
or a salt thereof.

175. The intermediate of either claim 169 or claim 174, wherein the intermediate is further defined as: 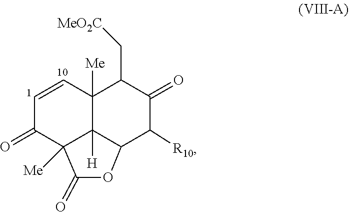
wherein:
R₁₀ is amino or hydroxy; or
alkoxy_{(C≤8)}, acyloxy_{(C≤8)}, alkylsilyloxy_{(C≤8)}, alkylamino_{(C≤8)}, dialkylamino_{(C≤8)}, amido_{(C≤8)}, or a substituted version of any of these groups;
or a salt thereof.

176. The intermediate according to any one of claims 169, 170, and 172, wherein the bond between atoms 1 and 2 is a double bond.

177. The intermediate of either claim 169 or claim 174, wherein the bond between atoms 1 and 10 is a double bond.

178. The intermediate according to any one of claims 169, 170, 172, 174, 176, and 177, wherein R₁ is oxo.

179. The intermediate according to any one of claims 169, 170, 172, 174, and 176-178, wherein R₂ is hydrogen.

180. The intermediate according to any one of claims 169, 170, 172, 174, and 176-179, wherein R₃ is alkyl_{(C≤8)} or substituted alkyl_{(C≤8)}.

181. The intermediate according to any one of claims 169, 170, 172, 174, and 176-180, wherein R₃ is substituted alkyl_{(C≤8)}.

182. The intermediate according to any one of claims 169, 170, 172, 174, and 176-181, wherein R₃ is (methoxycarbonyl)methyl.

183. The intermediate according to any one of claims 169, 170, 172, and 176-182, wherein X₁ is —O—.

184. The intermediate according to any one of claims 169-172, 174, and 176-182, wherein X₄ is ═O.

185. The intermediate according to any one of claims 169-172, 174, and 176-182, wherein X₄ is ═CH₂.

186. The intermediate according to any one of claims 169-185, wherein R₁₀ is hydroxy.

187. The intermediate according to any one of claims 169-185, wherein R₁₀ is alkylsilyloxy_{(C≤8)} or substituted alkylsilyloxy_{(C≤8)}.

188. The intermediate according to any one of claims 169-185 and 187, wherein R₁₀ is alkylsilyloxy_{(C≤8)}.

189. The intermediate according to any one of claims 169-185, 187, and 188, wherein R₁₀ is trimethylsilyloxy or triethylsilyloxy.

190. The intermediate according to any one of claims 169, 172, 173, and 176-189, wherein R₁₁ is hydroxy.

191. The intermediate according to any one of claims 169, 172, 173, and 176-189, wherein R₁₁ is alkylsilyloxy_{(C≤8)} or substituted alkylsilyloxy_{(C≤8)}.

192. The intermediate according to any one of claims 169, 172, 173, 176-189, and 191, wherein Ru is alkylsilyloxy_{(C≤8)}.

193. The intermediate according to any one of claims 169, 172, 173, 176-189, 191, and 192, wherein Rn is trimethylsilyloxy.

194. The intermediate according to any one of claims 169, 172, and 176-189, wherein R₁₂ is alkoxy_{(C≤8)} or substituted alkoxy_{(C≤8)}.

195. The intermediate according to any one of claims 169, 172, 176-189, and 194, wherein R₁₂ is alkoxy_{(C≤8)}.

196. The intermediate according to any one of claims 169, 172, 176-189, 194, and 195, wherein R₁₂ is methoxy.

197. The intermediate according to any one of claims 169-196, wherein the intermediate is further defined as: 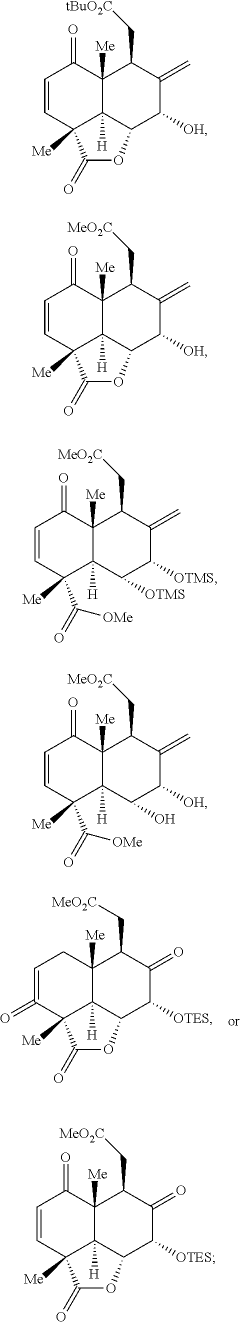
or a salt thereof.

198. A method of manufacturing a compound according to any one of claims 1-114 comprising contacting an intermediate according to any one of claims 169-197 with a base.

199. The method of claim 198, wherein the base is an inorganic base.

200. The method of either claim 198 or claim 199, wherein the base is a salt.

201. The method according to any one of claims 198-200, wherein the base is K₂CO₃.
